SEQUENCE LISTING (162 sequences)

SEQ ID NO: 1 - protein, 130 aa, Artificial Sequence
  Synthetic peptide
XXXXXXXXXXXXXXXXXXGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 2 - protein, 4 aa, Artificial Sequence
  Synthetic peptide
XXXX

SEQ ID NO: 3 - protein, 8 aa, Artificial Sequence
  Synthetic peptide
XXXXXXGX

SEQ ID NO: 4 - protein, 4 aa, Artificial Sequence
  Synthetic peptide
GLGF

SEQ ID NO: 5 - protein, 4 aa, Artificial Sequence
  Synthetic peptide
ESDV

SEQ ID NO: 6 - protein, 4 aa, Artificial Sequence
  Synthetic peptide
QTSV

SEQ ID NO: 7 - protein, 4 aa, Artificial Sequence
  Synthetic peptide
QTRV

SEQ ID NO: 8 - protein, 4 aa, Artificial Sequence
  Synthetic peptide
EYYV

SEQ ID NO: 9 - protein, 4 aa, Artificial Sequence
  Synthetic peptide
PVPV

SEQ ID NO: 10 - protein, 4 aa, Artificial Sequence
  Synthetic peptide
VDSV

SEQ ID NO: 11 - protein, 6 aa, Artificial Sequence
  Synthetic peptide
SLESCF

SEQ ID NO: 12 - protein, 94 aa, Artificial Sequence
  Synthetic peptide
VVRIVKSESGYGFNVRGQVSEGGQLRSINGELYAPLQHVSAVLPGGAADRAGVRKGDRILEVNGVNVEGATHKQVVDLIRAGEKELILTVLSVP

SEQ ID NO: 13 - protein, 6 aa, Artificial Sequence
  Synthetic peptide
LWETSI

SEQ ID NO: 14 - protein, 87 aa, Artificial Sequence
  Synthetic peptide
EITLERGNSGLGFSIAGGTDNPHIGDDPSIFITKIIPGGAAAQDGRLRVNDSILFVNEVDVREVTHSAAVEALKEAGSIVRLYVMRR

SEQ ID NO: 15 - protein, 6 aa, Artificial Sequence
  Synthetic peptide
TNDSLL

SEQ ID NO: 16 - protein, 81 aa, Artificial Sequence
  Synthetic peptide
LCCLEKGPNGYGFHLHGEKGKLGQYIRLVEPGSPAEKAGLLAGDRLVEVNGENVEKETHQQVVSRIRAALNAVRLLVVDPE

SEQ ID NO: 17 - protein, 6 aa, Artificial Sequence
  Synthetic peptide
YLVTSV

SEQ ID NO: 18 - protein, 86 aa, Artificial Sequence
  Synthetic peptide
EVELAKTDGSLGISVTGGVNTSVRHGGIYVKAIIPKGAAESDGRIHKGDRVLAVNGVSLEGATHKQAVETLRNTGQVVHLLLEKGQ

SEQ ID NO: 19 - protein, 6 aa, Artificial Sequence
  Synthetic peptide
WDETNL

SEQ ID NO: 20 - protein, 95 aa, Artificial Sequence
  Synthetic peptide
TVLLQKKDSEGFGFVLRGAKAQTPIEEFTPTPAFPALQYLESVDEGGVAWRAGLRMGDFLIEVNGQNVVKVGHRQVVNMIRQGGNTLMVKVVMVT

SEQ ID NO: 21 - protein, 6 aa, Artificial Sequence
  Synthetic peptide
SLETSL

SEQ ID NO: 22 - protein, 80 aa, Artificial Sequence
  Synthetic peptide
LCRLVRGEQGYGFHLHGEKGRRGQFIRRVEPGSPAEAAALRAGDRLVEVNGVNVEGETHHQVVQRIKAVEGQTRLLVVDQ

SEQ ID NO: 23 - protein, 81 aa, Artificial Sequence
  Synthetic peptide
RLCHLRKGPQGYGFNLHSDKSRPGQYIRSVDPGSPAARSGLRAQDRLIEVNGQNVEGLRHAEVVASIKAREDEARLLVVDP

SEQ ID NO: 24 - protein, 6 aa, Artificial Sequence
  Synthetic peptide
SLETSV

SEQ ID NO: 25 - protein, 81 aa, Artificial Sequence
  Synthetic peptide
KVVLHKGSTGLGFNIVGGEDGEGIFVSFILAGGPADLSGELQRGDQILSVNGIDLRGASHEQAAAALKGAGQTVTIIAQYQ

SEQ ID NO: 26 - protein, 88 aa, Artificial Sequence
  Synthetic peptide
EEIVLERGNSGLGFSIAGGIDNPHVPDDPGIFITKIIPGGAAAMDGRLGVNDCVLRVNEVDVSEVVHSRAVEALKEAGPVVRLVVRRR

SEQ ID NO: 27 - protein, 87 aa, Artificial Sequence
  Synthetic peptide
TVEMKKGPTDSLGISIAGGVGSPLGDVPIFIAMMHPTGVAAQTQKLRVGDRIVTICGTSTEGMTHTQAVNLLKNASGSIEMQVVAGG

SEQ ID NO: 28 - protein, 6 aa, Artificial Sequence
  Synthetic peptide
TLVSTV

SEQ ID NO: 29 - protein, 86 aa, Artificial Sequence
  Synthetic peptide
EVNLLKGPKGLGFSIAGGIGNQHIPGDNSIYITKIIEGGAAQKDGRLQIGDRLLAVNNTNLQDVRHEEAVASLKNTSDMVYLKVAK

SEQ ID NO: 30 - protein, 6 aa, Artificial Sequence
  Synthetic peptide
LFSTEV

SEQ ID NO: 31 - protein, 87 aa, Artificial Sequence
  Synthetic peptide
IITVTLKKQNGMGLSIVAAKGAGQDKLGIYVKSVVKGGAADVDGRLAAGDQLLSVDGRSLVGLSQERAAELMTRTSSVVTLEVAKQG

SEQ ID NO: 32 - protein, 6 aa, Artificial Sequence
  Synthetic peptide
LEITEL

SEQ ID NO: 33 - protein, 83 aa, Artificial Sequence
  Synthetic peptide
KVFISLVGSRGLGCSISSGPIQKPGIFISHVKPGSLSAEVGLEIGDQIVEVNGVDFSNLDHKEAVNVLKSSRSLTISIVAAAG

SEQ ID NO: 34 - protein, 6 aa, Artificial Sequence
  Synthetic peptide
RRTTPV

SEQ ID NO: 35 - protein, 87 aa, Artificial Sequence
  Synthetic peptide
EIKLIKGPKGLGFSIAGGVGNQHIPGDNSIYVTKIIEGGAAHKDGRLQIGDKILAVNSVGLEDVMHEDAVAALKNTYDVVYLKVAKP

SEQ ID NO: 36 - protein, 6 aa, Artificial Sequence
  Synthetic peptide
VQDTRL

SEQ ID NO: 37 - protein, 81 aa, Artificial Sequence; identical to SEQ ID NO: 16
  Synthetic peptide

SEQ ID NO: 38 - protein, 6 aa, Artificial Sequence
  Synthetic peptide
LNSTTL

SEQ ID NO: 39 - protein, 81 aa, Artificial Sequence
  Synthetic peptide
LCTMKKGPSGYGFNLHSDKSKPGQFIRSVDPDSPAEASGLRAQDRIVEVNGVCMEGKQHGDVVSAIRAGGDETKLLVVDRE

SEQ ID NO: 40 - protein, 6 aa, Artificial Sequence
  Synthetic peptide
HSTTRV

SEQ ID NO: 41 - protein, 81 aa, Artificial Sequence
  Synthetic peptide
RIVIHRGSTGLGFNIVGGEDGEGIFISFILAGGPADLSGELRKGDQILSVNGVDLRNASHEQAAIALKNAGQTVTIIAQYK

SEQ ID NO: 42 - protein, 6 aa, Artificial Sequence
  Synthetic peptide
YKQTSV

SEQ ID NO: 43 - protein, 6 aa, Artificial Sequence
  Synthetic peptide
WFDTDL

SEQ ID NO: 44 - protein, 98 aa, Artificial Sequence
  Synthetic peptide
RVEIHKLRQGENLILGFSIGGGIDQDPSQNPFSEDKTDKGIYVTRVSEGGPAEIAGLQIGDKIMQVNGWDMTMVTHDQARKRLTKRSEEVVRLLVTRQ

SEQ ID NO: 45 - protein, 6 aa, Artificial Sequence
  Synthetic peptide
KLMTTV

SEQ ID NO: 46 - protein, 73 aa, Artificial Sequence
  Synthetic peptide
TVTLNMEKYNFLGISIVGQSNERGDGGIYIGSIMKGGAVAADGRIEPGDMLLQVNDINFENMSNDDAVRVLRD

SEQ ID NO: 47 - protein, 6 aa, Artificial Sequence
  Synthetic peptide
GYSTRL

SEQ ID NO: 48 - protein, 81 aa, Artificial Sequence; identical to SEQ ID NO: 23
  Synthetic peptide

SEQ ID NO: 49 - protein, 6 aa, Artificial Sequence
  Synthetic peptide
EAQTRL

SEQ ID NO: 50 - protein, 95 aa, Artificial Sequence; identical to SEQ ID NO: 20
  Synthetic peptide

SEQ ID NO: 51 - protein, 6 aa, Artificial Sequence
  Synthetic peptide
KIATLV

SEQ ID NO: 52 - protein, 93 aa, Artificial Sequence
  Synthetic peptide
RVELWREPSKSLGISIVGGRGMGSRLSNGEVMRGIFIKHVLEDSPAGKNGTLKPGDRIVEVDGMDLRDASHEQAVEAIRKAGSPVVFMVQSIV

SEQ ID NO: 53 - protein, 84 aa, Artificial Sequence
  Synthetic peptide
TIEISKGQTGLGLSIVGGSDTLLGAIIIHEVYEEGAACKDGRLWAGDQILEVNGIDLRKATHDEAINVLRQTPQRVRLTLYRDE

SEQ ID NO: 54 - protein, 6 aa, Artificial Sequence
  Synthetic peptide
QDGTEV

SEQ ID NO: 55 - protein, 186 aa, Artificial Sequence
  Synthetic peptide
VVELMKKEGTTLGLTVSGGIDKDGKPRVSNLRQGGIAARSDQLDVGDYIKAVNGINLAKFRHDEIISLLKNVGERVVLEVEYELPPVSIQGSSVMFRTVEVTLHKEGNTFGFVIRGGAHDDRNKSRPVVITCVRPGGPADREGTIKPGDRLLSVDGIRLLGTTHAEAMSILKQCGQEATLLIEYDV

SEQ ID NO: 56 - protein, 6 aa, Artificial Sequence
  Synthetic peptide
EEESQL

SEQ ID NO: 57 - protein, 90 aa, Artificial Sequence
  Synthetic peptide
VLTLEKGDNQTFGFEIQTYGLHHREEQRVEMVTFVCRVHESSPAQLAGLTPGDTIASVNGLNVEGIRHREIVDIIKASGNVLRLETLYGT

SEQ ID NO: 58 - protein, 6 aa, Artificial Sequence
  Synthetic peptide
LGATGL

SEQ ID NO: 59 - protein, 87 aa, Artificial Sequence
  Synthetic peptide
EIKLIKGPKGLGFSIAGGVGNQHIPGDNSIYVTKIIEGGAAHKDGKLQIGDKLLAVNSVCLEEVTHEEAVTALKNTSDFVYLKAAKP

SEQ ID NO: 60 - protein, 6 aa, Artificial Sequence
  Synthetic peptide
RKETVA

SEQ ID NO: 61 - protein, 84 aa, Artificial Sequence
  Synthetic peptide
KVTLQKDAQNLIGISIGGGAQYCPCLYIVQVFDNTPAALDGTVAAGDEITGVNGRSIKGKTKVEVAKMIQEVKGEVTIHYNKLQ

SEQ ID NO: 62 - protein, 6 aa, Artificial Sequence
  Synthetic peptide
SIESDV

SEQ ID NO: 63 - protein, 87 aa, Artificial Sequence; identical to SEQ ID NO: 35
  Synthetic peptide

SEQ ID NO: 64 - protein, 6 aa, Artificial Sequence
  Synthetic peptide
QSSSSL

SEQ ID NO: 65 - protein, 90 aa, Artificial Sequence; identical to SEQ ID NO: 57
  Synthetic peptide

SEQ ID NO: 66 - protein, 6 aa, Artificial Sequence
  Synthetic peptide
PFSSSV

SEQ ID NO: 67 - protein, 182 aa, Artificial Sequence
  Synthetic peptide
EITLERGNSGLGFSIAGGTDNPHIGDDPSIFITKIIPGGAAAQDGRLRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKP

SEQ ID NO: 68 - protein, 6 aa, Artificial Sequence
  Synthetic peptide
LEDSVF

SEQ ID NO: 69 - protein, 76 aa, Artificial Sequence
  Synthetic peptide
TITMHKDSTGHVGFIFKNGKITSIVKDSSAARNGLLTEHNICEINGQNVIGLKDSQIADILSTSGTVVTITIMPAF

SEQ ID NO: 70 - protein, 160 aa, Artificial Sequence
  Synthetic peptide
EVILCKDQDGKIGLRLKSIDNGIFVQLVQANSPASLVGLRFGDQVLQINGENCAGWSSDKAHKVLKQAFGEKITMTIRDRPFERTITMHKDSTGHVGFIFKNGKITSIVKDSSAARNGLLTEHNICEINGQNVIGLKDSQIADILSTSGTVVTITIMPAF

SEQ ID NO: 71 - protein, 6 aa, Artificial Sequence
  Synthetic peptide
RRESAI

SEQ ID NO: 72 - protein, 98 aa, Artificial Sequence; identical to SEQ ID NO: 44
  Synthetic peptide

SEQ ID NO: 73 - protein, 6 aa, Artificial Sequence
  Synthetic peptide
SVSTVV

SEQ ID NO: 74 - protein, 87 aa, Artificial Sequence; identical to SEQ ID NO: 35
  Synthetic peptide

SEQ ID NO: 75 - protein, 6 aa, Artificial Sequence
  Synthetic peptide
AEDSFL

SEQ ID NO: 76 - protein, 81 aa, Artificial Sequence; identical to SEQ ID NO: 16
  Synthetic peptide

SEQ ID NO: 77 - protein, 6 aa, Artificial Sequence
  Synthetic peptide
LTASEV

SEQ ID NO: 78 - protein, 81 aa, Artificial Sequence
  Synthetic peptide
EVEVFKSEEALGLTITDNGAGYAFIKRIKEGSVIDHIQLISVGDMIEAINGQSLLGCRHYEVARLLKELPRGRTFTLKLTE

SEQ ID NO: 79 - protein, 6 aa, Artificial Sequence
  Synthetic peptide
EQVSAV

SEQ ID NO: 80 - protein, 85 aa, Artificial Sequence
  Synthetic peptide
EVELAKNDNSLGISVTGGVNTSVRHGGIYVKAVIPQGAAESDGRIHKGDRVLAVNGVSLEGATHKQAVETLRNTGQVVHLLLEKG

SEQ ID NO: 81 - protein, 6 aa, Artificial Sequence
  Synthetic peptide
VQQTRV

SEQ ID NO: 82 - protein, 87 aa, Artificial Sequence; identical to SEQ ID NO: 14
  Synthetic peptide

SEQ ID NO: 83 - protein, 87 aa, Artificial Sequence; identical to SEQ ID NO: 35
  Synthetic peptide

SEQ ID NO: 84 - protein, 81 aa, Artificial Sequence; identical to SEQ ID NO: 41
  Synthetic peptide

SEQ ID NO: 85 - protein, 6 aa, Artificial Sequence
  Synthetic peptide
ASGSSV

SEQ ID NO: 86 - protein, 88 aa, Artificial Sequence
  Synthetic peptide
EINLTRGPSGLGFNIVGGTDQQYVSNDSGIYVSRIKEDGAAARDGRLQEGDKILSVNGQDLKNLLHQDAVDLFRNAGYAVSLRVQHRL

SEQ ID NO: 87 - protein, 6 aa, Artificial Sequence
  Synthetic peptide
FRETEV

SEQ ID NO: 88 - protein, 87 aa, Artificial Sequence; identical to SEQ ID NO: 14
  Synthetic peptide

SEQ ID NO: 89 - protein, 87 aa, Artificial Sequence; identical to SEQ ID NO: 35
  Synthetic peptide

SEQ ID NO: 90 - protein, 6 aa, Artificial Sequence
  Synthetic peptide
LEDTEL

SEQ ID NO: 91 - protein, 83 aa, Artificial Sequence
  Synthetic peptide
EVRLDRLHPEGLGLSVRGGLEFGCGLFISHLIKGGQADSVGLQVGDEIVRINGYSISSCTHEEVINLIRTKKTVSIKVRHIGL

SEQ ID NO: 92 - protein, 6 aa, Artificial Sequence
  Synthetic peptide
RRETQL

SEQ ID NO: 93 - protein, 89 aa, Artificial Sequence
  Synthetic peptide
ECTVKRGPQGELGVTVLGGAEHGEFPYVGAVAAVEAAGLPGGGEGPRLGEGELLLEVQGVRVSGLPRYDVLGVIDSCKEAVTFKAVRQG

SEQ ID NO: 94 - protein, 6 aa, Artificial Sequence
  Synthetic peptide
RRETQV

SEQ ID NO: 95 - protein, 83 aa, Artificial Sequence
  Synthetic peptide
HTKLRKSSRGFGFTVVGGDEPDEFLQIKSLVLDGPAALDGKMETGDVIVSVNDTCVLGHTHAQVVKIFQSIPIGASVDLELCR

SEQ ID NO: 96 - protein, 89 aa, Artificial Sequence
  Synthetic peptide
ECTVKRGPQGELGVTVLGGAEHGEFPYVGAVAAAEAAGLPGGGEGPKLAEGELLLEVQGVRVSGLPRYDVLGVIDSCKEAVTFKAVRQG

SEQ ID NO: 97 - protein, 83 aa, Artificial Sequence; identical to SEQ ID NO: 95
  Synthetic peptide

SEQ ID NO: 98 - protein, 6 aa, Artificial Sequence
  Synthetic peptide
GGETRL

SEQ ID NO: 99 - protein, 89 aa, Artificial Sequence
  Synthetic peptide
PIIIHRAGKKYGFTLRAIRVYMGDSDVYTVHHMVWHVEDGGPASEAGLRQGDLITHVNGEPVHGLVHTEVVELILKSGNKVAISTTPLE

SEQ ID NO: 100 - protein, 6 aa, Artificial Sequence
  Synthetic peptide
TNETSL

SEQ ID NO: 101 - protein, 88 aa, Artificial Sequence
  Synthetic peptide
TLTILRQTGGLGISIAGGKGSTPYKGDDEGIFISRVSEEGPAARAGVRVGDKLLEVNGVALQGAEHHEAVEALRGAGTAVQMRVWRER

SEQ ID NO: 102 - protein, 90 aa, Artificial Sequence
  Synthetic peptide
EIRLPRAGGPLGLSIVGGSDHSSHPFGVQEPGVFISKVLPRGLAARSGLRVGDRILAVNGQDVRDATHQEAVSALLRPCLELSLLVRRDP

SEQ ID NO: 103 - protein, 6 aa, Artificial Sequence
  Synthetic peptide
EEDTHL

SEQ ID NO: 104 - protein, 80 aa, Artificial Sequence; identical to SEQ ID NO: 22
  Synthetic peptide

SEQ ID NO: 105 - protein, 6 aa, Artificial Sequence
  Synthetic peptide
EEDTHL

SEQ ID NO: 106 - protein, 81 aa, Artificial Sequence; identical to SEQ ID NO: 23
  Synthetic peptide

SEQ ID NO: 107 - protein, 6 aa, Artificial Sequence
  Synthetic peptide
LQQSNV

SEQ ID NO: 108 - protein, 83 aa, Artificial Sequence
  Synthetic peptide
LVDLQKKTGRGLGLSIVGKRSGSGVFISDIVKGGAADLDGRLIRGDQILSVNGEDMRHASQETVATILKCVQGLVQLEIGRLR

SEQ ID NO: 109 - protein, 83 aa, Artificial Sequence; identical to SEQ ID NO: 108
  Synthetic peptide

SEQ ID NO: 110 - protein, 83 aa, Artificial Sequence; identical to SEQ ID NO: 108
  Synthetic peptide

SEQ ID NO: 111 - protein, 6 aa, Artificial Sequence
  Synthetic peptide
SLESEV

SEQ ID NO: 112 - protein, 83 aa, Artificial Sequence; identical to SEQ ID NO: 108
  Synthetic peptide

SEQ ID NO: 113 - protein, 83 aa, Artificial Sequence; identical to SEQ ID NO: 108
  Synthetic peptide

SEQ ID NO: 114 - protein, 6 aa, Artificial Sequence
  Synthetic peptide
YLVTRL

SEQ ID NO: 115 - protein, 87 aa, Artificial Sequence
  Synthetic peptide
TVEIIRELSDALGISIAGGKGSPLGDIPIFIAMIQANGVAARTQKLKVGDRIVSINGQPLDGLSHTDAVNLLKNAFGRIILQVVADT

SEQ ID NO: 116 - protein, 6 aa, Artificial Sequence
  Synthetic peptide
GPATDL

SEQ ID NO: 117 - protein, 93 aa, Artificial Sequence
  Synthetic peptide
IVEIFREPNVSLGISIVGGQTVIKRLKNGEELKGIFIKQVLEDSPAGKTNALKTGDKILEVSGVDLQNASHAEAVEAIKSAGNPVVFVVQSLS

SEQ ID NO: 118 - protein, 6 aa, Artificial Sequence
  Synthetic peptide
EQPVYI

SEQ ID NO: 119 - protein, 167 aa, Artificial Sequence
  Synthetic peptide
DVFIEKQKGEILGVVIVESGWGSILPTVIIANMMHGGPAEKSGKLNIGDQIMSINGTSLVGLPLSTCQSIIKGLKNQSRVKLNIVRCPPVTTVLIRRPDLRYQLGFSVQNGIICSLMRGGIAERGGVRVGHRIIEINGQSVVATPHEKIVHILSNAVGEIHMKTMPA

SEQ ID NO: 120 - protein, 6 aa, Artificial Sequence
  Synthetic peptide
QEELII

SEQ ID NO: 121 - protein, 88 aa, Artificial Sequence
  Synthetic peptide
EVPLNDSGSAGLGVSVKGNRSKENHADLGIFVKSIINGGAASKDGRLRVNDQLIAVNGESLLGKANQEAMETLRRSMSTEGNKRGMIQ

SEQ ID NO: 122 - protein, 6 aa, Artificial Sequence
  Synthetic peptide
EEGIWA

SEQ ID NO: 123 - protein, 87 aa, Artificial Sequence
  Synthetic peptide
RTTMPKESGALLGLKVVGGKMTDLGRLGAFITKVKKGSLADVVGHLRAGDEVLEWNGKPLPGATNEEVYNIILESKSEPQVEIIVSR

SEQ ID NO: 124 - protein, 6 aa, Artificial Sequence
  Synthetic peptide
IESVKI

SEQ ID NO: 125 - protein, 84 aa, Artificial Sequence; identical to SEQ ID NO: 61
  Synthetic peptide

SEQ ID NO: 126 - protein, 6 aa, Artificial Sequence
  Synthetic peptide
VRTYSC

SEQ ID NO: 127 - protein, 83 aa, Artificial Sequence
  Synthetic peptide
TVELKRYGGPLGITISGTEEPFDPIIISSLTKGGLAERTGAIHIGDRILAINSSSLKGKPLSEAIHLLQMAGETVTLKIKKQT

SEQ ID NO: 128 - protein, 6 aa, Artificial Sequence
  Synthetic peptide
RKEYFI

SEQ ID NO: 129 - protein, 82 aa, Artificial Sequence
  Synthetic peptide
LIQFEKVTEEPMGITLKLNEKQSCTVARILHGGMIHRQGSLHVGDEILEINGTNVTNHSVDQLQKAMKETKGMISLKVIPNQ

SEQ ID NO: 130 - protein, 6 aa, Artificial Sequence
  Synthetic peptide
TNEFYA

SEQ ID NO: 131 - protein, 160 aa, Artificial Sequence; identical to SEQ ID NO: 70
  Synthetic peptide

SEQ ID NO: 132 - protein, 6 aa, Artificial Sequence
  Synthetic peptide
GLDVPV

SEQ ID NO: 133 - protein, 90 aa, Artificial Sequence
  Synthetic peptide
EIRVRVEKDPELGFSISGGVGGRGNPFRPDDDGIFVTRVQPEGPASKLLQPGDKIIQANGYSFINIEHGQAVSLLKTFQNTVELIIVREV

SEQ ID NO: 134 - protein, 6 aa, Artificial Sequence
  Synthetic peptide
DKEYYV

SEQ ID NO: 135 - protein, 87 aa, Artificial Sequence
  Synthetic peptide
VITVTLKKQNGMGLSIVAAKGAGQDKLGIYVKSVVKGGAADVDGRLAAGDQLLSVDGRSLVGLSQERAAELMTRTSSVVTLEVAKQG

SEQ ID NO: 136 - protein, 6 aa, Artificial Sequence
  Synthetic peptide
QEEFYA

SEQ ID NO: 137 - protein, 160 aa, Artificial Sequence; identical to SEQ ID NO: 70
  Synthetic peptide

SEQ ID NO: 138 - protein, 6 aa, Artificial Sequence
  Synthetic peptide
FHQFYI

SEQ ID NO: 139 - protein, 167 aa, Artificial Sequence; identical to SEQ ID NO: 119
  Synthetic peptide

SEQ ID NO: 140 - protein, 6 aa, Artificial Sequence
  Synthetic peptide
DREYYV

SEQ ID NO: 141 - protein, 87 aa, Artificial Sequence; identical to SEQ ID NO: 135
  Synthetic peptide

SEQ ID NO: 142 - protein, 6 aa, Artificial Sequence
  Synthetic peptide
NRPVPV

SEQ ID NO: 143 - protein, 84 aa, Artificial Sequence
  Synthetic peptide
IIEISKGRSGLGLSIVGGKDTPLDAIVIHEVYEEGAAARDGRLWAGDQILEVNGVDLRSSSHEEAITALRQTPQKVRLVVYRDE

SEQ ID NO: 144 - protein, 83 aa, Artificial Sequence; identical to SEQ ID NO: 108
  Synthetic peptide

SEQ ID NO: 145 - protein, 6 aa, Artificial Sequence
  Synthetic peptide
KTEFCA

SEQ ID NO: 146 - protein, 90 aa, Artificial Sequence
  Synthetic peptide
MVTLDKTGKKSFGICIVRGEVKDSPNTKTTGIFIKGIVPDSPAHLCGRLKVGDRILSLNGKDVRNSTEQAVIDLIKEADFKIELEIQTFD

SEQ ID NO: 147 - protein, 81 aa, Artificial Sequence
  Synthetic peptide
NVDLMKKAGKELGLSLSPNEIGCTIADLIQGQYPEIDSKLQRGDIITKFNGDALEGLPFQVCYALFKGANGKVSMEVTRPK

SEQ ID NO: 148 - protein, 6 aa, Artificial Sequence
  Synthetic peptide
VKVDSV

SEQ ID NO: 149 - protein, 83 aa, Artificial Sequence
  Synthetic peptide
SVRLFKRKVGGLGFLVKERVSKPPVIISDLIRGGAAEQSGLIQAGDIILAVNDRPLVDLSYDSALEVLRGIASETHVVLILRG

SEQ ID NO: 150 - protein, 6 aa, Artificial Sequence
  Synthetic peptide
FGGEPL

SEQ ID NO: 151 - protein, 86 aa, Artificial Sequence
  Synthetic peptide
EVVVPKKAGEPLGIVVVESGWGSMLPTVVLAHMNPVGPAAHSNKLNIGDQIININGISLVGLPLSAAQTQIKNMKTATAVRMTVVS

SEQ ID NO: 152 - protein, 6 aa, Artificial Sequence
  Synthetic peptide
AFFEEL

SEQ ID NO: 153 - protein, 80 aa, Artificial Sequence
  Synthetic peptide
EVILCKDQDGKIGLRLKSVDNGIFVQLVQANSPASLVGLRFGDQVLQINGENCAGWSSDKAHKVLKQAFGEKITMTIRDR

SEQ ID NO: 154 - protein, 6 aa, Artificial Sequence
  Synthetic peptide
SGKDYV

SEQ ID NO: 155 - protein, 84 aa, Artificial Sequence; identical to SEQ ID NO: 143
  Synthetic peptide

SEQ ID NO: 156 - protein, 29 aa, Artificial Sequence
  Synthetic peptide
XXXXXXXXXXXXXXXXXXXXXXXXXXXXX

SEQ ID NO: 157 - protein, 27 aa, Artificial Sequence
  Synthetic peptide
RGPEGFTTHLETTFTGDGTPKTIRVTQ

SEQ ID NO: 158 - protein, 4 aa, Artificial Sequence
  Synthetic peptide
ETTF

SEQ ID NO: 159 - protein, 159 aa, Artificial Sequence
  Synthetic peptide
XXXXXXXXXXXXXXXXXXGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 160 - protein, 6 aa, Artificial Sequence
  Synthetic peptide
SIESDV

SEQ ID NO: 161 - protein, 7 aa, Artificial Sequence
  Synthetic peptide
SSIESDV

SEQ ID NO: 162 - protein, 4 aa, Artificial Sequence
  Synthetic peptide
SSSV

(19) United States
(12) Patent Application Publication
(10) Pub. No.: US 2014/0329706 A1
(43) Pub. Date: Nov. 6, 2014

(54) AFFINITY TAGS, AND RELATED AFFINITY LIGANDS, ENGINEERED PROTEINS, MODIFIED SUPPORTS, COMPOSITIONS, METHODS AND SYSTEMS

(71) Applicant: CALIFORNIA INSTITUTE OF TECHNOLOGY, PASADENA, CA (US)

(72) Inventor: Ward Gale WALKUP, IV, MONROVIA, CA (US)

(73) Assignee: CALIFORNIA INSTITUTE OF TECHNOLOGY, PASADENA, CA (US)

(21) Appl. No.: 14/267,881

(22) Filed: May 1, 2014

Related U.S. Application Data
(60) Provisional application No. 61/818,317, filed on May 1, 2013.

(51) Int. Cl. G01N 33/58; G01N 33/573; G01N 33/543; C07K 14/00; C07K 1/22

(52) U.S. Cl. CPC G01N 33/58; C07K 14/00; C07K 1/22; G01N 33/54306; G01N 33/573
USPC 506/9; 530/300; 530/350; 530/413; 506/18; 436/501; 435/7.1; 435/7.21; 435/7.4; 252/184

(57) ABSTRACT

Affinity tags and ligands comprising a PDZ domain peptide and a PDZ binding carboxy terminal peptide and related engineered protein, engineered labels, compositions, methods and systems.

### CROSS REFERENCE TO RELATED APPLICATIONS

The present application claims priority to U.S. Provisional Patent Application No. 61/818,317, filed on May 1, 2013, the disclosure of which is incorporated herein by reference in its entirety.

### STATEMENT OF INTEREST

This invention was made with government support under MH095095 awarded by the National Institutes of Health and under 0703267 awarded by the National Science Foundation. The government has certain rights in the invention.

### FIELD

The present disclosure relates to affinity tags and related ligands, engineered proteins modified support, compositions, methods and systems.

### BACKGROUND

Affinity tags are peptides used to detect targets of interest and/or to separate biochemical mixtures based on a specific interaction among the tags and various compounds and molecules.

To date several affinity tags are known that are used e.g. for detection and/or purification from complex mixtures such as a crude preparation.

Despite the existence of several affinity tags and related methods and systems, selectivity and sensitivity to target molecules for target detection and/or separation, e.g. by affinity chromatography, can be challenging, as well as development of methods that are efficient in term of time and product yields

### SUMMARY

Provided herein are affinity tags and related affinity ligands, engineered proteins, engineered labels, chromatography solid phase, protein arrays, compositions methods and systems based on PDZ domains and related PDZ ligands that in several embodiments allow selective detection, separation of various proteins of interest, increased purity and/or increased yield with respect to other chromatography methods and systems.

According to a first aspect a PDZ domain peptide is described. The PDZ domain peptide has 70 to 130 amino acids and comprises a βA strand, a βB strand, a βC strand, a βD strand, a βE strand, a βF strand, an αA helix and an αB helix linked one to another by loop regions in a configuration wherein the βB strand, the βC strand and the αB helix form a ligand binding pocket ranging in size from approximately 400 Å³ to approximately 900 Å³ and wherein a GLGF loop linking the βA strand and the βB strand provides a steric block at the end of the ligand binding groove. The PDZ domain peptide has sequence X₁-X₂-X₃-X₄-X₅-X₆-X₇-X₈-X₉ X₁₀-X₁₁-X₁₂-X₁₃-X₁₄-X₁₅-X₁₆-X₁₇-X₁₈-G-X₂₀-X₂₁-X₂₂-X₂₃-X₂₄-X₂₅-X₂₆-X₂₇-X₂₈-X₂₉-X₃₀ X₃₁-X₃₂-X₃₃ X₃₄-X₃₅-X₃₆-X₃₇-X₃₈-X₃₉-X₄₀-X₄₁-X₄₂-X₄₃-X₄₄-X₄₅-X₄₆-X₄₇-X₄₈-X₄₉-X₅₀-X₅₁-X₅₂-X₅₃-X₅₄-X₅₅-X₅₆-X₅₇-X₅₈-X₅₉-X₆₀-X₆₁-X₆₂-X₆₃-X₆₄-X₆₅-X₆₆-X₆₇-X₆₈-X₆₉-X₇₀-X₇₁-X₇₂-X₇₃-X₇₄-X₇₅-X₇₆-X₇₇-X₇₈-X₇₉-X₈₀-X₈₁-X₈₂-X₈₃-X₈₄ X₈₅-X₈₆-X₈₈-X₈₉-X₉₀-X₉₁-X₉₂-X₉₃-X₉₄-X₉₅-X₉₆-X₉₇-X₉₈-X₉₉-X₁₀₀ X₁₀₁-X₁₀₂-X₁₀₃-X₁₀₄-X₁₀₅-X₁₀₆-X₁₀₇-X₁₀₈-X₁₀₉-X₁₁₀-X₁₁₁-X₁₁₂-X₁₁₃-X₁₁₄-X₁₁₅-X₁₁₆-X₁₁₇-X₁₁₈-X₁₁₉-X₁₂₀-X₁₂₁-X₁₂₂-X₁₂₃-X₁₂₄-X₁₂₅-X₁₂₆-X₁₂₇-X₁₂₈-X₁₂₉-X₁₃₀ wherein

X₁ to X₅ can be any amino acids defining an N-terminal segment wherein up to four of the X₁ to X₅ amino acids residues might be absent;
X₆ to X₁₁ can be any amino acids forming the βA beta strand wherein one of the X₆ to X₁ᵢ amino acid residues might be absent;
X₁₂ to X₂₁ form the GLGF loop wherein X₁₂ can be any amino acid, X₁₃ can be R or K, X₁₄ to X₁₆ can be any amino acid X₁₇ can be any amino acid, X₁₈ and X₂₀ are hydrophobic amino acids, X₂₁ can be any amino acid, and where up to two amino acids of X₁₂ X₁₄ to X₁₆ and X₂₁ might be absent;
X₂₂ to X₂₇ can be any amino acids forming the βB beta strand wherein up to two of the X₂₂ to X₂₇ amino acids might be absent;
X₂₈ to X₄₁ can be any amino acids forming a loop region wherein up to 13 of the X₂₈ to X₄₁ amino acids might be absent;
X₄₂ to X₄₈ can be any amino acids forming the βC beta strand wherein up to two of the X₄₂ to X₄₈ amino acids might be absent;
X₄₉ to X₅₃ can be any amino acids forming a loop region wherein one of the X₄₉ to X₅₃ amino acid residues might be absent;
X₅₄ to X₅₉ can be any amino acids forming the αA alpha helix wherein up to four of the X₅₄ to X₅₉ amino acid residues might be absent;
X₆₀ to X₆₇ can be any amino acids forming a loop region wherein up to three of the X₆₀ to X₆₇ amino acid residues might be absent;
X₆₈ to X₇₅ can be any amino acids forming the βD beta strand wherein up to three of the X₆₈ to X₇₅ amino acid residues might be absent;
X₇₆ to X₇₉ can be any amino acids forming a loop region wherein up to two of the X₇₆ to X₇₉ amino acid residues might be absent;
X₈₀ to X₈₃ can be any amino acids forming the βE beta strand wherein up to two of the X₈₀ to X₈₃ amino acid residues might be absent;
X₈₄ to X₉₂ can be any amino acids forming a loop region where up to four of the X₈₄ to X₉₂ amino acid residues might be absent;
X₉₃ to X₁₀₂ can be any amino acids forming a αB alpha helix wherein up to two of the X₉₃ to X₁₀₂ amino acid residues might be absent;
X₁₀₃ to X₁₁₀ can be any amino acids forming a loop region wherein up to two of the X₁₀₃ to X₁₁₀ amino acid residues might be absent;
X₁ᵢᵢ to X₁₁₈ can be any amino acids forming a βF beta strand wherein up to two of the X₁ᵢᵢ to X₁₁₈ amino acid residues might be absent;
X₁₁₉ to X₁₂₁ can be any amino acids forming a loop region wherein one of the X₁₁₉ to X₁₂₁ amino acid residues might be absent; and
X₁₂₂ to X₁₃₀ can be any amino acids forming a αC alpha helix wherein all of the X₁₂₂ to X₁₃₀ amino acid residues might be absent. (SEQ ID NO: 1).

According to a second aspect a PDZ binding carboxy terminal peptide is described. The PDZ binding carboxy terminal peptide having sequence X₁-X₂-X₃-X₄ wherein X₁ and X₃ can be any amino acids, X₂ can be S, T, D, E or a bulky hydrophobic amino acid and X₄ can be A, C or a bulky hydrophobic amino acid (SEQ ID NO: 2).

According to a third aspect, an affinity tag is described. The affinity tag essentially consists of a PDZ domain peptide, or a PDZ binding carboxy terminal peptide herein described. In the affinity tag herein described, the PDZ domain peptide configured so that a PDZ domain peptide tag is capable of specifically binding one or more corresponding PDZ domain peptide ligands and/or one or more PDZ binding C-terminal peptide ligands, and the PDZ C binding carboxy terminal peptide is configured so that a PDZ binding carboxy terminal peptide tag is capable of specifically binding a corresponding PDZ domain peptide ligand.

According to a fourth aspect, an affinity ligand is described. The affinity ligand comprises a peptide selected from the group consisting of a PDZ domain peptide, and a PDZ binding carboxy terminal peptide herein described. In the affinity ligand herein described, the PDZ domain peptide is configured so that a PDZ domain peptide ligand is capable of specifically binding one or more corresponding PDZ domain peptide tags and/or one or more PDZ binding C-terminal peptide tags, and the PDZ binding carboxy terminal peptide is configured so that a PDZ binding carboxy terminal peptide ligand is capable of specifically binding a corresponding PDZ domain peptide tag. In some embodiments, the affinity ligand can be a synthetic and/or a non-naturally occurring affinity ligand.

According to a fifth aspect, an engineered protein is described. The engineered protein comprises a target protein attaching at least one affinity tag selected from the group consisting of a PDZ domain peptide herein described and a PDZ binding carboxy terminal peptide herein described. In engineered proteins herein described, the affinity tag is attached to the target protein in a configuration where the affinity tag is presented for binding to a corresponding affinity ligand. In the engineered protein herein described, a PDZ domain peptide tag is presented for specific binding to one or more of corresponding PDZ domain peptide ligands and/or corresponding PDZ binding carboxy terminal peptide ligands. In the engineered protein herein described, a PDZ binding carboxy terminal tag is presented for specific binding to a corresponding PDZ domain peptide ligand.

According to a sixth aspect, an engineered label is described. The engineered label comprises a label attaching at least one affinity ligand selected from the group consisting of a PDZ domain peptide herein described and a PDZ binding carboxy terminal peptide herein described. In engineered labels herein described, the affinity ligand is attached to the label in a configuration where the affinity ligand is presented for binding to a corresponding affinity tag. In the engineered label herein described, a PDZ domain peptide ligand is presented for specific binding to one or more of corresponding PDZ domain peptide tags and/or corresponding PDZ binding carboxy terminal peptide tags. In the engineered label herein described, a PDZ binding carboxy terminal ligand is presented for specific binding to a corresponding PDZ domain peptide tag.

According to a seventh aspect, a chromatography stationary phase is described. The chromatography stationary phase comprising a solid support attaching at least one affinity ligand selected from the group consisting of a PDZ domain peptide herein described, and a PDZ domain binding carboxy terminal peptide herein described. In the chromatography stationary phase herein described, the solid support attaches one or more PDZ domain peptide ligand in a configuration presenting the one or more PDZ domain peptide ligand for specific binding to one or more of corresponding PDZ domain peptide tags and/or corresponding PDZ binding carboxy terminal peptide tags. In the chromatography stationary phase herein described, the solid support attaches one or more PDZ binding carboxy terminal ligands in a configuration wherein the PDZ binding carboxy terminal ligand is presented for specific binding to a corresponding PDZ domain peptide tag.

According to an eighth aspect a reagent for detaching a PDZ tag-PDZ ligand complex, is described, wherein the PDZ tag is a PDZ domain peptide tag or a PDZ binding carboxy terminal peptide tag and the PDZ ligand is a corresponding PDZ domain peptide ligand or a PDZ binding carboxy terminal peptide ligand. The reagent is selected from the group consisting of a PDZ domain peptide and a PDZ binding carboxy terminal peptide wherein the PDZ tag specifically binds the PDZ ligand with a first binding affinity K_{D}, and the PDZ ligand or PDZ Tag specifically binds the reagent with a second binding affinity K_{Dcomp}, the second binding affinity K_{Dcomp} lower, equal to, or higher than the first binding affinity K_{D} and allowing detachment of the PDZ ligand from the PDZ affinity tag following contacting of the PDZ affinity tag-PDZ ligand complex with the reagent at set elution conditions wherein ratio of eluted target with respect to total bound target is a function of Kd, KDcomp and L0 wherein L0 is the concentration of the PDZ ligand.

According to a ninth aspect, an affinity chromatography system is described. The affinity chromatography system comprises one or more engineered proteins herein described, one or more one or more chromatography stationary phase herein described and/or one or more reagents for detaching a PDZ ligand from a PDZ tag herein described, for simultaneous combined or sequential use in a method to separating a target protein herein described.

According to tenth aspect, a method for separating a target protein from a biochemical mixture is described. The method comprises providing the target protein in an engineered protein herein described attaching a PDZ affinity tag presented for binding to a corresponding PDZ affinity ligand. The method further comprises contacting the engineered protein with an affinity chromatography stationary phase comprising a solid support attaching the PDZ affinity ligand to allow specific binding between PDZ affinity tag and the PDZ affinity ligand; and separating the engineered protein by detaching the PDZ affinity tag from the PDZ affinity ligand.

According to an eleventh aspect, a protein array is described. The protein array comprises a solid support surface attaching at least one affinity ligand selected from the group consisting of a PDZ domain peptide, and a PDZ binding carboxy terminal peptide. In the protein array herein described, the solid support surface attaches one or more PDZ domain peptide ligand in a configuration presenting the one or more PDZ domain peptide ligands for specific binding to one or more of corresponding PDZ domain peptide tags and/or corresponding PDZ binding carboxy terminal peptide tags. In the protein array herein described, the solid support surface attaches one or more PDZ binding carboxy terminal ligand in a configuration wherein the PDZ binding carboxy terminal ligand is presented for specific binding to a corresponding PDZ domain peptide tag.

According to twelfth aspect a method for detecting a target in a biochemical mixture is described. The method comprises contacting the biochemical mixture with one or more engineered proteins herein described capable of specific binding to the target to form an engineered protein-target complex. In the method, the engineered protein-target complex presents the PDZ domain protein tag or the PDZ binding carboxy terminal peptide tag for binding to a corresponding PDZ domain protein ligand and/or to a PDZ binding carboxy terminal peptide ligand. The method further comprises contacting the engineered protein-target complex with an engineered label herein described presenting the corresponding PDZ domain peptide ligand and/or a PDZ binding carboxy terminal peptide ligand.

According to a thirteenth aspect, a target detection system is described. The target detection system comprises two or more of; one or more engineered proteins herein described, one or more engineered labels herein described and a protein array herein described for simultaneous combined or sequential use in a method to detect a target herein described.

In some embodiments the affinity tags and related affinity ligands, engineered proteins, engineered labels, compositions methods and systems can be used to perform affinity chromatography of biochemical mixtures such as samples.

In particular, in some embodiments, the engineered proteins, a chromatography stationary phase, reagents methods and systems and related compositions herein described allow performance of an affinity chromatography to purify and concentrate a substance from a mixture into a buffering solution.

Additionally, in some embodiments, the engineered proteins, a chromatography stationary phase, reagents methods and systems and related compositions herein described allow performance of an affinity chromatography to reduce the amount of a substance in a mixture

Also in some embodiments, the engineered proteins, a chromatography stationary phase, reagents methods and systems and related compositions herein described allow performance of affinity chromatography to discern what biological compounds bind to a particular substance.

Furthermore, in some embodiments, the engineered proteins, a chromatography stationary phase, reagents methods and systems and related compositions herein described allow performance of an affinity chromatography to purify and concentrate an enzyme solution.

In some affinity tags and related affinity ligands, engineered proteins, engineered labels, protein arrays, compositions methods and systems can be used to perform detection of targets of interest in biochemical mixtures.

The affinity tags and related affinity ligands, engineered proteins, engineered labels, chromatography solid phase, protein arrays, compositions methods and systems herein described can be used in connection with applications wherein detection of targets, separation of a target protein from a mixture is desired. Exemplary applications comprise laboratory applications, fundamental biological studies, pharmaceuticals, diagnostics and medical applications and additional applications identifiable by a skilled person upon reading of the present disclosure.

The details of one or more embodiments of the present disclosure are set forth in the description below. Other features, objects, and advantages will be apparent from the description and from the claims.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 illustrates an exemplary x-ray crystal structure, in a ribbon representation, of a PDZ3 domain of PSD-95 (1), including a cartoon representation of the domain in its particular position in a full length protein of PSD-95. In particular, the illustration of FIG. 1 was generated using the UCSF Chimera Molecular Modeling Program and the coordinate file 1bfe from the Protein Data Bank.

FIG. 2 illustrates x-ray crystal structure of the PSD-95 PDZ3 domain in a ribbon representation, a structure of its PDZ binding C-terminal peptide, and a PDZ-PDZ binding C-terminal peptide in a complex, which show the binding peptide within the binding pocket of the PDZ domain (1). In particular, FIG. 2 was generated using the UCSF Chimera Molecular Modeling Program and the coordinate files 1be9 and 1bfe from the Protein Data Bank.

FIG. 3 illustrates a cartoon depicting a PDZ domain interacting with a C-terminal PDZ binding peptide, in which the peptide binds in a “lock and key” configuration and is representative of FIG. 2.

FIG. 4 illustrates an x-ray crystal structure, in ribbon representation, of an nNOS PDZ domain with a C-terminal β-hairpin, and its positioning in the full length protein, Nitric Oxide Synthase (nNOS) (2). The ribbon representation of PDZ domain of nNOS (AA 14-125 of 1429 AA nNOS protein; Uniprot ID: P29476) was generated using the UCSF Chimera Molecular Modeling Program and the coordinate file 1qau from the Protein Data Bank.

FIG. 5 shows an x-ray crystal structure and ribbon representation of the syntrophin PDZ domain binding homotypically to an nNOS PDZ domain containing a C-terminal β-hairpin motif (2). In particular, FIG. 5 was generated using the UCSF Chimera Molecular Modeling Program and the coordinate file 1qav from the Protein Data Bank.

FIG. 6 illustrates a cartoon representation of a PDZ domain binding homotypically to an nNOS PDZ domain containing a C-terminal β-hairpin motif, representative of FIG. 5.

FIG. 7 illustrates a cartoon representation of a PDZ domain binding C-terminal peptide as a single affinity tag (Type A PDZ Affinity Tag) that is attached to a protein of interest.

FIG. 8 shows a cartoon representation of dual PDZ domain binding C-terminal peptide affinity tags (Type A PDZ Affinity Tags, Dual) that are fused to a bi-specific monoclonal antibody.

FIG. 9 illustrates a cartoon representation of several exemplary PDZ domains with β-hairpins as affinity tags (Type B PDZ Affinity Tags) attached to a protein of interest (POI).

FIG. 10 illustrates a cartoon representation of several exemplary PDZ domains as affinity tags (Type C PDZ Affinity Tags) attached to a protein of interest (POI).

FIGS. 11 and 12 illustrate exemplary cartoon representations of different types of PDZ affinity resins.

FIG. 13 illustrates several examples of PDZ Affinity Chromatography eluting agents.

FIG. 14 illustrates purification scheme 1, where the protein of interest (POI) is attached to a PDZ binding C-terminal peptide tag (Type A PDZ Affinity Tag) and the affinity matrix is a PDZ domain immobilized on a solid support.

FIG. 15 illustrates purification scheme 2, where the protein of interest (POI) is attached to a C-terminal PDZ domain with a β-hairpin (Type B PDZ Affinity Tag) and the affinity matrix is a PDZ domain immobilized on a solid support.

FIG. 16 illustrates purification scheme 3, where the protein of interest (POI) is attached to a N-terminal PDZ domain with β-hairpin (Type B PDZ Affinity Tag) and the affinity matrix is a PDZ domain immobilized on a solid support.

FIG. 17 illustrates purification scheme 4, where the protein of interest (POI) is attached to a C-terminal PDZ domain (Type C PDZ Affinity Tag) and the affinity matrix is a PDZ domain binding C-terminal peptide immobilized on a solid support.

FIG. 18 illustrates purification scheme 5, where the protein of interest (POI) is attached to a N-terminal PDZ domain (Type C PDZ Affinity Tag) and the affinity matrix is a PDZ domain binding C-terminal peptide immobilized on a solid support.

FIG. 19 illustrates purification scheme 6, where the protein of interest (POI) is attached to a PDZ binding C-terminal peptide tag (Type A PDZ Affinity Tag) and the affinity matrix is an anti-PDZ binding C-terminal peptide tag antibody immobilized on solid support.

FIG. 20 illustrates purification scheme 7, where the protein of interest (POI) is attached to a C-terminal PDZ domain with a β-hairpin (Type B PDZ Affinity Tag) and the affinity matrix is an anti-PDZ binding C-terminal peptide tag antibody immobilized on solid support.

FIG. 21 illustrates purification scheme 8 part 1, which shows purification of a bi-specific monoclonal antibody tagged with two distinct PDZ binding C-terminal peptide tags (Type A PDZ Affinity Tags).

FIG. 22 illustrates purification scheme 8 part 2, which shows purification of a bi-specific monoclonal antibody tagged with two distinct PDZ binding C-terminal peptide tags (Type A PDZ Affinity Tags).

FIG. 23 illustrates purification scheme 8 part 3, which shows purification of a bi-specific monoclonal antibody tagged with two distinct PDZ binding C-terminal peptide tags (Type A PDZ Affinity Tags).

FIG. 24 illustrates purification scheme 8 part 4, which shows purification of a bi-specific monoclonal antibody tagged with two distinct PDZ binding C-terminal peptide tags (Type A PDZ Affinity Tags).

FIG. 25 illustrates a PSD-95 PDZ2 affinity chromatography purification of the neuronal protein NR2B (GluN2B) heterologously expressed in E. coli fused to a N-terminal maltose binding protein (MBP) tag (3).

FIG. 26 illustrates the PSD-95 PDZ3 affinity chromatography purification of the neuronal protein synGAP heterologously expressed in E. coli fused to a N-terminal 6× Histidine tag (3).

FIG. 27 illustrates a PSD-95 PDZ1-PDZ2 affinity chromatography purification of the neuronal protein cypin heterologously expressed in E. coli in its native state (untagged) or fused to a N-terminal MBP tag (3).

FIG. 28 illustrates a PSD-95 PDZ3 affinity chromatography purification, in the presence and absence of reducing agents, of the neuronal protein CRIPT heterologously expressed in E. coli (3).

FIG. 29 illustrates a PSD-95 PDZ2 affinity chromatography purification of the PDZ Domain and its C-terminal β-Hairpin from neuronal Nitric Oxide Synthase (nNOS), heterologously expressed in E. coli (3).

FIG. 30 illustrates a PSD-95 PDZ2 Affinity Chromatography purification of heterologously expressed Thioredoxin (THX) protein fused to a truncation library of the C-terminal 10 amino acids of the Tail region of the NR2B (GluN2B) protein (3).

FIG. 31 illustrates a PSD-95 PDZ2 Affinity Chromatography purification of heterologously expressed MBP protein fused to a truncation library of the C-terminal 10 amino acids of the Tail region of the NR2B (GluN2B) protein (3).

FIG. 32 illustrates a PSD-95 PDZ2 Affinity Chromatography purification of heterologously expressed Glutathione S-Transferase (GST) protein fused to a truncation library of the C-terminal 10 amino acids of the Tail region of the NR2B (GluN2B) protein (3).

FIG. 33 illustrates a PSD-95 PDZ2 affinity chromatography purification of the PDZ Domain and its C-terminal β-Hairpin from neuronal Nitric Oxide Synthase (nNOS), heterologously expressed in E. coli (3).

FIG. 34 illustrates a PSD-95 PDZ2 affinity chromatography purification of 2× and 3× tandem nNOS PDZ with β-Hairpins heterologously expressed in E. coli.

FIG. 35 illustrates a PSD-95 PDZ2 affinity chromatography purification of MBP and GST fusion proteins containing N-terminal, internal or C-terminal 1× and 2× tandem nNOS PDZ domains with β-Hairpins (Type B PDZ Affinity Tags) heterologously expressed in E. coli (3).

FIG. 36 illustrates 1× and 2× tandem nNOS PDZ domains with β-Hairpins or PDZ domain binding C-terminal peptide affinity chromatography purification of MBP and GST fusion proteins containing N-terminal, internal or C-terminal PSD-95 PDZ2 domains (Type C PDZ Affinity Tags) heterologously expressed in E. coli (3).

FIG. 37 illustrates a PSD-95 PDZ2 affinity chromatography purification of dihydrofolate reductase (DHFR), Dasher green fluorescent protein (Dasher GFP), β-galactosidase (LacZ) and chloramphenicol (CAT) fusion proteins containing PDZ binding C-terminal peptides (Type A PDZ Affinity Tags) heterologously expressed in E. coli (3).

FIG. 38 illustrates a PSD-95 PDZ2 affinity chromatography purification of DHFR, Dasher GFP, LacZ and CAT fusion proteins containing N-terminal 1× nNOS PDZ domains with β-Hairpins (Type B PDZ Affinity Tags) heterologously expressed in E. coli (3).

FIG. 39 illustrates PSD-95 PDZ2 affinity chromatography purification of DHFR, Dasher GFP, LacZ and CAT fusion proteins containing N-terminal 2× nNOS PDZ domains with β-Hairpins (Type B PDZ Affinity Tags) heterologously expressed in E. coli (3).

FIG. 40 illustrates a C-terminal PDZ domain binding peptide affinity chromatography purification of DHFR, Dasher GFP, LacZ and CAT fusion proteins containing N-terminal PSD-95 PDZ2 domains (Type C PDZ Affinity Tags) heterologously expressed in E. coli (3).

FIG. 41 illustrates a functional analysis of purified DasherGFP and Dasher GFP PDZ Affinity tagged fusion proteins. Fluorescence excitation and emission spectra of DasherGFP and DasherGFP fused to PDZ Affinity Tags (Type A, B and C PDZ Affinity Tags) were plotted to assess the effects of PDZ Affinity Tags on Dasher GFP activity (3).

FIG. 42 illustrates a functional analysis of purified LacZ PDZ Affinity tagged fusion proteins. Michaelis-Menten plots for rates of o-nitrophenol-β-galactoside (ONPG) hydrolysis by purified LacZ fused to Type A and B PDZ Affinity Tags were plotted to assess the effects of PDZ Affinity Tags on LacZ activity (3).

FIG. 43 illustrates a functional analysis of purified CAT and PDZ Affinity tagged CAT fusion proteins. Michaelis-Menten plots for rates of 5,5′-dithiobis-2-nitrobenzoic acid (DTNB) reduction by purified CAT and CAT fused to Type A and B PDZ Affinity Tags (3).

FIG. 44 illustrates C-terminal PDZ domain binding peptide affinity chromatography purification of PSD-95 PDZ2 and coupling of purified PSD-95 PDZ2 to NHS-Activated Agarose to prepare PDZ Domain Affinity Resin (3).

FIG. 45 illustrates a pull down scheme to identify proteins that interact with a protein of interest (POI), where the POI is attached to a C-terminal PDZ domain (Type C PDZ Affinity Tag) and the pull down affinity matrix is a PDZ domain binding C-terminal peptide immobilized on a solid support.

FIG. 46 illustrates the use of an affinity matrix derivatized with multiple unique PDZ binding peptides to deplete PDZ domains in a mixture in order to enrich or purify a protein of interest (POI) that lacks any detectable affinity to the PDZ binding peptide matrix.

FIG. 47 illustrates the use of a PDZ domain tag fused to a protein of interest (POI) and a PDZ domain binding C-terminal peptide coupled to a detection agent to specifically detect the presence of the fusion protein in a mixture.

FIG. 48 Illustrates a protein array designed utilizing PDZ domains immobilized on a surface to detect the amount of POIs present in a sample. POIs are expressed as PDZ domain binding C-terminal peptide fusion proteins and are detected using antibodies coupled to a detection agent.

### DETAILED DESCRIPTION

Described herein are affinity tags and related affinity ligands, engineered proteins, engineered labels, chromatography solid phase, protein arrays, compositions methods and systems based on PDZ domains and related PDZ ligands.

“Affinity tags,” as used herein, refers to peptides or proteins fused to or grafted onto a recombinant protein. In particular affinity tags can be genetically fused or grafted on a target protein (for example, using ligation independent cloning (4,5), Gibson Assembly (6,7) or gene synthesis (8,9), co-translationally grafted (for example, using non-canonical amino acid insertion (10-12)) or post-translationally grafted using click chemistry (13-19) or an enzyme such as sortase (20-23). In some instances grafting can be performed by chemical modification (e.g. by use of a sortase or other enzymes as will be understood by a skilled person). Some tags are removable tags and can be removed by chemical agents or by enzymatic means, such as proteolysis or intein splicing. Tags are attached to proteins for various purposes, e.g. to purify and concentrate a substance from a mixture into a buffering solution, to reduce the amount of a substance in a mixture, to discern what biological compounds bind to a particular substance. Exemplary affinity tags comprise chitin binding protein (CBP), maltose binding protein (MBP), and glutathione-S-transferase (GST) and poly(His) tag. In some instances, affinity tags can also have additional properties and be used to also impart such properties to the tagged protein. For example, affinity tags such as MBP, and GST, can have a dual role as a solubilization tags where solubilization tags relates to protein or peptide (e.g. thioredoxin (TRX) and poly (NANP)) that can be used to assist in the proper folding in proteins and keep them from precipitating.

Affinity tags are usually used to perform “Affinity chromatography,” as described herein, refers to separation of a molecule from a mixture based on its high binding affinity to a specific molecule. In particular, Affinity Chromatography is a separation technique based upon molecular conformation, which frequently utilizes application of suitable solid phases. These solid phases have ligands attached to their surfaces which are specific for the compounds to be separated. In particular In in affinity chromatography a “stationary phase” is the substance fixed in place for the chromatography procedure, e.g. a silica layer in thin layer chromatography. An immobilized phase is a stationary phase that is immobilized on the support particles, or on the inner wall of the column tubing.

In particular affinity tags herein described, specifically bind a corresponding affinity ligand which is typically immobilized on a solid substrate forming the a chromatography solid phase.

The wording “specific” “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions etc. The term “specific” as used herein with reference to a molecular component of a complex, refers to the unique association of that component to the specific complex which the component is part of. The term “specific” as used herein with reference to a sequence of a polynucleotide refers to the unique association of the sequence with a single polynucleotide which is the complementary sequence. By “stable complex” is meant a complex that is detectable and does not require any arbitrary level of stability, although greater stability is generally preferred.

Affinity tags herein described are capable of specifically binding a corresponding affinity ligand through non covalent bonds, such as hydrogen bonds, Van der Waals contacts, Van der Waals/London dispersions, π-π stacking and ionic bonds (e.g. salt bridges) or through covalent bonds with the exception of covalent bonds that are targeted by reducing agent capable of cleaving the target covalent bond through addition of hydrogen.

The term “corresponding” as used herein with reference to tag-ligand binding or binding of other molecules, refers to a tag, ligand or other molecule that can react to another tag ligand or other molecule. Thus, an affinity ligand and affinity tag that can react with each other can be referred to as corresponding affinity tag and affinity ligand.

The term “ligand” as used herein indicates a compound with an affinity to bind to a target. This affinity can take any form. For example, such affinity can be described in terms of non-covalent interactions, such as the type of binding that occurs in enzymes that are specific for certain substrates and is detectable. Typically those interactions include several weak interactions, such as hydrophobic, van der Waals, and hydrogen bonding which typically take place simultaneously. Exemplary ligands in general include molecules comprised of multiple subunits taken from the group of amino acids, non-natural amino acids, and artificial amino acids, and organic molecules, each having a measurable affinity for a specific target (e.g. a protein target). More particularly, exemplary ligands include polypeptides and peptides, or other molecules which can possibly be modified to include one or more functional groups. The disclosed ligands, for example, can have an affinity for a target, can bind to a target, can specifically bind to a target, and/or can be bindingly distinguishable from one or more other ligands in binding to a target.

In particular, affinity tags-ligands herein described function in a fashion similar to that of antibody-antigen interactions, or glycoprotein-lectins interactions. This “lock and key” fit between the ligand and its target compound makes it highly specific, frequently generating a single peak, while all else in the sample is unretained. Accordingly for example, detergent-solubilized proteins can be allowed to bind to a chromatography resin that has been modified to have a covalently attached lectin. Proteins that do not bind to the lectin are washed away and then specifically bound glycoproteins can be eluted by adding a high concentration of a sugar that competes with the bound glycoproteins at the lectin binding site. Some lectins have high affinity binding to oligosaccharides of glycoproteins that is hard to compete with sugars, and bound glycoproteins need to be released by denaturing the lectin. In addition to a “lock and key” fit between a ligand and its target compound, additional interactions including hydrophobic, van der Waals, charge-charge and hydrogen bonding can form between the affinity tag and ligand in regions outside of the active site (e.g. side chains in the cyclic peptide ligand binding to amino acid side chains outside of the ligand binding domain) (24-27).

In embodiments herein described, affinity tags and affinity ligands can be formed by a PDZ domain peptide, or by a PDZ domain binding carboxy terminal peptide.

The term “PDZ domain”, as described herein, indicates a compact, globular region of 70-130 amino-acids in an antiparallel β sandwich configuration. A β sandwich configuration as used herein indicates a compact arrangement of six beta strands (βA-βF) and two alpha helices (αA-αB), linked one to another by eighth loop regions in a configuration wherein the βB strand, βC strand and αB helix form a ligand binding pocket and wherein the βA to βB loop referred to herein as the GLGF Loop provides a steric block at the end of the ligand binding groove (1,28). In a PDZ domain The ligand binding pocket can range in size from approximately 400-900 Å³, and the overall dimensions of the PDZ domain can be approximated by a sphere of radius 12-20 Å. Exemplary PDZ domains comprise domains found in the signaling proteins of bacteria, yeast, plants, viruses and animals. PDZ is an acronym combining the first letters of three proteins: post synaptic density protein (PSD95), Drosophila disc large tumor suppressor (D1g1), and zonula occludens-1 protein (zo-1), which were first discovered to include a domain with this conformation (29). PDZ domains have previously been referred to as DHR (Dlg homologous region) or GLGF (glycine-leucine-glycine-phenylalanine) domains. PDZ domains can occur in isolation or in multiple copies in proteins, and are typically found in multidomain scaffolding proteins that serve to link together large molecular complexes at specific locations within cells (30-33). PDZ domains typically bind to short peptide motifs at the extreme C-termini of other proteins, but have also been known to bind internal peptide motifs, calcium phospholipids and other PDZ domains (1,27,34,35). The a steric block provided by GLGF loop at the end of the ligand binding groove that necessitates chain termination or redirection in peptide sequences binding in the ligand-binding pocket

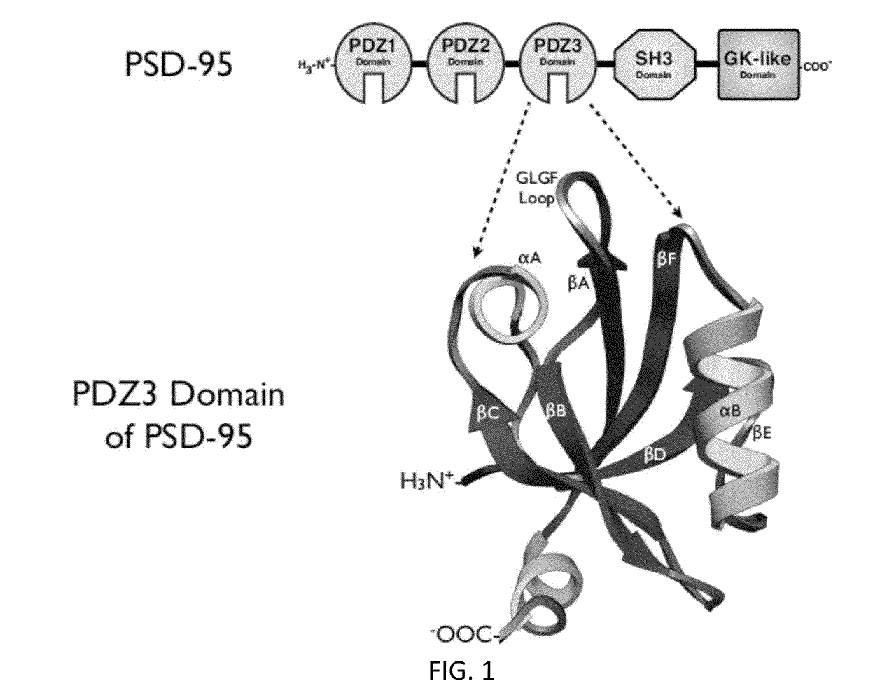

Reference is made to FIG. 1, which depicts an x-Ray crystal structure of the ribbon representation, of a PDZ Domain, and it's positioning within the protein, PSD-95. As shown, PDZ domains are small (≦110 AA), that can occur in isolation or in multiple copies in proteins, and are typically found in multi-domain proteins (1,28,30-32). In the illustration of FIG. 1 the antiparallel β-sandwich is shown where the six beta strands (βA-βF) and two alpha helices (αA & αB) are indicated, and with a ligand binding pocket formed by the βB (2nd), βC (3rd) and αB (2nd) secondary structure elements and the loop regions connecting αB to βF and βA to βB (GLGF Loop) also shown. Also shown in FIG. 1 is the structure of the PDZ3 domain from the protein PSD-95 in the absence of its C-terminal peptide binding partner (PSD-95 is 724 Amino Acids (AA); PDZ3 Domain is AA 302-402 of PSD-95) (1,28).

In a PDZ domain the secondary structure elements βA strand, βB strand, βC strand, βD strand, βE strand, βF strand, and αA helix and αB helix are arranged together with an N-terminal segment and an optional αC alpha helix from the N terminus to C-terminus in a configuration comprising an N-terminal segment of 1 to 5 residues linked to a βA beta strand of 5-6 residues linked to a first loop region containing GLGF loop of 8 to 10 amino acids, linked to a βB beta strand of 4 to 6 residues, linked to a loop region of 1-14 residues, βC beta strand of 5 to 7 residues, linked to a loop region of 4 to 5 residues, linked to αA alpha helix of 2 to 6 residues, linked to a loop region of 5 to 8 residues, linked to a βD beta strand of 5 to 8 residues, linked to a loop region of 2 to 4 residues, linked to a βE beta strand of 2 to 4 residues, linked to a loop region of 4 to 8 residues, linked to a αB alpha helix of 8 to 10 residues, linked to a loop region of 6 to 8 residues, linked to a βF beta strand of 6 to 8 residues, linked to a loop region of 2 to 3 residues, linked to an optional αC alpha helix of 7 to 9 residues.

In particular, in a PDZ domain the GLGF loop, also known as a carboxylate binding loop, has sequence X₁-X₂-X₃-X₄-X₅-X₆-G-X₈ wherein X₁ is R, K or A, X₂ to X₄ can be any amino acid, X₅ can be any amino acid, preferably G, X₆ and X₈ are hydrophobic amino acid and in particular can be V, I, L, F, or Y (1,28) (SEQ ID NO: 3). In some embodiments of the GLGF loop of SEQ ID NO: 3, X₅ is G, X6 is L and X8 is F and the loop comprises sequence GLGF (SEQ ID NO: 4)

In affinity tags, ligands, engineered proteins, modified supports of the disclosure, a PDZ domain has sequence SEQ ID NO:1 which comprises the GLFG loop between residues X₁₂-X₂₁

In particular PDZ domain peptide according to the disclosure and in particular the PDZ domain peptide of SEQ ID NO: 1 is configured to fold in a β sandwich configuration in which the ligand binding pocket can range in size from approximately 400 Å³ to approximately 900 Å³, and the overall dimensions of the PDZ domain are approximated by a sphere of radius 12-20 Å.

The PDZ domain peptide sequence can be originated by cleavage from naturally occurring protein or synthetically provided protein having a PDZ domain, chemical synthesis, recombinant DNA technology (e.g. by expression in cell or cell free systems of recombinant vectors such as plasmid) and by additional techniques identifiable by a skilled person.

In some embodiments, PDZ domains peptides can have the sequence of having 15% or more pairwise sequence identity to any naturally occurring or synthetic PDZ domain. In some embodiments, PDZ domain peptides can also have the structure of any crystal/NMR/Tomography/Microscopy structure or computationally modeled protein structure (e.g. ab initio, homology, and additional computation methods) possessing a root mean squared deviation of 10 angstroms or less over a 40 point (e.g. atom) comparison to any PDZ domain crystal/NMR/Tomography/Microscopy structure or computationally modeled PDZ domain protein structure. Additionally, in some embodiments a PDZ domain peptide can have any sequences classified as PDZ domains by automated, semi-automated or manually curated protein sequence or structure family, superfamily, domain, and other databases are considered to be PDZ domains.

The specific sequence of a PDZ domain peptide in the sense of the present disclosure can be provided by sequences of naturally occurring PDZ domains (e.g. the PDZ domain of nNOS and PSD-95) and/or by use of software programs able to provide amino acid sequences that can form the alpha helices, beta strands and GLGF loop arrangement herein described as a PDZ domain. Exemplary computational design methods that can be used to identify the sequences of PDZ domain peptides or create sequences that fold into PDZ domains, comprise structure prediction, sequence alignment and protein design packages such as Modeller (36), Rosetta (37), Robetta (38), iTasser (39,40), Orbit (41,42), ClustalW (43), MUSCLE (44,45), machine learning algorithms, statistical potentials, and additional methods identifiable by a skilled person. Additional methods comprise directed evolution, or directed evolution in combination with a computationally guided approach starting from a known naturally occurring or synthetic sequence as will be understood by a skilled person. Exemplary sequences of PDZ domains that can be provided in PDZ domain peptides of the disclosure are listed in Tables 1 to 4 of the present disclosure.

The term “peptide” and “oligopeptide” as used herein indicate a polypeptide with less than 200 amino acid monomers. As used herein the term “amino acid”, “amino acidic monomer”, or “amino acid residue” refers to any of the twenty naturally occurring amino acids, non-natural amino acids, and artificial amino acids and includes both D an L optical isomers. In particular, amino acid as refer to organic compounds composed of amine (—NH₂) and carboxylic acid (—COOH), along with a side-chain specific to each amino acid. Amino acids comprise proteinogenic amino acids (e.g. 23 amino acids), amino acids encoded by the nuclear genes of eukaryotes (e.g. 21 amino acids) or non-standard amino acids such as those not normally found in proteins or those not produced directly and in isolation by standard cellular machinery

The term “PDZ binding carboxy terminal peptide” or “PDZ binding C-terminal peptide” as used herein indicates a short peptide of 4 or more amino acids in length capable of binding to the ligand binding pocket of a PDZ domain.

In general a PDZ binding C-terminal peptide can specifically bind to the PDZ domain by beta sheet augmentation. When PDZ binding C-terminal peptide binds to a PDZ domain the beta sheet in the PDZ domain is extended by the addition of a further beta strand from the tail of the PDZ C-terminal peptide. Formation of hydrogen bonds between the GLGF loop and terminal carboxylate group of the PDZ binding carboxy terminal peptide is functional to a proper beta sheet augmentation as will be understood by a skilled person.

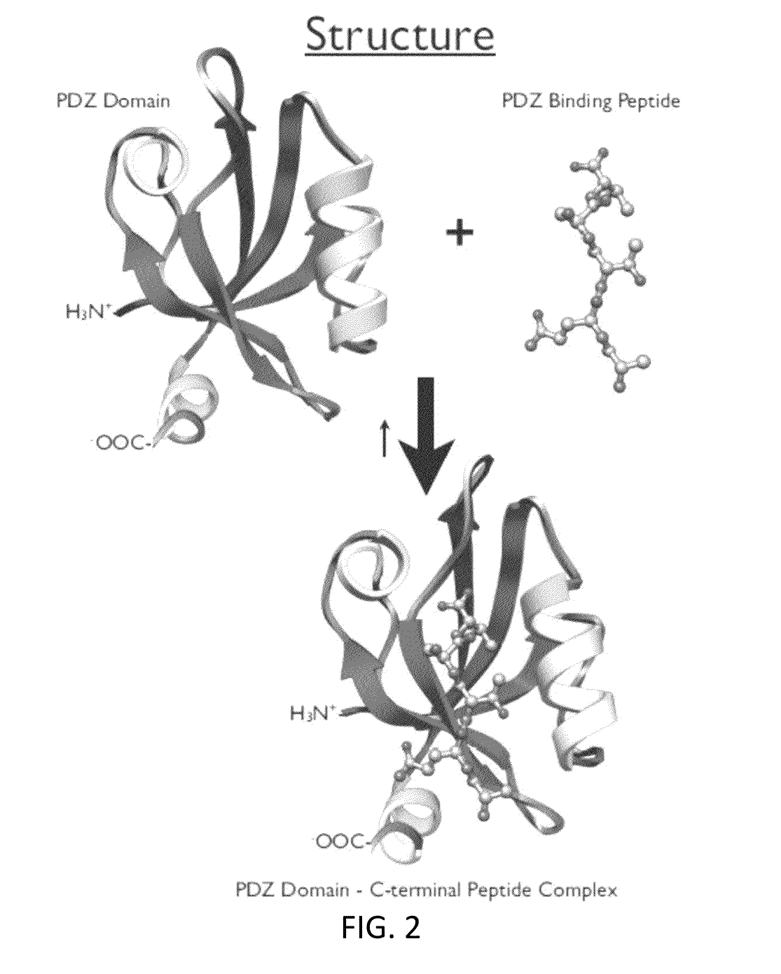

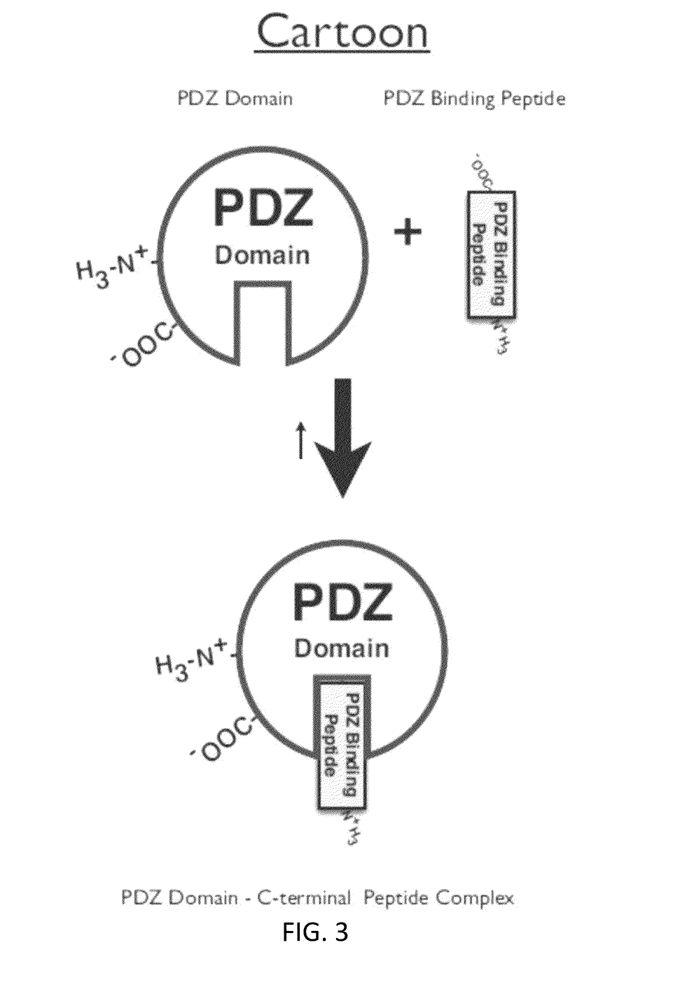

Reference is made to the illustration of FIG. 2 and FIG. 3 showing that the PDZ domain can bind to a PDZ binding C-terminal peptide with a high specificity and affinity rendering the PDZ domain useful as a tag in purification purposes.

In particular, reference is made to FIG. 2, which show the x-ray crystal structure, and FIG. 3, which shows the cartoon representations of a PDZ Domain binding to a C-terminal peptide. In particular, FIG. 2, shows a ribbon representation of x-ray crystal structure of PSD-95 PDZ3 domain (PSD-95 is 724 AA; PDZ3 domain is AA 302-402 of PSD-95), and the ball-and-stick drawing of the PDZ binding peptide, which binds into a binding pocket of the PDZ domain in a “lock and key” fashion. The structure and cartoon representations depict the most common mode of protein-protein interaction between PDZ domains and their binding partners. In this specific exemplary drawing, the ligand binding pocket of the PSD-95 PDZ3 domain is accepting the six C-terminal amino acids of the neuronal protein CRIPT (1) The binding reaction shown is essentially irreversible due to the sub-micromolar K_{D} affinity measurement (K_{D} ˜0.76 uM) between the PSD-95 PDZ3 domain and the C-terminal six amino acids of CRIPT (46,47).

In affinity tags, and affinity ligands, of the disclosure the PDZ binding carboxy terminal peptide is comprised as an isolated or synthetic peptide having sequence X₁-X₂-X₃-X₄ wherein X₁ and X₃ can be any amino acids, X₂ can be S, T, D, E or a bulky hydrophobic amino acid (herein also indicated with φ), and X₄ can be A, C or a bulky hydrophobic amino acid and in particular, V, I, L, F, or Y (SEQ ID NO: 2). In some embodiments, in PDZ binding C-terminal peptide of SEQ ID NO:2, X₂ can be V, Y, or F, and X₄ can be V, I, L, F, or Y. In some embodiments X3 does not include C.

In particular, in some embodiments, PDZ binding carboxy terminal peptides herein described comprise a PDZ binding carboxy terminal peptide of SEQ ID NO: 2 in which X2 is S or T and X4 is V, I or L (herein also Type I PDZ binding carboxy terminal peptide).

In some embodiments, PDZ binding carboxy terminal peptides herein described comprise a PDZ binding carboxy terminal peptide of SEQ ID NO: 2 in which X2 and X4 are independently a bulky hydrophobic amino acid and in particular one of V, Y, F, L and I, (herein also indicated as Type II PDZ binding carboxy terminal peptides).

In some embodiments, PDZ binding carboxy terminal peptides herein described comprise a PDZ binding carboxy terminal peptide of SEQ ID NO: 2 in which X2 is D or E and X4 is V or L (herein also indicated as Type III PDZ binding carboxy terminal peptides).

The peptide can be originated by cleavage from naturally occurring or synthetically provided protein having a PDZ binding carboxy terminal peptide, chemical synthesis, recombinant DNA technology (e.g. by expression in cell or cell free systems of recombinant vectors such as plasmid) and by additional techniques identifiable by a skilled person.

The specific sequence of a PDZ binding carboxy terminal peptide in the sense of the present disclosure can be provided by sequences of naturally occurring PDZ binding carboxy terminal peptide and/or by use of software programs able to provide amino acid sequences that can provide a peptide sequence in view of a set binding specificity with a corresponding PDZ domain peptide.

The PDZ binding carboxy terminal peptides of Type I, Type II and Type III have different binding specificities as will be apparent to a skilled person upon reading of the present disclosure.

In affinity tags and affinity ligands herein described the PDZ domain peptide and PDZ binding carboxy terminal peptides are capable to bind a corresponding PDZ domain or PDZ binding carboxy terminal peptide possibly comprised in other affinity tags and affinity ligands herein described, in target proteins, in protein arrays herein described or in engineered labels herein described.

In particular, in some embodiments, a PDZ domain peptide can specifically bind a corresponding PDZ binding carboxy terminal peptide

Exemplary PDZ binding C-terminal peptides and corresponding PDZ domain are illustrated in Table 1, wherein the PDZ binding C-terminal peptide and PDZ domains have been separated into three distinct classes (herein indicated as Type I, Type II and Type III) based upon their respective binding specificity.

### Table 1: PDZ Binding Carboxy Terminal Peptides and Corresponding PDZ Domain Peptides

TABLE 1
PDZ binding carboxy terminal peptides and corresponding PDZ domain peptides

| | PDZ C- | | Exemplary PDZ | |
|---|---|---|---|---|
| | terminal | | domain binding | Example PDZ |
| | Peptide | PDZ domain residue | partner including | domain binding |
| | consensus | requirements for specific | PDZ binding C- | PDZ C terminal |
| | sequence | binding to peptides | terminal peptide | peptide |
| Type I | X-S/T-X- | Amino acid residues in | NR2A [ESDV] | PSD-95 PDZ1/2 |
| | V/I/L | positions 1 and 3 of the βB | (SEQ ID NO: 5) | PSD-95 PDZ1/2 |
| | | strand (X₂₂ and X₂₄ in SEQ ID | NR2B [ESDV] | PSD-95 PDZ3 |
| | | NO: 1) are hydrophobic and the | (SEQ ID NO: 5) | PSD-95 PDZ1/2/3 |
| | | second residue in the GLGF | CRIPT [QTSV] | |
| | | loop (X₁₈ in SEQ ID NO: 1) is | (SEQ ID NO: 6) | |
| | | L/F/V/M/Y for specific binding | synGAP | |
| | | to peptides containing a | [QTRV] (SEQ | |
| | | hydrophobic residue at X₄. | ID NO: 7) | |
| | | Residue 1 of the αB helix (X₉₃ | | |
| | | in SEQ ID NO: 1) is | | |
| | | H/Y/N/Q/K for specific binding | | |
| | | to peptides containing S/T in | | |
| | | the X₂ position | | |
| Type II | X-φ-X-φ | Residues 1 and 3 in the βB | Neurexin-3 | Afadin |
| | | strand (X₂₂ and X₂₄ in SEQ ID | [EYYV] (SEQ | InaD-like protein |
| | | NO: 1) are hydrophobic and the | ID NO: 8) | (PDZ8) |
| | | second residue in the GLGF | 5- | |
| | | loop (X₁₈ in SEQ ID NO: 1) is | hydroxytryptamine | |
| | | L/F/V/M/Y for specific binding | receptor 4 | |
| | | to peptides containing a | [PVPV] (SEQ | |
| | | hydrophobic residue at X₄. | ID NO: 9) | |
| | | Residue 1 of the αB helix (X₉₃ | | |
| | | in SEQ ID NO: 1) is | | |
| | | A/C/D/E/F/I/L/M/P/R/ST/V/W | | |
| | | for specific binding to peptides | | |
| | | containing hydrophobic amino | | |
| | | acids φ in the X₂ position | | |
| Type III | X-D/E-X-V/L | Residues 1 and 3 in the βB | Melatonin | nNOS PDZ |
| | | strand (X₂₂ and X₂₄ in SEQ ID | receptor 1A | |
| | | NO: 1) is hydrophobic and the | [VDSV] (SEQ | |
| | | second residue in the GLGF | ID NO: 10) | |
| | | loop (X₁₈ in SEQ ID NO: 1) | | |
| | | must be L/F/V/M/Y for specific | | |
| | | binding to peptides containing a | | |
| | | hydrophobic residue at X₄. | | |
| | | Residue 5 of the αB helix (X₉₇ | | |
| | | in SEQ ID NO: 1) is R/K for | | |
| | | specific binding to peptides | | |
| | | containing acidic amino acids | | |
| | | (D/E) in the X₂ position | | |

Therefore in some embodiments, the PDZ domain peptide can have SEQ ID NO: 1 wherein X₁₈ is L, F, V, M or Y; X₂₂ and X₂₄ are hydrophobic amino acid and in particular V, I, L F, or Y; X₉₃ is H, Y, N, Q or K (Type I PDZ domain peptides).

In some embodiments, the PDZ domain peptide can have SEQ ID NO: 1 wherein X₁₈ is L, F, V, M or Y; X₂₂ and X₂₄ are hydrophobic amino acid and in particular V, I, L F, or Y; X₉₃ is A, C, D, E, F, I, L, M, P, R, S, T, V or W (Type II PDZ domain peptides).

In some embodiments, the PDZ domain peptide can have SEQ ID NO: 1 wherein X₁₈ is L, F, V, M or Y; X₂₂ and X₂₄ are hydrophobic amino acid and in particular V, I, L F, or Y; X₉₇ is R or K (Type III PDZ domain peptides).

Exemplary Type I PDZ carboxy terminal domains and corresponding PDZ domain peptides are illustrated in Table 2.

TABLE 2
PDZ binding carboxy terminal peptides and
corresponding PDZ domain proteins

| Type I C- | SEQ | PDZ domain Uniprot | SEQ |
|---|---|---|---|
| terminal | ID | Identifier and PDZ | ID |
| peptides | NO | Domain Sequence | NO |
| SLESCF | 11 | >sp\|Q3UHD6\|41-134 | 12 |
| | | VVRIVKSESGYGFNVRGQVSEGGQLRSI | |
| | | NGELYAPLQHVSAVLPGGAADRAGVRK | |
| | | GDRILEVNGVNVEGATHKQVVDLIRAG | |
| | | EKELILTVLSVP | |
| LWETSI | 13 | >sp\|P31016\|65-151 | 14 |
| | | EITLERGNSGLGFSIAGGTDNPHIGDDPSI | |
| | | FITKIIPGGAAAQDGRLRVNDSILFVNEV | |
| | | DVREVTHSAAVEALKEAGSIVRLYVMR | |
| | | R | |
| TNDSLL | 15 | >sp\|O14745\|14-94 | 16 |
| | | LCCLEKGPNGYGFHLHGEKGKLGQYIRL | |
| | | VEPGSPAEKAGLLAGDRLVEVNGENVE | |
| | | KETHQQVVSRIRAALNAVRLLVVDPE | |
| YLVTSV | 17 | >sp\|Q64512\|1357-1442 | 18 |
| | | EVELAKTDGSLGISVTGGVNTSVRHGGI | |
| | | YVKAIIPKGAAESDGRIHKGDRVLAVNG | |
| | | VSLEGATHKQAVETLRNTGQVVHLLLE | |
| | | KGQ | |
| WDETNL | 19 | >sp\|Q9WV48\|663-757 | 20 |
| | | TVLLQKKDSEGFGFVLRGAKAQTPIEEF | |
| | | TPTPAFPALQYLESVDEGGVAWRAGLR | |
| | | MGDFLIEVNGQNVVKVGHRQVVNMIRQ | |
| | | GGNTLMVKVVMVT | |
| SLETSL | 21 | >sp\|Q15599\|11-90 | 22 |
| | | LCRLVRGEQGYGFHLHGEKGRRGQFIRR | |
| | | VEPGSPAEAAALRAGDRLVEVNGVNVE | |
| | | GETHHQVVQRIKAVEGQTRLLVVDQ | |
| SLETSL | 21 | >sp\|Q15599\|150-230 | 23 |
| | | RLCHLRKGPQGYGFNLHSDKSRPGQYIR | |
| | | SVDPGSPAARSGLRAQDRLIEVNGQNVE | |
| | | GLRHAEVVASIKAREDEARLLVVDP | |
| SLETSV | 24 | >sp\|Q15700\|421-501 | 25 |
| | | KVVLHKGSTGLGFNIVGGEDGEGIFVSFI | |
| | | LAGGPADLSGELQRGDQILSVNGIDLRG | |
| | | ASHEQAAAALKGAGQTVTIIAQYQ | |
| SLETSV | 24 | >sp\|Q92796\|130-217 | 26 |
| | | EEIVLERGNSGLGFSIAGGIDNPHVPDDP | |
| | | GIFITKIIPGGAAAMDGRLGVNDCVLRV | |
| | | NEVDVSEVVHSRAVEALKEAGPVVRLV | |
| | | VRRR | |
| SLETSV | 24 | >sp\|O75970\|1862-1948 | 27 |
| | | TVEMKKGPTDSLGISIAGGVGSPLGDVPI | |
| | | FIAMMHPTGVAAQTQKLRVGDRIVTICG | |
| | | TSTEGMTHTQAVNLLKNASGSIEMQVV | |
| | | AGG | |
| TLVSTV | 28 | >sp\|Q92796\|130-217 | 26 |
| | | EEIVLERGNSGLGFSIAGGIDNPHVPDDP | |
| | | GIFITKIIPGGAAAMDGRLGVNDCVLRV | |
| | | NEVDVSEVVHSRAVEALKEAGPVVRLV | |
| | | VRRR | |
| TLVSTV | 28 | >sp\|Q92796\|226-311 | 29 |
| | | EVNLLKGPKGLGFSIAGGIGNQHIPGDNS | |
| | | IYITKIIEGGAAQKDGRLQIGDRLLAVNN | |
| | | TNLQDVRHEEAVASLKNTSDMVYLKVA | |
| | | K | |
| LFSTEV | 30 | >sp\|P55196\|1007-1093 | 31 |
| | | IITVTLKKQNGMGLSIVAAKGAGQDKLG | |
| | | IYVKSVVKGGAADVDGRLAAGDQLLSV | |
| | | DGRSLVGLSQERAAELMTRTSSVVTLEV | |
| | | AKQG | |
| LEITEL | 32 | >sp\|Q9Y6N9\|211-293 | 33 |
| | | KVFISLVGSRGLGCSISSGPIQKPGIFISHV | |
| | | KPGSLSAEVGLEIGDQIVEVNGVDFSNL | |
| | | DHKEAVNVLKSSRSLTISIVAAAG | |
| RRTTPV | 34 | >sp\|P31016\|65-151 | 14 |
| | | EITLERGNSGLGFSIAGGTDNPHIGDDPSI | |
| | | FITKIIPGGAAAQDGRLRVNDSILFVNEV | |
| | | DVREVTHSAAVEALKEAGSIVRLYVMR | |
| | | R | |
| RRTTPV | 34 | >sp\|P31016\|160-246 | 35 |
| | | EIKLIKGPKGLGFSIAGGVGNQHIPGDNSI | |
| | | YVTKIIEGGAAHKDGRLQIGDKILAVNS | |
| | | VGLEDVMHEDAVAALKNTYDVVYLKV | |
| | | AKP | |
| VQDTRL | 36 | >sp\|O14745\|14-94 | 37 |
| | | LCCLEKGPNGYGFHLHGEKGKLGQYIRL | |
| | | VEPGSPAEKAGLLAGDRLVEVNGENVE | |
| | | KETHQQVVSRIRAALNAVRLLVVDPE | |
| LNSTTL | 38 | >sp\|O14745\|154-234 | 39 |
| | | LCTMKKGPSGYGFNLHSDKSKPGQFIRS | |
| | | VDPDSPAEASGLRAQDRIVEVNGVCME | |
| | | GKQHGDVVSAIRAGGDETKLLVVDRE | |
| HSTTRV | 40 | >sp\|P31016\|313-393 | 41 |
| | | RIVIHRGSTGLGFNIVGGEDGEGIFISFILA | |
| | | GGPADLSGELRKGDQILSVNGVDLRNAS | |
| | | HEQAAIALKNAGQTVTIIAQYK | |
| YKQTSV | 42 | >sp\|P31016\|313-393 | 41 |
| | | RIVIHRGSTGLGFNIVGGEDGEGIFISFILA | |
| | | GGPADLSGELRKGDQILSVNGVDLRNAS | |
| | | HEQAAIALKNAGQTVTIIAQYK | |
| WFDTDL | 43 | >sp\|Q9DBG9\|15-112 | 44 |
| | | RVEIHKLRQGENLILGFSIGGGIDQDPSQ | |
| | | NPFSEDKTDKGIYVTRVSEGGPAEIAGL | |
| | | QIGDKIMQVNGWDMTMVTHDQARKRL | |
| | | TKRSEEVVRLLVTRQ | |
| KLMTTV | 45 | >sp\|P51142\|254-326 | 46 |
| | | TVTLNMEKYNFLGISIVGQSNERGDGGI | |
| | | YIGSIMKGGAVAADGRIEPGDMLLQVN | |
| | | DINFENMSNDDAVRVLRD | |
| GYSTRL | 55 | >sp\|Q15599\|150-230 | 48 |
| | | RLCHLRKGPQGYGFNLHSDKSRPGQYIR | |
| | | SVDPGSPAARSGLRAQDRLIEVNGQNVE | |
| | | GLRHAEVVASIKAREDEARLLVVDP | |
| EAQTRL | 49 | >sp\|Q9WV48\|663-757 | 58 |
| | | TVLLQKKDSEGFGFVLRGAKAQTPIEEF | |
| | | TPTPAFPALQYLESVDEGGVAWRAGLR | |
| | | MGDFLIEVNGQNVVKVGHRQVVNMIRQ | |
| | | GGNTLMVKVVMVT | |
| KIATLV | 51 | >sp\|O55164\|1138-1230 | 52 |
| | | RVELWREPSKSLGISIVGGRGMGSRLSN | |
| | | GEVMRGIFIKHVLEDSPAGKNGTLKPGD | |
| | | RIVEVDGMDLRDASHEQAVEAIRKAGSP | |
| | | VVFMVQSIV | |
| KIATLV | 51 | >sp\|O55164\|1613-1696 | 53 |
| | | TIEISKGQTGLGLSIVGGSDTLLGAIIIHEV | |
| | | YEEGAACKDGRLWAGDQILEVNGIDLR | |
| | | KATHDEAINVLRQTPQRVRLTLYRDE | |
| QDGTEV | 54 | >sp\|P97879\|53-136 | 55 |
| | | VVELMKKEGTTLGLTVSGGIDKDGKPR | |
| | | VSNLRQGGIAARSDQLDVGDYIKAVNGI | |
| | | NLAKFRHDEIISLLKNVGERVVLEVEYE | |
| | | LPPVSIQGSSVMFRTVEVTLHKEGNTFGF | |
| | | VIRGGAHDDRNKSRPVVITCVRPGGPAD | |
| | | REGTIKPGDRLLSVDGIRLLGTTHAEAM | |
| | | SILKQCGQEATLLIEYDV | |
| EEESQL | 56 | >sp\|Q8R4T5\|100-189 | 57 |
| | | VLTLEKGDNQTFGFEIQTYGLHHREEQR | |
| | | VEMVTFVCRVHESSPAQLAGLTPGDTIA | |
| | | SVNGLNVEGIRHREIVDIIKASGNVLRLE | |
| | | TLYGT | |
| LGATGL | 58 | >sp\|Q62696\|318-404 | 59 |
| | | EIKLIKGPKGLGFSIAGGVGNQHIPGDNSI | |
| | | YVTKIIEGGAAHKDGKLQIGDKLLAVNS | |
| | | VCLEEVTHEEAVTALKNTSDFVYLKAA | |
| | | KP | |
| RKETVA | 60 | >sp\|Q9EP80\|22-105 | 61 |
| | | KVTLQKDAQNLIGISIGGGAQYCPCLYIV | |
| | | QVFDNTPAALDGTVAAGDEITGVNGRSI | |
| | | KGKTKVEVAKMIQEVKGEVTIHYNKLQ | |
| SIESDV | 62 | >sp\|P31016\|65-151 | 14 |
| | | EITLERGNSGLGFSIAGGTDNPHIGDDPSI | |
| | | FITKIIPGGAAAQDGRLRVNDSILFVNEV | |
| | | DVREVTHSAAVEALKEAGSIVRLYVMR | |
| | | R | |
| SIESDV | 62 | >sp\|P31016\|160-246 | 63 |
| | | EIKLIKGPKGLGFSIAGGVGNQHIPGDNSI | |
| | | YVTKIIEGGAAHKDGRLQIGDKILAVNS | |
| | | VGLEDVMHEDAVAALKNTYDVVYLKV | |
| | | AKP | |
| SIESDV | 62 | >2sp\|P31016\|65-151 | 14 |
| | | EITLERGNSGLGFSIAGGTDNPHIGDDPSI | |
| | | FITKIIPGGAAAQDGRLRVNDSILFVNEV | |
| | | DVREVTHSAAVEALKEAGSIVRLYVMR | |
| | | R | |
| SIESDV | 62 | >sp\|P31016\|160-246 | 63 |
| | | EIKLIKGPKGLGFSIAGGVGNQHIPGDNSI | |
| | | YVTKIIEGGAAHKDGRLQIGDKILAVNS | |
| | | VGLEDVMHEDAVAALKNTYDVVYLKV | |
| | | AKP | |
| QSSSSL | 64 | >sp\|Q8R4T5\|100-189 | 65 |
| | | VLTLEKGDNQTFGFEIQTYGLHHREEQR | |
| | | VEMVTFVCRVHESSPAQLAGLTPGDTIA | |
| | | SVNGLNVEGIRHREIVDIIKASGNVLRLE | |
| | | TLYGT | |
| PFSSSV | 66 | >sp\|P31016\|65-246 | 67 |
| | | EITLERGNSGLGFSIAGGTDNPHIGDDPSI | |
| | | FITKIIPGGAAAQDGRLRVNDSILFVNEV | |
| | | DVREVTHSAAVEALKEAGSIVRLYVMR | |
| | | RKPPAEKVMEIKLIKGPKGLGFSIAGGV | |
| | | GNQHIPGDNSIYVTKIIEGGAAHKDGRL | |
| | | QIGDKILAVNSVGLEDVMHEDAVAALK | |
| | | NTYDVVYLKVAKP | |
| LEDSVF | 68 | >sp\|O00560\|198-273 | 69 |
| | | TITMHKDSTGHVGFIFKNGKITSIVKDSS | |
| | | AARNGLLTEHNICEINGQNVIGLKDSQIA | |
| | | DILSTSGTVVTITIMPAF | |
| LEDSVF | 68 | >sp\|O00560\|114-273 | 70 |
| | | EVILCKDQDGKIGLRLKSIDNGIFVQLVQ | |
| | | ANSPASLVGLRFGDQVLQINGENCAGW | |
| | | SSDKAHKVLKQAFGEKITMTIRDRPFER | |
| | | TITMHKDSTGHVGFIFKNGKITSIVKDSS | |
| | | AARNGLLTEHNICEINGQNVIGLKDSQIA | |
| | | DILSTSGTVVTITIMPAF | |
| RRESAI | 71 | >sp\|O14907\|15-112 | 72 |
| | | RVEIHKLRQGENLILGFSIGGGIDQDPSQ | |
| | | NPFSEDKTDKGIYVTRVSEGGPAEIAGL | |
| | | QIGDKIMQVNGWDMTMVTHDQARKRL | |
| | | TKRSEEVVRLLVTRQ | |
| SVSTVV | 73 | >sp\|P31016\|65-151 | 14 |
| | | EITLERGNSGLGFSIAGGTDNPHIGDDPSI | |
| | | FITKIIPGGAAAQDGRLRVNDSILFVNEV | |
| | | DVREVTHSAAVEALKEAGSIVRLYVMR | |
| | | R | |
| SVSTVV | 73 | >sp\|P31016\|160-246 | 74 |
| | | EIKLIKGPKGLGFSIAGGVGNQHIPGDNSI | |
| | | YVTKIIEGGAAHKDGRLQIGDKILAVNS | |
| | | VGLEDVMHEDAVAALKNTYDVVYLKV | |
| | | AKP | |
| AEDSFL | 75 | >sp\|O14745\|14-94 | 76 |
| | | LCCLEKGPNGYGFHLHGEKGKLGQYIRL | |
| | | VEPGSPAEKAGLLAGDRLVEVNGENVE | |
| | | KETHQQVVSRIRAALNAVRLLVVDPE | |
| LTASEV | 95 | >sp\|Q9Z0G0\|133-213 | 78 |
| | | EVEVFKSEEALGLTITDNGAGYAFIKRIK | |
| | | EGSVIDHIQLISVGDMIEAINGQSLLGCR | |
| | | HYEVARLLKELPRGRTFTLKLTE | |
| EQVSAV | 79 | >sp\|Q12923\|1368-1452 | 80 |
| | | EVELAKNDNSLGISVTGGVNTSVRHGGI | |
| | | YVKAVIPQGAAESDGRIHKGDRVLAVN | |
| | | GVSLEGATHKQAVETLRNTGQVVHLLL | |
| | | EKG | |
| VQQTRV | 81 | >sp\|P31016\|65-151 | 82 |
| | | EITLERGNSGLGFSIAGGTDNPHIGDDPSI | |
| | | FITKIIPGGAAAQDGRLRVNDSILFVNEV | |
| | | DVREVTHSAAVEALKEAGSIVRLYVMR | |
| | | R | |
| VQQTRV | 81 | >sp\|P31016\|160-246 | 83 |
| | | EIKLIKGPKGLGFSIAGGVGNQHIPGDNSI | |
| | | YVTKIIEGGAAHKDGRLQIGDKILAVNS | |
| | | VGLEDVMHEDAVAALKNTYDVVYLKV | |
| | | AKP | |
| VQQTRV | 81 | >sp\|P31016\|313-393 | 84 |
| | | RIVIHRGSTGLGFNIVGGEDGEGIFISFILA | |
| | | GGPADLSGELRKGDQILSVNGVDLRNAS | |
| | | HEQAAIALKNAGQTVTIIAQYK | |
| ASGSSV | 85 | >sp\|Q9WVJ4\|13-100 | 86 |
| | | EINLTRGPSGLGFNIVGGTDQQYVSNDS | |
| | | GIYVSRIKEDGAAARDGRLQEGDKILSV | |
| | | NGQDLKNLLHQDAVDLFRNAGYAVSLR | |
| | | VQHRL | |
| FRETEV | 87 | >sp\|P31016\|65-151 | 88 |
| | | EITLERGNSGLGFSIAGGTDNPHIGDDPSI | |
| | | FITKIIPGGAAAQDGRLRVNDSILFVNEV | |
| | | DVREVTHSAAVEALKEAGSIVRLYVMR | |
| | | R | |
| FRETEV | 87 | >sp\|P31016\|160-246 | 89 |
| | | EIKLIKGPKGLGFSIAGGVGNQHIPGDNSI | |
| | | YVTKIIEGGAAHKDGRLQIGDKILAVNS | |
| | | VGLEDVMHEDAVAALKNTYDVVYLKV | |
| | | AKP | |
| LEDTEL | 90 | >sp\|Q9Y6N9\|87-169 | 91 |
| | | EVRLDRLHPEGLGLSVRGGLEFGCGLFIS | |
| | | HLIKGGQADSVGLQVGDEIVRINGYSISS | |
| | | CTHEEVINLIRTKKTVSIKVRHIGL | |
| RRETQL | 92 | >sp\|Q96QZ7\|17-105 | 93 |
| | | ECTVKRGPQGELGVTVLGGAEHGEFPY | |
| | | VGAVAAVEAAGLPGGGEGPRLGEGELL | |
| | | LEVQGVRVSGLPRYDVLGVIDSCKEAVT | |
| | | FKAVRQG | |
| RRETQV | 94 | >sp\|Q6RHR9\|464-546 | 95 |
| | | HTKLRKSSRGFGFTVVGGDEPDEFLQIK | |
| | | SLVLDGPAALDGKMETGDVIVSVNDTC | |
| | | VLGHTHAQVVKIFQSIPIGASVDLELCR | |
| RRETQV | 94 | >sp\|Q6RHR9\|17-105 | 96 |
| | | ECTVKRGPQGELGVTVLGGAEHGEFPY | |
| | | VGAVAAAEAAGLPGGGEGPKLAEGELL | |
| | | LEVQGVRVSGLPRYDVLGVIDSCKEAVT | |
| | | FKAVRQG | |
| RRETQV | 94 | >sp\|Q6RHR9\|464-546 | 97 |
| | | HTKLRKSSRGFGFTVVGGDEPDEFLQIK | |
| | | SLVLDGPAALDGKMETGDVIVSVNDTC | |
| | | VLGHTHAQVVKIFQSIPIGASVDLELCR | |
| GGETRL | 98 | >sp\|Q6P0Q8\|1104-1192 | 99 |
| | | PIIIHRAGKKYGFTLRAIRVYMGDSDVYT | |
| | | VHHMVWHVEDGGPASEAGLRQGDLITH | |
| | | VNGEPVHGLVHTEVVELILKSGNKVAIS | |
| | | TTPLE | |
| TNETSL | 100 | >sp\|Q14160\|728-815 | 101 |
| | | TLTILRQTGGLGISIAGGKGSTPYKGDDE | |
| | | GIFISRVSEEGPAARAGVRVGDKLLEVN | |
| | | GVALQGAEHHEAVEALRGAGTAVQMR | |
| | | VWRER | |
| TNETSL | 125 | >sp\|Q14160\|1004-1093 | 102 |
| | | EIRLPRAGGPLGLSIVGGSDHSSHPFGVQ | |
| | | EPGVFISKVLPRGLAARSGLRVGDRILAV | |
| | | NGQDVRDATHQEAVSALLRPCLELSLLV | |
| | | RRDP | |
| EEDTHL | 103 | >sp\|Q15599\|11-90 | 104 |
| | | LCRLVRGEQGYGFHLHGEKGRRGQFIRR | |
| | | VEPGSPAEAAALRAGDRLVEVNGVNVE | |
| | | GETHHQVVQRIKAVEGQTRLLVVDQ | |
| EEDTHL | 105 | >sp\|Q15599\|150-230 | 106 |
| | | RLCHLRKGPQGYGFNLHSDKSRPGQYIR | |
| | | SVDPGSPAARSGLRAQDRLIEVNGQNVE | |
| | | GLRHAEVVASIKAREDEARLLVVDP | |
| LQQSNV | 107 | >sp\|Q63ZW7\|1568-1650 | 108 |
| | | LVDLQKKTGRGLGLSIVGKRSGSGVFIS | |
| | | DIVKGGAADLDGRLIRGDQILSVNGEDM | |
| | | RHASQETVATILKCVQGLVQLEIGRLR | |
| SIESDV | 62 | >sp\|Q63ZW7\|1568-1650 | 109 |
| | | LVDLQKKTGRGLGLSIVGKRSGSGVFIS | |
| | | DIVKGGAADLDGRLIRGDQILSVNGEDM | |
| | | RHASQETVATILKCVQGLVQLEIGRLR | |
| SIESDV | 62 | >sp\|Q63ZW7\|1568-1650 | 110 |
| | | LVDLQKKTGRGLGLSIVGKRSGSGVFIS | |
| | | DIVKGGAADLDGRLIRGDQILSVNGEDM | |
| | | RHASQETVATILKCVQGLVQLEIGRLR | |
| SLESEV | 111 | >sp\|Q63ZW7\|1568-1650 | 112 |
| | | LVDLQKKTGRGLGLSIVGKRSGSGVFIS | |
| | | DIVKGGAADLDGRLIRGDQILSVNGEDM | |
| | | RHASQETVATILKCVQGLVQLEIGRLR | |
| SLESEV | 111 | >sp\|Q63ZW7\|1568-1650 | 113 |
| | | LVDLQKKTGRGLGLSIVGKRSGSGVFIS | |
| | | DIVKGGAADLDGRLIRGDQILSVNGEDM | |
| | | RHASQETVATILKCVQGLVQLEIGRLR | |
| YLVTRL | 114 | >sp\|Q63ZW7\|1709-1795 | 115 |
| | | TVEIIRELSDALGISIAGGKGSPLGDIPIFI | |
| | | AMIQANGVAARTQKLKVGDRIVSINGQP | |
| | | LDGLSHTDAVNLLKNAFGRIILQVVADT | |
| GPATDL | 116 | >sp\|Q63ZW7\|1074-1166 | 117 |
| | | IVEIFREPNVSLGISIVGGQTVIKRLKNGE | |
| | | ELKGIFIKQVLEDSPAGKTNALKTGDKIL | |
| | | E | |
| | | VSGVDLQNASHAEAVEAIKSAGNPVVF | |
| | | VVQSLS | |

Exemplary Type II PDZ carboxy terminal domains and corresponding PDZ domain peptides are illustrated in Table 3.

TABLE 3
Type II PDZ binding carboxy terminal pep-
tides and corresponding PDZ domain peptides

| Type II C- | SEQ | PDZ domain Uniprot | SEQ |
|---|---|---|---|
| terminal | ID | Identifier and PDZ | ID |
| peptides | NO | Domain Sequence | NO: |
| EQPVYI | 118 | >sp\|Q02410\|656-822 | 119 |
| | | DVFIEKQKGEILGVVIVESGWGSILPTVII | |
| | | ANMMHGGPAEKSGKLNIGDQIMSINGTS | |
| | | LVGLPLSTCQSIIKGLKNQSRVKLNIVRC | |
| | | PPVTTVLIRRPDLRYQLGFSVQNGIICSL | |
| | | MRGGIAERGGVRVGHRIIEINGQSVVAT | |
| | | PHEKIVHILSNAVGEIHMKTMPA | |
| QEELII | 120 | >sp\|Q99NH2\|590-677 | 121 |
| | | EVPLNDSGSAGLGVSVKGNRSKENHAD | |
| | | LGIFVKSIINGGAASKDGRLRVNDQLIAV | |
| | | NGESLLGKANQEAMETLRRSMSTEGNK | |
| | | RGMIQ | |
| EEGIWA | 122 | >sp\|Q9JIR4\|1619-705 | 123 |
| | | RTTMPKESGALLGLKVVGGKMTDLGRL | |
| | | GAFITKVKKGSLADVVGHLRAGDEVLE | |
| | | WNGKPLPGATNEEVYNIILESKSEPQVEI | |
| | | IVSR | |
| IESVKI | 124 | >sp\|Q9EP80\|22-105 | 125 |
| | | KVTLQKDAQNLIGISIGGGAQYCPCLYIV | |
| | | QVFDNTPAALDGTVAAGDEITGVNGRSI | |
| | | KGKTKVEVAKMIQEVKGEVTIHYNKLQ | |
| VRTYSC | 126 | >sp\|P97879\|672-754 | 127 |
| | | TVELKRYGGPLGITISGTEEPFDPIIISSLT | |
| | | KGGLAERTGAIHIGDRILAINSSSLKGKP | |
| | | LSEAIHLLQMAGETVTLKIKKQT | |
| RKEYFI | 128 | >sp\|Q00013\|71-152 | 129 |
| | | LIQFEKVTEEPMGITLKLNEKQSCTVARI | |
| | | LHGGMIHRQGSLHVGDEILEINGTNVTN | |
| | | HSVDQLQKAMKETKGMISLKVIPNQ | |
| TNEFYA | 130 | >sp\|O00560\|114-273 | 131 |
| | | EVILCKDQDGKIGLRLKSIDNGIFVQLVQ | |
| | | ANSPASLVGLRFGDQVLQINGENCAGW | |
| | | SSDKAHKVLKQAFGEKITMTIRDRPFER | |
| | | TITMHKDSTGHVGFIFKNGKITSIVKDSS | |
| | | AARNGLLTEHNICEINGQNVIGLKDSQIA | |
| | | DILSTSGTVVTITIMPAF | |
| GLDVPV | 132 | >sp\|Q96RT1\|1321-1410 | 133 |
| | | EIRVRVEKDPELGFSISGGVGGRGNPFRP | |
| | | DDDGIFVTRVQPEGPASKLLQPGDKIIQA | |
| | | NGYSFINIEHGQAVSLLKTFQNTVELIIV | |
| | | REV | |
| DKEYYV | 134 | >sp\|O35889\|1014-1100 | 135 |
| | | VITVTLKKQNGMGLSIVAAKGAGQDKL | |
| | | GIYVKSVVKGGAADVDGRLAAGDQLLS | |
| | | VDGRSLVGLSQERAAELMTRTSSVVTLE | |
| | | VAKQG | |
| QEEFYA | 136 | >sp\|O00560\|114-273 | 137 |
| | | EVILCKDQDGKIGLRLKSIDNGIFVQLVQ | |
| | | ANSPASLVGLRFGDQVLQINGENCAGW | |
| | | SSDKAHKVLKQAFGEKITMTIRDRPFER | |
| | | TITMHKDSTGHVGFIFKNGKITSIVKDSS | |
| | | AARNGLLTEHNICEINGQNVIGLKDSQIA | |
| | | DILSTSGTVVTITIMPAF | |
| FHQFYI | 138 | >sp\|Q02410\|656-822 | 139 |
| | | DVFIEKQKGEILGVVIVESGWGSILPTVII | |
| | | ANMMHGGPAEKSGKLNIGDQIMSINGTS | |
| | | LVGLPLSTCQSIIKGLKNQSRVKLNIVRC | |
| | | PPVTTVLIRRPDLRYQLGFSVQNGIICSL | |
| | | MRGGIAERGGVRVGHRIIEINGQSVVAT | |
| | | PHEKIVHILSNAVGEIHMKTMPA | |
| DREYYV | 140 | >sp\|O35889\|1014-1100 | 141 |
| | | VITVTLKKQNGMGLSIVAAKGAGQDKL | |
| | | GIYVKSVVKGGAADVDGRLAAGDQLLS | |
| | | VDGRSLVGLSQERAAELMTRTSSVVTLE | |
| | | VAKQG | |
| NRPVPV | 142 | >sp\|Q63ZW7\|1472-1555 | 143 |
| | | IIEISKGRSGLGLSIVGGKDTPLDAIVIHE | |
| | | VYEEGAAARDGRLWAGDQILEVNGVDL | |
| | | RSSSHEEAITALRQTPQKVRLVVYRDE | |
| DKEYYV | 134 | >sp\|Q63ZW7\|1568-1650 | 144 |
| | | LVDLQKKTGRGLGLSIVGKRSGSGVFIS | |
| | | DIVKGGAADLDGRLIRGDQILSVNGEDM | |
| | | RHASQETVATILKCVQGLVQLEIGRLR | |
| KTEFCA | 145 | >sp\|Q24008\|17-106 | 146 |
| | | MVTLDKTGKKSFGICIVRGEVKDSPNTK | |
| | | TTGIFIKGIVPDSPAHLCGRLKVGDRILSL | |
| | | NGKDVRNSTEQAVIDLIKEADFKIELEIQ | |
| | | TFD | |
| KTEFCA | 145 | >sp\|Q24008\|584-664 | 147 |
| | | NVDLMKKAGKELGLSLSPNEIGCTIADLI | |
| | | QGQYPEIDSKLQRGDIITKFNGDALEGLP | |
| | | FQVCYALFKGANGKVSMEVTRPK | |

Exemplary Type III PDZ carboxy terminal domains and corresponding PDZ domain peptides are illustrated in Table 4.

TABLE 4
Type III PDZ binding carboxy terminal pep-
tides and corresponding PDZ domain peptides

| Type III C- | SEQ | PDZ domain Uniprot | SEQ |
|---|---|---|---|
| terminal | ID | Identifier and PDZ | ID |
| peptides | NO | Domain Sequence | NO |
| VKVDSV | 148 | >sp\|P29476\|17-99 | 149 |
| | | SVRLFKRKVGGLGFLVKERVSKPPV | |
| | | IISDLIRGGAAEQSGLIQAGDIILAVN | |
| | | DRPLVDLSYDSALEVLRGIASETHV | |
| | | VLILRG | |
| FGGEPL | 150 | >sp\|O17583\|801-886 | 151 |
| | | EVVVPKKAGEPLGIVVVESGWGSM | |
| | | LPTVVLAHMNPVGPAAHSNKLNIGD | |
| | | QIININGISLVGLPLSAAQTQIKNMKT | |
| | | ATAVRMTVVS | |
| AFFEEL | 152 | >sp\|Q9JI92\|116-195 | 153 |
| | | EVILCKDQDGKIGLRLKSVDNGIFVQ | |
| | | LVQANSPASLVGLRFGDQVLQINGE | |
| | | NCAGWSSDKAHKVLKQAFGEKITM | |
| | | TIRDR | |
| SGKDYV | 154 | >sp\|Q63ZW7\|1472-1555 | 155 |
| | | IIEISKGRSGLGLSIVGGKDTPLDAIVI | |
| | | HEVYEEGAAARDGRLWAGDQILEV | |
| | | NGVDLRSSSHEEAITALRQTPQKVR | |
| | | LVVYRDE | |

In embodiments, herein described a PDZ binding carboxy terminal peptide can also be formed by a derivative of SEQ ID NO: 2 by possibly cyclizing the SEQ ID NO: 2 in residues X₁ and X₃ and/or modifying the residues for example by stripping side chains and introducing beta carbon or other modifications identifiable by a skilled person (e.g. to produce pseudopeptides) while maintaining the carboxy terminus and at least one amide bond between amino acid residues and therefore maintain the PDZ binding carboxy terminal peptide ability to specifically bind to a PDZ domain peptide with a set dissociation K_{D}.

The term “pseudopeptides” as used herein indicates a peptide in which one or more amide bonds have been replaced. Such definition is broad and further expansive then the original intended use to only refer to—CH₂S-modification. Examples include introduction of ester bonds (COO), thioester amide bonds (COS), ketomethylene amide bonds (DOCH₂), thioamide bonds (CSNH), reduced amide bonds (CH₂NH), thioether bonds (CH₂S), methylene sulfoxide bonds (CH₂SO), methylene sulfoxide amid bonds (CH₂SO), dimethylene amide bonds (CHₛCH₂).

In embodiments herein described, PDZ binding C-terminal peptide-PDZ domain peptide interactions can have a dissociation constant (K_{D}) values between 0.1-10 uM (35,47-52). The strength of interactions between candidate PDZ binding carboxy terminal peptides and PDZ domain peptide can be measured by several techniques such as Surface Plasmon Resonance (SPR)/Biacore, fluorescence polarization, Fluorescence Resonance Energy Transfer (FRET), Isothermal Titration calorimetry (ITC), bead based binding assays and additional techniques identifiable by a skilled person. Dissociation constant (K_{D}) values from 0.1 uM to zero uM (Covalent binding) can also obtained by directed evolution of PDZ domain and ligand pairs, with a dissociation constant (K_{D}) value of zero being obtained by designing a PDZ domain and Ligand pair that make a covalent bond to one another.

In some embodiments, a PDZ domain protein can bind a corresponding PDZ domain through binding of a PDZ domain with an internal PDZ binding peptide motif (herein referred to as a β-hairpin) to a PDZ domain binding grove. In those embodiments, at least one of the corresponding PDZ domains is a PDZ domain with β-hairpin.

The term “β-Hairpin” and “PDZ β-Hairpin” as used herein refers to a peptide sequence attached to PDZ domain that mimics the shape of a C-terminal peptide PDZ ligand, and can dock into the ligand binding groove of a corresponding PDZ domain (e.g. syntrophin/PSD-95). In particular, β-Hairpin refers to the hairpin shaped region formed by two beta strands (β1 and β2) linked by a loop, that can be attached at the carboxy terminal end of certain PDZ domain (2). The linear amino acid sequence of a β-Hairpin comprise the secondary structure elements β1 and β2, and loop together with a N-terminal segment, in a linear N-terminus carboxy terminus arrangement wherein the N-terminal loop segment of 3 to 6 residues is linked to the β1 beta strand of 8 to 10 residues, the β1 beta strand linked to the loop of 4 to 5 amino acids forming a beta turn changing direction of the peptide, the loop is linked to the β2 beta strand of 8 residues. In the β-Hairpin β1 contains a PDZ binding C-terminal peptide mimic. The term “mimic” and “mimicking” as used herein indicate a compound or a region that models the molecular structure, spectroscopic properties, or reactivity of a referenced molecule or region. Accordingly a mimic of the PDZ binding C-terminal peptide is a region that presents the same structural topology and binding properties and reactivity of the PDZ binding C-terminal peptide.

In PDZ domains containing a β-Hairpin the remaining portion of the PDZ domain (six beta strands (βA-βF), two alpha helices (αA & αB), and GLGF loop define a region herein also indicated as PDZ domain core to which the β-Hairpin is covalently attached. In particular the N-terminal segment of the of-β-Hairpin is covalently attached at the carboxy terminus of the PDZ domain core. The C-terminus (also known as the carboxyl-terminus, carboxy-terminus, C-terminal tail, C-terminal end, or COOH-terminus) indicates the end of an amino acid chain (protein or polypeptide), terminated by a free carboxyl group (—COOH) or a by a carboxyl group not involved in a peptide bond.

In a PDZ domain comprising a β-Hairpin, the PDZ domain core and β-Hairpin fold can be stabilized by a salt bridge formed by an acidic amino acid in the PDZ domain core located in its βD strand and a basic amino acid in the β-Hairpin located in its β-2 strand. In particular, in some embodiments β2 comprise a basic amino acid, typically R or K in a position adjacent to the C-terminus of the PDZ domain core.

In some embodiments a β-Hairpin can have the following sequence X₁-X₂-X₃-X₄-X₅-X₆-X₇-X₈-X₉ X₁₀-X₁₁-X₁₂-X₁₃-X₁₄-X₁₅-X₁₆-X₁₇-X₁₈-X₁₉-X₂₀-X₂₁-X₂₂-X₂₃-X₂₄ X₂₅-X₂₆-X₂₇-X₂₈-X₂₉ wherein

X₁ to X₆ can be any amino acids defining an N-terminal segment where up to three of the X₁ to X₆ amino acids residues might be absent;
X₇ to X₁₆ can be any amino acids forming a 131 beta strand mimicking SEQ ID NO: 2 where up to two of the X₇ to X₁₆ amino acid residues might be absent;
X₁₇ to X₂₁ can be any amino acids forming a loop region where one of the X₁₇ to X₂₁ amino acids might be absent; and
X₂₂ to X₂₉ can be any amino acids forming a β2 beta strand wherein one of the X₂₅ to X₂₉ is R or K. (SEQ ID NO: 156).

In some embodiments, in SEQ ID NO: 156, X₇ to X₁₆ can comprise SEQ ID NO: 4 or a derivative thereof. In some embodiments, X₇ to X₁₆ can comprise the sequence of a Type I or Type II PDZ binding carboxy terminal peptides or a derivative thereof.

In several embodiments, β-Hairpin can comprise sequence RGPEGFTTHLETTFTGDGTPKTIRVTQ SEQ ID NO:157, wherein the region mimicking the PDZ binding C-terminal peptide is ETTF (SEQ ID NO: 158).

In the β-Hairpin regions the SEQ ID NO: 156 or derivative thereof attaches at the carboxy terminus of a protein, a six to ten amino acid residue region forming a β-turn and the remaining portion of the β-Hairpin. In the second β-strand located in the β-Hairpin region SEQ ID NO: 156 or derivative thereof, a salt bridge is formed between a conserved Arginine or Lysine residue and an adjacent acidic residue (Aspartic or glutamic acid) in the PDZ domain core sequence of the PDZ domain containing the PDZ-BH (2). The term “adjacent” as used herein indicate proximity within the length of 1 to 5 amino acid residues.

Accordingly in some embodiments a PDZ domain comprising a β-Hairpin has sequence

X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-G-X₂₀-X₂₁-X₂₂-X₂₃-X₂₄-
X₂₅-X₂₆-X₂₇ X₂₈-X₂₉-X₃₀-X₃₁-X₃₂-X₃₃-X₃₄-X₃₅-X₃₆-X₃₇-X₃₈-X₃₉-X₄₀-X₄₁-X₄₂ X₄₃-X₄₄-X₄₅-X₄₆-
X₄₇-X₄₈-X₄₉-X₅₀-X₅₁-X₅₂-X₅₃-X₅₄-X₅₅-X₅₆-X₅₇-X₅₈-X₅₉-X₆₀-X₆₁-X₆₂-X₆₃-X₆₄-X₆₅-X₆₆-X₆₇-X₆₈-
X₆₉-X₇₀-X₇₁-X₇₂₋-X₇₃-X₇₄-X₇₅-X₇₆-X₇₇-X₇₈-X₇₉-X₈₀-X₈₁-X₈₂-X₈₃-X₈₄-X₈₅-X₈₆-X₈₈-X₈₉-X₉₀-X₉₁-
X₉₂-X₉₃-X₉₄-X₉₅-X₉₆-X₉₇-X₉₈-X₉₉-X₁₀₀-X₁₀₁-X₁₀₂-X₁₀₃-X₁₀₄-X₁₀₅-X₁₀₆-X₁₀₇-X₁₀₈-X₁₀₉-X₁₁₀-X₁₁₁-
X₁₁₂-X₁₁₃-X₁₁₄-X₁₁₅-X₁₁₆-X₁₁₇-X₁₁₈-X₁₁₉ X₁₂₀-X₁₂₁-X₁₂₂-X₁₂₃-X₁₂₄-X₁₂₅-X₁₂₆-X₁₂₇-X₁₂₈-X₁₂₉-X₁₃₀-
X₁₃₁-X₁₃₂-X₁₃₃-X₁₃₄-X₁₃₅-X₁₃₆-X₁₃₇-X₁₃₈-X₁₃₉-X₁₄₀-X₁₄₁-X₁₄₂-X₁₄₃-X₁₄₄-X₁₄₅-X₁₄₆-X₁₄₇-X₁₄₈-
X₁₄₉-X₁₅₀-X₁₅₁-X₁₅₂-X₁₅₃-X₁₅₄-X₁₅₅-X₁₅₆-X₁₅₇-X₁₅₈-X₁₅₉

wherein residues X₁ to X₁₃₀ have sequence SEQ ID NO: 1 and therefore
X₁ to X₅ can be any amino acids defining an N-terminal segment wherein up to four of the X₁ to X₅ amino acids residues might be absent;
X₆ to X₁₁ can be any amino acids forming the βA beta strand wherein one of the X₆ to X₁₁ amino acid residues might be absent;
X₁₂ to X₂₁ form the GLGF loop wherein X₁₂ can be any amino acid, X₁₃ can be R or K, X₁₄ to X₁₆ can be any amino acid X₁₇ can be any amino acid, X₁₈ and X₂₀ are hydrophobic amino acids, X₂₁ can be any amino acid, and where up to two amino acids of X₁₂ X₁₄ to X₁₆ and X₂₁ might be absent;
X₂₂ to X₂₇ can be any amino acids forming the βB beta strand wherein up to two of the X₂₂ to X₂₇ amino acids might be absent;
X₂₈ to X₄₁ can be any amino acids forming a loop region wherein up to 13 of the X₂₈ to X₄₁ amino acids might be absent;
X₄₂ to X₄₈ can be any amino acids forming the βC beta strand wherein up to two of the X₄₂ to X₄₈ amino acids might be absent;
X₄₉ to X₅₃ can be any amino acids forming a loop region wherein one of the X₄₉ to X₅₃ amino acid residues might be absent;
X₅₄ to X₅₉ can be any amino acids forming the αA alpha helix wherein up to four of the X₅₄ to X₅₉ amino acid residues might be absent;
X₆₀ to X₆₇ can be any amino acids forming a loop region wherein up to three of the X₆₀ to X₆₇ amino acid residues might be absent;
X₆₈ to X₇₅ can be any amino acids forming the βD beta strand wherein up to three of the X₆₈ to X₇₅ amino acid residues might be absent;
X₇₆ to X₇₉ can be any amino acids forming a loop region wherein up to two of the X₇₆ to X₇₉ amino acid residues might be absent;
X₈₀ to X₈₃ can be any amino acids forming the βE beta strand wherein up to two of the X₈₀ to X₈₃ amino acid residues might be absent;
X₈₄ to X₉₂ can be any amino acids forming a loop region where up to four of the X₈₄ to X₉₂ amino acid residues might be absent;
X₉₃ to X₁₀₂ can be any amino acids forming a αB alpha helix wherein up to two of the X₉₃ to X₁₀₂ amino acid residues might be absent;
X₁₀₃ to X₁₁₀ can be any amino acids forming a loop region wherein up to two of the X₁₀₃ to X₁₁₀ amino acid residues might be absent;
X₁ᵢᵢ to X₁₁₈ can be any amino acids forming a βF beta strand wherein up to two of the X₁₁₁ to X₁₁₈ amino acid residues might be absent;
X₁₁₉ to X₁₂₁ can be any amino acids forming a loop region wherein one of the X₁₁₉ to X₁₂₁ amino acid residues might be absent; and
X₁₂₂ to X₁₃₀ can be any amino acids forming a αC alpha helix wherein all of the X₁₂₂ to X₁₃₀ amino acid residues might be absent;
and wherein residues X₁₃₁ to X₁₅₉ have SEQ ID NO: 156 and therefore
X₁₃₁ to X₁₃₆ can be any amino acids defining an N-terminal segment where up to three of the X₁₃₁ to X₁₃₆ amino acids residues might be absent;
X₁₃₇ to X₁₄₆ can be any amino acids forming a β1 beta strand mimicking SEQ ID NO: 2 where up to two of the X₁₃₇ to X₁₄₆ amino acid residues might be absent;
X₁₄₇ to X₁₅₁ can be any amino acids forming a loop region where one of the X₁₄₇ to X₁₅₁ amino acids might be absent; and
X₁₅₂ to X₁₅₉ can be any amino acids forming a β2 beta strand wherein one of the X₁₅₂ to X₁₅₉ is R or K (SEQ ID NO: 159)

In a PDZ domain comprising a β-Hairpin a β-Hairpin binds a corresponding PDZ domain in a “lock and key” fit via the GLGF loop of the corresponding PDZ domain, the GLGF loop having sequence SEQ ID NO: 3 in which X₁ is the amino acids K or A, and a highly ordered water molecule constrained by the partially buried side chains of conserved R or K immediately following the βA strand of the corresponding PDZ in the interior of its ligand binding pocket. In the PDZ BH-corresponding PDZ domain interaction the GLGF loop of the corresponding PDZ domain acts as a steric block for Beta Hairpin sequence, and selective recognition of β-Hairpin sequence is provided by van der Waals contacts between the hydrophobic pocket in the ligand binding pocket of the PDZ domain and the hydrophobic side chain of the a β-Hairpin that mimics the carboxy terminal residue in a Type I or II PDZ domain ligand. The term “PDZ BH” as used herein refers to a combined PDZ domain and carboxy terminal β-Hairpin peptide sequence attached to a PDZ domain that mimics the shape of a C-terminal peptide PDZ ligand, and can dock into the peptide binding groove of another (e.g. syntrophin/PSD-95) PDZ domain (2).

Identification of specific β-Hairpin sequences in a PDZ domain protein in the sense of the present disclosure can be provided by sequences of naturally occurring PDZ domains with β-Hairpins (e.g. the PDZ domains of nNOS (Uniprot ID: Q9Z0J4) (2,53). and/or by use of software or sequence alignment programs (ClustalW, MUSCLE, BLAST-P, PSI-BLAST, Threader) able to provide amino acid sequences that can form the hairpin shaped region comprised the two beta strands and having the conserved arginine or lysine adjacent to the carboxy terminus of the PDZ core domains as herein described. Exemplary computational design methods comprise methods using structure prediction and protein design packages such as Modeller, Rosetta, Robetta, iTasser, Orbit, machine learning algorithms, statistical potentials, and additional methods identifiable by a skilled person. Additional methods comprise directed evolution, or directed evolution in combination with a computationally guided approach starting from a known naturally occurring or synthetic sequence as will be understood by a skilled person.

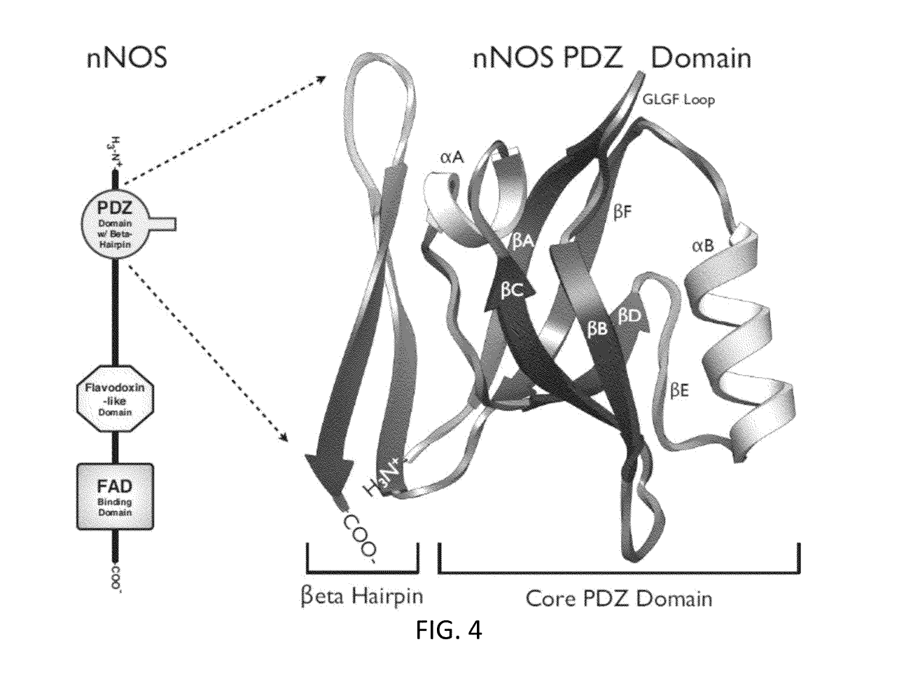

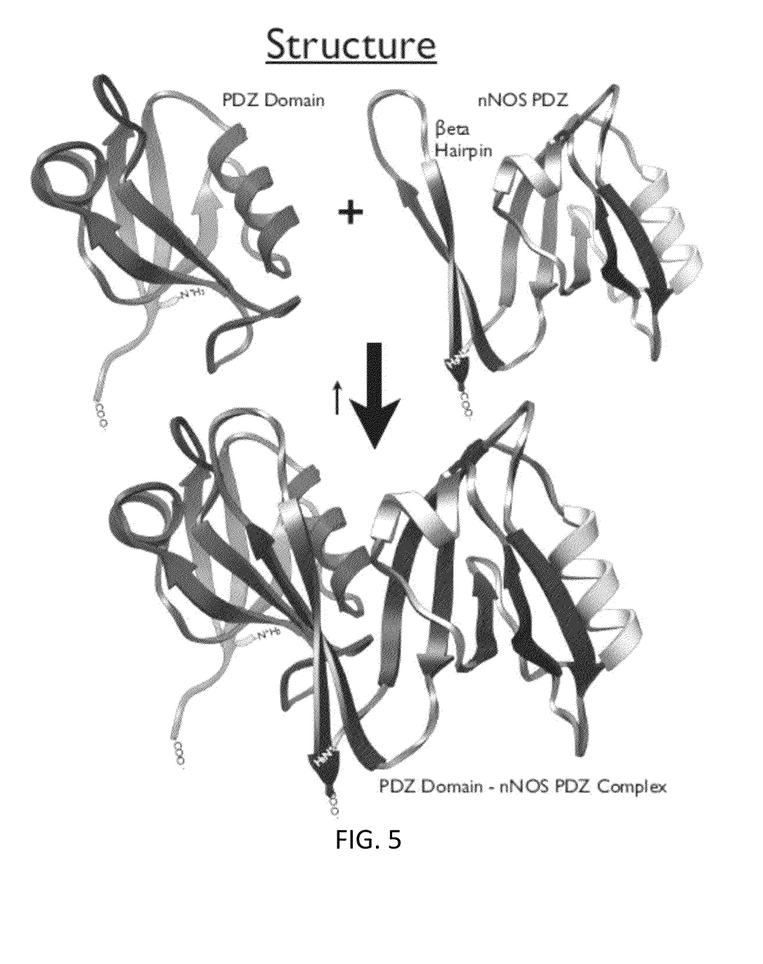

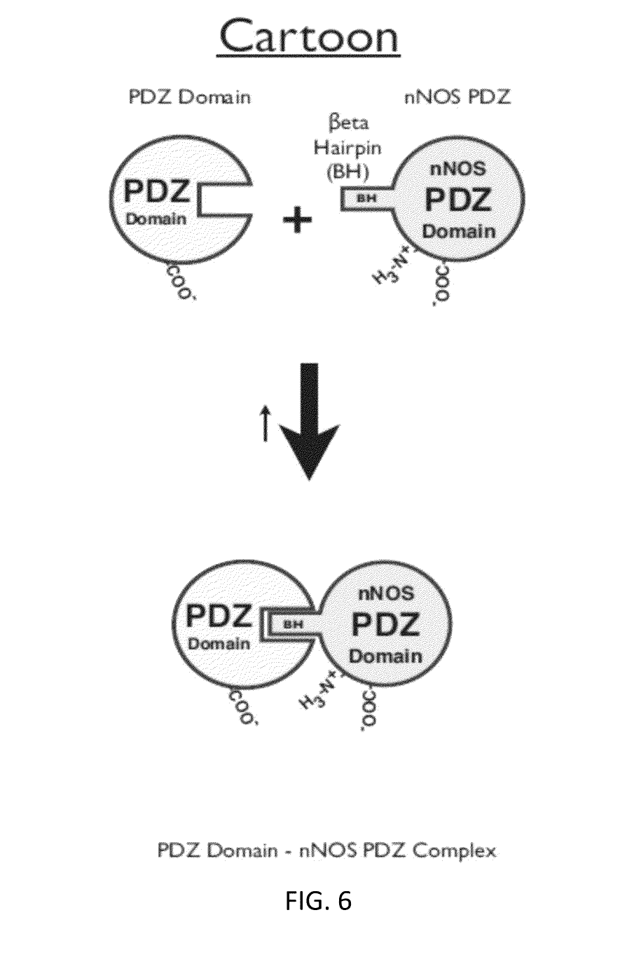

Reference is made to the illustration of FIG. 4 which also shows an x-ray crystal structure and ribbon representation, of a PDZ domain with a C-terminal β-hairpin, from the protein neuronal Nitric Oxide Synthase (nNOS), and its positioning in the full length nNOS protein. As shown, in several embodiments herein, in addition to binding short, C-terminal peptide sequences, several PDZ domains have been shown to engage in homotypic interactions, by binding to other PDZ domains through internal peptide motifs. As shown in FIG. 4, this type of PDZ domain-PDZ domain interaction involves the PDZ domain from neuronal nNOS and either the PDZ2 domain from PSD-95 or the PDZ domain from syntrophin (2,53). The nNOS PDZ domain can form a linear head to tail arrangement with PSD-95 PDZ2 or syntrophin PDZ through a short C-terminal structural motif referred to as a β-hairpin (BH). In this interaction, the canonical ligand binding groove of nNOS PDZ is exposed to solution whereas the other face of the nNOS PDZ has a β-hairpin that can dock into the ligand binding groove of syntrophin or PSD-95 PDZ2. The β-hairpin mimics the structure and shape of a normal C-terminal peptide ligand, however a sharp Beta turn exists in place of the essential peptide carboxy terminus, forming a strong, sub-micromolar (K_{D} of ˜0.6 uM) PDZ domain-PDZ domain interaction (2,53). The GLGF loop in the acceptor syntrophin or PSD-95 PDZ2 domains, provides a steric block at the end of the ligand binding groove that necessitates chain termination or a sharp turn in peptide sequences immediately following the peptide recognition motif. The 3-D structure of the PDZ domain in complex with nNOS is shown in detail in FIG. 5 and in a cartoon representation in FIG. 6.

Therefore in the exemplary structural configuration of a PDZ-BH providing by the naturally occurring nNOS protein, in which a two-stranded β-hairpin augments an antiparallel beta sheet in the PDZ domain ligand binding pocket, forming a composite four-stranded beta sheet (2). The first strand of the nNOS β-hairpin mimics the structure and shape of a canonical COOH-terminal peptide ligand in its sequence-specific interactions with the PDZ domain, however, a sharp Beta turn exists in place of the essential peptide carboxy terminus, forming a strong, sub-micromolar (K_{D} of ˜0.6 uM) PDZ domain-PDZ domain interaction (2,53).

In the PDZ-BH of nNOS, a core interaction that pins the base of the beta hairpin against its associated domain is a buried salt bridge between an arginine (121 in nNOS) in the β-hairpin and aspartic acid (62 in nNOS) in the main PDZ domain. Stabilization of the nNOS Beta Hairpin structure is performed by the remaining portion of nNOS PDZ domain as the entire nNOS PDZ domain is required for proper functioning of the Beta Hairpin (2). In the exemplary binding of nNOS PDZ domain with the PDZ2 domain of the acceptor syntrophin or PSD-95, the GLGF loop of the PDZ domain of PSD-95 provides a steric block at the end of the ligand binding groove that necessitates chain termination or a sharp turn in peptide sequences immediately following the peptide recognition motif.

Exemplary PDZ BH comprise RGPEGFTTHLETTFTGDGTPKTIRVTQ (SEQ ID NO: 157) from nNOS.

In embodiments herein described, PDZ domain-PDZ domain peptide interactions through β-hairpin can have a dissociation constant (K_{D}) values between 0.1-10 uM. Selection of an appropriate strength of interactions between candidate PDZ binding carboxy terminal peptide and PDZ domain peptide and candidate PDZ domain and a PDZ domain-β-hairpins can be measured by several techniques such as Surface Plasmon Resonance (SPR)/Biacore, fluorescence polarization, Fluorescence Resonance Energy Transfer (FRET), Isothermal Titration calorimetry (ITC) bead based binding assays and additional techniques identifiable by a skilled person. Dissociation constant (K_{D}) values from 0.1 uM to zero uM (Covalent binding) can also obtained by directed evolution of PDZ domain and ligand pairs, with a dissociation constant (K_{D}) value of zero being obtained by designing a PDZ domain and Ligand pair that make a covalent bond to one another.

In various embodiments herein described PDZ domains peptides, and in particular PDZ domains peptides containing a β-hairpin, and a PDZ binding carboxy terminal peptide can be used either as an affinity tag or as an affinity ligand based on the ability to specifically bind one to another and to PDZ domains and/or PDZ binding carboxy terminal peptides, possibly naturally occurring, present in biochemical mixtures and in particular, in samples.

In particular, in some embodiments PDZ domains peptides, and in particular PDZ domains peptides containing a β-hairpin, and a PDZ binding carboxy terminal peptide allow separation and/or detection of targets such as POIs in an unprepared sample, where the term “unprepared sample” as used herein indicates a sample that has not been subjected to sample preparation, wherein the term “sample preparation” refers to the way a sample is treated prior to its analysis to increase availability of the target of the analysis e.g. by separate fractions based on solubility. Accordingly, an unprepared sample in the sense of the disclosure can comprise an unclarified sample comprising soluble and insoluble fractions. Exemplary unprepared samples in the sense of the disclosure comprise biological materials such as cell lysates prepared by sonication, non-ionic detergent lysates, microfluidization, freeze thaw, and French press.

A specific pair of PDZ affinity tags and corresponding ligands can be selected based on the related dissociation constant, peptide structure and sequence, sequence of a target, reaction conditions and/or additional variables identifiable by a skilled person upon reading of the present disclosure. For example, given a set PDZ binding carboxy terminal peptide, a relatively high binding affinity for the PDZ binding carboxy terminal peptide, e.g. K_{D} lower than 50 uM (The approximate K_{D} for GST is 40-170 uM (54-58) is the primary determinant for effectiveness of a PDZ domain as an affinity tag or affinity ligand. Also, the presence of unique binding specificity towards a set of PDZ binding carboxy terminal peptides, the absence of surface exposed primary amines (other than the N-terminus of the protein) or the presence of a single surface exposed thiol residue, can be additional factors to be considered for selection of the PDZ domain to be used in connection with the set PDZ binding carboxy terminal peptide (e.g. when used to prepare NHS or Thiol coupled affinity matrices, respectively).

In some embodiments the PDZ element PDZ domain, PDZ binding C-terminal peptide and PDZ BH can be used as affinity tags and or affinity ligand in an affinity chromatography system depending on the experimental design.

In particular in some embodiments of the affinity chromatography system herein described, a target protein can be engineered to introduce a PDZ affinity tag which is formed by a PDZ domain, a PDZ domain containing a β-hairpin or a PDZ domain binding carboxy terminal peptide, as will be understood in view of the present disclosure.

The term “target” as used herein indicates an analyte of interest to be captured. The term “analyte” can refer to a substance, compound, moiety, or component whose presence or absence in a sample is to be detected or captured. Analytes include but are not limited to biomolecules and in particular biomarkers. The term “biomolecule” as used herein indicates a substance, compound or component associated with a biological environment including but not limited to sugars, amino acids, peptides, proteins, oligonucleotides, polynucleotides, polypeptides, organic molecules, haptens, epitopes, biological cells, parts of biological cells, vitamins, hormones and the like. The “biological environment” refers to any biological setting, including, for example, ecosystems, orders, families, genera, species, subspecies, organisms, tissues, cells, viruses, organelles, cellular substructures, prions, and samples of biological origin.

The term “protein” as used herein indicates a polypeptide with a particular secondary and tertiary structure that can interact with and in particular bind another analyte and in particular, with other biomolecules including other proteins, DNA, RNA, lipids, metabolites, hormones, chemokines, and small molecules. The term “polypeptide” as used herein indicates an amino acid polymer of any length including full-length proteins and peptides, as well as analogs and fragments thereof. A polypeptide of three or more amino acids is also called a protein oligomer, peptide or oligopeptide.

In several embodiments described herein, the disclosure relates to “target protein” or “protein of interest” or “POI” which can be engineered to introduce one or more PDZ tags as will be understood by a skilled person. In particular a target protein can be engineered to introduce one or more affinity tags by diverse means for example, using ligation independent cloning (4,5), Gibson Assembly (6,7) or gene synthesis (8,9), co-translationally grafted (for example, using non-canonical amino acid insertion (10-12)) or post-translationally grafted using click chemistry (13-19) or an enzyme such as sortase (20-23). In some instances grafting can be performed by chemical modification (e.g. by use of a sortase or other enzymes as will be understood by a skilled person).

In particular, attachment to produce engineered proteins herein described is performed by grafting the PDZ affinity tag on any portion of the protein that is presented on the outside environment. Determination of portions of a POI that can be grafted with a an PDZ affinity tag here described can be determined by computer programs, or an experimental approach directed to detect a portion in the POI as folded that is presented for binding and can be used as a substrate for grafting. Typically an affinity tag is attached to one of the end of a POI typically the C-terminus end of a target protein that is selected as a POI which is linked to the N terminus of either the PDZ domain peptide or the PDZ C-terminal peptide.

Possible POIs in the sense of the disclosure comprise any protein which presents the N terminus or the C-terminus for binding with a corresponding protein through formation of a covalent bond (e.g. a peptide bond). In addition or in the alternative a suitable POI can present a region configured to be presented for binding to an affinity tag according to the disclosure through a covalent bond (e.g. a peptide bond).

In some embodiments, the affinity tag can be usefully attached to a POI formed by membrane proteins, and in particular GPCRs, Glutamate receptors, potassium channels, hydroxyl tryptamine receptors, beta adrenergic receptors, voltage dependent calcium channels and additional membrane protein identifiable by a skilled person upon reading of the present disclosure.

In some embodiments, the affinity tag can be attached to a POI formed by a protein which includes a PDZ domain and/or a PDZ binding C-terminal peptide, such as naturally occurring proteins (e.g. tight junction proteins, scaffold proteins, Homer family, Shank family, and Maguk family)

In particular in some of those embodiments, the interactions between affinity tag of the disclosure comprised in engineered POIs described herein, have been proven and are expected to be very specific (e.g. Kd between 0.1 and 10 μM) to the extent of increasing sensitivity of binding between affinity tags and corresponding ligand compared to certain affinity tags and ligands identifiable by a skilled person. In particular, affinity tags of the disclosure allow purification of an engineered POI, and in particular of a membrane protein and of a protein comprising a PDZ domain, at least 65% and in certain embodiments up to 99%.

Exemplary POIs herein described comprise Green fluorescent proteins (GFP). GFP is a 238 residue protein isolated from the Pacific Northwest jellyfish, Aequeorea victoria (59). GFP transmutes blue chemiluminescence from a primary photoprotein (aequorin) into green fluorescence (60), utilizing a p-hydroxybenzylidene-imidazolidone chromophore derived from its S65, Y66 and G67 residues (61). It has been used in the production of biosensors for monitoring intracellular pH (62-64), calcium concentration (65), redox potential (66,67), membrane potential (68) and temperature (69). Proper folding of GFP around the chromophore is necessary for fluorescence, as evidenced by the fact that synthetic p-hydroxybenzylidene-imidazolidone chromophores are devoid of fluorescence (70). When fused to the C-terminus of a POI, productive folding of the downstream GFP and formation of the fluorescent chromophore has been shown to depend on the robustness of folding of the upstream protein (71). DasherGFP is a 26.6 kDa, synthetic, non-aequorea fluorescent protein, developed by DNA2.0, with excitation and emission wavelengths of 505 and 525 nm, respectively, and was used to verify the effect of PDZ Affinity Tags on protein folding. DasherGFP is an exemplar from the class of fluorescent proteins including GFP, RFP, YFP, CFP, etc. regarding protein functionality and overall protein fold. Attachment of PDZ Affinity Tags (N-terminally and C-terminally) to DasherGFP and subsequent testing of its activity, verify that PDZ Affinity Tags do not inhibit the fluorescence emission of DasherGFP, and thus do not inhibit the function of fluorescent proteins.

Exemplary POIs herein described comprise β-Galactosidase (LacZ). LacZ is a 1024 residue protein isolated from E. coli (72,73). It catalyzes the cleavage of the bond between the anomeric carbon and glycosyl oxygen of a β-D-galactopyranoside (74). In vivo LacZ catalyzes the cleavage of the disaccharide lactose to form glucose and galactose (75). It is often used experimentally as a reporter of gene expression, of spontaneous or directed genetic changes in coding sequences, and of protein-protein interactions because of its activity in myriad cell lines (76-79), the availability of an array of substrates, inducers and inhibitors (80-82), its structural malleability (83-85) and the large dynamic range of its gene expression (86). LacZ is an exemplar from the class of enzymes known as hydrolases and a representative enzyme used as a genetic reporter. Attachment of PDZ Affinity Tags (N-terminally and C-terminally) to LacZ and subsequent testing of its activity, verify that PDZ Affinity Tags do not inhibit the hydrolytic activity of LacZ, and thus do not inhibit the function of other hydrolases or genetic reporters.

Exemplary POIs herein described comprise Chloramphenicol acetyltransferase (CAT). CAT is a ˜219 residue protein isolated from E. coli and S. aureus. It catalyzes the inactivation of the antibiotic chloramphenicol, by acylating chloramphenicol in the presence of acetyl-CoA to produce chloramphenicol-3-acetate and reduced CoA (87,88). It has been used extensively as an in vitro reporter of gene expression levels in eukaryotic cell lines because of its stability, absence of competing activities in eukaryotic cells, ease of use and sensitivity (89-91). CAT is an exemplar from the class of enzymes known as transferases and a representative enzyme used as a genetic reporter. Attachment of PDZ Affinity Tags (N-terminally and C-terminally) to CAT and subsequent testing of its activity, verify that PDZ Affinity Tags do not inhibit the transferase activity of CAT, and thus do not inhibit the function of other transferases or genetic reporters.

In particular in some embodiments herein described, the PDZ Affinity Tag of the disclosure can be grafted in various portions of the target protein, e.g. via a PDZ N-terminal tag, PDZ C-terminal tag, or by an internal PDZ tag as will be understood by a skilled person. In particular the PDZ Affinity Tags can be grafted onto the target protein by diverse means, for example, using ligation independent cloning (4,5), Gibson Assembly (6,7) or gene synthesis (8,9), co-translationally grafted (for example, using non-canonical amino acid insertion (10-12)) or post-translationally grafted using click chemistry (13-19) or an enzyme such as sortase (20-23). In some instances grafting can be performed by chemical modification (e.g. by use of a sortase or other enzymes as will be understood by a skilled person).

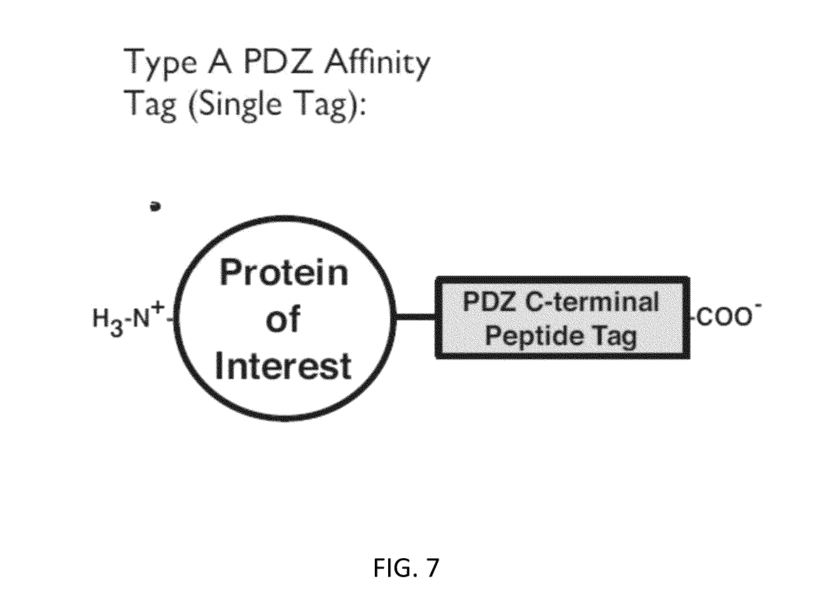

Attention is drawn to FIG. 7 which shows an element of the PDZ Affinity Chromatography system, PDZ binding C-terminal peptide tag (e.g. Type A PDZ Affinity Tag). The most common mode of protein-protein interaction between PDZ domains and their partners involves insertion of approximately six C-terminal amino acids of the PDZ binding partner into the ligand binding pocket of the PDZ domain, as previously described. Due to the high specificity and affinity of this mode of interaction, the PDZ domain and a PDZ binding C-terminal peptide can be exploited as a tool for the affinity purification of any POI. The gene for a POI can be genetically fused to a gene fragment coding for a PDZ binding C-terminal peptide (e.g. Type A PDZ Affinity Tag). Appending a PDZ binding C-terminal peptide to a POI will confer PDZ domain binding ability to the POI and allow its purification when combined with an affinity resin comprising of a complementary PDZ domain immobilized on a solid support. Because PDZ binding C-terminal peptides (Affinity Tag) and their respective corresponding PDZ domains (Affinity ligand) exist in naturally occurring, complementary pairs, the PDZ binding C-terminal peptides and immobilized PDZ domains can be derived from naturally occurring protein binding partners, or if needed, can be computationally designed. Computational design programs for protein structures and modeling are known to those skilled in the art. First generation PDZ binding C-terminal peptide Tag-PDZ Domain pairs have been derived from naturally occurring PDZ binding C-terminal peptide sequences and their known PDZ domain binding partners. Second generation and beyond PDZ binding C-terminal peptide Tags-PDZ Domain pairs will be computationally designed and optimized by directed evolution methodologies to confer increased specificity and affinity over existing PDZ C-terminal Peptide-PDZ Domain pairs derived from nature. In an embodiment described herein, the C-terminal 7 amino acids from the NR2B Tail protein (SSIESDV) (SEQ ID NO: 161) have been chosen and established as a the First Generation PDZ binding C-terminal peptide Tag (e.g. Type A PDZ Affinity Tag). The PDZ2 domain from PSD-95 has been purified and immobilized on a solid support to generate PDZ Affinity Resin. The immobilized PDZ2 domain is then used to capture any POI fused to a PDZ binding C-terminal peptide Tag (e.g. Type A PDZ Affinity Tag).

“Immunoaffinity chromatography,” as described herein, refers to the specific binding of an antibody to the target protein to selectively purify the protein. The procedure involves immobilizing an antibody to a column material, which then selectively binds the protein, while everything else flows through. The protein can be eluted by changing the pH or the salinity. Because this method does not involve engineering in a tag, it can be used for proteins from natural sources. In several embodiments described herein, purification techniques utilize a solid support with a covalently bound antibody to bind to the target protein or protein of interest.

In several embodiments described herein, proteins are purified by a specific tag attached to the POI. Another way to tag proteins is to engineer an antigen peptide tag onto the protein, and then purify the protein on a column in which the column is comprised of beads that are conjugated to a protein that has a high affinity for the specific antigen peptide tag or by incubating the protein of interest (POI) with a loose resin that is coated with an immobilized antibody. This particular procedure is known as immunoprecipitation. “Immunoprecipitation,” as described herein, refers to the use of an antibody that is specific for a known protein to isolate that particular protein out of a solution containing many different proteins. These solutions will often be in the form of a crude lysate of a plant or animal tissue. Other sample types could be body fluids or other samples of biological origin. Immunoprecipitation is capable of generating an extremely specific interaction which usually results in binding only the desired protein. The purified tagged proteins can then easily be separated from the other proteins in solution and later eluted back into clean solution. When the tags are not needed anymore, the tags can be cleaved off by a protease. This often involves engineering a protease cleavage site between the tag and the protein, which are methods known to those skilled in the art. Proteases that can be used to remove fusion tags can be for example, can be enterokinase, Factor X, Thrombin, and other proteases that are known to those skilled in the art.

The term “antibody” as used herein refers to a protein of the kind that is produced by activated B cells after stimulation by an antigen and can bind specifically to the antigen promoting an immune response in biological systems. Full antibodies typically consist of four subunits including two heavy chains and two light chains. The term antibody includes natural and synthetic antibodies, including but not limited to monoclonal antibodies, polyclonal antibodies or fragments thereof or derivative thereof. The terms “fragment” as used herein with reference to antibody indicates any portion of an antibody that retain an immunogenic activity characteristic of the antibody. The term “derivative” as used herein with reference to an antibody, indicates a molecule that is structurally related to the antibody and is derivable from the antibody by a modification that introduces a feature that is not present in the antibody while retaining functional properties of the antibody. Accordingly, a derivative antibody, or of any fragment thereof, FaB or scFv, usually differs from the original antibody or fragment thereof by modification of the amino acidic sequence that might or might not be associated with an additional function not present in the original antibody or fragment thereof. Methods to provide derivative and to test the ability of the derivative to retain the one or more functional properties are identifiable by a skilled person. Exemplary antibodies include IgA, IgD, IgG1, IgG2, IgG3, IgM and the like. Exemplary fragments include Fab Fv, Fab′ F(ab′)2 scFV, single chain antibodies and the like. A monoclonal antibody is an antibody that specifically binds to and is thereby defined as complementary to a single particular spatial and polar organization of another biomolecule which is termed an “epitope”. In some forms, monoclonal antibodies can also have the same structure. A polyclonal antibody refers to a mixture of different monoclonal antibodies. In some forms, polyclonal antibodies can be a mixture of monoclonal antibodies where at least two of the monoclonal antibodies binding to a different antigenic epitope. The different antigenic epitopes can be on the same target, different targets, or a combination. Antibodies can be prepared by techniques that are well known in the art, such as immunization of a host and collection of sera (polyclonal) or by preparing continuous hybridoma cell lines and collecting the secreted protein (monoclonal).

In embodiments herein described, an antibody can be monoclonal or polyclonal or an antibody preparation thereof and have a specific binding to a target. In some embodiments, the antibody can be used in detectors, methods and systems herein described as antibody preparation in which the antibody, including fragments and derivative thereof, is comprised in a solution together with a suitable vehicle.

In some embodiments, antibodies can be binding compound and/or targets; in some embodiments, antibody can also be raised against targets such as a ligand, or an antibody and used as binding compound. In some embodiments, the antibody can be an IgG antibody.

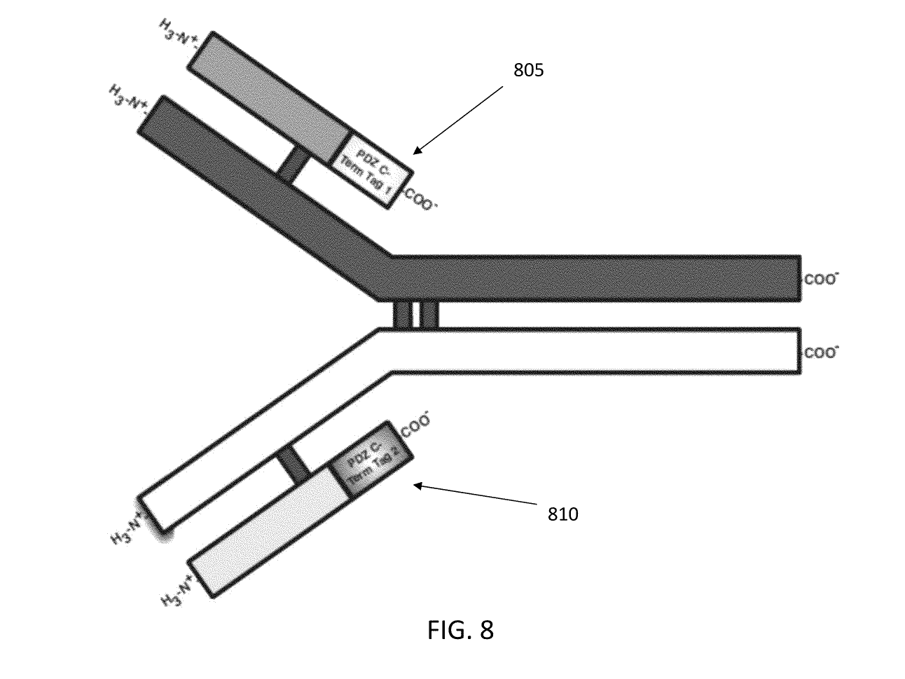

As shown in FIG. 8, is a cartoon representation of one of the embodiments described herein, of dual PDZ domain binding C-terminal peptide affinity tags (e.g. Type A PDZ Affinity Tags, Dual) fused to a bi-specific monoclonal antibody. In this exemplary embodiment, the genes coding for each unique light chain of a bi-specific mono-clonal antibody can be genetically fused to gene fragments coding for PDZ C-terminal Peptide Tags (e.g. Type A PDZ Affinity Tags). Each unique light chain will be genetically fused to a different Type A PDZ Affinity Tag, conferring a specific PDZ domain binding ability to each light chain, allowing purification of the bi-specific mono-clonal antibody utilizing two distinct PDZ Affinity Resins, each consisting of a different PDZ domain immobilized on a solid support. Purification of bi-specific mono-clonal antibodies (Mono-clonal antibodies with two different light chains) is currently difficult, as current purification methodologies are inadequate for selection of each unique light chain. A chromatography method capable of purifying bi-specific mono-clonal antibodies would be incredibly useful in the pharmaceutical industry and biomedical research industries as many bi-specific mono-clonal antibodies have excellent anti-viral and anti-oncogenic properties. As shown in FIG. 8, the bi-specific antibody has two different PDZ binding C-terminal peptide tags (805, 810), one on each light chain (tandem).

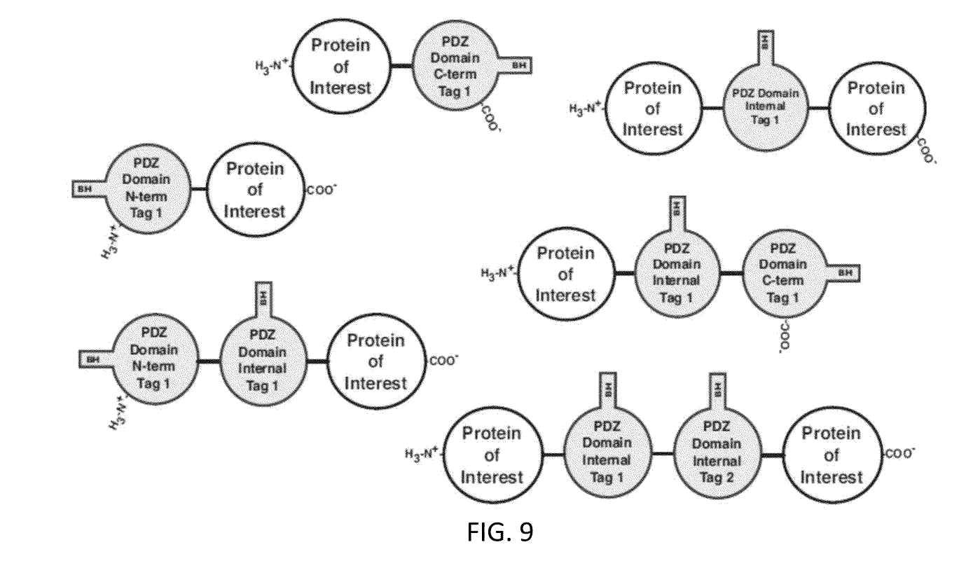

In several embodiments described herein, a PDZ domain is attached to a target protein or POI in order to capture the protein from a crude solution. As shown in FIG. 9 (Cartoon Representation of PDZ Domain with β-Hairpin Affinity Tags (e.g. Type B PDZ Affinity Tag), the PDZ can be attached to a POI through multiple ways. For example, FIG. 9 shows a cartoon diagram of the POI with a Type B tag, i) with an attached PDZ domain and β-Hairpin at the C- or N-terminus, ii) a POI with two PDZ domains and β-Hairpins at the N- or C-terminus of the POI, iii) a PDZ and β-Hairpin internal tag that is positioned between an N-terminal and a C-terminal POI, iv) a POI with two N- or C-terminal PDZ domains and β-Hairpin, and a protein comprising two POI with two internal PDZ domains with β-Hairpins between the POI. In particular, the gene for a POI can be genetically fused to a gene fragment coding for a PDZ Domain with a and β-Hairpin motif (e.g. Type B PDZ Affinity Tag). Appending a PDZ-β-Hairpin domain to a POI confers PDZ domain and PDZ binding C-terminal peptide binding ability to the POI and allow its separation when combined with an affinity resin having either a PDZ domain or PDZ binding C-terminal peptide immobilized on a solid support. Because PDZ-β-Hairpin domains (Affinity Tag) and their respective PDZ binding C-terminal peptides (Affinity ligand) or PDZ domain binding partners (Affinity ligand) exist in naturally occurring, complementary pairs, Affinity Tags and Affinity ligands can be derived from naturally occurring protein corresponding PDZ binding partners, can be computationally designed. First generation PDZ-β-Hairpin domain and C-terminal PDZ Peptide/PDZ domain pairs have been derived from naturally occurring PDZ-β-Hairpin domains their known PDZ binding C-terminal peptide/PDZ domain binding partners. Second generation and beyond PDZ-β-Hairpin domain and C-terminal Peptide Tags/PDZ domain pairs will be computationally designed and optimized by directed evolution methodologies to confer increased specificity and affinity over existing PDZ-β-Hairpin domain and C-terminal PDZ binding peptide/PDZ domain pairs derived from nature. In the specific examples shown in later Figures, the PDZ-β-Hairpin domain from neuronal Nitric Oxide Synthase (nNOS PDZ domain) has been chosen as the First Generation PDZ-β-Hairpin Domain Affinity Tag (e.g. Type B PDZ Affinity Tag). The PDZ2 domain from PSD-95 has been purified and immobilized on a solid support to generate PDZ Affinity Resin. The immobilized PDZ2 domain is used to capture any POI fused to the First Generation PDZ-β-Hairpin Domain Affinity Tag (e.g. Type B PDZ Affinity Tag).

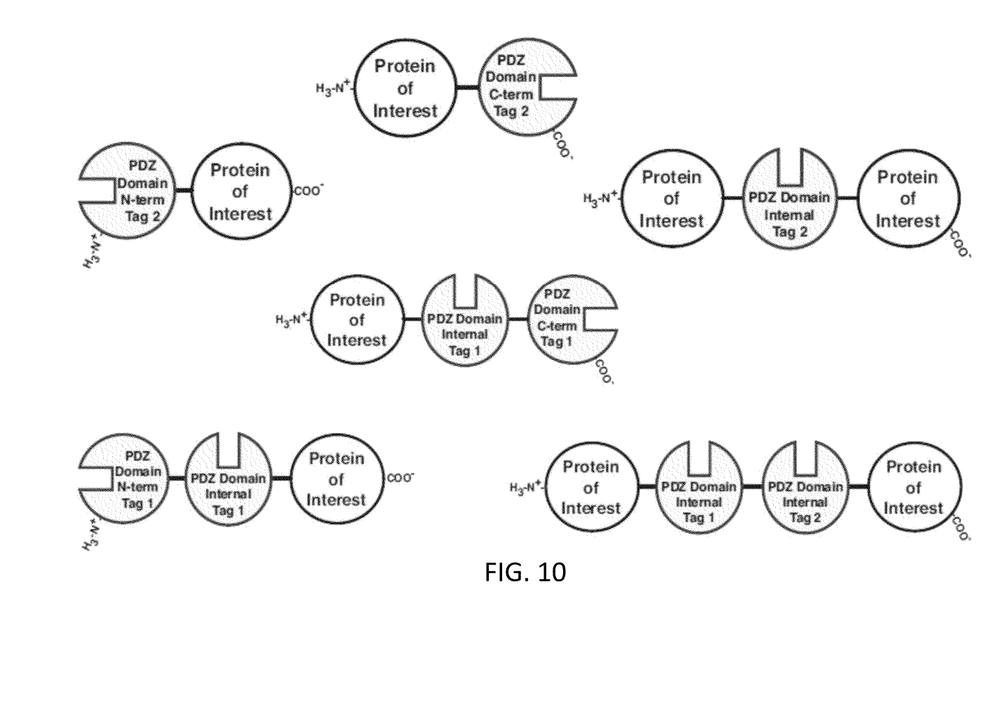

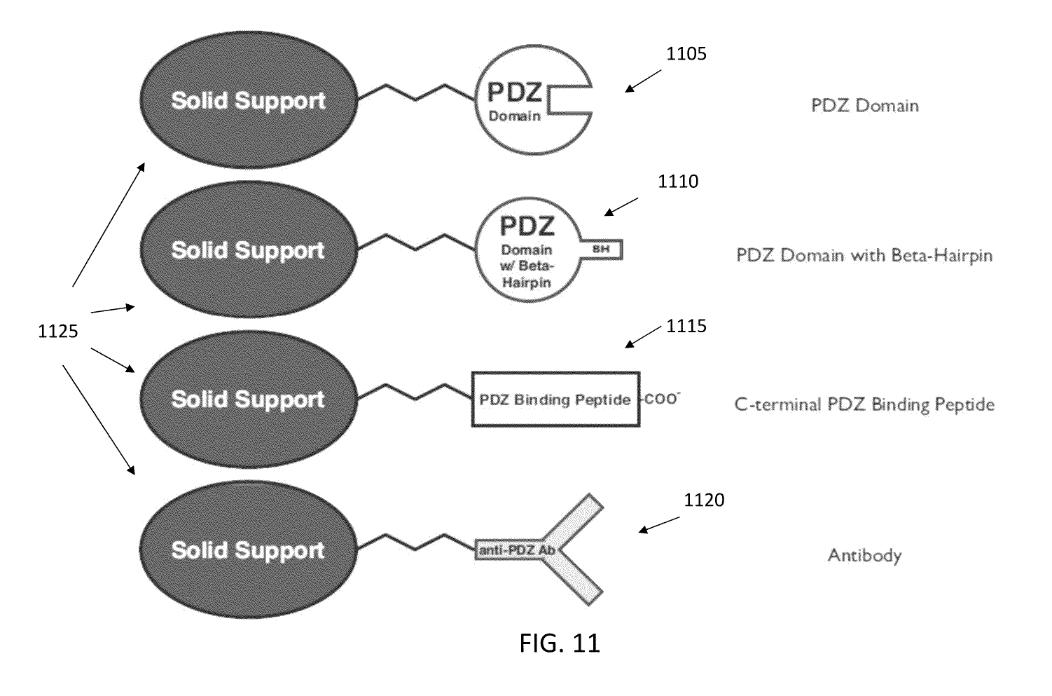

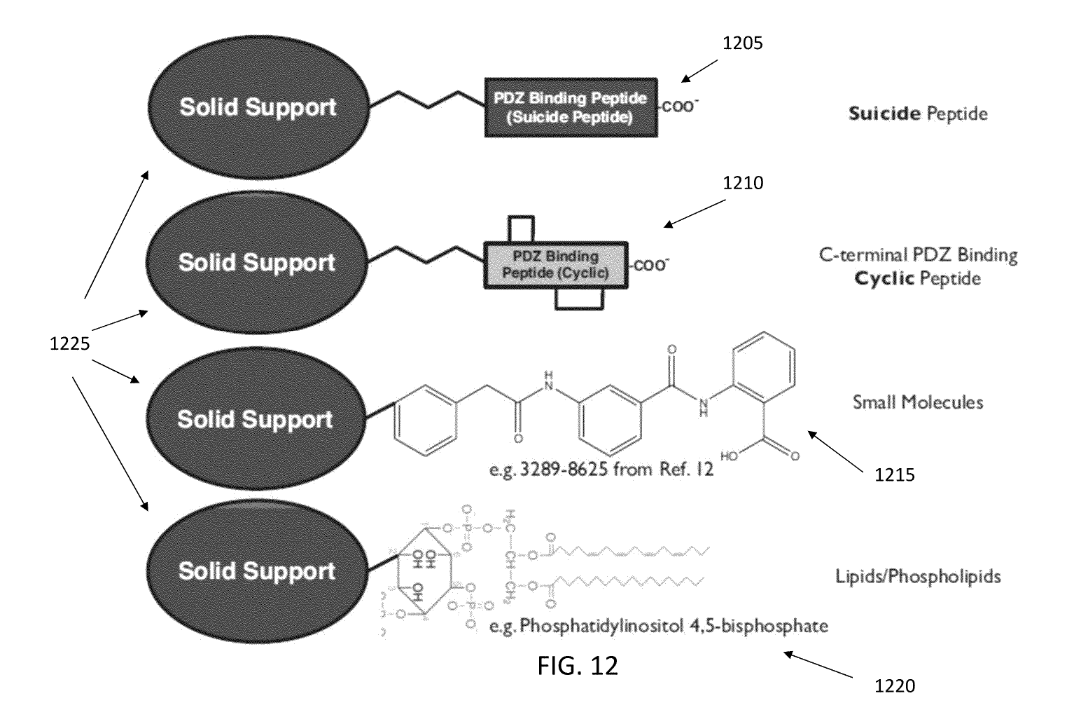

FIG. 10 shows an exemplary embodiment described herein, of the POI with N-terminal, Internal or C-terminal PDZ Domain Tags (e.g. Type C). In particular, the gene for a POI can be genetically fused to a gene fragment coding for a PDZ domain (e.g. Type C PDZ Affinity Tag). Appending a PDZ domain to a POI will confer PDZ-β-Hairpin domain and PDZ binding C-terminal peptide binding ability to the POI and allow its purification when combined with an affinity resin consisting of either a PDZ-β-Hairpin domain or PDZ binding C-terminal peptide immobilized on a solid support. Because PDZ domain (Affinity Tag) and their respective PDZ binding peptides (Affinity ligand) or PDZ-β-Hairpin domain binding partners (Affinity ligand) exist in naturally occurring, complementary pairs, Affinity Tags and Affinity ligands can be derived from naturally occurring protein binding partners, or if needed, can be computationally designed. First generation PDZ domain and C-terminal PDZ binding peptide/PDZ-β-Hairpin domain pairs have been derived from naturally occurring PDZ domains their known PDZ binding C-terminal peptide/PDZ-β-Hairpin domain binding partners. Second generation and beyond PDZ domain and PDZ binding C-terminal Peptide Tags/PDZ-β-Hairpin domain pairs will be computationally designed and optimized by directed evolution methodologies to confer increased specificity and affinity over existing PDZ domain and PDZ binding C-terminal peptide/PDZ-β-Hairpin domain pairs derived from nature. In the specific examples shown in later Figures, the PDZ2 domain from PSD-95 has been chosen as the First Generation PDZ Domain Affinity Tag (e.g. Type C PDZ Affinity Tag). The PDZ-β-Hairpin domain from nNOS has been purified and immobilized on a solid support to generate PDZ Affinity Resin. The immobilized nNOS PDZ-β-Hairpin domain is used to capture any POI fused to the First Generation PDZ Domain Affinity Tag (e.g. Type C PDZ Affinity Tag). Additionally a short N-terminal linker sequence (GAG) and the C-terminal 7 amino acids from the NR2B (GluN2B) Tail protein (SSIESDV) have been synthesized as a peptide (H3N+-GAGSSIESDV-COO—) and immobilized on a solid support to generate PDZ Binding C-terminal Peptide Affinity Resin. The immobilized PDZ binding C-terminal peptide is used to capture any POI fused to the First Generation PDZ Domain Affinity Tag (e.g. Type C PDZ Affinity Tag).

Selection of a specific affinity tag and ligand in accordance with the disclosure can be performed by a combined search of the Mint (92), IntAct (92), or ELM (93) protein interaction databases to identify affinity tag and ligand pairs followed by examination of the referenced sequences in the Uniprot (94) and RCSB databases (95,96) and peer reviewed journal articles.

Alternatively, selection of a specific affinity tag and ligand in accordance with the disclosure can be performed by computational design methods using structure prediction and protein design packages to identify PDZ affinity tags and ligand interactions such as Modeller, Rosetta, Robetta, iTasser, Orbit, machine learning algorithms, statistical potentials, and additional methods identifiable by a skilled person performed to also take into account the dissociation constant K_{D} for each pair.

Selection of the PDZ element to be used as PDZ affinity tag in methods and systems herein described can also be performed by systematic trial and error. For example, upon choosing a target protein for study, the target protein can be cloned using high throughput cloning methodologies (Polymerase Incomplete Primer Extenstion (PIPE), Gibson Assembly, Gene synthesis, etc.) in frame with N-terminal, C-terminal or Internal PDZ domain, PDZ domain with β-Hairpin or PDZ binding C-terminal peptide ligand affinity tags. Each tagged target protein can be purified in small scale with its respective ligand affinity matrix and eluted with the corresponding ligand (A denaturant will be used following elution with ligand to confirm that target protein was eluted from the affinity matrix). Following completion of PDZ affinity tag and affinity matrices, the correct tag/ligand pair will be identified. This approach is common to all of protein purification, as prediction of optimal tag and matrix conditions for purification of a target protein is non-obvious and difficult to predict (e.g. trial and error is usually required to select the best tag/matrix pair for purification).

Selection of the PDZ element to be used as PDZ affinity tag can be performed also based on the elution condition. The term “elution” indicates the process of extracting one material from another by washing with a solvent (as in washing of loaded ion-exchange resins to remove captured ions). Therefore n “elution conditions” as used herein refers to conditions such as pH, salt content, or competitor content allowing the extraction of a protein from a molecular complex In particular in peptide-peptide or protein-peptide or protein-protein complexes elution conditions can allow removal of unbound proteins initially (e.g. proteins that are not the protein of interest) and at higher concentration release the protein of interest. In various embodiments salt solution of various concentration could be used as well as analogues for the bound sample. In various embodiments a gradient will be used such as to allow gently raise to the concentration need for the protein of interest.

In some embodiments, the PDZ domain, PDZ binding C-terminal peptide and/or PDZ-BH can be attached to a solid support to provide a chromatography solid phase.

The term “attach” or “attached” as used herein, refers to connecting or uniting by a bond, link, force or tie in order to keep two or more components together, which encompasses either direct or indirect attachment where, for example, a first molecule is directly bound to a second molecule or material, or one or more intermediate molecules are disposed between the first molecule and the second molecule or material. Exemplary attachments comprise fusions attached at the C-terminal or N-terminal end of a protein of interest, chemical coupling to a target for example via a maleimide cysteine bond linkage or cyanate ester reaction with a protein amine, or affinity coupling, (e.g. via super high affinity antibody).

Peptide and protein based PDZ elements comprised of amino acids can be attached to supports or other molecules through covalent bonds formed between reactive groups in their amino acids and reactive groups on the solid supports or molecules. Specific examples include attachment of primary amine functional groups present on the peptide N-terminus and the amino acid side chains of lysine, arginine to hydroxylated functional groups on the support or molecule activated with CNBr (97,98), and CDAP (98) to produce isourea bonds. Reaction of primary amine functional groups present on the peptide N-terminus and the amino acid side chains of lysine, arginine to hydroxylated functional groups on the support or molecule activated with N,N′-disuccinimidylcarbonate (DSC) (99), carbonyldiimidazole (CDI) (100) produce carbamate bonds, whereas matrices activated with tosyl or tresylchloride (101) or N-hydroxysuccinimide form secondary amine and amide bonds, respectively, with amine groups of peptides. Additional sites of reactivity present in peptide and protein based PDZ elements include the sulfhydryl groups of cysteine residues, the carboxylic acid groups of the peptide and protein C-terminus and side chains of aspartic and glutamic acid residues. Sulfhydryl groups of cysteine residues can react with hydroxylated functional groups on the support or molecule activated with tosyl or tresylchloride (101), Bisoxiranes (102), epichlorohydrine (103), divinylsulphone (DVS) to form thioether bonds, and can also react with thiol bonds present on the support to form disulfide bonds. Carboxylic acid groups of the peptide and protein C-terminus and side chains of aspartic and glutamic acid residues can, in the presence of the carbodiimides such as EDC, can react with immobilized primary amines on support surfaces to form amide bonds. Derivatized cyclic peptides, lipids and small molecules can all undergo the aforementioned reactions provided they possess the required functional groups; if not they can be synthesized to contain the required functional groups. Additionally, peptides can be modified by chemicals or enzymes, such as sortase, to append additional functional groups to allow alternate chemical means for attachment to supports and molecules. In lieu of chemically activating matrices for attachment, one can also purchase preactivated matrices designed for coupling to amine groups (e.g. CNBr-activated Sepharose 4B, Activated CH Sepharose 4B, Tresylactivated Sepharose 4B, AffiGel 10 Gel, etc.), carboxylic acid groups (e.g. NHS Activated Agarose, AH-Sepharose 4B, EAH-Sepharose 4B, AF-Amino 650 Fractogel TSK, etc.) and sulfhydryl groups (e.g. Thiopropyl Sepharose 6B, AF-Epoxy 650 Fractogel TSK, etc.)

The term “support” as used herein indicates an underlying solid substratum. Exemplary substratums include solid substrates, such as glass plates, chromatography resin (e.g. Sephadex, Sepharose, Agarose, silica, etc.), microtiter well plates, magnetic beads, silicon wafers and additional substrates or surfaces identifiable by a skilled person upon reading of the present disclosure In particular, the term “support” as used herein indicates an inert material, usually solid, that is configured to attach one or more compounds of interest and to allow flow of a fluid through the material (e.g. by capillary flow, lateral flow, gravity flow, or by a peristaltic pump). Exemplary supports in the sense herein described can be beads configured to attach binding compounds, polymer gel structure, or other platforms identifiable by a skilled person. In several embodiments described herein, the support to capture the POI can be a solid support conjugated to a PDZ domain, PDZ-BH or to a PDZ binding peptide.

Exemplary supports for chromatography applications include inorganic materials (e.g. pourous silica, controlled pore glass, hydroxyapatite), synthetic organic polymers (e.g. polyacrylamide, polymethacrylate, polystyrene) and polysaccharides (e.g. cellulose, dextran, agarose). Idealized chromatographic matrices for protein chromatography should not contain groups that spontaneously bind protein molecules, however they should contain functional groups to allow controlled synthesis of a wide variety of protein adsorbents. Idealized matrices should be chemically and physically stable enough to withstand extreme conditions during maintenance and derivatization and be rigid enough to maintain high linear flow rates in columns packed with particles with diameters in the micrometer range. Additionally, matrix substances should allow the production of gels with a broad range of controllable porosities. Since no matrix fulfills all idealized criteria for protein chromatography, a combination of hydrophilicity and chemical and physical inertness is best achieved using alcohol hydroxyls or amido groups, thus neutral polysaccharide and polyacrylamide matrices are the most commonly used supports for protein chromatography. Exemplary polysaccharide matrices for use as solid supports for coupling to PDZ domain, PDZ-BH or PDZ binding peptides include Sephadex, dextran, agarose, Sepharose, Superose, Ultrogel A, BioGel A, Superdex, and AffiGel.

In particular, grafting of the PDZ domain, PDZ binding C-terminal peptide and/or PDZ BH or attachment of the PDZ domain, PDZ binding C-terminal peptide and/or PDZ BH is performed in methods and systems herein disclosed to have PDZ domain, PDZ binding C-terminal peptide and/or PDZ BH presented on the support or the POI for binding with a corresponding PDZ domain, PDZ binding C-terminal peptide and/or PDZ BH.

The term “present” as used herein with reference to a compound or functional group indicates attachment performed to maintain the chemical reactivity of the compound or functional group as attached. Accordingly, a functional group presented on a ligand, is able to perform under the appropriate conditions the one or more chemical reactions that chemically characterize/define the functional group.

In various embodiments, the affinity tags and affinity ligands can be used, for example in an affinity chromatography system, to obtain separation of a target protein from a mixture and in particular from a sample.

The term “mixture” as used herein indicates a material system made up of two or more different substances which are mixed but are not combined chemically. A mixture therefore can refers to the physical combination of two or more substances on which the identities are retained and are mixed in various forms identifiable by a skilled person and in particular in form of solutions, suspensions, and colloids.

In particular, in some embodiments, the mixture can be a sample. The term “sample” as used herein indicates a limited quantity of something that is indicative of a larger quantity of that something, including but not limited to fluids from a biological environment, a bodily fluid, a specimen, cultures, tissues, harvested cells, commercial recombinant proteins, synthetic compounds or portions thereof. The term “bodily fluid” can include blood, lymphatic fluid, cerebrospinal fluid, urine, saliva, vaginal fluid, digestive fluids and additional fluids identifiable by a skilled person. In several embodiments described herein, the sample is a crude preparation.

In some embodiments, the sample can be provided in the form of a crude preparation. The term “crude preparation” can refer to a crude lysate solution produced when cells are destroyed by disrupting their cell membranes, often with detergent, other chaotropic agents, mechanical shearing, or freeze-thawing (liquid nitrogen, 37° C., 1 min vortex), in a process known as cytolysis. This process releases the contents within the cell. Crude cell lysates are routinely produced in biochemistry and cell biology laboratories during the process of protein purification, although purified cellular organelles can also be retrieved from the solution. After a crude lysate has been generated, the first step in processing a crude lysate is often ultracentrifugation, which separates the solution into distinct bands containing organelles, membrane lipids, proteins, and nucleic acids. A crude preparation can come from cells or tissues and is not initially purified and will be recognized by those skilled in the art. In several embodiments described herein, the sample in which the target protein or POI is purified, comes from lysed cells that are harvested.

In several embodiments described herein, a protein of interest (POI) can be separated from a sample through affinity methods. In particular, a POI is genetically fused to a proteinaceous PDZ element and separated from a mixture and in particular, a crude preparation, by proper selection of a corresponding PDZ ligand.

The term “protein separation” as described herein describes a series of steps that are intended to isolate one or a few proteins from a complex mixture, usually cells, tissues or whole organisms, as well as a crude preparation that can be a cell lysate. Protein purification is vital for the characterization of the function, structure and interactions of the protein of interest. The purification process can separate the protein and non-protein parts of the mixture, and finally separate the desired protein from all other proteins. Separation of one protein from all others is typically the most laborious aspect of protein purification. Separation steps usually exploit differences in protein size, physico-chemical properties, binding affinity and biological activity. Separation and purification of POI can occur by use of a support, and is described in several embodiments herein.

In some embodiments of the affinity chromatography methods and systems herein described, the PDZ affinity tags can be selected based on their ability to bind a corresponding PDZ ligand. Some exemplary types provided herein for illustrative purposes are: PDZ affinity tags comprising C-terminal peptide sequences known to bind in the ligand binding pockets of PDZ domains (herein also Type A tags); PDZ domains capable of binding ligands via their ligand binding pocket or binding other PDZ domains via their β-hairpin finger motif (herein also Type B tags); PDZ domains capable of binding ligands via their ligand binding pocket (herein also Type C tags). In the examples of the present disclosure, several embodiments described herein comprise PDZ domains with types A, B, and C tags. Additionally exemplary PDZ affinity tags and corresponding PDZ affinity ligand can be selected among the PDZ domain and PDZ binding C-terminal peptide grouped as Type I, Type II and Type III illustrated in Table 1 herein provided.

In some embodiments a PDZ domain tag and related PDZ peptide ligand according to one grouping can be selected with one or more criteria here provided. For example a PDZ affinity Type A tag can also be selected to be one of the Type I, Type II and Type III groups. A PDZ affinity Type C tag can be selected to be of the Type I, II and III groups based upon its specificity for PDZ domain C-terminal peptide ligands. Additionally, a PDZ domain containing a β-hairpin (Type B affinity tag) can be selected to be of the Type I, II and III groups based upon its specificity for PDZ domain C-terminal peptide ligands and Type I, II and III groups based upon the specificity of its associated β-hairpin for Type I, II and III PDZ domains.

In some embodiments, because of the high specificity and affinity of the PDZ domain for its C-terminal peptide binding partner, attaching a PDZ binding C-terminal peptide sequence to any POI can direct binding of the POI to a PDZ domain immobilized on a solid support. This allows separation of the POI from the starting mixture including crude biological preparations.

Detachment of the tagged POI from the free or immobilized PDZ domain can then be accomplished by the addition of concentrated free PDZ binding peptide, salt, a change in pH, and of additional parameters identifiable by a skilled person, which compete for the interactions with the PDZ domain. Additionally, instead of attaching a PDZ binding C-terminal peptide sequence to the POI, the POI can also be fused to a PDZ Domain and purified using a PDZ binding peptide immobilized on a solid support.

The term “detachment” and “detach” as used herein, indicate the act of separation of a complex formed by two or more molecules. Detachment of POI from free or immobilized PDZ domain can be achieved by weakening the bonds holding the complex together through the use of chaotropes (e.g. ionic detergents such as SDS, urea, guanidinium), kosmotropes (e.g. sulfate, magnesium, etc.), salts (104), proteases (e.g. TEV protease, Factor Xa, Thrombin, etc.), pH changes (104). Detachment of the complex can also occur by the addition of concentrated free PDZ binding peptide (3), concentrated PDZ domain or PDZ-BH domains, cyclic peptides (24,25), small molecules (26) or lipids (27), or additional techniques identifiable by a skilled person.

In some embodiments herein described a PDZ tag specifically binds the PDZ ligand with a first binding affinity K_{D}, and the PDZ ligand or PDZ tag specifically binds the reagent with a second binding affinity K_{Dcomp}. The second binding affinity K_{Dcomp} can be lower, equal to, or higher than the first binding affinity K_{D} and can be selected in combination with other parameters to allow detachment of the PDZ ligand from the PDZ affinity tag following contacting of the PDZ affinity tag-PDZ ligand complex. In particular the detachment can be performed at set elution conditions wherein ratio of eluted target with respect to total bound target is a function of K_{D}, K_{Dcomp} and L0, wherein L0 is the concentration of the PDZ ligand.

In some embodiments, the ratio of eluted target with respect to total bound target is a function of (KD comp*L0)/Kd. In some of those embodiments, the ratio of eluted target with respect to total target is a maximized by minimizing (KD comp*L0)/Kd.

In some embodiments, the ratio of eluted target with respect to total target is further a function of the ratio between the volume of reagent added and the pore volume of a porous support.

In some embodiments, the competitive elution of a target protein/peptide by adding a competitive free ligand can be described by the ratio of the amount of eluted target protein/peptide to total bound target protein/peptide as follows:

$$\frac{Eluted target}{Total bound target} \approx ( p / p + 1 ) * [ \frac{{pC}_{0}}{{pC}_{0} + \frac{{K}_{DComp} * L 0}{{K}_{D}}} ]$$

where, p is the ratio between the volume of competitor added and the pore volume of a porous support (e.g. gel), K_{D} is the dissociation constant for coupled ligand, K_{Dcomp} is the dissociation constant for free competing ligand, C₀ is the concentration of competing ligand and L₀ is the concentration of the coupled ligand.

In principle using a peptide (I) to knock off a peptide (S) bound to a PDZ domain either of the latter two bound to a solid phase resin, would be equivalent to competitive binding to a protein or competitive inhibition of an enzyme. As per the equation above, the degree of elution depends on the two K_{D} values and concentrations of the I and S. Assuming that the binding and release of both I and S from the domain are rapid, a large excess concentration of I should reverse the binding of S to the Domain. For example, if both K_{D} and K_{Dcomp} are similar in magnitude, then the concentrations of the competing and coupled ligand must be similar to achieve efficient elution. If on the other hand K_{Dcomp} is five times larger than K_{D} (e.g. 5*K_{D}) then the concentration of competing ligand must be five time higher to achieve successful elution.

Deviations from the theoretical concentrations/amounts needed to elute the peptide from the PDZ domain, would arise from a variety of factors, such as but not limited to the heterogeneous environment, the exact composition of the solid support resin, diminished access of the I to the S-bound PDZ domain, whether the elution is performed as a batch process or flow through column process, etc.

Because K_{D} values are greatly impacted by pH and ionic strength, changes in the pH and ionic strength of the buffer solution should greatly affect the affinity between PDZ domains and their ligands. It has previously been shown that the association rate constant, kₒₙ, for the PDZ-peptide binding reaction decreases approximately 2 fold as the ionic strength of the buffer is increased from 100 uM to 1M. Additionally different effects on binding affinity are obtained based on the counter ion identity (e.g. fluoride, chloride, nitrate), with chloride having a negligible effect on PDZ domain and PDZ binding C-terminal binding affinity over the range of NaCl concentrations from 0 mM to 1500 mM. The pH of solution is expected to negatively affect binding affinity when pH has changed enough to protonate, and thus neutralize, acidic groups that are involved in specific PDZ domain and PDZ binding C-terminal peptide recognition. A pH below 5 would be expected to negatively affect PDZ domain-PDZ binding C-terminal peptide complexation, due to the protonation of Aspartic Acid (pK_{A} 3.86) and Glutamic Acid (pK_{A} 4.25). Alternatively, an excessively high pH greater than 10.5 could result in partial neutralization of Lysine residues in PDZ domains involved in PDZ binding C-terminal peptide recognition.

Exemplary detachments can be performed by affinity competition (e.g. flow over the substrate surface of a protein that binds the substrate with higher affinity than the POI), by interference of the binding interface of the POI and the substrate (e.g. high salt concentrations), or detached by denaturation of the POI or the substrate and therefore disruption of the binding interface between the POI and the substrate (e.g. urea).

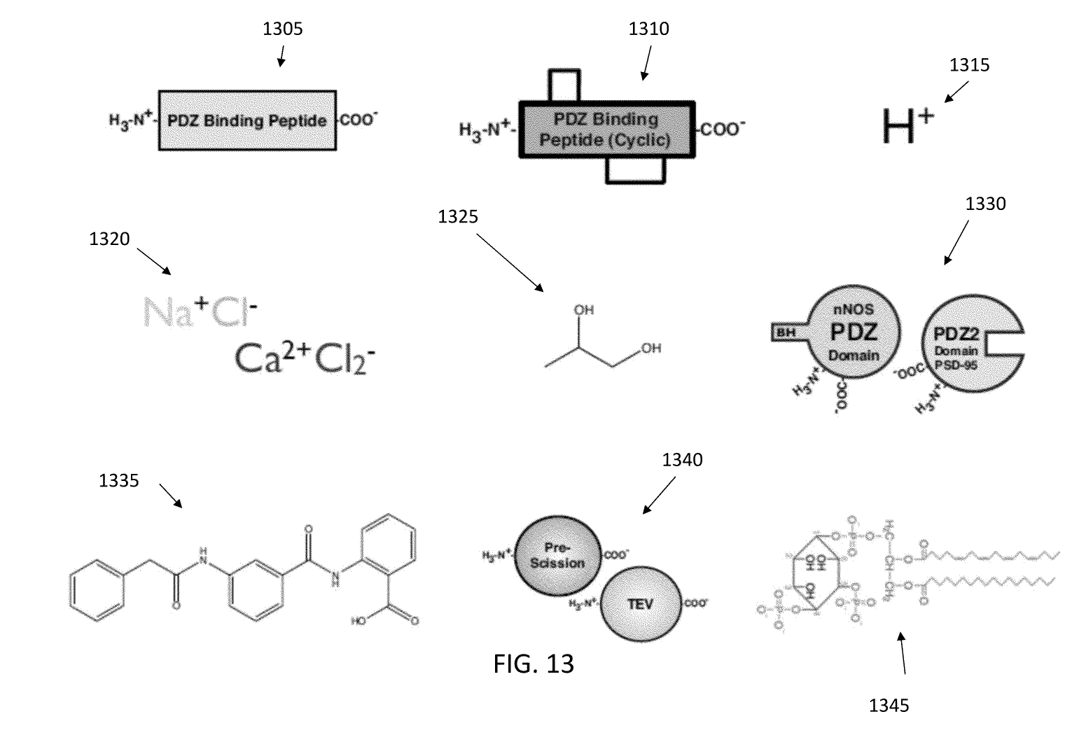

In several embodiments described herein, there are elution buffers or buffers that are used to flow over the solid support in order to elute the POI. There are different types of elution buffer used depending on type of molecules that are attached to the solid support, as shown in FIG. 13. The elution buffers can comprise i) PDZ Binding Peptide (1305), ii) Cyclic PDZ Binding Peptide (1310), iii) a specific pH (1315), iv) a specific salt (1320), v) a polyol and salt (1325) vi) PDZ Domains (1330), v) specific small molecules (1335), vi) proteases (1340) or vii) lipids (1345).

For elution buffers comprising PDZ binding C-terminal peptide (1305), the most common mode of PDZ domain protein-protein interaction involves the recognition of C-terminal peptide motifs present on protein binding partners. A molar excess of free PDZ binding C-terminal peptide will be used to elute bound protein from PDZ affinity resin.

For elution buffers comprising a cyclic PDZ binding C-terminal peptide (1310), cyclic peptides have been found to exhibit increased affinity for PDZ domain ligand binding pockets relative to noncyclic peptides (24,26). A molar excess of free Cyclic PDZ binding C-terminal peptide will be used to elute bound protein from PDZ affinity resin.

For elution buffers that are based on using pH for eluting specific proteins (1315), the specificity and strength of interactions between the ligand binding pocket and cognate ligands can be heavily dependent on electrostatic attraction/repulsion, therefore changes in pH resulting in protonation/deprotonation of charged groups could be used to elute molecules from PDZ affinity resin.

For elution buffers that are based using salt concentrations for elution (1320), the specificity and strength of interactions between the ligand binding pocket and cognate ligands can be heavily dependent on electrostatic attraction/repulsion, therefore changes ionic strength can dampen or strength attractive/repulsive forces between charged groups and could be used to elute molecules from PDZ affinity resin. Salts such as potassium chloride, sodium chloride, and other salts known to those skilled in the art can be used for elution.

For elution buffers that are based on polyol and salt concentrations (1325), a specific type of naturally occurring monoclonal antibodies exist that are capable of binding antigens with high affinity under binding conditions, yet exhibit low affinity for their cognate antigen in the presence of a combination of non-chaotropic salts and low molecular weight hydroxylated compounds (polyols) (105). Polyols and non-chaotropic salts will be utilized to elute tagged POI bound to PDZ affinity resin composed of immobilized polyol antibodies.

For elution buffers that are based on having a PDZ domain concentration in the buffer (1330), a molar excess of free PDZ Domain will be used to elute bound protein from PDZ affinity resin, which can strip of the protein due to protein binding competition.

For elution buffers that are based on having small molecules for elution (1335), the small molecules are selected to have a high affinity for binding the PDZ domain ligand binding pockets (CITATION). A molar excess of free small molecule will be used to elute bound protein from PDZ affinity resin.

For elution buffers that are based on having protease for removing a POI (1340), first this involves insertion of protease recognition sequences in between a POI and a PDZ Affinity Tag, through molecular biology techniques, which are known to those skilled in the art. Treatment with the appropriate Protease will result in cleavage of peptide bonds and will allow separation of the POI and the PDZ affinity Tag (especially useful if the PDZ Affinity Tag affects POI functionality). All PDZ Affinity Tag expression vectors will be designed to include protease recognition sites to allow for tag removal following purification on PDZ Affinity Resin. If elution from PDZ Affinity Resin by other elution agents listed on this page has a deleterious effect on POI function, the POI can be eluted from the PDZ Affinity Resin by treatment with a protease capable of cleaving a complementary protease recognition sequence included in the PDZ Affinity Tag-POI fusion protein.

In the elution buffer that is based on elution by a lipid concentration (1345), a molar excess of free lipid will be used to elute bound protein from the affinity resin, as lipids have been found to bind in the PDZ domain ligand binding pockets with a high affinity (27).

The PDZ Affinity Chromatography system can provide an alternative to currently available affinity chromatography products, as it is suitable for the purification of several key neuronal POI, and can provide increased purity and yield of POI relative to existing affinity chromatography methods. Neuronal proteins have been shown to be challenging in terms of their purification. In the present disclosure, the purity of neuronal POI is improved when attached to a PDZ domain for purification. The affinity chromatography system can comprise protein affinity tags, peptide affinity tags, an affinity resin and elution agents.

Affinity tags and affinity resin can be derived from naturally occurring protein sequences. In other embodiments, bioinformatics and directed evolution can be utilized to design new PDZ domain-peptide sequence, PDZ domain-PDZ domain with β-hairpin and PDZ domain-small molecule interactions. Programs and methods such as PyMOL, MacPyMOL, UCSF Chimera, and MolMol in order to model high affinity binding are known to those skilled in the art.

Protein domains and peptide sequences used as affinity tags in PDZ Affinity Chromatography can have the advantage of being small (for example, less than 12 kDa for domain tags and less than 0.6 kDa for peptide tags) and monomeric (domain and peptide tags), which are advantageous characteristics in affinity tags and pairwise and beyond protein-protein interactions. In several embodiments described herein, the protein of interest can have one tag (12 kDa) or up to two tags (24 kDa), which can be internal, at the C-terminal end, or at the N-terminal end of the POI.

Additionally, the methods of the present disclosure can be utilized to purify lipids, peptides, small molecules, etc. capable of binding to PDZ affinity resin. The methods of the present disclosure can also be used for protein purification, as well as to force pairwise and beyond protein-protein interactions by tagging each pair of proteins with complementary N- or C-terminal tags.

Affinity chromatography exploits the natural specific recognition between biological molecules to allow purification of a single protein of interest (POI) from a crude preparation. In most affinity chromatography procedures known in the art, the POI is genetically fused to a proteinaceous affinity tag that confers the ability to bind a specific molecule immobilized on a solid support. In several embodiments described herein, a fusion protein can be created that has either a C-terminal, N-terminal, or internal PDZ domain in order for the capture of the protein of interest. The use of cloning is known to those skilled in the art for the engineering and expression of proteins of interest that contain a PDZ domain.

In some exemplary embodiments, to form a fusion protein PCR techniques can be used. Two sets of primers can be provided, a first primer with sequence overlap of the first protein of interest with a second protein of interest, a second primer with sequence overlap of the second protein of interest with the first protein of interest. A round of PCR can be run on the first protein of interest with the first primer. A round of PCR is run on the second protein of interest with the second primer. The result from the two PCR rounds are combined with and the overlaps of the two respective primers used to prime an additional round of PCR resulting in a gene fusion of the first protein of interest and the second protein of interest. The mixture containing the gene fusion is run on a gel for purification. The resulting purified product can be introduced for example into a pJexpress vector.

Due to the high specificity and affinity of the PDZ domain for its C-terminal peptide binding partners or other molecules, attaching a PDZ binding C-terminal peptide sequence to any POI could direct binding of the POI to a PDZ domain immobilized on a solid support, allowing separation of the POI from crude biological preparations. Elution of the tagged POI from the immobilized PDZ domain can be accomplished by the addition of concentrated free PDZ binding peptide, salt, a change in pH, etc.

First generation pairs formed with a PDZ binding C-terminal peptide tag and a PDZ Domain have been derived from naturally occurring C-terminal PDZ binding peptide sequences and their known PDZ domain binding partners. Second generation and beyond pairs formed with PDZ binding C-terminal peptide tags and PDZ domain pairs can be computationally designed and optimized by directed evolution methodologies to confer increased specificity and affinity over existing PDZ binding C-terminal peptide-PDZ domain pairs derived from nature.

In some embodiments, the C-terminal 7 amino acids from the NR2B tail protein (SSIESDV) can be used as a first generation PDZ binding C-terminal peptide tag (e.g. Type A PDZ affinity tag). The PDZ2 domain from PSD-95 can be purified and immobilized on a solid support to generate PDZ affinity resin. The immobilized PDZ2 domain can then be used to capture any POI fused to a PDZ binding C-terminal peptide tag (e.g. Type A PDZ affinity tag).

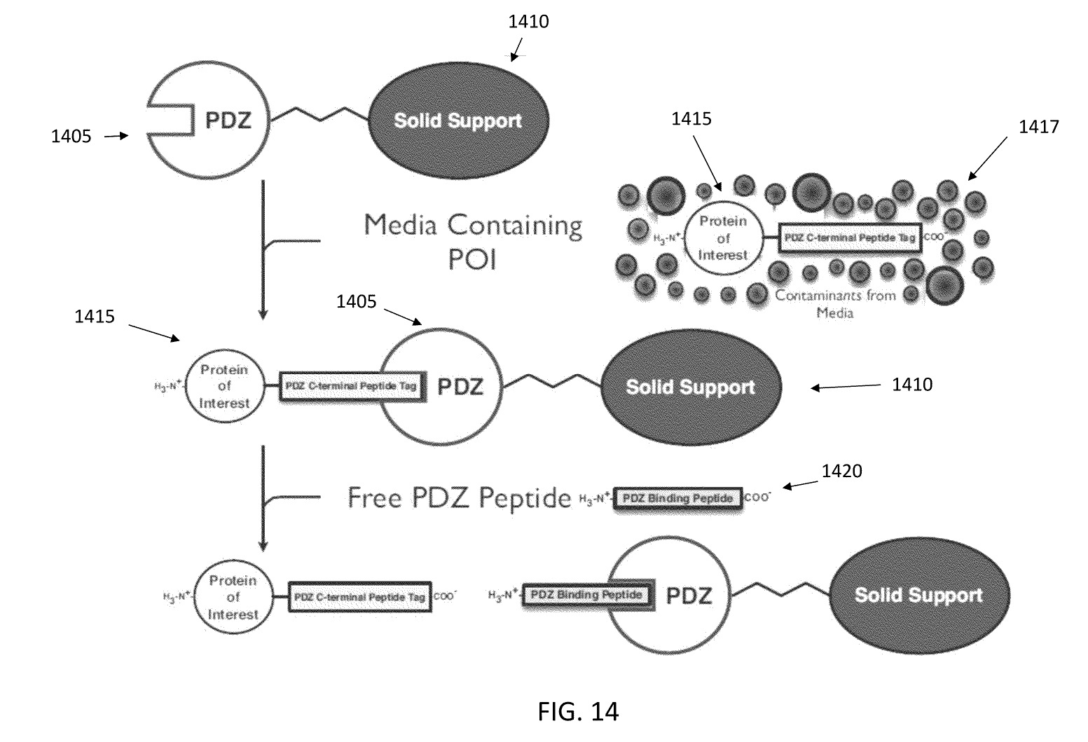

Purification schemes for several of the embodiments have been described herein and are exemplified in FIG. 14 to FIG. 24.

FIG. 14 illustrates an example of a purification scheme of one of the embodiments described herein, wherein the affinity matrix is a PDZ domain (1405) immobilized on a solid support (1410)—for example PDZ2 from PSD-95. The protein tag (1415) can be a Type A PDZ affinity tag, a PDZ binding C-terminal peptide—for example, a POI fused to SSIESDV from NR2B tail. The POI with the tag (1415) can be surrounded by contaminants in the media (1417).

The tag (1415) attaches to the PDZ domain (1405) on the support (1410). Media contaminants are removed by washing the support with buffer. The elution agent can be a free PDZ binding C-terminal peptide—for example H₃N+-SIESDV-COO— (SEQ ID NO: 160) from NR2B tail.

A PDZ domain can be purified by a combination of chromatographic steps, and is covalently attached (e.g. NHS/EDC coupling) to a solid support (e.g. agarose beads) to generate a PDZ domain affinity resin. A recombinant Protein of Interest (POI), genetically fused to a PDZ binding C-terminal peptide affinity tag (a Type A PDZ affinity tag) can be expressed in a cell line of choice (e.g. E. coli) or in vitro translation system. The cells can be lysed and, if needed, clarified by centrifugation. Clarified lysate containing the POI can then be incubated with the PDZ domain affinity resin.

The PDZ domain affinity resin can then be washed with a buffer to remove weakly and non-specifically bound host cell proteins. The POI fused to the PDZ binding C-terminal peptide affinity tag (a Type A tag) can then be eluted from the resin by incubation with a buffer containing free PDZ binding peptide (1420) or other elution agents (for example, salt, pH, denaturant, and additional agents identifiable by a skilled person).

The PDZ domain affinity resin can then be regenerated for repeat use by stripping away bound free PDZ binding peptide and washing by the use, for example, of salt, pH, denaturants, etc. Any POI containing a PDZ binding C-terminal peptide capable of binding the domain attached to this resin could be purified with this scheme.

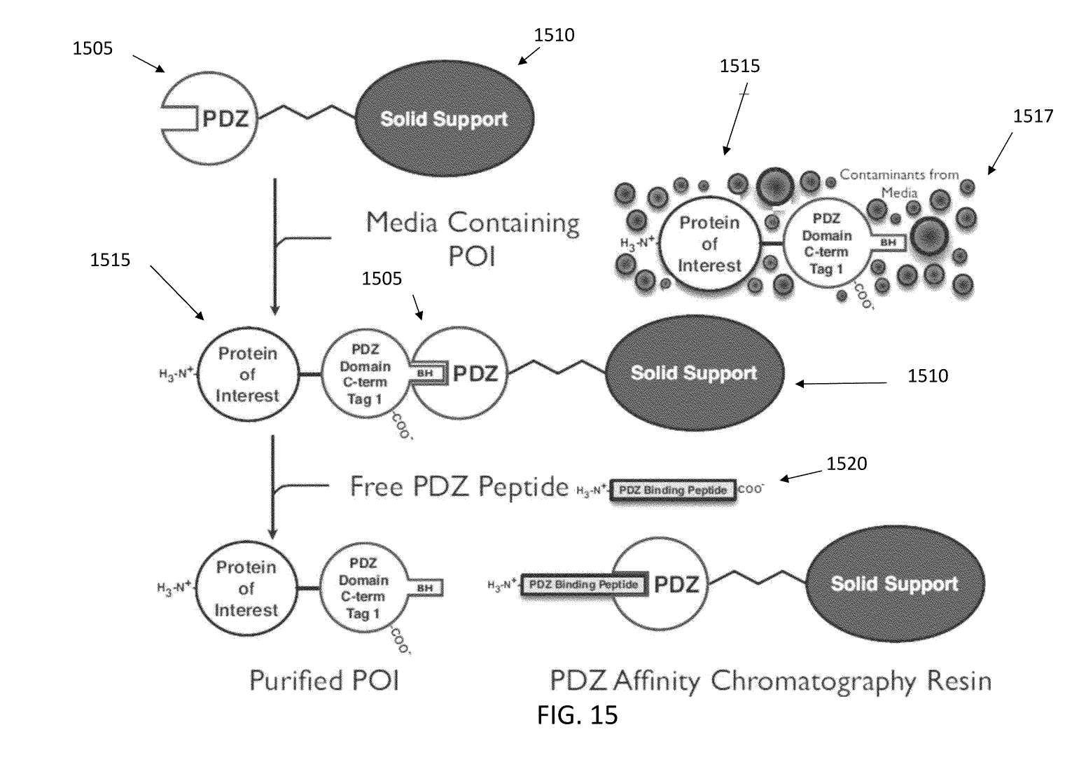

FIG. 15 illustrates another example of a purification scheme, of one of the embodiments described herein. In this embodiment, the affinity matrix is a PDZ domain (1505) immobilized on a solid support (1510)—for example PDZ2 from PSD-95. The protein tag (1515) can be a Type B PDZ affinity tag, a C-terminal PDZ domain with a β-hairpin—for example, a POI fused to nNOS PDZ domain. The POI with the tag (1515) can be surrounded by contaminants in the media (1517).

The tag (1515) attaches to the PDZ domain (1505) on the support (1510). Media contaminants are removed by washing the support with buffer. The elution agent can be a free PDZ binding C-terminal peptide—for example H₃N+-SIESDV-COO— (SEQ ID NO: 160) from NR2B tail.

A PDZ domain can be purified by a combination of chromatographic steps, and is covalently attached (e.g. NHS/EDC coupling) to a solid support (e.g. agarose beads) to generate a PDZ domain affinity resin. A POI genetically fused to a C-terminal PDZ domain with β-hairpin tag (a Type B PDZ affinity tag) can be expressed in a cell line of choice (e.g. E. coli) or in vitro translation system. The cells can be lysed and, if needed, clarified by centrifugation. Clarified lysate containing the POI can then be incubated with the PDZ domain affinity resin.

The PDZ domain affinity resin can then be washed with a buffer to remove weakly and non-specifically bound host cell proteins. The POI fused to the PDZ binding C-terminal peptide affinity tag (a Type B tag) can then be eluted from the resin by incubation with a buffer containing free PDZ binding peptide (1520) or other elution agents (for example, salt, pH, denaturant, etc.).

The PDZ domain affinity resin can then be regenerated for repeat use by stripping away bound free PDZ binding peptide and washing by the use, for example, of salt, pH, denaturants, etc. Any POI containing a C-terminal PDZ domain with β-hairpin capable of binding the domain attached to this resin could be purified with this scheme.

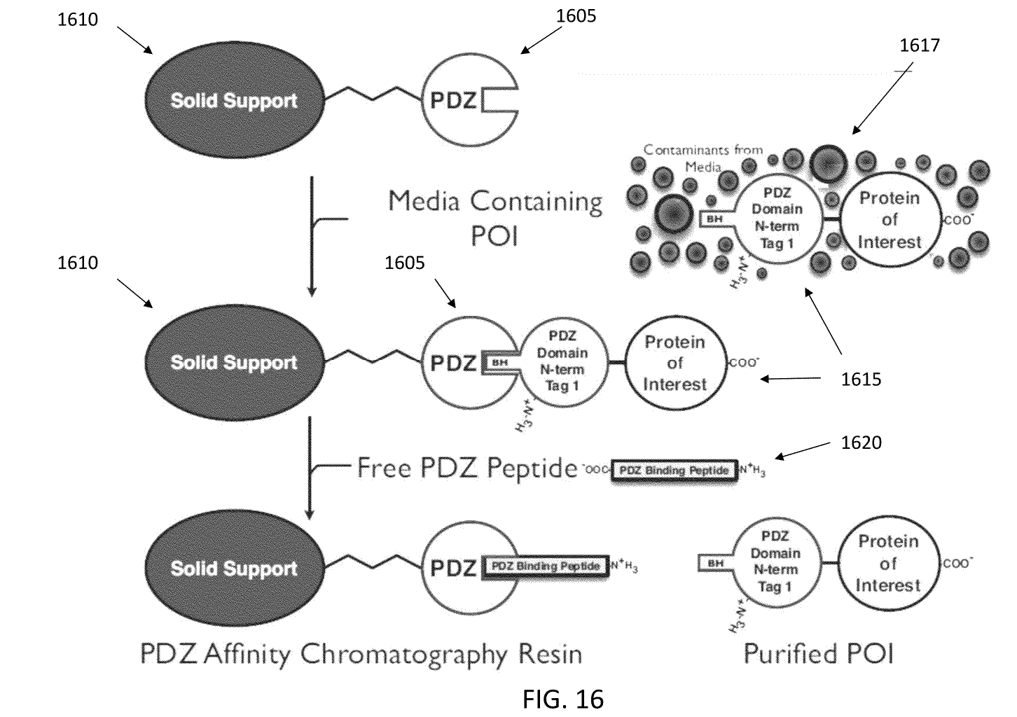

FIG. 16 illustrates another example of a purification scheme of one of the embodiments. In the embodiment exemplified in FIG. 16, the affinity matrix is a PDZ domain (1605) immobilized on a solid support (1610)—for example PDZ2 from PSD-95. The protein tag (1615) can be a Type B PDZ affinity tag, an N-terminal PDZ domain with a β-hairpin—for example, a POI fused to nNOS PDZ domain. The POI with the tag (1615) can be surrounded by contaminants in the media (1617).

The tag (1615) attaches to the PDZ domain (1605) on the support (1610). Media contaminants are removed by washing the support with buffer. The elution agent can be a free PDZ binding C-terminal peptide—for example H₃N⁺-SIESDV-COO⁻ (SEQ ID NO: 160) from NR2B tail.

A PDZ domain can be purified by a combination of chromatographic steps, and is covalently attached (e.g. NHS/EDC coupling) to a solid support (e.g. agarose beads) to generate a PDZ domain affinity resin. A recombinant Protein of Interest (POI), genetically fused to an N-terminal PDZ domain with β-hairpin tag (a Type B PDZ affinity tag) can be expressed in a cell line of choice (e.g. E. coli) or in vitro translation system. The cells can be lysed and, if needed, clarified by centrifugation. Clarified lysate containing the POI can then be incubated with the PDZ domain affinity resin.

The PDZ domain affinity resin can then be washed with a buffer to remove weakly and non-specifically bound host cell proteins. The POI fused to the N-terminal PDZ binding peptide affinity tag (a Type B tag) can then be eluted from the resin by incubation with a buffer containing free PDZ binding peptide (1620) or other elution agents (for example, salt, pH, denaturant, etc.).

The PDZ domain affinity resin can then be regenerated for repeat use by stripping away bound free PDZ binding peptide and washing by the use, for example, of salt, pH, denaturants, etc. Any POI containing a C-terminal PDZ domain with β-hairpin capable of binding the domain attached to this resin could be purified with this scheme.

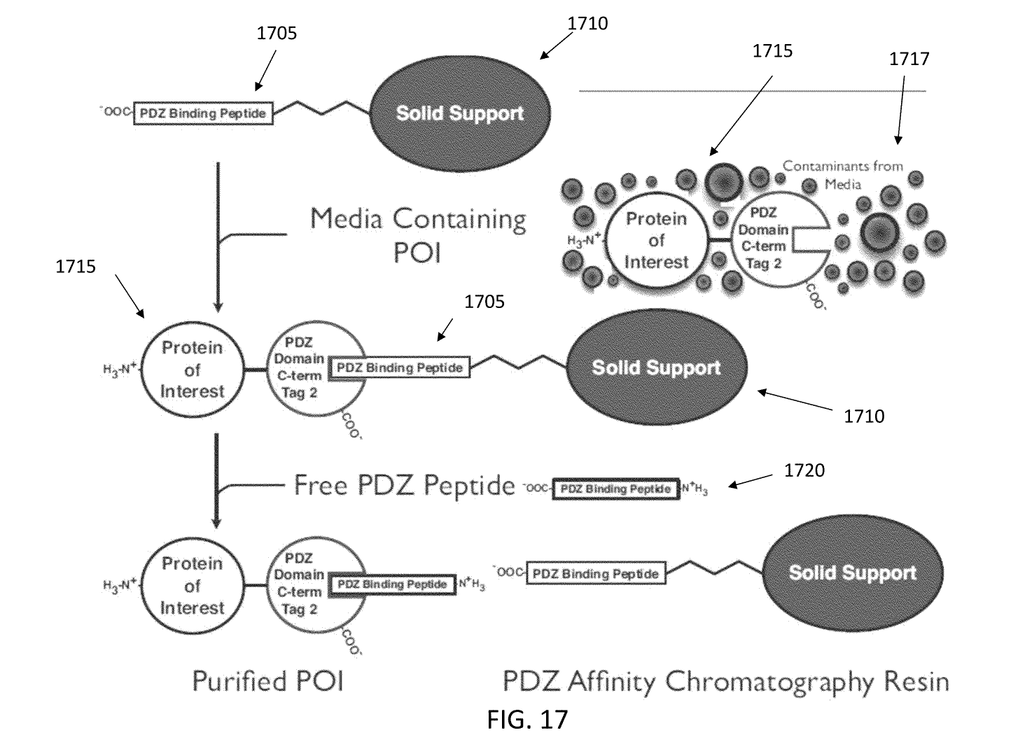

FIG. 17 illustrates another example of a purification scheme of one of the embodiments described herein. In the embodiment of FIG. 17, the affinity matrix is a PDZ binding C-terminal peptide (1705) immobilized on a solid support (1710), for example H₃N+-SIESDV-COO— (SEQ ID NO: 160) from NR2B Tail. The protein tag (1715) can be a Type C PDZ affinity tag, a C-terminal PDZ domain, for example, a POI fused to PDZ2 from PSD-95. The POI with the tag (1715) can be surrounded by contaminants in the media (1717).

The tag (1715) attaches to the binding peptide (1705) on the support (1710). Media contaminants are removed by washing the support with buffer. The elution agent can be a free PDZ binding C-terminal peptide, for example H₃N⁺-SIESDV-COO⁻ (SEQ ID NO: 160) from the NR2B tail. The PDZ binding C-terminal peptide can be synthesized, and is covalently attached (e.g. NHS/EDC coupling) to a solid support (e.g. agarose beads) to generate a PDZ binding C-terminal peptide affinity resin. A recombinant Protein of Interest (POI), genetically fused to a C-terminal PDZ domain tag (a Type C PDZ affinity tag) can be expressed in a cell line of choice (e.g. E. coli) or in vitro translation system. The cells can be lysed and, if needed, clarified by centrifugation. Clarified lysate containing the POI can then be incubated with the PDZ binding C-terminal peptide affinity resin.

The PDZ binding C-terminal peptide affinity resin can then be washed with a buffer to remove weakly and non-specifically bound host cell proteins. The POI fused to the C-terminal PDZ domain tag (a Type C tag) can then be eluted from the resin by incubation with a buffer containing free PDZ binding peptide (1720) or other elution agents (for example, salt, pH, denaturant, etc.). The PDZ binding C-terminal peptide affinity resin can then be regenerated for repeat use by stripping away bound free PDZ binding peptide and washing by the use, for example, of salt, pH, denaturants, etc. Any POI containing a PDZ domain with or without β-hairpin capable of binding the PDZ binding C-terminal peptide attached to this resin could be purified with this scheme.

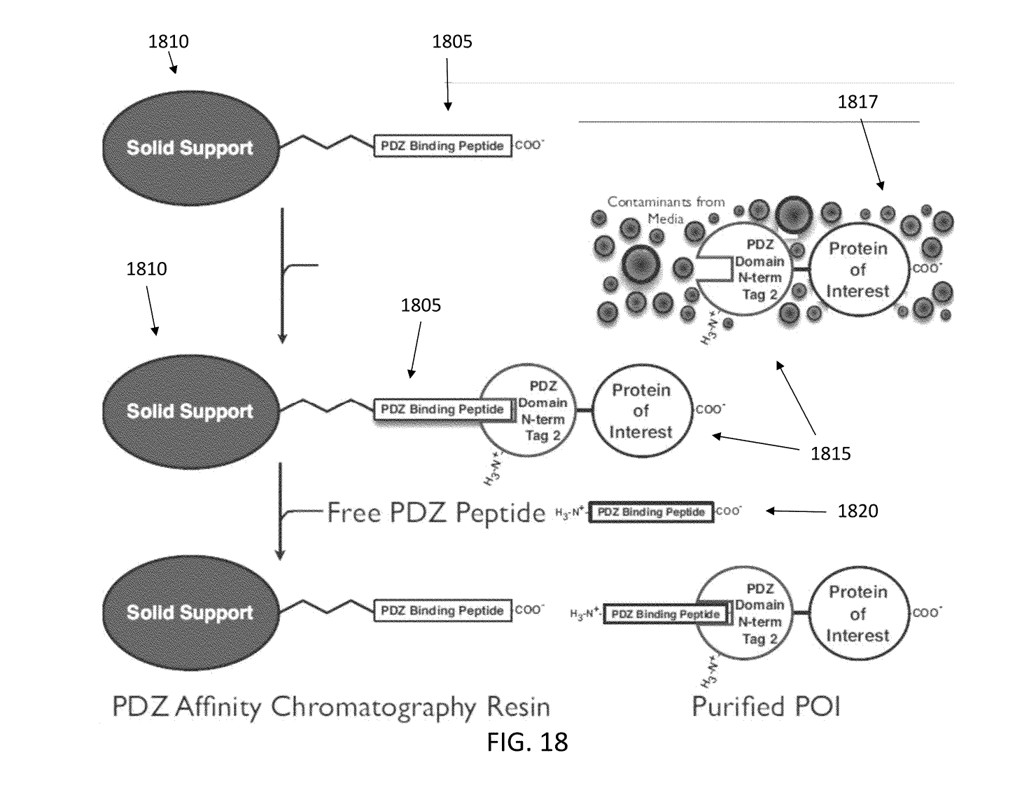

FIG. 18 illustrates another example of a purification scheme. In the embodiment of FIG. 18, the affinity matrix is a PDZ binding C-terminal peptide (1805) immobilized on a solid support (1810)—for example H₃N⁺-SIESDV-COO⁻ (SEQ ID NO: 160) from NR2B Tail. The protein tag (1815) can be a Type C PDZ affinity tag, an N-terminal PDZ domain—for example, a POI fused to PDZ2 from PSD-95. The POI with the tag (1815) can be surrounded by contaminants in the media (1817).

The tag (1815) attaches to the binding peptide (1805) on the support (1810). Media contaminants are removed by washing the support with buffer. The elution agent can be a free PDZ binding C-terminal peptide, for example H₃N⁺-SIESDV-COO⁻ (SEQ ID NO: 160) from NR2B tail.

A PDZ binding C-terminal peptide can be synthesized, and is covalently attached (e.g. NHS/EDC coupling) to a solid support (e.g. agarose beads) to generate an N-terminal PDZ binding peptide affinity resin. A recombinant Protein of Interest (POI), genetically fused to an N-terminal PDZ domain tag (a Type C PDZ affinity tag) can be expressed in a cell line of choice (e.g. E. coli) or in vitro translation system. The cells can be lysed and, if needed, clarified by centrifugation. Clarified lysate containing the POI can then be incubated with the PDZ binding C-terminal peptide affinity resin.

The PDZ binding C-terminal peptide affinity resin can then be washed with a buffer to remove weakly and non-specifically bound host cell proteins. The POI fused to the N-terminal PDZ domain tag (a Type C tag) can then be eluted from the resin by incubation with a buffer containing free PDZ binding peptide (1820) or other elution agents (for example, salt, pH, denaturant, etc.).

The PDZ binding C-terminal peptide affinity resin can then be regenerated for repeat use by stripping away bound free PDZ binding peptide and washing by the use, for example, of salt, pH, denaturants, etc. Any POI containing a PDZ domain with or without β-hairpin capable of binding the PDZ binding C-terminal peptide attached to this resin could be purified with this scheme.

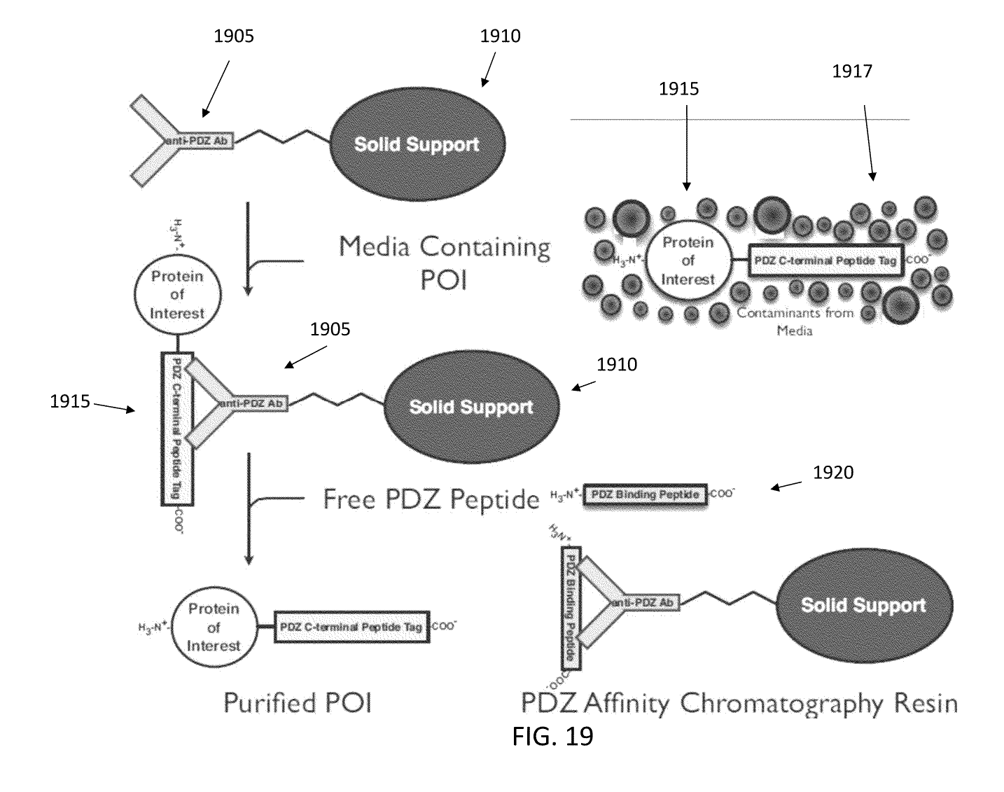

FIG. 19 illustrates another example of a purification scheme. In the embodiment of FIG. 19, the affinity matrix is an anti-PDZ C-terminal tag antibody (1905) immobilized on a solid support (1910)—for example an antibody that recognizes H₃N⁺-SIESDV-COO⁻ (SEQ ID NO: 160) peptide antigen from NR2B tail. The protein tag (1915) can be a Type A PDZ affinity tag, a PDZ binding C-terminal peptide—for example, a POI fused to SIESDV from NR2B Tail. The POI with the tag (1915) can be surrounded by contaminants in the media (1917).

The tag (1915) attaches to the anti-PDZ C-terminal tag antibody (1905) on the support (1910). Media contaminants are removed by washing the support with buffer. The elution agent can be a free PDZ binding C-terminal peptide—for example H₃N⁺-SIESDV-COO⁻ (SEQ ID NO: 160) from NR2B tail.

An antibody recognizing a PDZ binding C-terminal peptide tag antigen can be purified by a combination of chromatography steps, and covalently attached (e.g. NHS/EDC coupling) to a solid support (e.g. agarose beads) to generate an anti-PDZ binding C-terminal peptide affinity resin. A recombinant Protein of Interest (POI), genetically fused to a PDZ binding C-terminal peptide affinity tag (a Type A PDZ affinity tag) can be expressed in a cell line of choice (e.g. E. coli) or in vitro translation system. The cells can be lysed and, if needed, clarified by centrifugation. Clarified lysate containing the POI can then be incubated with the antibody-based PDZ affinity resin.

The antibody-based PDZ affinity resin can then be washed with a buffer to remove weakly and non-specifically bound host cell proteins. The POI fused to the PDZ binding C-terminal peptide affinity tag (a Type A tag) can then be eluted from the resin by incubation with a buffer containing free PDZ binding peptide (1920) or other elution agents (for example, salt, pH, denaturant, etc.).

The anti-PDZ binding C-terminal peptide antibody-based affinity resin can then be regenerated for repeat use by stripping away bound free PDZ binding peptide and washing by the use, for example, of salt, pH, denaturants, etc. Any POI containing a PDZ binding C-terminal peptide capable of binding the antibody attached to this resin could be purified with this scheme.

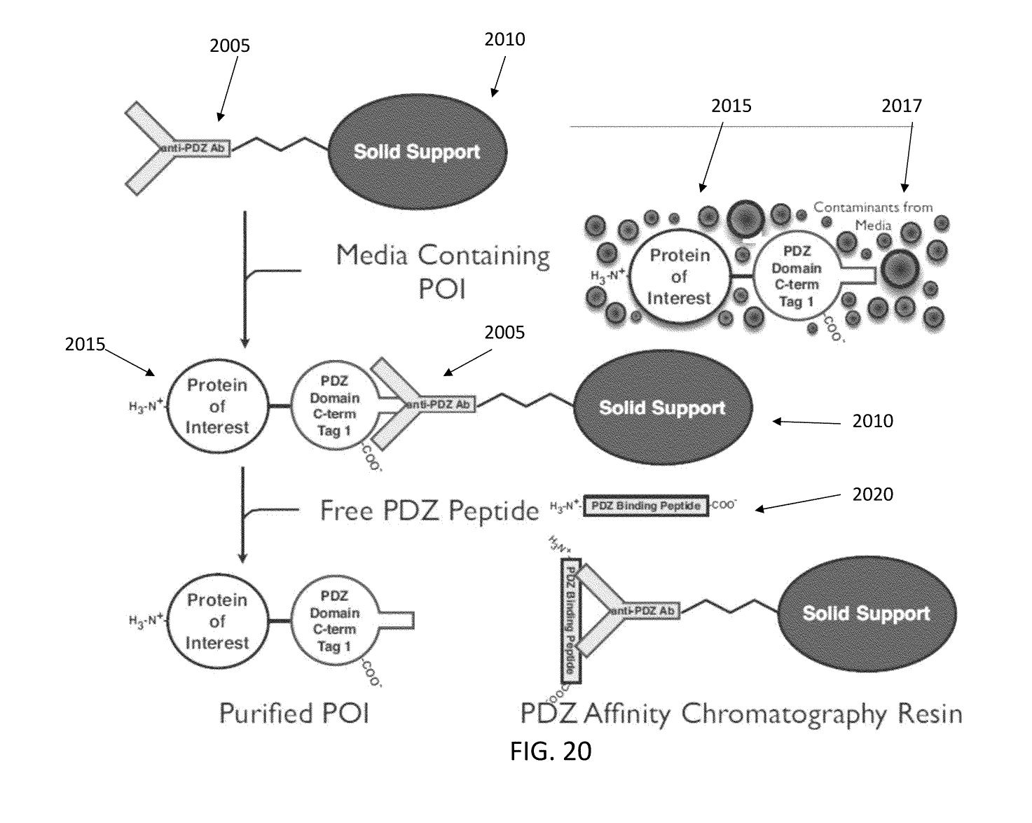

FIG. 20 illustrates another example of a purification scheme. In the embodiment of FIG. 20, the affinity matrix is an anti-PDZ domain tag antibody (2005) immobilized on a solid support (2010), for example an antibody that recognizes nNOS PDZ domain antigen. The protein tag (2015) can be a Type B PDZ affinity tag, a C-terminal PDZ domain with β-hairpin, for example, a POI fused to nNOS PDZ domain. The POI with the tag (2015) can be surrounded by contaminants in the media (2017).

The tag (2015) attaches to the anti-PDZ domain tag antibody (2005) on the support (2010). Media contaminants are removed by washing the support with buffer. The elution agent can be a free PDZ binding C-terminal peptide, for example H₃N⁺-SIESDV-COO⁻ (SEQ ID NO: 160) from NR2B tail.

An antibody recognizing a PDZ domain with β-hairpin can be purified by a combination of chromatography steps, and covalently attached (e.g. NHS/EDC coupling) to a solid support (e.g. agarose beads) to generate an anti-PDZ domain with β-hairpin binding antibody affinity resin. A recombinant Protein of Interest (POI), genetically fused to a PDZ domain with β-hairpin affinity tag (a Type B PDZ affinity tag) can be expressed in a cell line of choice (e.g. E. coli) or in vitro translation system. The cells can be lysed and, if needed, clarified by centrifugation. Clarified lysate containing the POI can then be incubated with the antibody-based PDZ affinity resin.

The antibody-based PDZ affinity resin can then be washed with a buffer to remove weakly and non-specifically bound host cell proteins. The POI fused to the C-terminal PDZ domain with β-hairpin affinity tag (a Type B tag) can then be eluted from the resin by incubation with a buffer containing free PDZ binding peptide (1920) or other elution agents (for example, salt, pH, denaturant and additional elution agents identifiable by skilled person).

The anti-PDZ domain with β-hairpin antibody-based affinity resin can then be regenerated for repeat use by stripping away bound free PDZ binding peptide and washing by the use, for example, of salt, pH, denaturants, etc. Any POI containing a PDZ domain with β-hairpin capable of binding the antibody attached to this resin could be purified with this scheme.

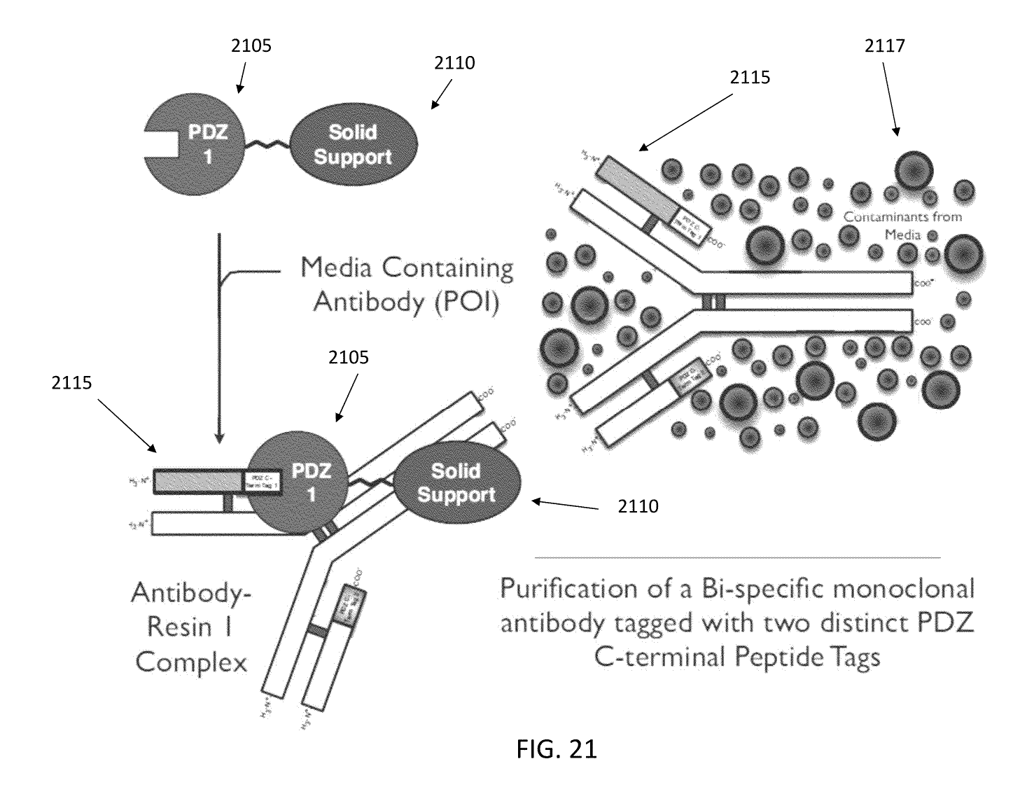

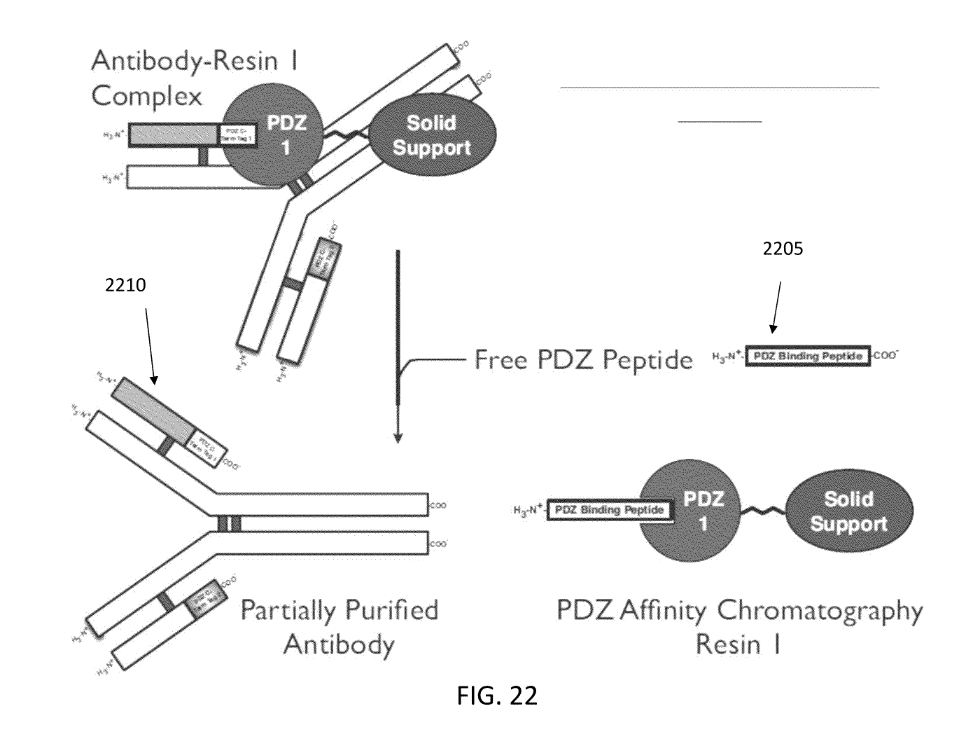

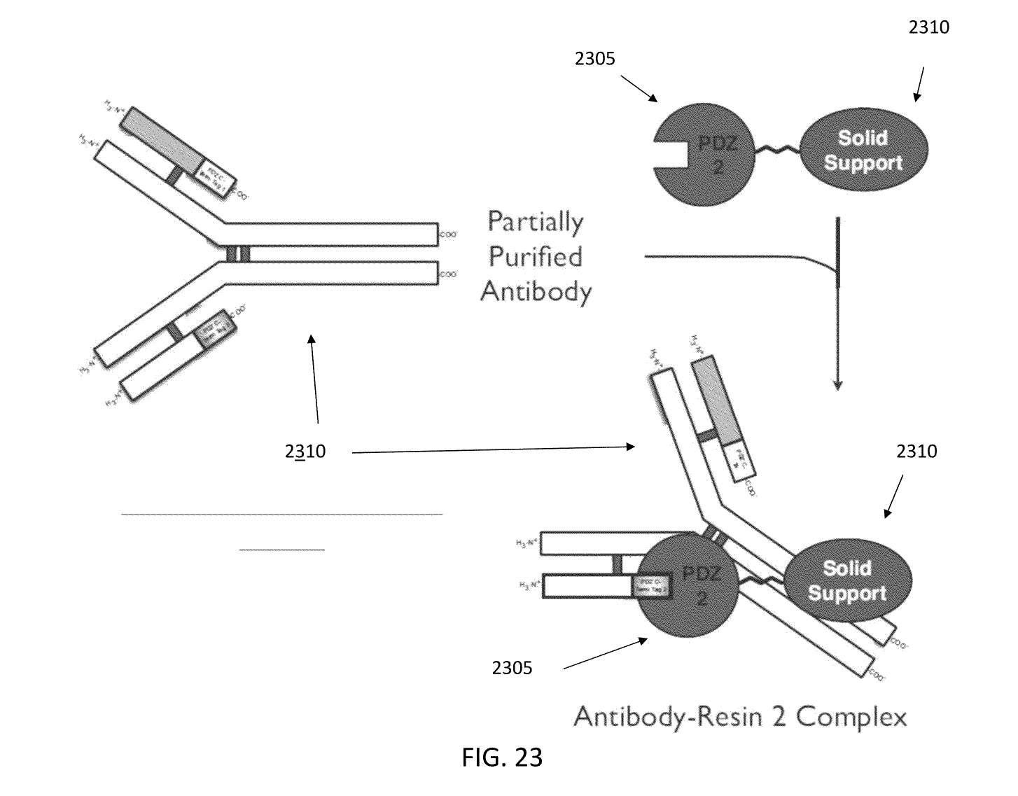

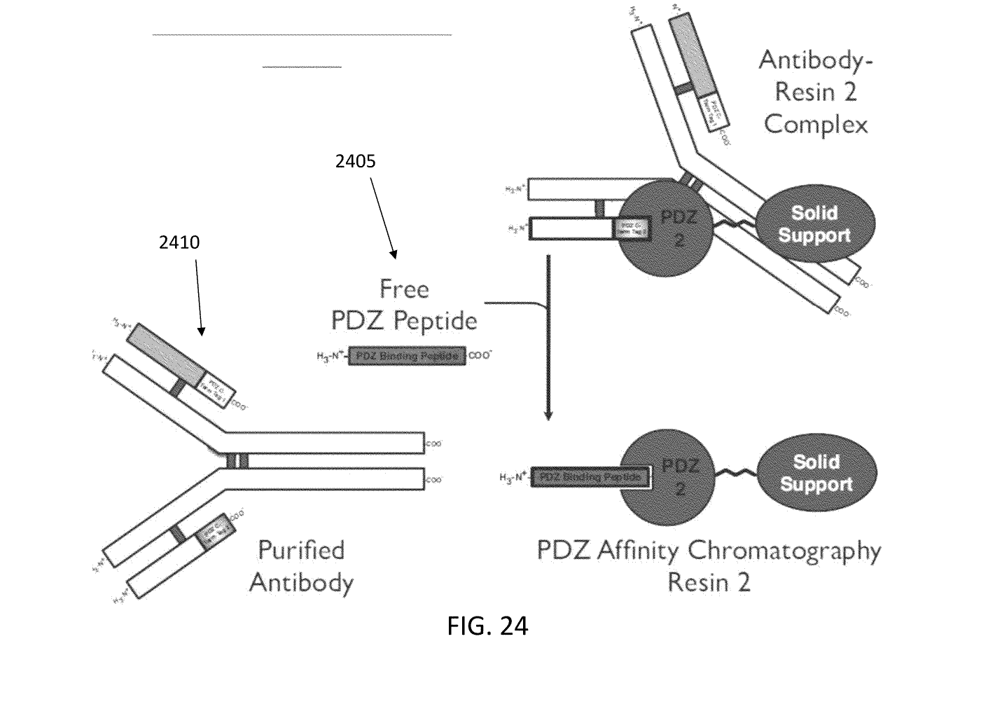

FIGS. 21-24 illustrate a two-step purification process. FIGS. 21-22 relate to step 1, while FIGS. 23-24 relate to step 2. The affinity matrix for step 1 can be a PDZ domain (2105) immobilized on a solid support (2110), for example, PDZ2 from PSD-95. For step 2, the affinity matrix can be PDZ3 from PSD-95. The protein tag for step 1 can be a PDZ binding C-terminal peptide, for example a light chain 1 fused to SIESDV from NR2B tail. For step 2, the protein tag can be a light chain 2 fused to YKQTSV from CRIPT. For step 1, the elution agent can be a free

PDZ binding C-terminal peptide, for example H₃N⁺-SIESDV-COO⁻ (SEQ ID NO: 160) from NR2B tail. For step 2, the binding peptide can be H₃N⁺-YKQTSV-COO⁻ from CRIPT.

In separate purifications, two PDZ domains are purified by a combination of chromatographic steps, and are covalently attached (e.g. by NHS/EDC coupling) to solid supports (e.g. agarose beads) to generate two unique PDZ domain affinity resins. A recombinant Protein of Interest (POI), in this case a bi-specific antibody (2115), genetically fused to two different PDZ binding C-terminal peptide affinity tags (one on each unique light chain) is expressed in a cell line of choice (e.g. E. coli) or in vitro translation system. The cells can be lysed and, if needed, clarified by centrifugation. Clarified lysate containing the bi-specific antibody is then incubated with the first PDZ domain affinity resin to select for the PDZ binding C-terminal peptide tag present on light chain 1. The first PDZ domain affinity resin is washed with a buffer to remove weakly and non-specifically bound host cell proteins. The bi-specific antibody can then be eluted from the first resin by incubation with buffer containing free PDZ binding peptide or other elution agent (salt, pH, denaturant, etc.). The PDZ domain affinity resin can then be regenerated for repeat use by stripping away bound free PDZ binding peptide and washing.

The procedure outlined above for the first step can then be repeated using the second PDZ domain affinity resin to select for the presence of light chain 2, which can be tagged with a second PDZ binding C-terminal peptide tag that can have a different sequence than the tag present on light chain 1. This two-step purification process can easily select for bi-specific antibodies in a pool of antibodies with two identical light chains. The antibody tag (2115) in FIG. 21 can be surrounded by contaminant (2117), and will need to be purified from the contaminants.

In a first step, referring to FIG. 21, the bi-specific antibody tag (2115) attaches to the PDZ domain (2105) which is attached to the solid support (2110). Subsequently, referring to FIG. 22, a free PDZ peptide (2205) can be used, similarly to the previous examples as detailed above in the present disclosure. A partially purified antibody (2210) is obtained.

Subsequently, referring to FIG. 23, the partially purified antibody (2210) attaches to the second PDZ domain (2305) which is attached to the solid support (2310). Again, now referring to FIG. 24, a free PDZ peptide (2405) can be used, similarly to the previous examples as detailed above in the present disclosure. A purified antibody (2410) is then obtained.

Affinity chromatography in some embodiments involves the use of an affinity column. In an affinity column according to embodiments herein described the stationary phase is contained within a cylinder and comprises a solid support attached to at least one affinity ligand selected from the group consisting of a PDZ domain peptide herein described and a PDZ domain binding carboxy terminal peptide. The stationary phase can comprise in particular commercial resins such as Profanity™ epoxide resin from bio-rad, and matrices such as carbonyldimidazole, cyanogen bromide, epichlorohydrin, epoxy(bis), N-Hydrozy-succinimdyl Chloroformate, p-Nitrophenyl Chloroformate, Tresyl Chloride, and Vinyl Sulfone, N-Hydroxysuccinimide Ester, and Disulfide. In various embodiments spacer arms can be employed to increase the distance from the matrix and the PDZ domain peptide and a PDZ domain binding carboxy terminal peptide. The increased distance can in some embodiments result in increased binding probability by reducing the steric hindrance that can occur when directly coupled to the bead. The cylinder is configured such as to permit the protein of interest to enter the column and become affixed to the stationary phase. Other contents of the solution not of interest flow through the column. A different solution is later passed over the column releasing the protein of interest.

In embodiments of the methods and systems herein described the affinity chromatography systems and methods can be used for protein purification.

The methods used in protein purification can roughly be divided into analytical and preparative methods. The distinction is not exact, but the deciding factor is the amount of protein that can practically be purified with that method. Analytical methods aim to detect and identify a protein in a mixture, whereas preparative methods aim to produce large quantities of the protein for other purposes, such as structural biology or industrial use. In general, the preparative methods can be used in analytical applications, but not the other way around. In several embodiments described herein, the protein purification can be done with a crude preparation, and the purification can be either analytical or for preparative methods.

Protein purification can be either preparative or analytical. Preparative purifications aim to produce a relatively large quantity of purified proteins for subsequent use. Examples include the preparation of commercial products such as enzymes (e.g. lactase), nutritional proteins (e.g. soy protein isolate), and certain biopharmaceuticals (e.g. insulin). Analytical purification produces a relatively small amount of a protein for a variety of research or analytical purposes, including identification, quantification, and studies of the protein's structure, post-translational modifications and function. In the embodiments described herein, the purification can be both preparative and analytical.

Depending on the source of the protein, the protein has to be brought into solution by breaking the tissue or cells containing it. There are several methods to achieve this: Repeated freezing and thawing, sonication, homogenization by high pressure, filtration, or permeabilization by organic solvents. The method of choice depends on how fragile the protein is and how sturdy the cells are. Usually for most of the conventional purposes, column chromatography is used to achieve purification. After this extraction process soluble proteins will be in the solvent, and can be separated from cell membranes, DNA etc. by centrifugation. The extraction process also extracts proteases, which will start digesting the proteins in the solution. If the protein is sensitive to proteolysis, it is usually desirable to proceed quickly, and keep the extract cooled, to slow down proteolysis.

In a purification process, the starting material is known. In a plant or animal, a particular protein usually isn't distributed homogeneously throughout the body; different organs or tissues have higher or lower concentrations of the protein. Use of only the tissues or organs with the highest concentration decreases the volumes needed to produce a given amount of purified protein. If the protein is present in low abundance, or if it has a high value, scientists can use recombinant DNA technology to develop cells that will produce large quantities of the desired protein, this is known as an expression system, and is known to those skilled in the art. Recombinant expression allows the protein to be tagged, e.g. by a His-tag, to facilitate purification, which means that the purification can be done in fewer steps. In addition, recombinant expression usually starts with a higher fraction of the desired protein than is present in a natural source. In several embodiments described herein, the target protein or protein of interest (POI) is tagged with a PDZ peptide tag in order to separate and purify the target or POI. Design of the purification process is dependent on the protein and its environment and is known to those skilled in the art.

An analytical purification generally can utilize several properties to separate proteins. First, proteins can be purified according to their isoelectric points by running them through a pH graded gel or an ion exchange column. Second, proteins can be separated according to their size or molecular weight via size exclusion chromatography or by SDS-PAGE (sodium dodecyl sulfate-polyacrylamide gel electrophoresis) analysis. Proteins are often purified by using 2D-PAGE and are then analyzed by peptide mass fingerprinting to establish the protein identity. This is very useful for scientific purposes and the detection limits for protein are very low and nanogram amounts of protein are sufficient for their analysis. Thirdly, proteins can also be separated by polarity/hydrophobicity via high performance liquid chromatography or reversed-phase chromatography. In the methods described herein, proteins are examined by 2D-gel analysis and can be performed by other methods known to those skilled in the art.

Usually a protein purification protocol contains one or more chromatographic steps. The basic procedure in chromatography is to flow the solution containing the protein through a column packed with various materials. Different proteins interact differently with the column material, and can thus be separated by the time required to pass the column, or the conditions required to elute the protein from the column. Proteins can be detected as they are coming off the column by their absorbance at 280 nm. Many different chromatographic methods exist and are known to those skilled in the art.

In the context of protein purification, the eluent is usually pooled in different test tubes. All test tubes containing no measurable trace of the protein to purify are discarded. The remaining solution is thus made of the protein to purify and any other similarly-sized proteins. The concentration of the protein that is eluted into the test tubes can be determined by UV spectroscopy and other methods that are known to those skilled in the art.

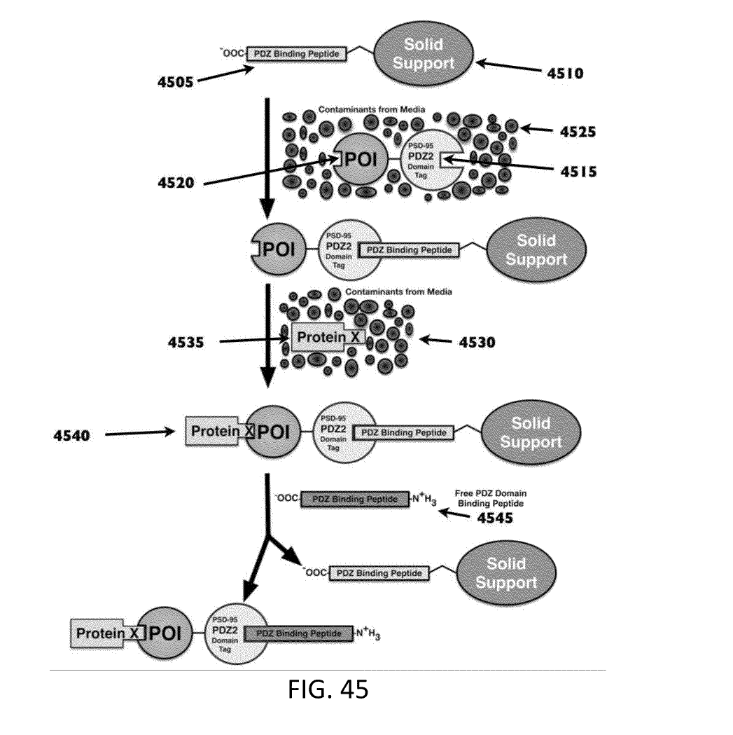

FIG. 45 illustrates an example of a pull down scheme of one of the embodiments described herein. In the embodiment of FIG. 45, the affinity matrix is a PDZ binding C-terminal peptide (4505) immobilized on a solid support (4510), for example H₃N+-SIESDV-COO— (SEQ ID NO: 160) from NR2B Tail. The protein tag (4515) can be a Type C PDZ affinity tag, a C-terminal PDZ domain, for example, a POI fused to PDZ2 from PSD-95. The POI with the tag (4520) can be surrounded by contaminants in the media (4525).

The tag (4515) attaches to the binding peptide (4505) on the support (4510). Media contaminants are removed by washing the support with buffer. A second mixture can then be added (4530). This mixture can be comprised of cellular lysate, a partially purified protein, a membrane preparation, clarified cell lysate, etc., and contains proteins that can interact with the POI (for example, Protein X (4535)).

Protein X binds to the POI immobilized on the solid support via the PDZ Type C Tag-PDZ Binding Peptide linkage (e.g. PSD-95 PDZ2 Tag bound to NR2B tail C-terminal peptide) (4540). The PDZ binding C-terminal peptide affinity resin can then be washed with a buffer to remove weakly and non-specifically bound host cell proteins. The POI fused to the C-terminal PDZ domain tag (a Type C tag) (4520) and the POI's binding partner (4535) can then be co-eluted from the resin by incubation with a buffer containing free PDZ binding peptide (4545) or other elution agents (for example, salt, pH, denaturant, etc.). Following purification of the POI and its protein binding partner, the binding partner can be subsequently identified by a number of techniques (e.g. by SDS-PAGE and Coomassie staining, Western Blotting, protein digestion and mass spectrometry, etc.). The PDZ binding C-terminal peptide affinity resin can then be regenerated for repeat use by stripping away bound free PDZ binding peptide and washing by the use, for example, of salt, pH, denaturants, etc. Any POI containing a PDZ domain with or without β-hairpin capable of binding the PDZ binding C-terminal peptide attached to this resin could be purified with this scheme.

The PDZ binding C-terminal peptide can be synthesized, and is covalently attached (e.g. NHS/EDC coupling) to a solid support (e.g. agarose beads) to generate a PDZ binding C-terminal peptide affinity resin. A recombinant Protein of Interest (POI), genetically fused to a C-terminal PDZ domain tag (a Type C PDZ affinity tag) can be expressed in a cell line of choice (e.g. E. coli) or in vitro translation system. The cells can be lysed and, if needed, clarified by centrifugation. Clarified lysate containing the POI can then be incubated with the PDZ binding C-terminal peptide affinity resin.

In additional embodiments of this procedure, the POI can be N-terminally, C-terminally or Internally attached to a Type B or C PDZ affinity tag and immobilized on PDZ domain binding peptide resin (Type B and C) or PDZ domain resin. The POI can additionally be fused to a C-terminal Type A PDZ affinity tag and immobilized on PDZ domain or PDZ domain-βeta Hairpin resin. The POI can additionally be fused N-terminally, C-terminally or Internally to a Type B PDZ affinity tag and immobilized on PDZ domain or PDZ domain binding peptide resin.

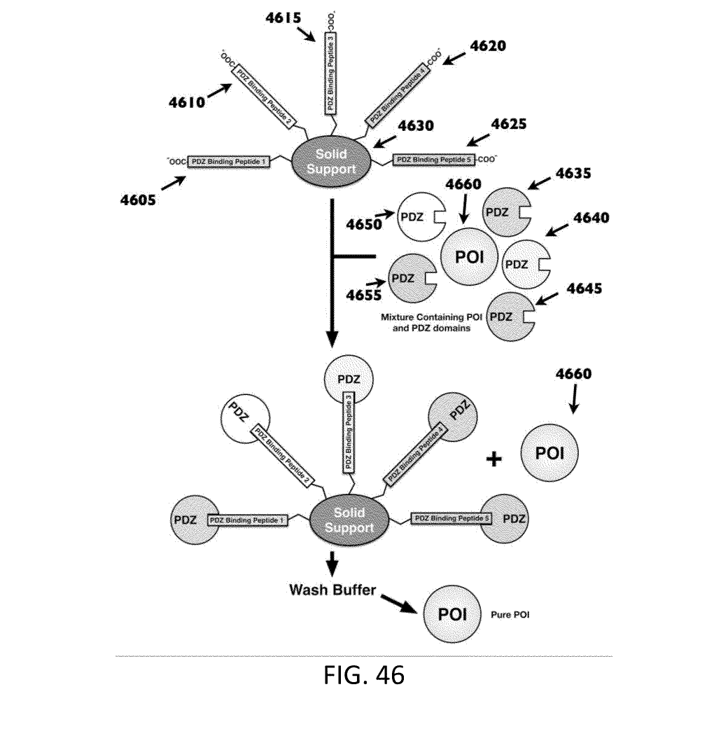

FIG. 46 illustrates an example of using a PDZ affinity matrix for substance depletion of one of the embodiments described herein. In the embodiment of FIG. 46, the affinity matrix is comprised of multiple PDZ binding C-terminal peptides (4605-4625) immobilized on a solid support (4630), for example H₃N⁺-SIESDV-COO⁻ (SEQ ID NO: 160) from NR2B Tail, H₃N⁺-QEELII-COO⁻ from Cadherin-5, etc. The contaminating PDZ domain proteins in the mixture (4635-4655) can be free PDZ domains, for example, PDZ2 from PSD-95, PDZ domain from Partitioning defective 3 homolog, etc. The untagged POI (4660) can be surrounded by these contaminant PDZ domains in the media.

The free contaminate PDZ domains present in the mixture (4635-4655) attach to the binding peptides (4605-4625) on the support (4630). PDZ domain media contaminants are removed by adsorption to the support, and the POI, lacking any PDZ binding peptide affinity (4660), can be eluted from the column by washing.

The PDZ binding C-terminal peptide affinity resin can then be regenerated for repeat use by stripping away bound free PDZ binding peptide and washing by the use, for example, of salt, pH, denaturants, etc. Any POI lacking a PDZ domain with or without β-hairpin capable of binding the PDZ binding C-terminal peptide attached to this resin could be enriched or purified with this scheme, assuming contaminant PDZ domains are adsorbed to the resin.

The PDZ binding C-terminal peptides can be synthesized, and are covalently attached (e.g. NHS/EDC coupling) to a solid support (e.g. agarose beads) to generate a mixed PDZ binding C-terminal peptide affinity resin. A Protein of Interest (POI), can be present in any type of mixture, including a cell line of choice (e.g. E. coli), in vitro translation system, cellular or tissue preparation, etc. Contaminant PDZ domains can be present in the mixture, including, but not limited to, a cell line of choice (e.g. E. coli), in vitro translation system, cellular or tissue preparation, etc.

In additional embodiments of this procedure, the free PDZ domains can be selectively adsorbed onto PDZ domain-βeta Hairpin resin. Additionally free PDZ-βeta Hairpin domains can be selectively adsorbed onto PDZ domain or PDZ domain binding peptide resin. Contaminants containing PDZ domain binding C-terminal peptide sequences can also be selectively adsorbed onto PDZ domain or PDZ domain-βeta Hairpin resin.

In some embodiments, the engineered protein herein described can be modified to add further chromatography tags.

The term “Chromatography,” as used herein, refers to a technique used to separate proteins in solution or denaturing conditions. A type of chromatography called “size exclusion chromatography using porous gels can be used to separate molecules by size. The principle is that smaller molecules have to traverse a larger volume in a porous matrix. Consequentially, proteins of a certain range in size will require a variable volume of eluent (solvent) before being collected at the other end of the column of gel, in which the gel is the support for the size exclusion chromatography.

“Chromatography tags” are used to alter chromatographic properties of the protein to afford different resolution across a particular separation technique. In some instances, chromatography tags consist of polyanionic amino acids, such as FLAG-tag. In some instances chromatography tag comprise epitope tags which are short peptide sequences which are chosen because high-affinity antibodies can be reliably produced in many different species. These are usually derived from viral genes, which explain their high immunoreactivity. Epitope tags include V5-tag, Myc-tag, and HA-tag. These tags are particularly useful for western blotting, immunofluorescence and immunoprecipitation experiments, although they also find use in antibody purification. Fluorescence tags are used to give visual readout on a protein. GFP and its variants are the most commonly used fluorescence tags. More advanced applications of GFP include using it as a folding reporter (fluorescent if folded, colorless if not). Protein tags find many other usages, such as specific enzymatic modification (such as biotin ligase tags) and chemical modification (FlAsH) tag. Often tags are combined to produce multifunctional modifications of the protein. However, with the addition of each tag comes the risk that the native function of the protein can be abolished or compromised by interactions with the tag. Therefore, after purification, tags are commonly removed by specific proteolysis. Removal of tags can be performed by washing an affinity ligand with the proteolytic enzyme, and then purifying out the proteolytic enzyme from the elution buffer, which is a technique known to those skilled in the art.

In some embodiments, PDZ affinity tags herein described can be used as epitope tags which are short peptide sequences which are chosen because high-affinity antibodies can be reliably produced in many different species and for western blotting, immunofluorescence and immunoprecipitation experiments. In some embodiments, PDZ affinity tags herein described can be used to track proteins in the cell or following purification by binding to fluorescent or colorimetric substrates. In some embodiments, PDZ affinity tags herein described can be used to assemble protein/enzyme complexes in a defined order or orientation, by utilizing corresponding PDZ affinity ligand for example immobilized on a solid surface. Accordingly, PDZ affinity tags and ligands can also be used in connection with additional chromatography system such as Hydrophobic Interaction Chromatography, Ion exchange chromatography as will be understood by a skilled person.

“Hydrophobic Interaction Chromatography” (HIC) refers to purification via the hydrophobicity of molecules. HIC media is amphiphilic, with both hydrophobic and hydrophilic regions, allowing for separation of proteins based on their surface hydrophobicity. In pure water, the interactions between the resin and the hydrophobic regions of protein would be very weak, but this interaction is enhanced by applying a protein sample to HIC resin in high ionic strength buffer. The ionic strength of the buffer is then reduced to elute proteins in order of decreasing hydrophobicity.

“Ion exchange chromatography,” refers to the separation of molecules due to their charges. Ion exchange chromatography separates compounds according to the nature and degree of their ionic charge. The column to be used is selected according to its type and strength of charge. Anion exchange resins have a positive charge and are used to retain and separate negatively charged compounds, while cation exchange resins have a negative charge and are used to separate positively charged molecules. Before the separation begins a buffer is pumped through the column to equilibrate the opposing charged ions. Upon injection of the sample, solute molecules will exchange with the buffer ions as each competes for the binding sites on the resin. The length of retention for each solute depends upon the strength of its charge. The most weakly charged compounds will elute first, followed by those with successively stronger charges. Because of the nature of the separating mechanism, pH, buffer type, buffer concentration, and temperature all play important roles in controlling the separation. Ion exchange chromatography is a very powerful tool for use in protein purification and is frequently used in both analytical and preparative separations.

The methods and systems described in the present disclosure can be used for the purification of naturally occurring proteins containing PDZ domains or C-terminal PDZ domain binding peptide sequences.

In some embodiments, the methods and systems can purify neuronal proteins that contain naturally occurring PDZ domains or C-terminal PDZ domain binding peptide sequences. For several of the expressed neuronal POIs, the methods and systems described in the present disclosure provide the first known process for the purification of these proteins, while for other proteins the method and systems described herein can provide increased purity and yield of the POI relative to other affinity chromatography methods.

In some embodiments, the affinity tags and ligands herein described can be used for detection of targets in a mixture possibly in combination with antibody or additional capture agents. The term “capture agent” as used herein indicates a compound configured to specifically bind to a target, and include but are not limited to organic molecules, such as polypeptides, polynucleotides and other non polymeric molecules that are identifiable to a skilled person.

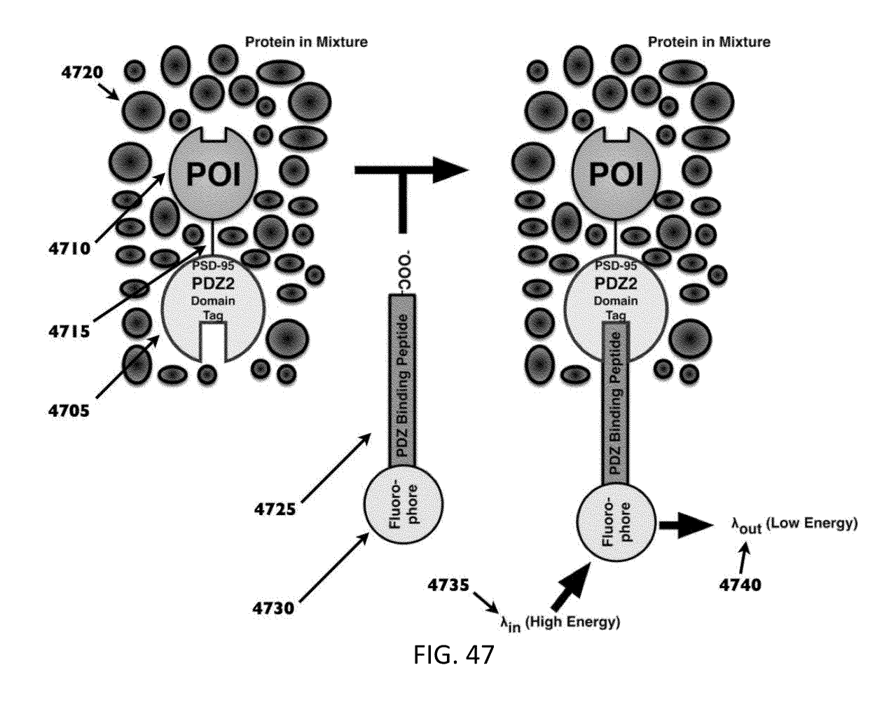

FIG. 47 illustrates an example of using a PDZ domain affinity tag (Type C) and a free PDZ domain binding C-terminal peptide bound to a detection agent, to specifically detect the presence of a the fusion protein in a mixture. In the embodiment of FIG. 47, POI (4710) fused to a Type C PDZ affinity tag (4705) (e.g. PSD-95 PDZ2 domain) is heterologously expressed in a cell line or tissue. The POI with the tag (4715) will be surrounded by contaminants in the cell or media (4720). In order to detect the presence of the tagged POI in the mixture, a PDZ binding C-terminal peptide (4725) fused to a detection agent (e.g. a fluorophore, alkaline phosphatase, quantum dot, horseradish peroxidase, etc.) (4730) is added to the mixture to bind to the Type C PDZ affinity tag and form a specific and high affinity complex with the PDZ Type C affinity tagged POI. A stimulus (4735) is then applied to the mixture to produce a response (4740) specific only to the complex formed between the PDZ Type C tagged POI and the PDZ binding C-terminal peptide.

The PDZ binding C-terminal peptide can be synthesized, and is covalently attached (e.g. NHS/EDC coupling) to a detection agent (e.g. fluorophore, alkaline phosphatase, quantum dot, horseradish peroxidase, etc.) to generate a specific probe for detecting PDZ affinity tagged POIs. In this example, a recombinant Protein of Interest (POI), can be genetically fused to a C-terminal PDZ domain tag (a Type C PDZ affinity tag) can be expressed in a cell line of choice (e.g. E. coli) or in vitro translation system. The cells can be lysed and, if needed, clarified by centrifugation.

In additional embodiments of this procedure, the POI can be N-terminally, C-terminally or Internally attached to a Type B or C PDZ affinity tag and probed with a PDZ domain binding peptide coupled to a detection agent. The POI can additionally be fused to a C-terminal Type A PDZ affinity tag and probed with a PDZ domain or PDZ domain-βeta-Hairpin coupled to a detection agent. The POI can additionally be fused N-terminally, C-terminally or Internally to a Type B PDZ affinity tag and probed with a PDZ domain coupled to a detection agent.

In some embodiments the affinity ligand can be attached to a solid support surface to form a protein array. Exemplary solid supports suitable for a protein array comprise solid material shaped to have at least one surface, such as a glass slide, nitrocellulose membrane, bead, or microtitre plate.

In protein arrays herein described the solid support surface attaches at least one affinity ligand selected from the group consisting of a PDZ domain peptide, PDZ domain peptide comprising β-Hairpin and a PDZ domain carboxy terminal peptide. In particular, in embodiments herein described wherein in the protein array, the solid support surface attaches one or more PDZ domain peptide ligand, attachment is performed to result in an attached PDZ domain peptide ligand for specific binding to one or more of corresponding PDZ domain peptide tags and/or corresponding PDZ binding carboxy terminal peptide tags. Embodiments herein described wherein in the protein array, the solid support surface attaches one or more PDZ binding carboxy terminal peptide ligand, attachment is performed to result in an attached PDZ binding carboxy terminal peptide ligand in a configuration wherein the PDZ binding carboxy terminal ligand is presented for specific binding to a corresponding PDZ domain peptide tag.

In embodiments herein described PDZ affinity ligand can detect corresponding PDZ domain e.g. comprised in affinity tag herein described or in naturally occurring POI, with high specificity to the extent of allowing detection of a POI comprising a PDZ domain at POI concentration in the nanomolar range (e.g. 1 to 100 nM or in some embodiments 1 to 10 nM) in mixture such a lysate.

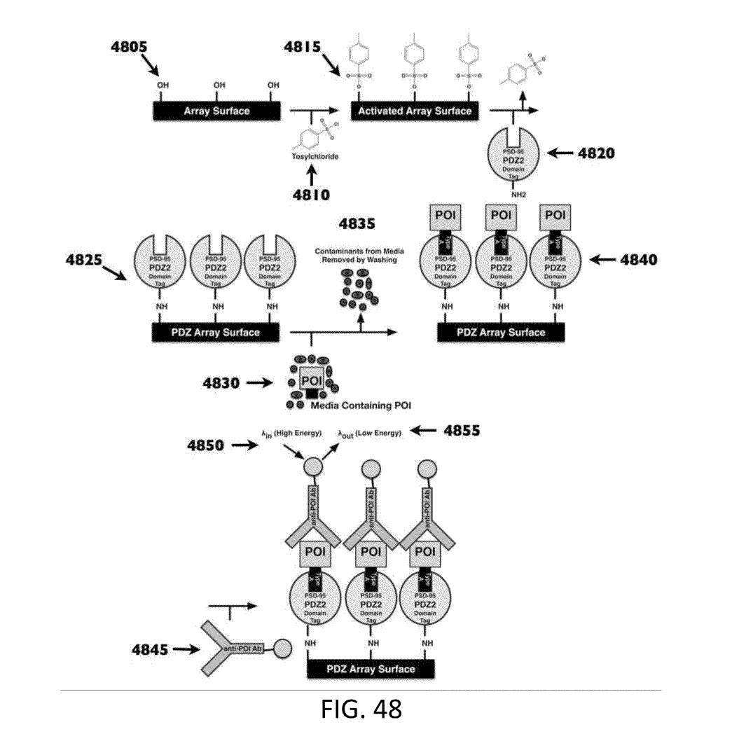

FIG. 48 illustrates an example of using a PDZ domain (e.g. PSD-95 PDZ2) immobilized on a solid surface, expressed POI fusion proteins containing C-terminal PDZ domain binding peptides (Type A Tags) and monoclonal antibodies bound to a detection agent to monitor the amount of POI present in a mixture. In the embodiment of FIG. 48, a solid surface containing reactive groups (e.g. hydroxyl groups) (4805) is reacted with Tosylchloride (4810) to produce an activated surface (4815). The activated surface is then reacted with an amine terminus present on a PDZ domain (e.g. PSD-95 PDZ2) (4820) to generate a surface derivatized with PDZ domains (4825).

Heterologously expressed POIs fused to a Type A PDZ affinity tag (4830) (e.g. PDZ domain binding C-terminal peptide sequence from NR2B Tail) are added to the array surface and form a complex with immobilized PDZ domains (4840). The tagged POIs will be surrounded by contaminants in the cell or media (4835), thus washing of the array surface must be performed to remove contaminants. In order to quantitatively detect the presence of the tagged POI in the mixture (Immobilized on the array), a monoclonal antibody specific to each POI fused to a detection agent (e.g. a fluorophore, alkaline phosphatase, quantum dot, horseradish peroxidase, etc.) (4845) is added to the mixture to form a specific and high affinity complex with the POI. After washing to remove nonspecifically bound antibody, a stimulus (4850) is then applied to the mixture to produce a response (4855) specific only to the complex formed between the PDZ Type A tagged POI and antibody.

The monoclonal antibodies can be purchased or synthesized, and are covalently attached (e.g. NHS/EDC coupling) to a detection agent (e.g. fluorophore, alkaline phosphatase, quantum dot, horseradish peroxidase, etc.) to generate a specific probe for detecting PDZ affinity tagged POIs. In this example, a recombinant POI, can be genetically fused to a C-terminal PDZ domain binding peptide tag (a Type A PDZ affinity tag) and expressed in a cell line of choice (e.g. E. coli) or in vitro translation system. The cells can be lysed and, if needed, clarified by centrifugation.

In additional embodiments of this procedure, the POI can be N-terminally, C-terminally or Internally attached to a Type B or C PDZ affinity tag and bound to a PDZ domain binding peptide covalently immobilized on the array surface. The POI can additionally be fused to an N-terminal, C-terminal or internal Type B PDZ affinity tag and bound to a PDZ domain covalently immobilized on the array surface. Additionally, in this example, array surfaces can affix different PDZ domains to the surface to bind different Tagged POIs with corresponding PDZ Type A affinity tags to multiplex assays. Surface coupling chemistry can also be altered to couple different functional groups to array surfaces.

In some embodiments, affinity ligand can be used to provide, an engineered label.

The terms “label” and “labeled molecule” as used herein as a component of a complex or molecule referring to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion there of which is capable of exhibiting fluorescence in a detectable image. As a consequence, the wording “labeling signal” as used herein indicates the signal emitted from the label that allows detection of the label, including but not limited to radioactivity, fluorescence, chemiluminescence, production of a compound in outcome of an enzymatic reaction and the additional reaction identifiable by a skilled person.

Exemplary labels comprise Alexafluor dyes, Fluorescein, horseradish peroxidase, alkaline phosphatase, glucose oxidase and additional labels identifiable by a skilled person upon reading of the present disclosure.

In embodiments of the present disclosure, an engineered label comprises a label attaching at least one affinity ligand selected from the group consisting of a PDZ domain peptide and a PDZ binding carboxy terminal peptide. In engineered labels herein described, the affinity ligand is attached to the label in a configuration where the affinity ligand is presented for binding to a corresponding affinity tag. In the engineered label herein described, a PDZ domain peptide ligand is presented for specific binding to one or more of corresponding PDZ domain peptide tags and/or corresponding PDZ binding carboxy terminal peptide tags. In the engineered label herein described, a PDZ carboxy terminal ligand is presented for specific binding to a corresponding PDZ domain peptide tag.

Engineered label herein described can be used to detect a PDZ domain, a PDZ carboxy terminal ligand, e.g. comprised in a naturally occurring protein, a PDZ affinity tag herein described (e.g. presented on an engineered protein) and/or a PDZ affinity ligand herein described (e.g. presented on a protein array herein described).

The terms “detect” or “detection” as used herein indicates the determination of the existence, presence or fact of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

In some embodiments, detection of an engineered label herein described can be carried with techniques identifiable by a skilled person and that depend on the specific label that is engineered. For example when the label is a fluorophore detection can be performed for example via fluorescent based readouts which includes, but not exhaustively, small molecular dyes, protein chromophores, quantum dots, and gold nanoparticles.

In various embodiments the PDZ system herein described can be used in various applications depending on the conjugate of PDZ including for example, immunological assays, flow cytometry, cell sorting, immunohistochemistry, western blots, nucleic acid hybridization (e.g. conjugate of PDZ being an enzyme-alkaline phosphatase, horseradish peroxidase, β-galactosidase, glucose oxidase, etc.; conjugate of PDZ being fluorophores—fluorescein, coumarins, rhodamines, phycoerthrin, Texas Red; conjugate of PDZ being Eu 3⁺.) In various embodiments the PDZ system herein described can be used in electron microscopy (e.g. conjugate of PDZ being Ferritin, gold). In various embodiments the PDZ system herein described can be used in affinity chromatography (e.g. conjugate of PDZ being agarose). In various embodiments the PDZ system herein described can be used in DNA sequences (e.g. conjugate of PDZ being magnetic particles).

In various embodiments the PDZ system herein described can be used in various applications depending on the conjugate of the PDZ binding peptide including for example, immunological assays, flow cytometry, cell sorting, immunochemistry, western blots (e.g. conjugate being anti-immunoglobulins, Protein A, Protein G). In various embodiments the PDZ binding peptide can be used when conjugated to lectins (e.g. glycoconjugate studies, mitogenic simulation studies). In various embodiments the PDZ binding peptide can be used when conjugated to antilectins (e.g. localization of lectin receptors). In various embodiments the PDZ binding peptide can be used when conjugated to enzyme such as alkaline phosphatase, horseradish peroxidase, glucose oxidase, and β-galactosidase for immunological assays and nucleic acid hybrization. In various embodiments the PDZ binging peptide can be used when conjugated to ferritin or hemocyanin for electromicroscopy. In various embodiments the PDZ binding peptide can be used when conjugated to agarose or cellulose for affinity chromatography. In various embodiments the PDZ binding peptide can be used when conjugated to anti-avidin or anti-streptavidin for amplification assays. In various embodiments the PDZ binding peptide can be used when conjugated to nucleotides for nucleic acid hybridization, molecular mass markets, or DNA sequencing. In various embodiments the PDZ binding peptide can be used when conjugated to hormones for affinity chromatography or receptor-ligand interaction studies. In various embodiments when coupled to cells the PDZ binding peptide can be used for hybridoma production.

Additional techniques are identifiable by a skilled person upon reading of the present disclosure and will not be further discussed in detail.

In some embodiments, affinity tags or affinity ligand here described can be used detection of a target in a biochemical mixture.

The method comprises contacting the biochemical mixture and in particular a sample with one or more engineered proteins herein described capable of specific binding to the target to form an engineered protein-target complex presenting the PDZ domain peptide tag or the PDZ binding carboxy terminal peptide tag for binding to a corresponding PDZ domain peptide ligand and/or to a PDZ binding carboxy terminal peptide ligand.

In methods herein described the method can further comprise contacting the engineered protein-target complex with an engineered label herein described presenting the corresponding PDZ domain peptide ligand and/or a PDZ binding carboxy terminal peptide ligand.

As disclosed herein, the affinity tags and/or affinity ligands herein described and/or related engineered protein, engineered label and protein arrays can be provided as a part of systems to perform any assay, including any of the assays described herein. The systems can be provided in the form of arrays or kits of parts. An array, sometimes referred to as a “microarray”, can include any one, two or three dimensional arrangement of addressable regions bearing a particular molecule associated to that region. Usually, the characteristic feature size is micrometers.

In a kit of parts, the affinity tags and/or affinity ligands, related engineered protein, engineered label and protein arrays and other reagents to perform the assay can be comprised in the kit independently. The affinity tags and/or affinity can be included in one or more compositions, and each capture agent can be in a composition together with a suitable vehicle.

Additional components can include labeled molecules and in particular, labeled polynucleotides, labeled antibodies, labels, microfluidic chip, reference standards, and additional components identifiable by a skilled person upon reading of the present disclosure.

In some embodiments, detection of an affinity tags and/or affinity ligands, related engineered protein, engineered label and protein arrays can be carried either via fluorescent based readouts, in which the labeled antibody is labeled with fluorophore, which includes, but not exhaustively, small molecular dyes, protein chromophores, quantum dots, and gold nanoparticles. Additional techniques are identifiable by a skilled person upon reading of the present disclosure and will not be further discussed in detail.

In particular, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

In some embodiments, a target detection system is described. The target detection system comprises two or more of; one or more engineered proteins herein described, one or more engineered labels herein described and a protein array herein described for simultaneous combined or sequential use in a method to detect a target herein described.

The methods and systems herein described are further illustrated in the following examples, which are provided by way of illustration and are not intended to be limiting.

### EXAMPLES

The methods and systems herein described are further illustrated in the following examples, which are provided by way of illustration and are not intended to be limiting.

The methods and devices for purifying a POI in a crude sample, related supports, and a method and/or device using assays herein disclosed are further illustrated in the following examples, which are provided by way of illustration and are not intended to be limiting. The following exemplary methods, devices and methods for POI purification are illustrated in connection with experimental procedures and with neuronal proteins and other POIs. Shown are examples of purification of naturally occurring proteins containing PDZ domains or C-terminal PDZ domain binding peptide sequences. A person skilled in the art will appreciate the applicability and the necessary modifications to adapt the features described in detail in the present section, to methods and devices according to embodiments of the present disclosure.

### Examples 1-5

### PSD-95 PDZ Affinity Chromatography Purification of Neuronal Proteins Heterologously Expressed in E. coli as an MBP Fusion Protein

To show the utility of PDZ Domain Affinity Resin, a panel of proteins known to interact with the PDZ domains of PSD-95 was purified. The proteins were NR2B (GluN2B) Tail (Example 1), synGAP (Example 2), cypin (Example 3), CRIPT (Example 4) and nNOS (Example 5).

PSD-95 is a scaffold protein located in the postsynaptic density of glutamatergic synapses that contains three PDZ domains in addition to an SH3 and a guanylate kinase-like domain (30,31). PSD-95 is the first protein in which PDZ domains were recognized. Because of its critical role in receptor clustering and protein localization at the synapse (106-109), it is also one the most well characterized PDZ domain-containing proteins.

### Example 1

### PSD-95 PDZ2 Affinity Chromatography Purification of the neuronal protein NR2B (GluN2B) heterologously expressed in E. coli as an MBP Fusion Protein

Many difficult to purify proteins containing naturally occurring PDZ binding capabilities exist in nature. To test the utility of PDZ Domain Affinity Resin for purifying proteins with endogenous PDZ domain binding ability, a portion of the NR2B (also known as GluN2B) subunit of the N-methyl-D-aspartate (NMDA) Receptor (Mus musculus NR2B Tail, AA 840-1482) was purified using a PDZ2 PSD-95 PDZ Affinity Resin.

The N-methyl-D-aspartate type glutamate receptor (NMDAR) is one of three major classes of receptors for glutamate, the principal excitatory neurotransmitter in the central nervous system and has a seminal role in learning and memory through its actions as a “coincidence detector” that initiates changes in synaptic strength leading to the formation of new neural networks (110-113). It is comprised of four subunits, including two from the GluN2 subfamily (GluN2A-D) (114). The GluN2 subunits are unique among ligand-gated channels in having extended intracellular C-terminal domains or “tails,” which extend into the cytoplasm and serve as nucleation sites for scaffolds and signal transducing enzymes (115). The cytoplasmic tails of NR2 subunits have not previously been expressed in soluble form in significant quantities, or purified. Expression and purification of the cytoplasmic tail of the GluN2B subunit (“GluN2B Tail”: Mus musculus GluN2B residues 840-1482) which contains a C-terminal PDZ ligand that binds to PDZ2 of PSD-95, was selected.

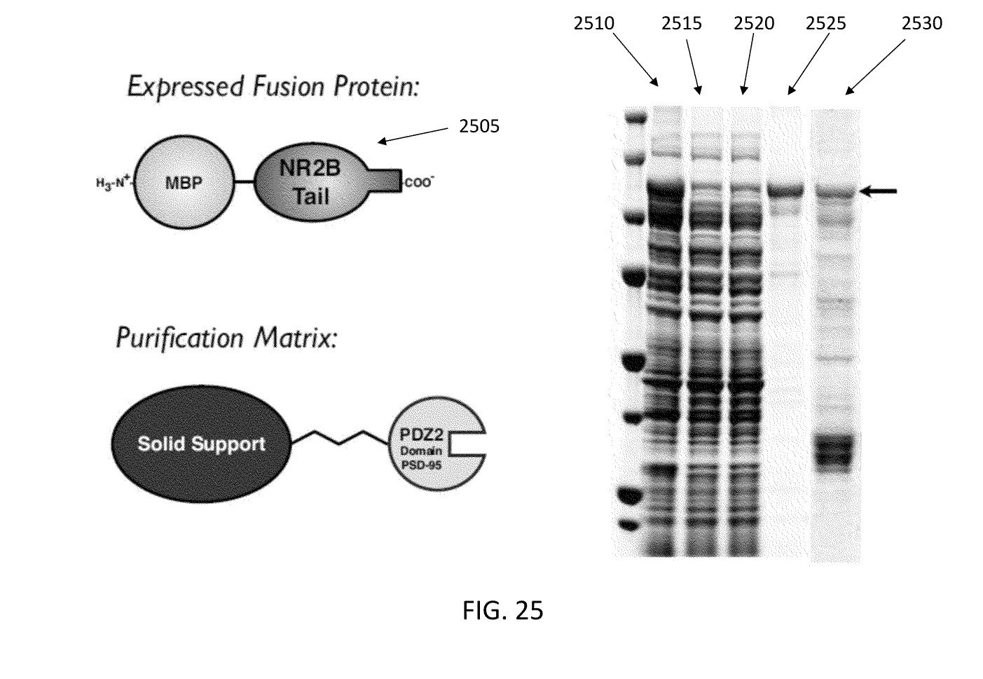

The selected region of NR2B purified in FIG. 25 had never before been purified. When expressed in isolation in E. coli, the NR2B Tail forms insoluble protein aggregates known as inclusion bodies. The NR2B Tail protein was expressed in soluble form in E. coli when fused to an N-terminal Maltose Binding protein. When the MBP-NR2B Tail fusion protein was incubated with PDZ2 PSD-95 PDZ Affinity Resin, the MBP-NR2B Tail fusion protein was purified to >70% homogeneity. PDZ Domain Affinity Chromatography was the method of choice for purifying the GluN2B Tail, compared to alternative methods. A single column step using Amylose (MBP Tag) Affinity resin, results in eluted NR2B Tail protein of approximately 10% purity, while attempts to purify the MBP-GluN2B Tail fusion protein with Streptactin and Ni-NTA resins after addition of N- or C-terminal StrepII or 8×His Tags, respectively, were unsuccessful; no protein was present in the column eluate.

For the experiment, fusion proteins containing MBP-NR2B (GluN2B) Tail (Mus musculus, AA 840-1482) were expressed in BL21(DE3) E. coli cells. Single colonies of BL21(DE3) cells harboring pJExpress414 plasmids containing the MBP-NR2B gene were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into LB medium, and grown at 37° C. until cultures reached an O.D.₆₀₀ of 0.8. Cultures were then chilled to 18° C., and IPTG was added to a final concentration of 1 mM. Cultures were grown for an additional 24 hours at 18° C. before being pelleted by centrifugation, and flash frozen in liquid nitrogen. Bacterial cell pellets (0.4-2.5 grams) containing fusions of MBP-NR2B Tail were resuspended in 5 ml/g cell-paste Purification Buffer (50 mM HEPES, pH 7.5, 150 mM NaCl, 5 mM TCEP, 0.5 mM EDTA, 10 mM MgCl2, 2 mM PMSF, Complete Protease Inhibitor) supplemented with 25 U/ml Benzonase and 10 U/ml ReadyLyse. The resuspended cells were sonicated (Branson, Danbury Conn.) 2× for 90 s/pass (15% power, 1.0 s on, 1.5 s off) before clarification by centrifugation at 16,000×g for 30 min at 4° C. Clarified lysate (1.8-14 ml) containing POIs was added to 100-200 ul of PDZ2 domain of PSD-95 coupled to a solid support pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. Eluted protein was analyzed on 4-12% bis-tris SDS-PAGE gels and proteins were detected by Gel Code Blue staining.

As shown, FIG. 25 relates to the purification of a portion of the NR2B (GluN2B) subunit of the N-methyl-D-aspartate (NMDA) Receptor (NR2B tail, AA 840-1482) using PSD-95 PDZ2 PDZ affinity resin. Until recently, this selected region of NR2B had never before been purified.

In FIG. 25, the expressed protein is NR2B (GluN2B) subunit of the N-methyl-D-aspartate (NMDA) Receptor (NR2B tail, AA 840-1482) (2505) and the purification matrix is PSD-95 PDZ3 domain coupled to a solid support. A GelCode Blue stained 4-12% bis-tris SDS-PAGE gel was used. The columns in FIG. 25 are: unclarified cell lysate (2510); soluble or clarified cell lysate (2515); unbound protein (2520); peptide eluate (2525) and for comparison, eluate from an amylose affinity chromatography column (2530). As shown in the peptide eluate, the protein is purified from the crude lysate.

The results of FIG. 25 show the potential for PDZ affinity chromatography to be utilized for the purification of proteins with endogenous PDZ domain binding affinity which has not been previously purified by other means.

### Example 2

### PSD-95 PDZ3 Affinity Chromatography Purification of the Neuronal Protein synGAP Heterologously Expressed in E. coli Fused to a N-Terminal 6× Histidine Tag

Many difficult to purify proteins containing naturally occurring PDZ binding capabilities exist in nature. To test the utility of PDZ Domain Affinity Resin for purifying proteins with endogenous PDZ domain binding ability, a portion of the neuronal dual Ras & Rap GTPase Activating Protein, synGAP (Rattus norvegicus, AA 103-1293) was purified using a PDZ3 PSD-95 PDZ Affinity Resin. Until recently, the selected region of synGAP had never before been purified.

SynGAP (Synaptic GTPase Activating Protein) is a dual Ras and Rap GTPase activating protein that is highly concentrated in the postsynaptic density of excitatory synapses (116). Homozygous deletion of synGAP is lethal in mice (117,118), and a heterozygous deletion confers behavioral phenotypes associated with cognitive disability and mental illness (119). Synaptic plasticity is disrupted in heterozygous knockout mice and the formation of spine synapses during development is accelerated (118-120). Mutations in the human SYNGAP1 gene appear to cause non-syndromic intellectual disability and certain forms of autism (121). SynGAP is regulated by phosphorylation in a disordered region carboxyl to the GAP domain (122). Attempts to understand the effects of phosphorylation have been hampered by the difficulty of purifying a soluble form of synGAP containing the disordered region. It was previously shown that removal of the N-terminal 102 residues of synGAP prevents the association with membranes of a recombinant form containing the PH, C2, and GAP domains (residues 103-725), but lacking the disordered region (123). A truncated form of synGAP that also contains the disordered region beyond the GAP domain (AA 103-1293), remains soluble after expression in E. coli. This form of synGAP contains a carboxyl terminal ligand that binds to PDZ3 of PSD-95.

When expressed in isolation in E. coli and purified on PSD-95 PDZ3 Affinity Resin, the selected region of synGAP can be purified to to >70% homogeneity. When this same synGAP construct was tagged with an N-terminal 6× Histidine tag and purified by a similar procedure on Talon Affinity Resin, the purity of the recovered protein was less, approximately 25%, but the yield was considerably greater than after PDZ3 Domain Affinity Chromatography. In addition to an appended N-terminal 6× Histidine tag, this synGAP construct contains a naturally occurring internal 10× polyhistidine domain which markedly increases its affinity for Talon resin; This is expected to contribute to its high yield from the Talon resin.

synGAP is protein to aggregation, has poor solubility and its enzymatic activity is easily inactivated. When alternative tags (Strep II, GST, MBP) were fused to synGAP and used for purification, eluted protein was either impure or was not active. The presence of a naturally occurring PDZ domain ligand at its C-terminus, allowed purification of synGAP using PDZ Affinity Chromatography. Of note, synGAP was purified using Cobalt-CMA chromatography (IMAC), however this was likely due to the presence of a naturally occurring internal 10× histidine stretch in synGAP in addition to an N-terminal appended 6× Histidine tag.

For the experiment, fusion proteins containing synGAP (Rattus norvegicus, AA 103-1293) in frame with an N-terminal 6× Histidine tag were expressed in BL21(DE3) E. coli cells. Single colonies of BL21(DE3) cells harboring pJExpress414 plasmids containing the synGAP gene were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into LB medium, and grown at 37° C. until cultures reached an O.D.₆₀₀ of 0.8. Cultures were then chilled to 18° C., and IPTG was added to a final concentration of 0.2 mM. Cultures were grown for an additional 24 hours at 18° C. before being pelleted by centrifugation, and flash frozen in liquid nitrogen. Bacterial cell pellets (0.4-2.5 grams) containing synGAP were resuspended in 5 ml/g cell-paste Purification Buffer (50 mM HEPES, pH 7.5, 150 mM NaCl, 5 mM TCEP, 0.5 mM EDTA, 10 mM MgCl2, 2 mM PMSF, Complete Protease Inhibitor) supplemented with 0.02% Tergitol Type NP-40, 25 U/ml Benzonase and 10 U/ml ReadyLyse. The resuspended cells were sonicated (Branson, Danbury Conn.) 2× for 90 s/pass (15% power, 1.0 s on, 1.5 s off) before clarification by centrifugation at 16,000×g for 30 min at 4° C. Clarified lysate (1.8-14 ml) containing POIs was added to 100-200 ul of PSD-95 PDZ3 affinity chromatography resin pre-equilibrated with Purification Buffer supplemented with 0.02% Tergitol Type NP-40, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, 0.02% Tergitol Type NP-40, Roche Complete Protease Inhibitor, and 400 ug/ml YKQTSV peptide. Eluted protein was analyzed on 4-12% bis-tris SDS-PAGE gels and proteins were detected by Gel Code Blue staining.

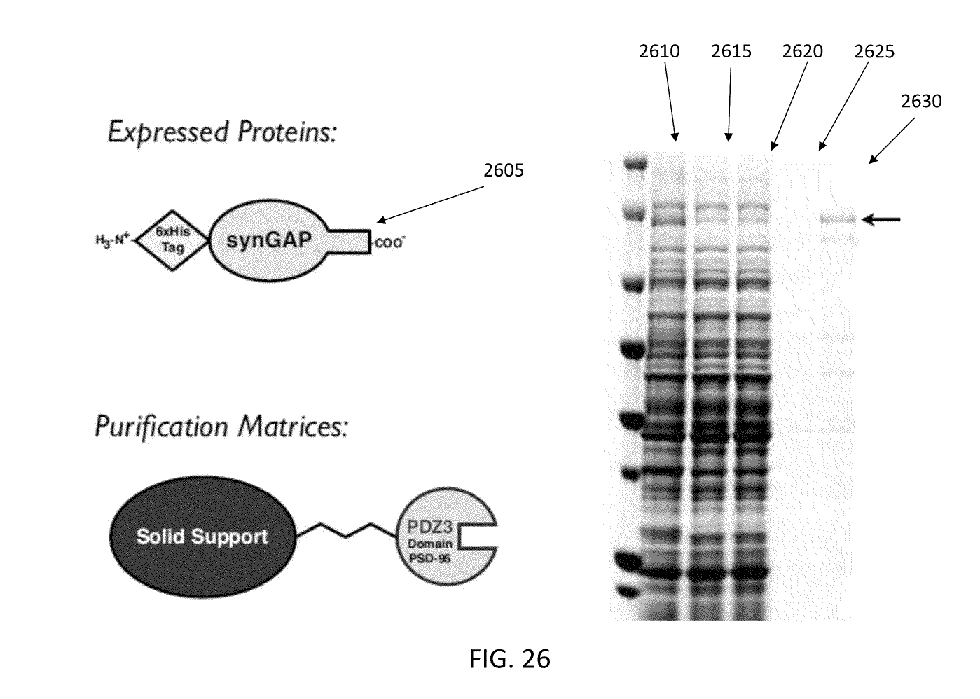

As shown, FIG. 26 relates to the above experiment of the PDZ3 affinity chromatography purification of the neuronal protein synGAP heterologously expressed in E. coli fused to a N-terminal 6× Histidine tag.

In FIG. 26, the expressed protein is synGAP 103-1293 (2605) and the purification matrix is PSD-95 PDZ3 domain coupled to a solid support. A GelCode Blue stained 4-12% bis-tris SDS-PAGE gel was used. The columns in FIG. 26 are: unclarified cell lysate (2610); soluble or clarified cell lysate (2615); unbound protein (2620); wash (2625); peptide eluate (2630). As shown in the peptide eluate, the protein is purified from the crude lysate.

### Example 3

### PSD-95 PDZ1-PDZ2 Affinity Chromatography Purification of the Neuronal Protein Cypin Heterologously Expressed in E. coli Untagged or Fused to a N-Terminal Maltose Binding Protein

Cypin was originally discovered and characterized as an abundant cytosolic protein that interacts directly with PSD-95 (124). When overexpressed in neurons, it appears to trap PSD-95 in the cytosol and reduces the targeting of PSD-95 to synapses. During neural development, it contributes to regulation of dendritic branching, in part by interfering with the interaction between PSD-95 and microtubules (125). Cypin binds to the PSD-95 family of proteins via a C-terminal SSSV (SEQ ID NO: 162) sequence and has the unique property that it requires the presence of both PDZ1 and PDZ2 for detectable binding (124). Cypin has been partially purified from brain extracts in small quantities by affinity purification with glutathione-beads bound to GST-PSD-95 (124). More recently, both GST- and MBP-fusion proteins of cypin have been purified by standard affinity chromatography (126).

An affinity column was used substituted with a HaloTag-PDZ1+PDZ2 fusion protein (residues 61 to 249 of Mus musculus PSD-95) to purify heterologously expressed human cypin. Cypin was expressed in E. coli either as the free protein or fused to an N-terminal MBP, and purified from the bacterial supernate on the PDZ1-PDZ2 Domain-Affinity Resin. Both cypin and MBP-cypin were purified to >99% homogeneity.

For the experiment, full length cypin (guanine deaminase, Homo sapiens: AA 1-454) or MBP-cypin fusion proteins were expressed in BL21(DE3) E. coli cells. Single colonies of BL21(DE3) cells harboring pJExpress414 plasmids containing cypin or MBP-cypin genes were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing cypin or MBP-cypin were resuspended in 5 ml/g cell paste of BugBuster Lysis Buffer [1× BugBuster (Cat. No. 70921, EMD Millipore, Billerica Mass.), 50 mM HEPES, 150 mM NaCl, 5 mM TCEP, 0.5 mM EDTA, 10 mM MgCl₂, 2 mM PMSF, Complete Protease Inhibitor supplemented with 25 U/ml Benzonase and 10 U/ml ReadyLyse]. Resuspended cells were incubated on a shaking platform at low speed for 20 minutes at room temperature before the lysate was clarified by centrifugation at 16,000×g for 30 min at 4° C. Clarified lysate (1.8-14 ml) containing POIs was added to 100-200 ul of PSD-95 PDZ1-PDZ2 (AA 4-192 of PSD-95) PDZ affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. Eluted protein was analyzed on 4-12% bis-tris SDS-PAGE gels and proteins were detected by Gel Code Blue staining.

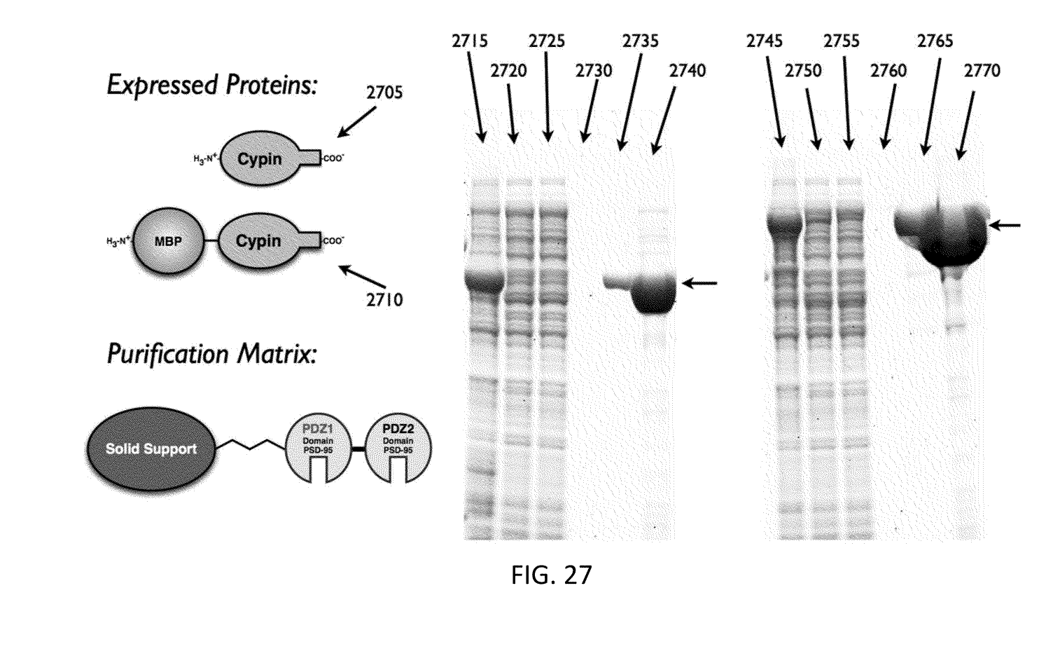

FIG. 27 illustrates a PSD-95 PDZ1-PDZ2 affinity chromatography purification of the neuronal protein cypin heterologously expressed in E. coli as an untagged protein or fused to a N-terminal maltose binding protein, and the analysis of the purification on and SDS PAGE gel.

In FIG. 27, the expressed protein is untagged cypin (2705) or MBP-cypin (2710) and the purification matrix is PSD-95 PDZ1-PDZ2 domaind coupled to a solid support. A GelCode Blue stained 4-12% bis-tris SDS-PAGE gel was used. The columns in FIG. 27 are: unclarified lysate (2715, 2745); clarified lysate or soluble protein (2720, 2750); unbound protein (2725, 2755); wash (2730, 2760); peptide eluate (2735, 2765); concentrated peptide eluate (2740, 2770). As shown, in the peptide eluates, the proteins are purified from the crude preparation to 98% purity. Both untagged and MBP-cypin proteins were able to bind with strong affinity to the solid support PDZ1 and PDZ2 domains and were washed out with PDZ peptide in a similar fashion.

### Example 4

### PSD-95 PDZ3 Affinity Chromatography Purification of the Neuronal Protein CRIPT Heterologously Expressed in E. coli

CRIPT (Cysteine Rich Interactor of PDZ Three) is a small (101 residue), highly conserved, cysteine-rich protein concentrated in the PSD of glutamatergic synapses in pyramidal neurons (46,47,127). CRIPT is one of three proteins known to bind to PDZ3 of PSD-95. It also binds the homologous PDZ3 domains of PSD-93 (chapsyn-110), SAP102 and SAP97 (47). It can bind simultaneously to polymerized tubulin, thus linking PSD-95 family proteins to the microtubule cytoskeleton (46). CRIPT contains 8 cysteine residues, all confined to CXXC repeats. To test whether PDZ3 Affinity Chromatography can be carried out in the presence of reducing agents, recombinant CRIPT was chosen for purification. CRIPT was previously purifed as a THX-fusion protein (46), so purification of CRIPTt in the absence of exogenous affinity tags was attempted. The purification was carried out in presence and absence of a reducing agent (5 mM TCEP).

When expressed in isolation in E. coli, CRIPT can be purified to more than 95% homogeneity when incubated with PDZ3 PSD-95 PDZ affinity resin. This resulting purity of CRIPT is independent of the presence or absence of the reducing agent TCEP. The 101 AA CRIPT protein contains 16 cysteine residues of unknown function. Reducing and non-reducing conditions were used during purification to account for any structural changes in the CRIPT protein caused by disulfide bond formation. Thus, unlike the NorpA-InaD affinity chromatography system (128), affinity chromatography with the PDZ domains from PSD-95 can be carried out either in the presence or absence of reducing agents.

For the experiment, full length CRIPT (Rattus norvegicus, AA 1-101) was expressed in BL21(DE3) E. coli cells. Single colonies of BL21(DE3) cells harboring pJExpress414 plasmids containing the CRIPT gene were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing CRIPT were resuspended in 5 ml/g cell paste of BugBuster Lysis Buffer [1× BugBuster (Cat. No. 70921, EMD Millipore, Billerica Mass.), 50 mM HEPES, 150 mM NaCl, 0 or 5 mM TCEP, 0.5 mM EDTA, 10 mM MgCl₂, 2 mM PMSF, Complete Protease Inhibitor supplemented with 25 U/ml Benzonase and 10 U/ml ReadyLyse]. Resuspended cells were incubated on a shaking platform at low speed for 20 minutes at room temperature before the lysate was clarified by centrifugation at 16,000×g for 30 min at 4° C. Clarified lysate (1.8-14 ml) containing POIs was added to 100-200 ul of PSD-95 PDZ3 affinity chromatography resin pre-equilibrated with Purification Buffer (with 0 or 5 mM TCEP), and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer (with 0 or 5 mM TCEP), transferred to a BioSpin chromatography column (Cat. No. 732-6008, BioRad, Hercules, Calif.), capped, and allowed to settle for 15 min. The cap was removed and the resin washed with 20 column volumes of Purification Buffer (with 0 or 5 mM TCEP). Bound protein was eluted by application of 4 column volumes of Peptide Elution Buffer [50 mM HEPES, pH 7.5, 150 mM NaCl, 0 or 5 mM TCEP, Roche Complete Protease Inhibitor, and 400 ug/ml YKQTSV peptide. Eluted protein was analyzed on 4-12% bis-tris SDS-PAGE gels and proteins were detected by Gel Code Blue staining.

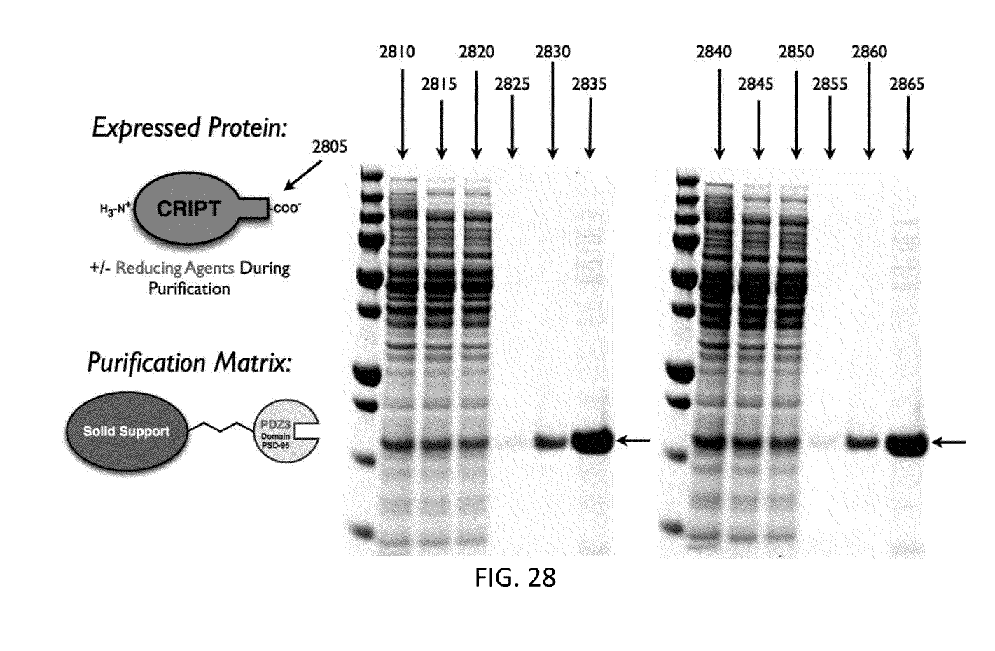

FIG. 28 relates to the purification of the cysteine rich interactor of PDZ3 (CRIPT) protein under reducing and non-reducing conditions using PDZ3 PSD-95 PDZ affinity resin. Until recently, the CRIPT protein has only been purified as a Thioredoxin fusion protein.

The results of FIG. 28 show the potential for PDZ affinity chromatography to be utilized for the purification of proteins with endogenous PDZ domain binding affinity. Additionally, this experiment shows that PDZ domain columns and their respective interacting peptide sequences (in this example the C-terminus of CRIPT) is compatible with the use of reducing agents (TCEP in this specific purification), which is contrary to the work published by Kimple and Sondek (128).

In FIG. 28, the expressed protein is reduced or non-reduced CRIPT (2805) and the purification matrix is PSD-95 PDZ3 domain coupled to a solid support. The columns in FIG. 28 are (−TCEP, +TCEP): unclarified lysate (2810, 2840); clarified lysate or soluble protein (2815, 2845); unbound protein (2820, 2850); wash (2825, 2855); peptide eluate (2830, 2860); concentrated peptide eluate (2835, 2865). As shown in the eluate, the CRIPT protein was purified to 98% purity for both the reduced and the non-reduced forms of CRIPT.

### Example 5

### PSD-95 PDZ2 Affinity Chromatography Purification of the PDZ Domain from Neuronal Nitric Oxide Synthase (nNOS), Heterologously Expressed in E. coli

Neuronal nitric oxide synthase (nNOS) (129,130) catalyzes the formation of nitric oxide and citrulline from arginine. Activation of nNOS by binding of Ca²⁺/calmodulin leads to formation of NO, which, in turn, activates guanylyl cyclase to form cGMP. NO generation can modulate synaptic plasticity by acting as a retrograde synaptic transmitter, and, in some circumstances provide neuroprotection against excitoxicity (131). It contains a single PDZ domain (AA 11-129 of Mus musculus nNOS) with an unusual structure that includes a β-Hairpin fold that itself acts as a PDZ domain ligand and can bind to both the syntrophin PDZ domain and the first two PDZ domains from PSD-95 (PDZ1 and PDZ2) in vivo and in vitro (2).

The PDZ β-Hairpin domain from nNOS was expressed in E. coli and purified to >95% homogeneity on the PDZ2 Domain Affinity Resin. Because of its small, modular structure and high solubility and expression level, it was hypothesized that the nNOS PDZ domain can make an excellent N-terminal, C-terminal or internal affinity or solubility enhancement tag for use in affinity chromatography.

For the experiment, nNOS PDZ domain (AA 11-129 of Mus musculus nNOS) was expressed in BL21(DE3) E. coli cells. Single colonies of BL21(DE3) cells harboring pJExpress414 plasmids containing nNOS PDZ were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing nNOS PDZ were resuspended in 5 ml/g cell paste of BugBuster Lysis Buffer [1× BugBuster (Cat. No. 70921, EMD Millipore, Billerica Mass.), 50 mM HEPES, 150 mM NaCl, 5 mM TCEP, 0.5 mM EDTA, 10 mM MgCl₂, 2 mM PMSF, Complete Protease Inhibitor supplemented with 25 U/ml Benzonase and 10 U/ml ReadyLyse]. Resuspended cells were incubated on a shaking platform at low speed for 20 minutes at room temperature before the lysate was clarified by centrifugation at 16,000×g for 30 min at 4° C. Clarified lysate (1.8-14 ml) containing POIs was added to 100-200 ul of PSD-95 PDZ2 affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. Eluted protein was analyzed on 4-12% bis-tris SDS-PAGE gels and proteins were detected by Gel Code Blue staining.

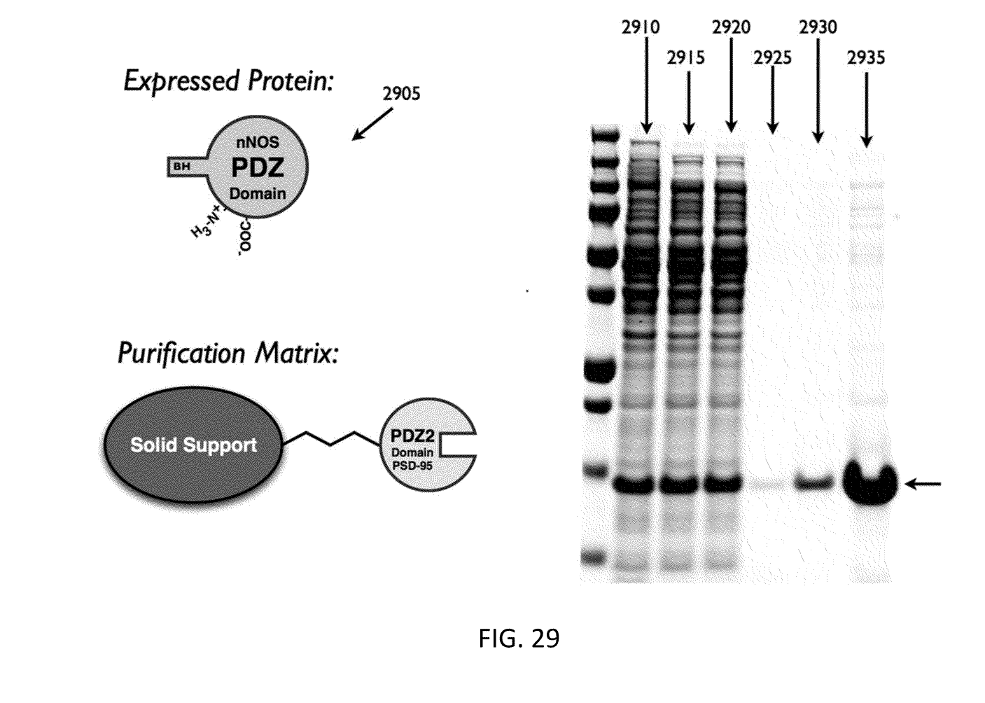

FIG. 29 relates to the purification of the nNOS PDZ domain expressed in isolation using an affinity matrix composed of the PDZ2 domain of PSD-95 coupled to a solid support. When expressed, and incubated with PSD-95 PDZ2 PDZ affinity chromatography resin, the nNOS PDZ domain was purified to more than 95% homogeneity when assessed by SDS-PAGE. This data shows the utility of the PDZ affinity chromatography matrix for protein purification and the utility of a single (1×) nNOS PDZ domain as a potential affinity tag.

FIG. 29 illustrates the results of the above experiment. As shown, the expressed protein is 1× nNOS PDZ domain (2905) and the purification matrix is PSD-95 PDZ2 coupled to a solid support. The columns in FIG. 29 are: cell lysate (2910); clarified cell lysate or soluble protein (2915); unbound protein (2920); wash (2925); peptide eluate (2930); concentrated peptide eluate (2935). As shown the purified POI in the eluate is close to 98% purity. As evident from the gel results, the expressed protein was able to bind to PDZ2 domain PSD-95 that was conjugated to the solid support. The expressed protein was then purified from the crude lysate and eluted from the column using PDZ binding peptide allowing a pure sample to be obtained.

### Example 6

### PSD-95 PDZ2 Affinity Chromatography Purification of Heterologously Expressed Thioredoxin (THX) Protein Fused to a Truncation Library of the C-Terminal 10 Amino Acids of the Tail Region of the NR2B Protein

In the experiment described herein, the development of PDZ Affinity Chromatography Tags (e.g. Type A-C) and how it is implemented is shown. In Examples 1-5, shown above, several neuronal proteins containing naturally occurring PDZ domains or C-terminal PDZ domain binding peptide sequences were purified using a known PDZ domain binding partner immobilized on a solid support. Neuronal proteins purified in the previous data set (Examples 1-5) contained naturally occurring PDZ domains or C-terminal PDZ domain binding peptide sequences, which can be attached to any Recombinant Protein of Interest (POI) and used to purify the POI via a known PDZ domain binding partner immobilized on a solid support. All POIs fused to PDZ domains or C-terminal PDZ domain binding peptide sequences were purified using PDZ Affinity Chromatography Resin and eluted using free PDZ binding C-terminal peptide.

The C-terminal six amino acids (SIESDV) of the N-methyl-D-aspartate (NMDAR) receptor subunit, NR2B (GluN2B), have been shown to form a high affinity (K_{D} of 2.3 and 0.7 uM, respectively) interaction with the PDZ1 and PDZ2 domains of PSD-95, in vitro and in vivo (49). It was hypothesized that if the six C-terminal amino acids of NR2B were appended to a Protein of Interest (POI), they would facilitate binding to the PDZ1 or PDZ2 domains of PSD-95 (Or additional PDZ domains known to bind NR2B including PDZ1 and 2 of SAP102 and SAP97). This forced interaction could be utilized to purify a POI if the tagged protein is passed over or incubated a solid support comprised of immobilized PDZ1 and/or PDZ2 domain(s) of PSD-95.

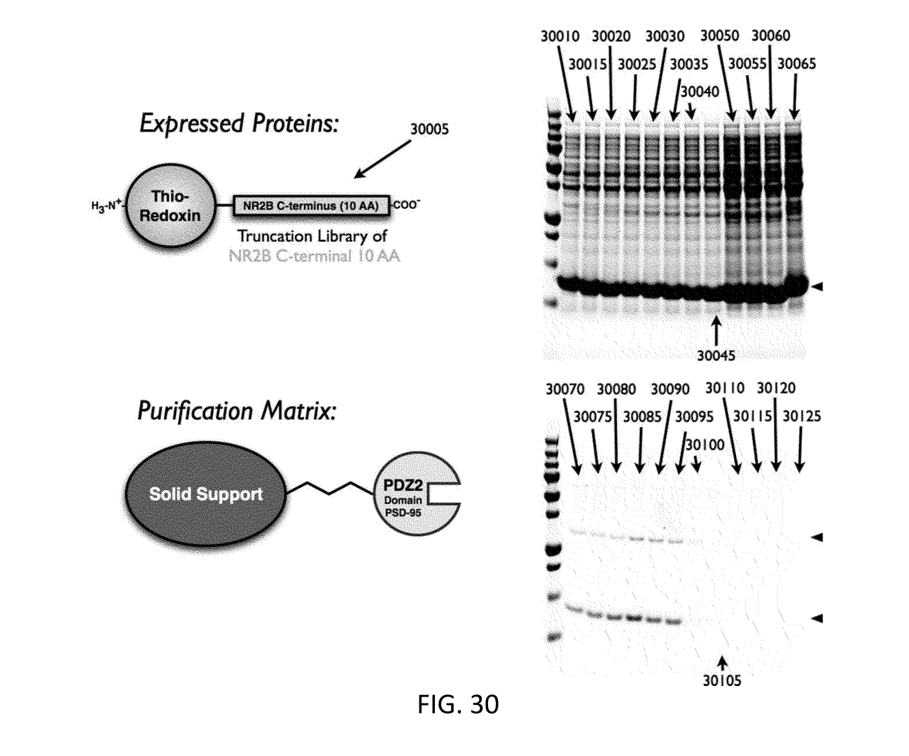

In order to test this hypothesis, the C-terminal 10 amino acids of the NR2B protein (EKLSSIESDV) were attached to the Thioredoxin (THX) protein (FIG. 30). THX was chosen as a fusion partner due to its high solubility and expression level. The size of the tag, 10 C-terminal amino acids of NR2B, was selected to account for any potential binding interference caused by steric hinderance due to fusion with THX. Specific truncations of the NR2B C-terminal sequence in THX fusion proteins were made to determine the minimum length of C-terminal NR2B sequence required to direct binding to and allow purification using PSD-95 PDZ2 coupled to a solid support (PDZ Affinity Chromatography Resin).

When soluble THX proteins carrying the C-terminal 5-10 AA of NR2B were incubated with PSD-95 PDZ2 coupled to a solid support, they were purified to >95% homogeneity when assessed by SDS-PAGE (FIG. 30). Interestingly, only the C-terminal five amino acids (IESDV) were required for effective binding to and elution from the PDZ2 PSD-95 matrix. In order to limit chances of structural perturbation or enzyme inhibition in fusion proteins containing the C-terminus of NR2B, the C-terminal PDZ Peptide Affinity tag was designed to consist of the seven amino acid sequence SSIESDV (SEQ ID NO: 161).

For the experiment, fusion proteins containing THX fused to varying numbers of residues from the NR2B Tail (EKLSSIESDV) were expressed in BL21(DE3) E. coli cells. Single colonies of BL21(DE3) cells harboring pJExpress414 plasmids containing genes of interest were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing fusions of THX fused to varying numbers of residues from the NR2B Tail (EKLSSIESDV) were resuspended in 5 ml/g cell paste of BugBuster Lysis Buffer [1× BugBuster (Cat. No. 70921, EMD Millipore, Billerica Mass.), 50 mM HEPES, 150 mM NaCl, 5 mM TCEP, 0.5 mM EDTA, 10 mM MgCl₂, 2 mM PMSF, Complete Protease Inhibitor supplemented with 25 U/ml Benzonase and 10 U/ml ReadyLyse]. Resuspended cells were incubated on a shaking platform at low speed for 20 minutes at room temperature before the lysate was clarified by centrifugation at 16,000×g for 30 min at 4° C. Clarified lysate (1.8-14 ml) containing POIs was added to 100-200 ul of PSD-95 PDZ2 affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. Eluted protein was analyzed on 4-12% bis-tris SDS-PAGE gels and proteins were detected by Gel Code Blue staining.

FIG. 30 illustrates the results of the above experiment of a PSD-95 PDZ2 affinity chromatography purification of heterologously expressed thioredoxin (THX) protein fused to a truncation library of the C-terminal 10 amino acids of the tail region of the NR2B protein (30005). The columns on the top gel in FIG. 30 are clarified lysate fractions: THX-EKLSSIESDV (30010); THX-KLSSIESDV (30015); THX-LSSIESDV (30020); THX-SSIESDV (30025); THX-SIESDV (30030); THX-IESDV (30035); THX-ESDV (30040); THX-SDV (30045); THX-DV (30050); THX-V (30055); THX- (30060); THX-EKLSSIESD (30065). The columns on the bottom gel in FIG. 30 are peptide eluate fractions: THX-EKLSSIESDV (30070); THX-KLSSIESDV (30075); THX-LSSIESDV (30080); THX-SSIESDV (30085); THX-SIESDV (30090); THX-IESDV (30095); THX-ESDV (30100); THX-SDV (30105); THX-DV (30110); THX-V (30115); THX- (30120); THX-EKLSSIESD (30125).

### Example 7

### PSD-95 PDZ2 Affinity Chromatography Purification of Heterologously Expressed Maltose Binding Protein (MBP) Protein Fused to a Truncation Library of the C-Terminal 10 Amino Acids of the Tail Region of the NR2B Protein

The C-terminal six amino acids (SIESDV) of the N-methyl-D-aspartate (NMDAR) receptor subunit, NR2B (GluN2B), have been shown to form a high affinity (K_{D} of 2.3 and 0.7 uM, respectively) interaction with the PDZ1 and PDZ2 domains of PSD-95, in vitro and in vivo (49). It was hypothesized that if the six C-terminal amino acids of NR2B were appended to a Protein of Interest (POI), they would facilitate binding to the PDZ1 or PDZ2 domains of PSD-95. This forced interaction could be utilized to purify a POI if the tagged protein is passed over or incubated a solid support comprised of immobilized PDZ1 and/or PDZ2 domain(s) of PSD-95.

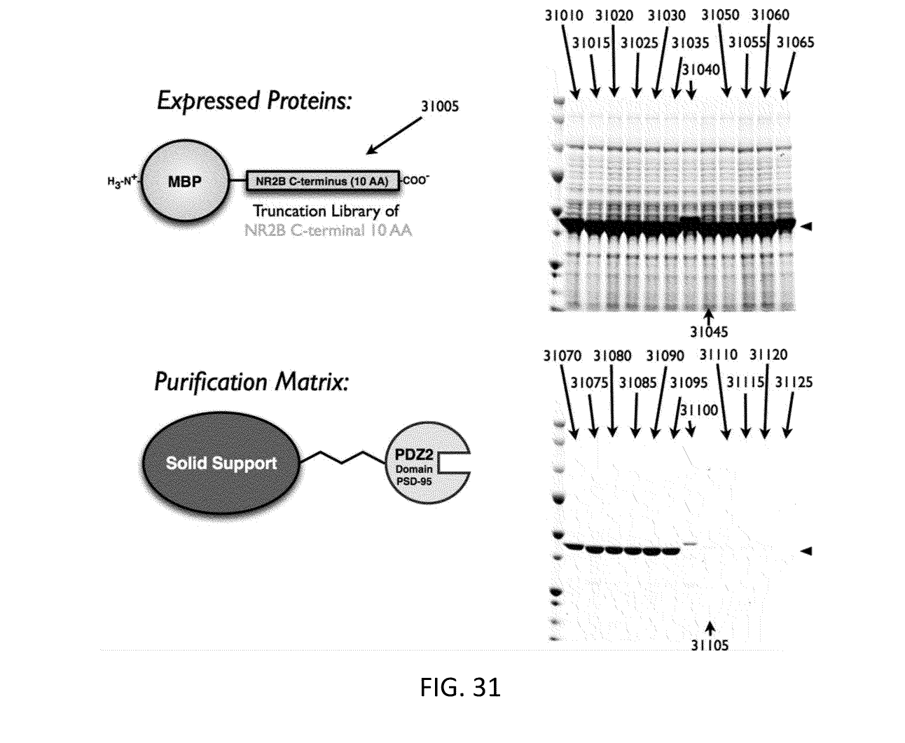

In order to test this hypothesis, the C-terminal 10 amino acids of the NR2B protein (EKLSSIESDV) were attached to the Maltose Binding Protein (MBP) protein (FIG. 31). MBP was chosen as a fusion partner due to its high solubility and expression level. The size of the tag, 10 C-terminal amino acids of NR2B, was selected to account for any potential binding interference caused by steric hinderance due to fusion with MBP. Specific truncations of the NR2B C-terminal sequence in MBP fusion proteins were made to determine the minimum length of C-terminal NR2B sequence required to direct binding to and allow purification using PSD-95 PDZ2 coupled to a solid support (PDZ Affinity Chromatography Resin).

When soluble MBP proteins carrying the C-terminal 5-10 AA of NR2B were incubated with PSD-95 PDZ2 coupled to a solid support, they were purified to >95% homogeneity when assessed by SDS-PAGE (FIG. 31). Interestingly, only the C-terminal five amino acids (IESDV) were required for effective binding to and elution from the PDZ2 PSD-95 matrix. In order to limit chances of structural perturbation or enzyme inhibition in fusion proteins containing the C-terminus of NR2B, the C-terminal PDZ Peptide Affinity tag was designed to consist of the seven amino acid sequence SSIESDV (SEQ ID NO: 161).

For the experiment, fusion proteins containing MBP fused to varying numbers of residues from the NR2B Tail (EKLSSIESDV) were expressed in BL21(DE3) E. coli cells. Single colonies of BL21(DE3) cells harboring pJExpress414 plasmids containing genes of interest were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing fusions of MBP fused to varying numbers of residues from the NR2B Tail (EKLSSIESDV) were resuspended in 5 ml/g cell paste of BugBuster Lysis Buffer [1× BugBuster (Cat. No. 70921, EMD Millipore, Billerica Mass.), 50 mM HEPES, 150 mM NaCl, 5 mM TCEP, 0.5 mM EDTA, 10 mM MgCl₂, 2 mM PMSF, Complete Protease Inhibitor supplemented with 25 U/ml Benzonase and 10 U/ml ReadyLyse]. Resuspended cells were incubated on a shaking platform at low speed for 20 minutes at room temperature before the lysate was clarified by centrifugation at 16,000×g for 30 min at 4° C. Clarified lysate (1.8-14 ml) containing POIs was added to 100-200 ul of PSD-95 PDZ2 affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. Eluted protein was analyzed on 4-12% bis-tris SDS-PAGE gels and proteins were detected by Gel Code Blue staining.

FIG. 31 illustrates the results of the above experiment, using a PSD-95 PDZ2 affinity chromatography purification of maltose binding protein fused to a truncation library of the C-terminal 10 amino acids of the tail region of the NR2B protein, MBP-NR2B tail C-terminal 10 AA (31005). The columns on the top gel in FIG. 31 are clarified lysate fractions: MBP-EKLSSIESDV (31010); MBP-KLSSIESDV (31015); MBP-LSSIESDV (31020); MBP-SSIESDV (31025); MBP-SIESDV (31030); MBP-IESDV (31035); MBP-ESDV (31040); MBP-SDV (31045); MBP-DV (31050); MBP-V (31055); MBP- (31060); MBP-EKLSSIESD (31065). The columns on the bottom gel in FIG. 31 are peptide eluate fractions: MBP-EKLSSIESDV (31070); MBP-KLSSIESDV (31075); MBP-LSSIESDV (31080); MBP-SSIESDV (31085); MBP-SIESDV (31090); MBP-IESDV (31095); MBP-ESDV (31100); MBP-SDV (31105); MBP-DV (31110); MBP-V (31115); MBP- (31120); MBP-EKLSSIESD (31125). As shown, several proteins were purified to a high purity if the tag length was kept at the longer length for specific capture. However decreasing the tag at the C-terminus abolished binding to the column even though the truncation was only one amino acid.

### Example 8

### PSD-95 PDZ2 Affinity Chromatography Purification of Heterologously Expressed Glutathion S-Transferase (GST) Protein Fused to a Truncation Library of the C-Terminal 10 Amino Acids of the Tail Region of the NR2B Protein

The C-terminal six amino acids (SIESDV) of the N-methyl-D-aspartate (NMDAR) receptor subunit, NR2B, have been shown to form a high affinity (K_{D} of 2.3 and 0.7 uM, respectively) interaction with the PDZ1 and PDZ2 domains of PSD-95, in vitro and in vivo (49). If the six C-terminal amino acids of NR2B are appended to a Protein of Interest (POI), they would facilitate binding to the PDZ1 or PDZ2 domains of PSD-95. This forced interaction could be utilized to purify a POI if the tagged protein is passed over or incubated a solid support comprised of immobilized PDZ1 and/or PDZ2 domain(s) of PSD-95.

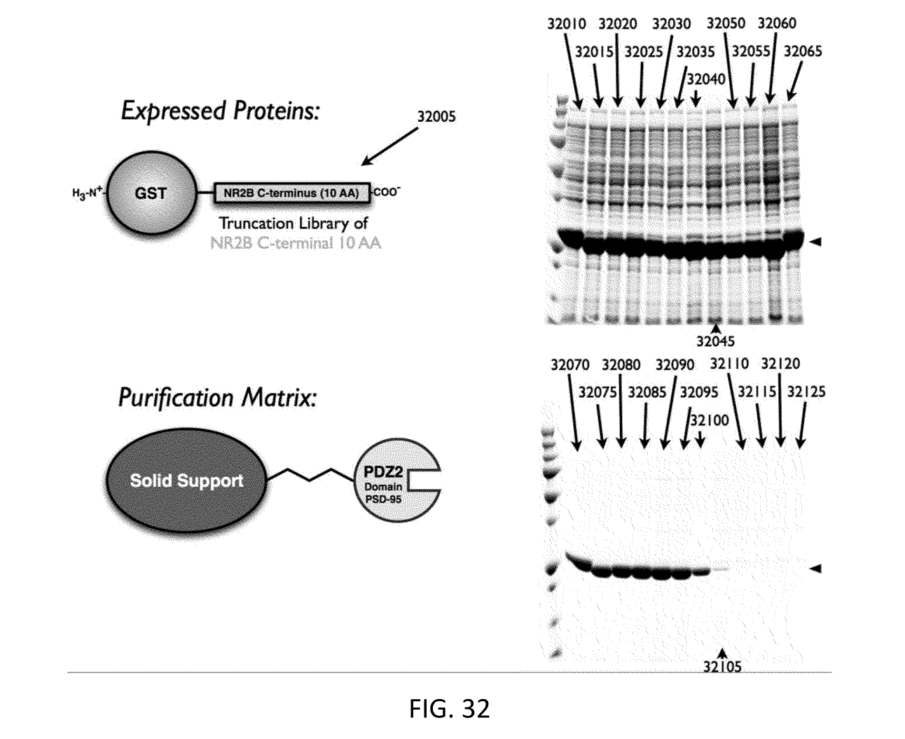

To test this hypothesis, the C-terminal 10 amino acids of the NR2B protein (EKLSSIESDV) were attached to the Glutathione S-Transferase (GST) protein (FIG. 32). GST was chosen as a fusion partner due to its high solubility and expression level. The size of the tag, 10 C-terminal amino acids of NR2B, was selected to account for any potential binding interference caused by steric hinderance due to fusion with GST. Specific truncations of the NR2B C-terminal sequence in GST fusion proteins were made to determine the minimum length of C-terminal NR2B sequence required to direct binding to and allow purification using PSD-95 PDZ2 coupled to a solid support (PDZ affinity chromatography resin).

When soluble GST proteins carrying the C-terminal 5-10 AA of NR2B were incubated with PSD-95 PDZ2 coupled to a solid support, they were purified to more than 95% homogeneity when assessed by SDS-PAGE (FIG. 32). Interestingly, only the C-terminal five amino acids (IESDV) were required for effective binding to and elution from the PDZ2 PSD-95 matrix. In order to limit chances of structural perturbation or enzyme inhibition in fusion proteins containing the C-terminus of NR2B, the C-terminal PDZ peptide affinity tag was designed to consist of the seven amino acid sequence SSIESDV (SEQ ID NO: 161).

For the experiment, fusion proteins containing GST fused to varying numbers of residues from the NR2B Tail (EKLSSIESDV) were expressed in BL21(DE3) E. coli cells. Single colonies of BL21(DE3) cells harboring pJExpress414 plasmids containing genes of interest were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing fusions of GST fused to varying numbers of residues from the NR2B Tail (EKLSSIESDV) were resuspended in 5 ml/g cell paste of BugBuster Lysis Buffer [1× BugBuster (Cat. No. 70921, EMD Millipore, Billerica Mass.), 50 mM HEPES, 150 mM NaCl, 5 mM TCEP, 0.5 mM EDTA, 10 mM MgCl₂, 2 mM PMSF, Complete Protease Inhibitor supplemented with 25 U/ml Benzonase and 10 U/ml ReadyLyse]. Resuspended cells were incubated on a shaking platform at low speed for 20 minutes at room temperature before the lysate was clarified by centrifugation at 16,000×g for 30 min at 4° C. Clarified lysate (1.8-14 ml) containing POIs was added to 100-200 ul of PSD-95 PDZ2 affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. Eluted protein was analyzed on 4-12% bis-tris SDS-PAGE gels and proteins were detected by Gel Code Blue staining.

FIG. 32 illustrates the results of the above experiment, using a PSD-95 PDZ2 affinity chromatography purification of glutathione S-transferase protein fused to a truncation library of the C-terminal 10 amino acids of the tail region of the NR2B protein, MBP-NR2B tail C-terminal 10 AA (32005). The columns on the top gel in FIG. 32 are clarified lysate fractions: GST-EKLSSIESDV (32010); GST-KLSSIESDV (32015); GST-LSSIESDV (32020); GST-SSIESDV (32025); GST-SIESDV (32030); GST-IESDV (32035); GST-ESDV (32040); GST-SDV (32045); GST-DV (32050); GST-V (32055); GST- (32060); GST-EKLSSIESD (32065). The columns on the bottom gel in FIG. 32 are peptide eluate fractions: GST-EKLSSIESDV (32070); GST-KLSSIESDV (32075); GST-LSSIESDV (32080); GST-SSIESDV (32085); GST-SIESDV (32090); GST-IESDV (32095); GST-ESDV (32100); GST-SDV (32105); GST-DV (32110); GST-V (32115); GST- (32120); GST-EKLSSIESD (32125). As shown, the purity of the GST is increased with the 10 amino acid tag and decreases when the tag is decreased by one amino acid at the C-terminal end. As shown, several proteins were purified to a high purity if the tag length was kept at the longer length for specific capture. However decreasing the tag at the C-terminus abolished binding to the column even though the truncation was only one amino acid.

### Example 9

### PSD-95 PDZ2 Affinity Chromatography Purification the PDZ Domain from the Neuronal Nitric Oxide Synthase (nNOS), Heterologously Expressed in E. coli

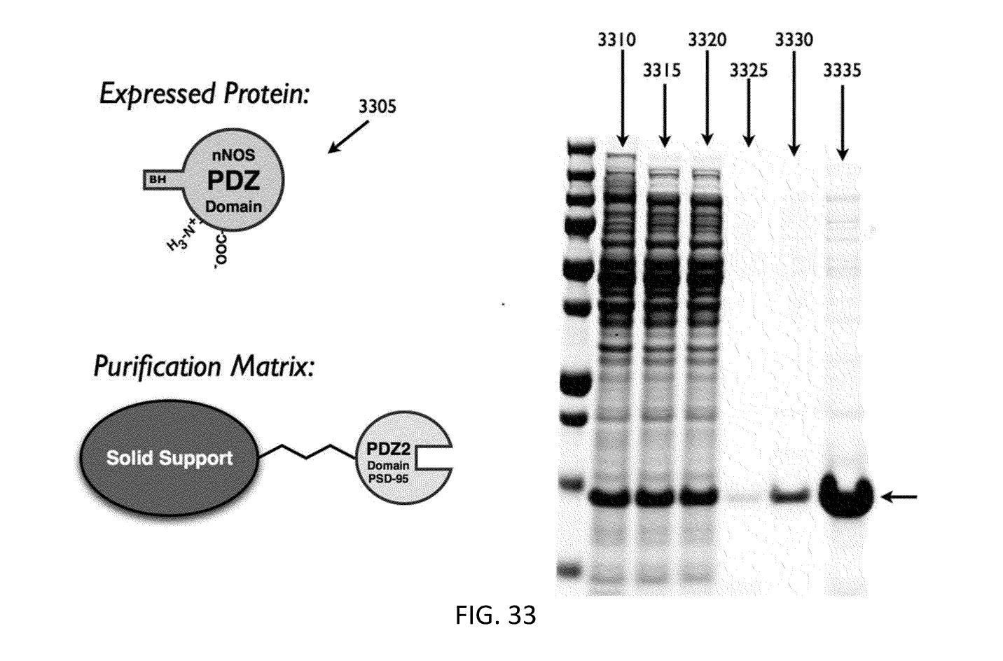

The nNOS PDZ domain (AA 11-129 of Mus musculus nNOS) has been shown to bind both the syntrophin PDZ domain and the first two PDZ domains from PSD-95 (PDZ1 and PDZ2) in vivo and in vitro through the use of its C-terminal β-hairpin motif. Because of its small, modular structure and high solubility and expression level, the nNOS PDZ domain would make an excellent N-terminal, C-terminal or internal affinity or solubility enhancement tag for use in affinity chromatography. When expressed, and incubated with PSD-95 PDZ2 PDZ affinity chromatography resin (FIG. 33), the nNOS PDZ domain was purified to more than 95% homogeneity when assessed by SDS-PAGE (FIG. 33). Data acquired shows the utility of the PDZ affinity chromatography matrix for protein purification and the utility of a single (1×) nNOS PDZ domain as a potential affinity tag.

For the experiment, nNOS PDZ domain (AA 11-129 of Mus musculus nNOS) was expressed in BL21(DE3) E. coli cells. Single colonies of BL21(DE3) cells harboring pJExpress414 plasmids containing the nNOS PDZ gene were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing nNOS PDZ were resuspended in 5 ml/g cell paste of BugBuster Lysis Buffer [1× BugBuster (Cat. No. 70921, EMD Millipore, Billerica Mass.), 50 mM HEPES, 150 mM NaCl, 5 mM TCEP, 0.5 mM EDTA, 10 mM MgCl₂, 2 mM PMSF, Complete Protease Inhibitor supplemented with 25 U/ml Benzonase and 10 U/ml ReadyLyse]. Resuspended cells were incubated on a shaking platform at low speed for 20 minutes at room temperature before the lysate was clarified by centrifugation at 16,000×g for 30 min at 4° C. Clarified lysate (1.8-14 ml) containing POIs was added to 100-200 ul of PSD-95 PDZ2 affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. Eluted protein was analyzed on 4-12% bis-tris SDS-PAGE gels and proteins were detected by Gel Code Blue staining.

FIG. 33 illustrates the results of the experiment. In FIG. 33, the expressed protein is 1× nNOS PDZ domain (3305) and the purification matrix is PSD-95 PDZ2 coupled to a solid support. A GelCode Blue stained 4-12% bis-tris SDS-PAGE gel was used. The columns in FIG. 33 are: cell lysate (3310); clarified cell lysate or soluble protein (3315); unbound protein (3320); wash (3325); peptide eluate (3330); concentrated peptide eluate (3335). As shown, in the peptide eluate, the protein of interest was purified from the extraneous proteins that are shown in the lysage column to an almost 98% purity.

### Example 10

### PSD-95 PDZ2 Affinity Chromatography Purification of 2× and 3× Tandem nNOS PDZ Domains Heterologously Expressed in E. coli

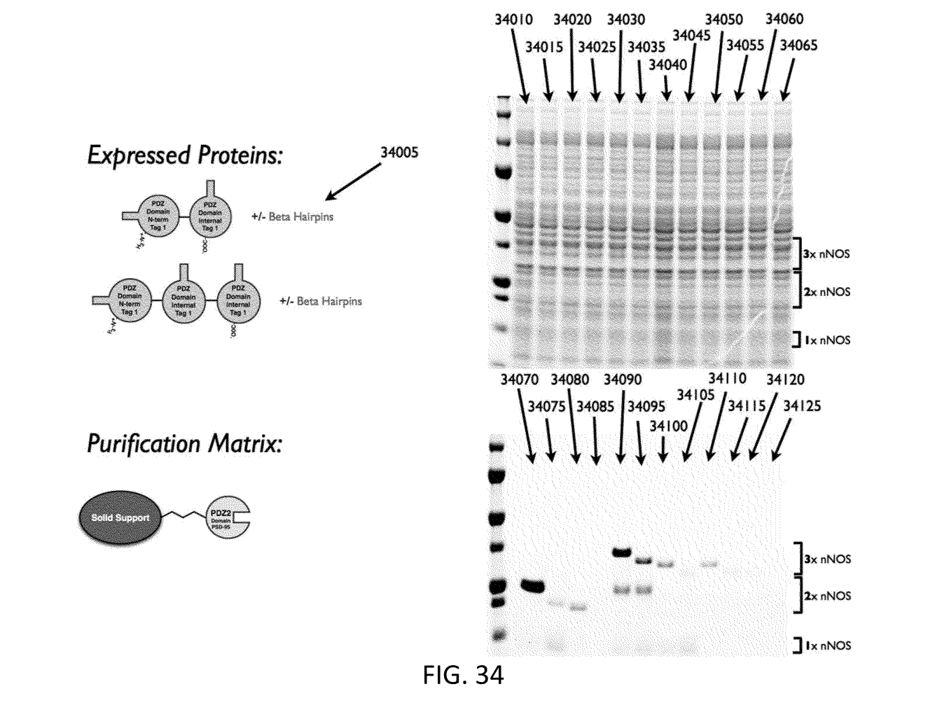

As shown in Example 9 and in the data in FIG. 33 the utility of a single nNOS PDZ (1× nNOS) domain as a potential affinity tag when purified using PSD-95 PDZ2 PDZ affinity chromatography resin is very clear. While one nNOS PDZ domain can function as an affinity tag, fusing additional nNOS PDZ domains (e.g. 2×, 3× nNOS) in tandem with the first domain could increase the affinity of the nNOS PDZ domain fusion protein for the PSD-95 PDZ2 PDZ affinity chromatography resin. Additionally, fusing multiple nNOS PDZ domains in tandem might also increase the potential solubility enhancing affects of the nNOS PDZ domain. Because the β-hairpin (BH) domain contained in the nNOS PDZ domain is responsible for binding to the PDZ2 PDZ affinity chromatography resin, incorporation of additional nNOS PDZ domains in tandem can result in increased affinity for the PDZ2 Resin and thus potentially decrease the capacity for peptide elution from the matrix. To account for the potential increased avidity of tandem 2× and 3× nNOS PDZ domains for the PSD-95 PDZ2 matrix, a library of 2× and 3× nNOS tandem PDZ domains with a varying number of internal and C-terminal beta hairpin domains (0, 1, 2 or 3 C-terminal BH per library member) can be used (FIG. 34).

Fusion proteins consisting of tandem nNOS PDZ domains and BHs can successfully be purified using PSD-95 PDZ2 coupled to a solid support (FIG. 34). Interestingly, the only construct purified to more than 95% homogeneity, when assessed by SDS-PAGE, is the 2× nNOS PDZ domain fusion protein that contains a BH after each nNOS PDZ domain. All other tandem nNOS PDZ fusion proteins eluted from the PDZ2 affinity resin show either extensive proteolysis at domain junctions or premature release from the ribosome during protein expression (cannot discern between the two). In some embodiments designed to assay the solubility enhancing and affinity purification properties of the nNOS PDZ domain, only the 1× nNOS PDZ domain-1× BH and 2× nNOS PDZ domain-2× BH protein tags will be used.

For the experiment, fusion proteins consisting of 2-3 nNOS PDZ domains and 0-3 nNOS BH domains in tandem were expressed in BL21(DE3) E. coli cells. Single colonies of BL21(DE3) cells harboring pJExpress414 plasmids containing the tandem nNOS PDZ and BH genes were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing the proteins of interest were resuspended in 5 ml/g cell paste of BugBuster Lysis Buffer [1× BugBuster (Cat. No. 70921, EMD Millipore, Billerica Mass.), 50 mM HEPES, 150 mM NaCl, 5 mM TCEP, 0.5 mM EDTA, 10 mM MgCl₂, 2 mM PMSF, Complete Protease Inhibitor supplemented with 25 U/ml Benzonase and 10 U/ml ReadyLyse]. Resuspended cells were incubated on a shaking platform at low speed for 20 minutes at room temperature before the lysate was clarified by centrifugation at 16,000×g for 30 min at 4° C. Clarified lysate (1.8-14 ml) containing POIs was added to 100-200 ul of PSD-95 PDZ2 affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. Eluted protein was analyzed on 4-12% bis-tris SDS-PAGE gels and proteins were detected by Gel Code Blue staining.

FIG. 34 illustrates a PSD-95 PDZ2 affinity chromatography purification of 2× and 3× tandem nNOS PDZ heterologously expressed in E. coli. The expressed protein is 2×/3× nNOS PDZ tandem variants (34005) and the purification matrix is PSD-95 PDZ2 domain coupled to a solid support. A GelCode Blue stained 4-12% bis-tris SDS-PAGE gel was used. The columns on the top gel in FIG. 34 are clarified lysate fractions: nNOS-BH-nNOS-BH (34010); nNOS-BH-nNOS (34015); nNOS-nNOS-BH (34020); nNOS-nNOS (34025); nNOS-BH-nNOS-BH-nNOS-BH (34030); nNOS-BH-nNOS-BH-nNOS (34035); nNOS-BH-nNOS-nNOS-BH (34040); nNOS-BH-nNOS-nNOS (34045); nNOS-nNOS-BH-nNOS-BH (34050); nNOS-nNOS-BH-nNOS (34055); nNOS-nNOS-nNOS-BH (34060); nNOS-nNOS-nNOS (34065). The columns on the bottom gel in FIG. 34 are peptide eluate fractions: nNOS-BH-nNOS-BH (34070); nNOS-BH-nNOS (34075); nNOS-nNOS-BH (34080); nNOS-nNOS (34085); nNOS-BH-nNOS-BH-nNOS-BH (34090); nNOS-BH-nNOS-BH-nNOS (34095); nNOS-BH-nNOS-nNOS-BH (34100); nNOS-BH-nNOS-nNOS (34105); nNOS-nNOS-BH-nNOS-BH (34110); nNOS-nNOS-BH-nNOS (34115); nNOS-nNOS-nNOS-BH (34120); nNOS-nNOS-nNOS (34125). As shown in the results proteins having the beta hairpins increased the purity and the yield of the protein of interest. Additionally, the lack of the beta hairpins showed a decrease in the retention of the POI onto the purification matrix.

### Example 11

### PSD-95 PDZ2 Affinity Chromatography Purification of MBP and GST Fusion Proteins Containing N-Terminal, Internal or C-Terminal Single (1×) or Tandem (2×) nNOS PDZ Domains (e.g. Type B PDZ Affinity Tags) Heterologously Expressed in E. coli

When naturally occurring PDZ domains are expressed in isolation or tandem they are able to be purified using a known PDZ domain binding partner immobilized on a solid support. In addition to trying to purify PDZ domains in isolation or fused to one another, PDZ domains could function as purification tags when positioned N-terminally or C-terminally to a POI, or internally sandwiched between two POIs. Additionally, when naturally occurring C-terminal PDZ domain binding peptide sequences (ligands) were expressed attached to a recombinant Protein of Interest (POI), the fusion proteins were also able to be purified using a known PDZ domain binding partner immobilized on a solid support. In several embodiments described, all POIs fused to PDZ domains or C-terminal PDZ domain binding peptide sequences purified using PDZ affinity chromatography resin were eluted using free PDZ binding C-terminal peptides.

Data in FIG. 33 showed the utility of a single nNOS PDZ (1× nNOS) domain as a potential affinity tag when purified using PSD-95 PDZ2 PDZ affinity chromatography resin (more than 95% purity), whereas data in FIG. 34 showed that fusion proteins consisting of two tandem nNOS PDZ domains and BHs (nNOS-BH-nNOS-BH) could successfully be purified to more than 95% purity using PSD-95 PDZ2 coupled to a solid support.

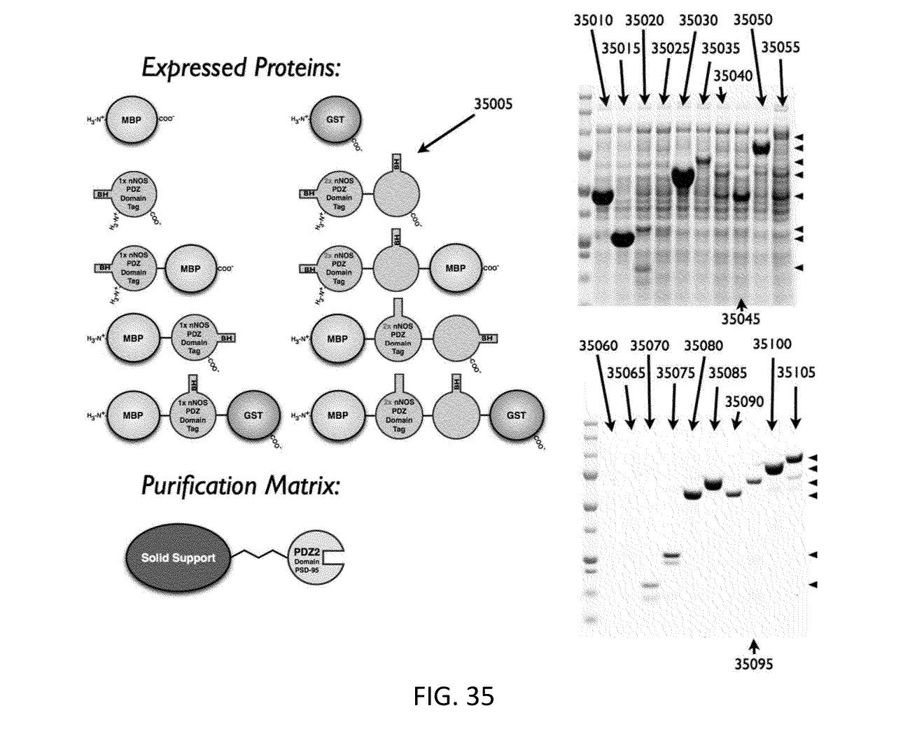

In both FIGS. 33 and 34, 1× and 2× nNOS PDZ-BH proteins were purified as isolated proteins (e.g. not fused to a POI) using PSD-95 PDZ2 PDZ affinity chromatography resin. In FIG. 35, 1× or 2× nNOS PDZ-BH proteins were fused N- or C-terminally to MBP, or internally between an MBP and GST proteins and purified using PSD-95 PDZ2 PDZ affinity chromatography resin. In all cases, whether fused N-terminally, C-terminally or internally, 1× or 2× nNOS PDZ-BH PDZ affinity tags (e.g. Type B PDZ affinity tags) were able to purify the attached POI(s) to >95% purity. The 1× and 2× nNOS PDZ-BH tags performed similarly across all orientations, however the 1× and 2× nNOS PDZ-BH tags appeared to function slightly better (increased yield) when fused as a C-terminal tag to MBP instead of an N-terminal tag.

For the experiment, MBP, GST, 1× and 2× tandem nNOS PDZ domains protein or fusion proteins consisting of MBP and/or GST fused in frame with N-terminal, internal or C-terminal 1× and 2× tandem nNOS PDZ domains (e.g. Type B PDZ affinity tags) were expressed in BL21(DE3) E. coli cells. Single colonies of BL21(DE3) cells harboring pJExpress414 plasmids containing genes of interest were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing the proteins of interest were resuspended in 5 ml/g cell paste of BugBuster Lysis Buffer [1× BugBuster (Cat. No. 70921, EMD Millipore, Billerica Mass.), 50 mM HEPES, 150 mM NaCl, 5 mM TCEP, 0.5 mM EDTA, 10 mM MgCl₂, 2 mM PMSF, Complete Protease Inhibitor supplemented with 25 U/ml Benzonase and 10 U/ml ReadyLyse]. Resuspended cells were incubated on a shaking platform at low speed for 20 minutes at room temperature before the lysate was clarified by centrifugation at 16,000×g for 30 min at 4° C. Clarified lysate (1.8-14 ml) containing POIs was added to 100-200 ul of PSD-95 PDZ2 affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. Eluted protein was analyzed on 4-12% bis-tris SDS-PAGE gels and proteins were detected by Gel Code Blue staining.

FIG. 35 illustrates the results of the experiment above, a PSD-95 PDZ2 affinity chromatography purification of MBP, GST, 1× and 2× tandem nNOS PDZ domains protein or fusion proteins consisting of MBP and/or GST fused in frame with N-terminal, internal or C-terminal 1× and 2× tandem nNOS PDZ domains (e.g. Type B PDZ affinity tags).

In FIG. 35, the expressed proteins are MBP, GST, 1× and 2× tandem nNOS PDZ domains protein or fusion proteins consisting of MBP and/or GST fused in frame with N-terminal, internal or C-terminal 1× and 2× tandem nNOS PDZ domains (e.g. Type B PDZ affinity tags) (35005) and the purification matrix is PSD-95 PDZ2 domain coupled to a solid support. A GelCode Blue stained 4-12% bis-tris SDS-PAGE gel was used. The columns on the top gel in FIG. 35 are clarified cell lysate fractions: MBP (35010); GST (35015); 1× nNOS tag (35020); 2× nNOS tag (35025); MBP-1× nNOS tag (35030); MBP-2× nNOS tag (35035); 1× nNOS tag-MBP (35040); 2× nNOS tag-MBP (35045); MBP-1× nNOS tag-GST (35050); MBP-1× nNOS tag-GST (35055). The columns on the top gel in FIG. 35 are peptide eluate fractions: MBP (35060); GST (35065); 1× nNOS tag (35070); 2× nNOS tag (35075); MBP-1× nNOS tag (35080); MBP-2× nNOS tag (35085); 1× nNOS tag-MBP (35090); 2× nNOS tag-MBP (35095); MBP-1× nNOS tag-GST (35100); MBP-1× nNOS tag-GST (35105). As indicated from the results, addition of the nNOS-BH tag increases the purity and the yield of the POI.

### Example 12

### 1× and 2× Tandem nNOS PDZ-BH or C-Terminal PDZ Domain Binding Peptide Affinity Chromatography Purification of MBP and GST Fusion Proteins Containing N-Terminal, Internal or C-Terminal PSD-95 PDZ2 Domains (e.g. Type C PDZ Affinity Tags) Heterologously Expressed in E. coli

The PDZ2 domain of PSD-95 is known in the art to form a high affinity interaction with the C-terminal peptide sequence of the NR2B Tail (K_{D} 0.7 uM) (49) and the nNOS PDZ with β-hairpin domain (K_{D} ˜0.6 uM) (34,132,133). Previous experiments highlighted in FIGS. 30-32 and 33-35 have shown that appending the NR2B tail sequence (e.g. Type A PDZ affinity tag) or 1×-2× nNOS PDZ-β-hairpin domains (e.g. Type B PDZ affinity tag), respectively, to a POI can facilitate purification of the POI when passed over PSD-95 PDZ2 PDZ affinity chromatography resin and eluted with free NR2B tail C-terminal peptides (H3N+-SIESDV-COO—). Part of the hypothesized diversity of the PDZ affinity chromatography system stems from the ability to switch affinity tag and resin components (e.g. instead of using a PSD-95 PDZ2 domain and NR2B Tail PDZ binding C-terminal peptide as the affinity tag and resin, respectively, it can be possible to use the NR2B tail PDZ binding C-terminal peptide and PSD-95 PDZ2 domain as the affinity tag and resin, respectively).

In order to test the hypothesis, PSD-95 PDZ2 domains (e.g. Type C PDZ affinity tags) were fused N- or C-terminally to MBP, or internally between an MBP and GST proteins and purified using affinity chromatography resins comprised of NR2B C-terminal PDZ domain binding peptide (H3N+-GAGSSIESDV-COO—), 1× nNOS PDZ-β-hairpin domains or 2× nNOS PDZ-β-hairpin domains. In all cases, whether fused N-terminally, C-terminally or internally, PSD-95 PDZ2 affinity tags (e.g. Type C PDZ affinity tags) were able to purify the attached POI(s) to more than 95% purity. Interestingly, NR2B C-terminal PDZ domain binding peptide affinity resin yielded purified POIs in yields of 25-120 times that of POIs purified using the 1× and 2× nNOS PDZ-β-hairpin PDZ domain affinity resin. Tags performed equally when fused internally or C-terminally, however N-terminal tag fusions were purified in slightly lower yield. This data highlights the utility of PDZ domain binding peptide resin as a purification matrix; this is especially important, as the PDZ domain binding peptide resin is inexpensive and easy to prepare, especially in large quantities. Note that control MBP or GST expressed alone without a PDZ domain did not bind to any of the three affinity resins.

For the experiment, MBP, GST and PSD-95 PDZ2 domains protein or fusion proteins consisting of MBP and/or GST fused in frame with N-terminal, internal or C-terminal PSD-95 PDZ2 domains (e.g. Type C PDZ affinity tags) were expressed in BL21(DE3) E. coli cells. Single colonies of BL21(DE3) cells harboring pJExpress414 plasmids containing genes of interest were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing the proteins of interest were resuspended in 5 ml/g cell paste of BugBuster Lysis Buffer [1× BugBuster (Cat. No. 70921, EMD Millipore, Billerica Mass.), 50 mM HEPES, 150 mM NaCl, 5 mM TCEP, 0.5 mM EDTA, 10 mM MgCl₂, 2 mM PMSF, Complete Protease Inhibitor supplemented with 25 U/ml Benzonase and 10 U/ml ReadyLyse]. Resuspended cells were incubated on a shaking platform at low speed for 20 minutes at room temperature before the lysate was clarified by centrifugation at 16,000×g for 30 min at 4° C. Clarified lysate (1.8-14 ml) containing POIs was added to 100-200 ul of NR2B C-terminal PDZ domain binding peptide, 1× nNOS PDZ-β-hairpin domains or 2× nNOS PDZ-β-hairpin domain affinity chromatography resins pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. Eluted protein was analyzed on 4-12% bis-tris SDS-PAGE gels and proteins were detected by Gel Code Blue staining.

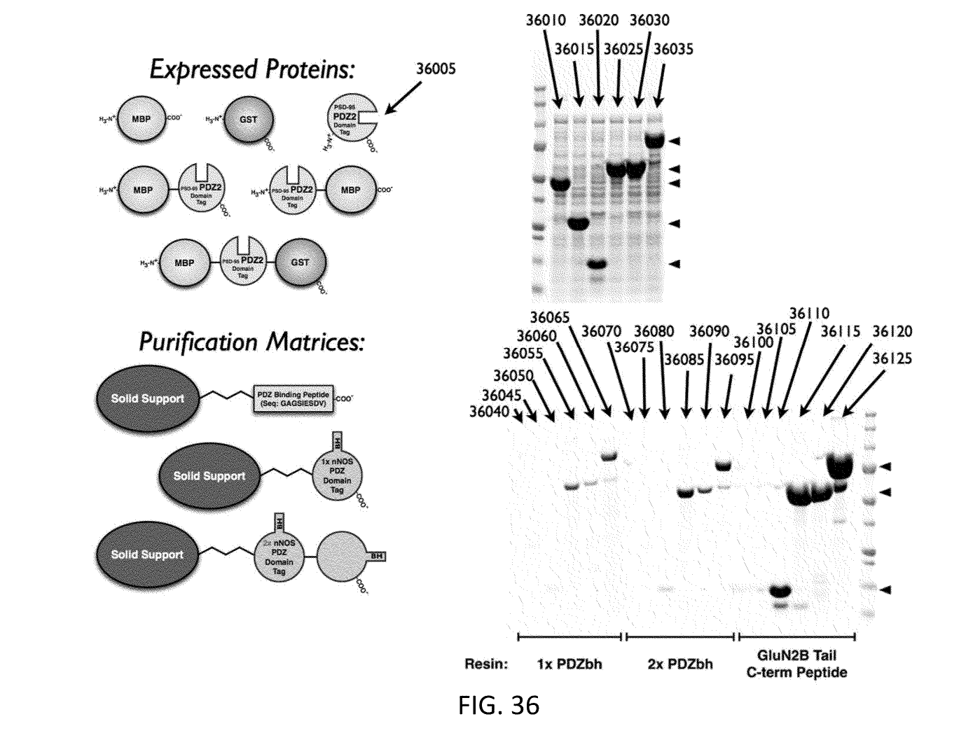

FIG. 36 illustrates a NR2B C-terminal PDZ domain binding peptide, 1× nNOS PDZ-β-hairpin domains or 2× nNOS PDZ-β-hairpin domain affinity chromatography purification of MBP and GST fusion proteins containing N-terminal, internal and C-terminal PSD-95 PDZ2 domains (e.g. Type C PDZ affinity tags) heterologously expressed in E. coli. The expressed proteins are MBP and GST with N-terminal, internal and C-terminal with PSD-95 PDZ2 domains (36005) and the purification matrix are: NR2B (GluN2B) peptide ligand (GAGSSIESDV) coupled to a solid support; 1× nNOS PDZ domain coupled to a solid support; and 2× nNOS PDZ domain coupled to a solid support. A GelCode Blue stained 4-12% bis-tris SDS-PAGE gel was used. The columns on the top gel in FIG. 36 are clarified cell lysate fractions: MBP (36010); GST (36015); PDZ2 tag (36020); MPB-PDZ2 tag (36025); PDZ2 tag-MBP (36030); MBP-PDZ2 tag-GST (36035). The columns on the bottom gel in FIG. 36 are peptide eluate fractions: MBP (36040); GST (36045); PDZ2 tag (36050); MPB-PDZ2 tag (36055); PDZ2 tag-MBP (36060); MBP-PDZ2 tag-GST (36065); MBP (36070); GST (36075); PDZ2 tag (36080); MPB-PDZ2 tag (36085); PDZ2 tag-MBP (36090); MBP-PDZ2 tag-GST (36095); MBP (36100); GST (36105); PDZ2 tag (36110); MPB-PDZ2 tag (36115); PDZ2 tag-MBP (36120); MBP-PDZ2 tag-GST (36125). As shown in the gel, the protein yield was increased when using the C-terminal PDZ domain binding peptide affinity resin.

### Example 13

### PSD-95 PDZ2 Affinity Chromatography Purification of DHFR, Dasher GFP, LacZ and CAT Fusion Proteins Containing PDZ Binding C-Terminal Peptides (e.g. Type A PDZ Affinity Tags) Heterologously Expressed in E. coli

The C-terminal six amino acids (SIESDV) of the N-methyl-D-aspartate (NMDAR) receptor subunit, NR2B, have been shown to form a high affinity (K_{D} of 9.7 and 1.3 uM, respectively) interaction with the PDZ1 and PDZ2 domains of PSD-95, in vitro and in vivo (134). Data in FIGS. 30-32 has shown that when the six C-terminal amino acids of NR2B are appended to a Protein of Interest (POI), the POI can be purified using a PDZ affinity resin composed of PSD-95 PDZ2 attached to a solid support (FIG. 36 covers the reverse, POI-PSD-95 PDZ2 fusion proteins purified on NR2B PSD-95 PDZ2 domain binding peptide resin). PDZ binding C-terminal peptide PDZ affinity chromatography tags (e.g. Type A) have been shown to function well as affinity tags when fused to easily purified MBP or GST proteins. PDZ binding C-terminal peptide PDZ affinity chromatography tags (e.g. Type A) can also function when fused to more difficult to purify proteins, such as DiHydroFolate Reductase (DHFR), Dasher Green Fluorescent Protein (GFP), Beta Galactosidase (LacZ) and Chloramphenicol Acetyl Transferase (CAT).

When DHFR, CAT, LacZ and Dasher GFP proteins were tagged with Type A PDZ affinity tags (the seven C-terminal amino acids of the NR2B tail) and incubated with PSD-95 PDZ2 coupled to a solid support, they were all purified to more than 95% homogeneity when assessed by SDS-PAGE. Interestingly, yields of purified protein were directly proportional to expression level, suggesting PDZ affinity resin was not saturated under the current conditions. Purified proteins were then tested for enzymatic activity to confirm the effects of fusing PDZ affinity tags to DHFR, CAT, LacZ and Dasher GFP proteins.

For the experiment, fusion proteins containing DHFR, Dasher GFP, CAT or LacZ in frame with PDZ domain binding C-terminal peptides (e.g. Type A PDZ affinity tags) were expressed in BL21(DE3) E. coli cells. Single colonies of BL21(DE3) cells harboring pJExpress414 plasmids containing the genes of interest were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing fusions of functional proteins (DasherGFP, LacZ, and CAT) to PDZ domain binding C-terminal peptides (e.g. Type A PDZ affinity tags) were resuspended in 5 ml/g cell-paste Purification Buffer (50 mM HEPES, pH 7.5, 150 mM NaCl, 5 mM TCEP, 0.5 mM EDTA, 10 mM MgCl2, 2 mM PMSF, Complete Protease Inhibitor) supplemented with 25 U/ml Benzonase and 10 U/ml ReadyLyse. The resuspended cells were sonicated (Branson, Danbury Conn.) 2× for 90 s/pass (15% power, 1.0 s on, 1.5 s off) before clarification by centrifugation at 16,000×g for 30 min at 4° C. Clarified lysate (1.8-14 ml) containing POIs was added to 100-200 ul of PSD-95 PDZ2 PDZ affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. The concentration of the eluted protein could be increased by decreasing the volume of buffer added for elution, increasing the concentration of peptide from 400 to 800 ug/ml, and/or by incubating the column resin with Peptide Elution Buffer without flow for 20 min before collecting the eluate.

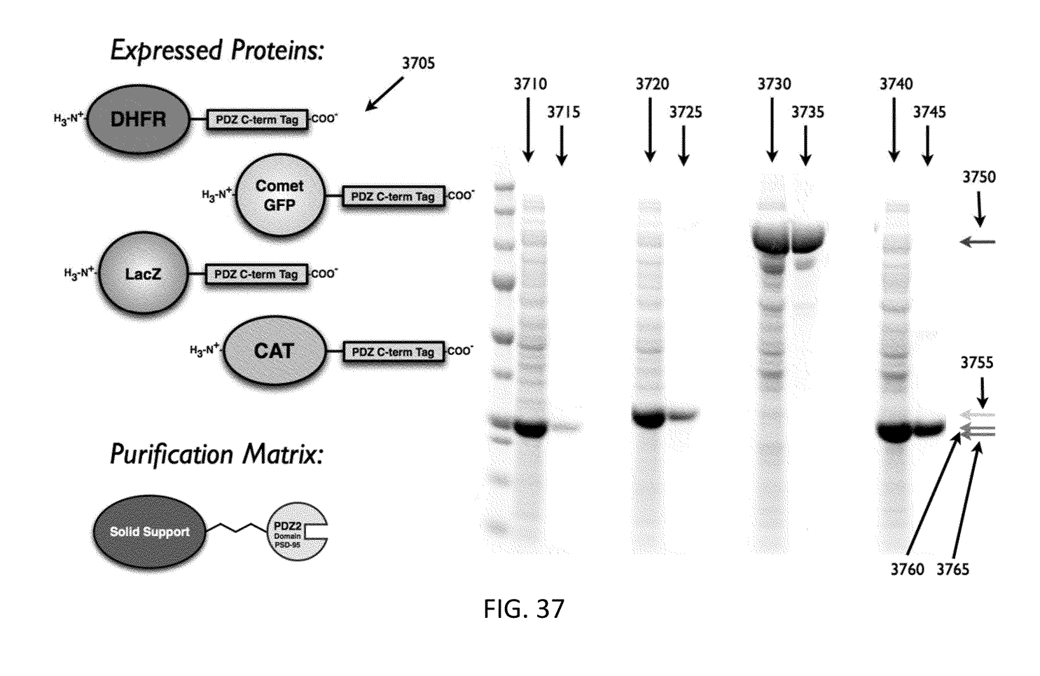

In FIG. 37, the expressed proteins are DHFR, Dasher GFP, LacZ and CAT (3705) and the purification matrix is PSD-95 PDZ2 domain coupled to a solid support. A GelCode Blue stained 4-12% bis-tris SDS-PAGE gel was used. The columns on the gel in FIG. 37 are: DHFR soluble protein (3710) and peptide eluate (3715); Dasher GFP soluble protein (3720) and peptide eluate (3725); LacZ soluble protein (3730) and peptide eluate (3735); CAT soluble protein (3740) and peptide eluate (3745). The arrows relate to DHFR (3765), Dasher GFP (3755), LacZ (3750), and CAT (3760). As shown, yields of POI were directly related to their expression level and were purified as such to high levels of purity.

### Example 14

### PSD-95 PDZ2 Affinity Chromatography Purification of DHFR, Dasher GFP, LacZ and CAT Fusion Proteins Containing N-Terminal 1× nNOS PDZ-BH Domains (Type B PDZ Affinity Tags) Heterologously Expressed in E. coli

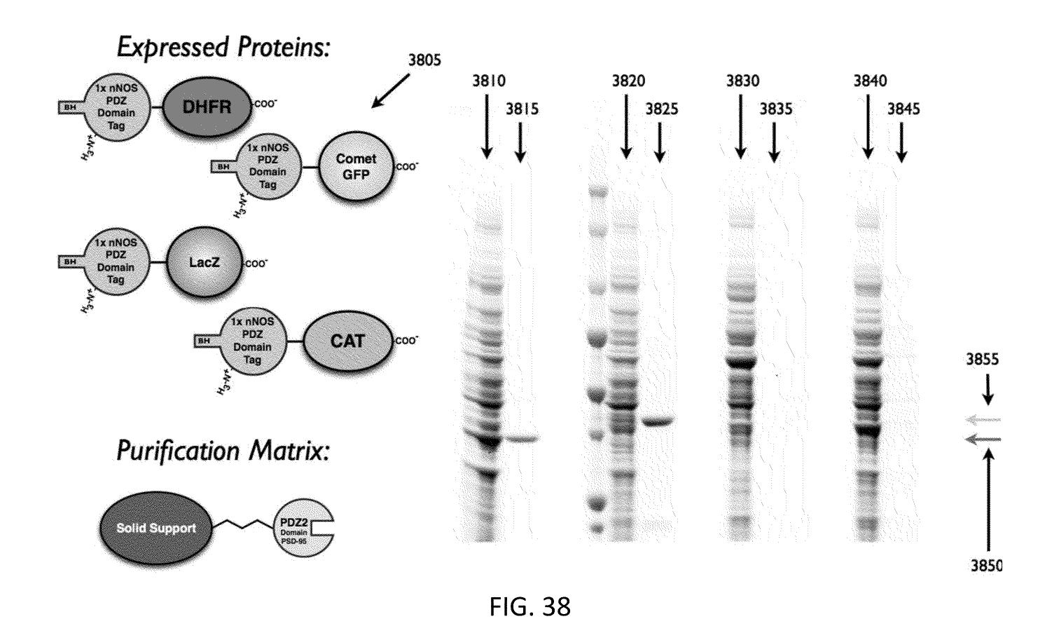

The nNOS PDZ domain (Amino Acids 14-125 of nNOS) is known in the art to bind both the syntrophin PDZ domain and the first two PDZ domains from PSD-95 (PDZ1 and PDZ2) in vivo and in vitro through the use of its C-terminal B-hairpin motif. Data in FIGS. 29 and 33-35 has shown that when 1× or 2× nNOS PDZ-BH domains in tandem are appended to a Protein of Interest (POI), the POI can be purified using a PDZ affinity resin composed of PSD-95 PDZ2 attached to a solid support. N-terminal, C-terminal or internal 1× and 2× nNOS PDZ-BH Domain PDZ affinity chromatography tags (Type B) have been shown to function well as affinity tags when fused to easily purified MBP or GST proteins. FIG. 38 relates to how the 1× nNOS PDZ-BH Domain PDZ affinity chromatography tags (Type B) function when fused to more difficult to purify proteins, such as DiHydroFolate Reductase (DHFR), Dasher Green Fluorescent Protein (GFP), Beta Galactosidase (LacZ) and Chloramphenicol Acetyl Transferase (CAT).

As shown in the experimental, when DHFR, CAT, LacZ and Dasher GFP proteins were tagged with Type B PDZ affinity tags (1× nNOS PDZ-BH) and incubated with PSD-95 PDZ2 coupled to a solid support, DHFR and Dasher GFP were purified to more than 95% homogeneity when assessed by SDS-PAGE, whereas LacZ and CAT fused to Type B PDZ affinity tags were unable to be purified. Purified Dasher GFP proteins were then tested for fluorescence to confirm the effects of fusing Type B PDZ affinity tags on their activity. The difficulty in purifying LacZ and CAT when fused to 1× nNOS PDZ-BH (Type B PDZ affinity tags) likely stems from occlusion of the BH domain by the LacZ and CAT proteins.

For the experiment, fusion proteins containing DHFR, Dasher GFP, CAT or LacZ in frame with N-terminal 1× nNOS PDZ-BH Domain PDZ affinity chromatography tags (e.g. Type B PDZ affinity tags) were expressed in BL21(DE3) E. coli cells. Single colonies of BL21(DE3) cells harboring pJExpress414 plasmids containing genes of interest were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing fusions of functional proteins (DasherGFP, LacZ, and CAT) to N-terminal 1× nNOS PDZ-BH Domain PDZ affinity chromatography tags (e.g. Type B PDZ affinity tags) were resuspended in 5 ml/g cell-paste Purification Buffer (50 mM HEPES, pH 7.5, 150 mM NaCl, 5 mM TCEP, 0.5 mM EDTA, 10 mM MgCl2, 2 mM PMSF, Complete Protease Inhibitor) supplemented with 25 U/ml Benzonase and 10 U/ml ReadyLyse. The resuspended cells were sonicated (Branson, Danbury Conn.) 2× for 90 s/pass (15% power, 1.0 s on, 1.5 s off) before clarification by centrifugation at 16,000×g for 30 min at 4° C. Clarified lysate (1.8-14 ml) containing POIs was added to 100-200 ul of PSD-95 PDZ2 PDZ affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. The concentration of the eluted protein could be increased by decreasing the volume of buffer added for elution, increasing the concentration of peptide from 400 to 800 ug/ml, and/or by incubating the column resin with Peptide Elution Buffer without flow for 20 min before collecting the eluate.

In FIG. 38, the expressed proteins are DHFR, Dasher GFP, LacZ and CAT fused to N-terminal 1× nNOS PDZ-BH Domain PDZ affinity chromatography tags (e.g. Type B PDZ affinity tags) (3805) and the purification matrix is PSD-95 PDZ2 domain coupled to a solid support. A GelCode Blue stained 4-12% bis-tris SDS-PAGE gel was used. The columns on the gel in FIG. 38 are: DHFR soluble protein (3810) and peptide eluate (3815); Dasher GFP soluble protein (3820) and peptide eluate (3825); LacZ soluble protein (3830) and peptide eluate (3835); CAT soluble protein (3840) and peptide eluate (3845). The arrows relate to DHFR (3850) and Dasher GFP (3855). As shown, DHFR and Dasher GFP were purified to a high level of purity (>95%)

### Example 15

### PSD-95 PDZ2 Affinity Chromatography Purification of DHFR, Dasher GFP, LacZ and CAT Fusion Proteins Containing N-Terminal 2× nNOS PDZ-BH Domains (Type B PDZ Affinity Tags) Heterologously Expressed in E. coli

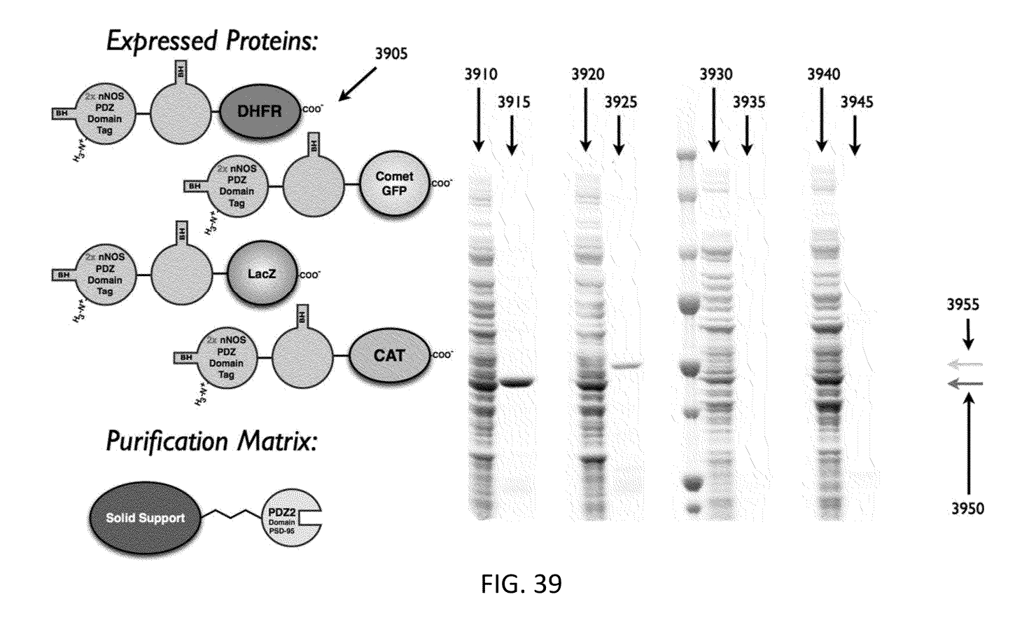

The nNOS PDZ domain (Amino Acids 14-125 of nNOS) is known in the art to bind both the syntrophin PDZ domain and the first two PDZ domains from PSD-95 (PDZ1 and PDZ2) in vivo and in vitro through the use of its C-terminal B-hairpin motif. Data in FIGS. 29 and 33-35 has shown that when 1× or 2× nNOS PDZ-BH domains in tandem are appended to a Protein of Interest (POI), the POI can be purified using a PDZ affinity resin composed of PSD-95 PDZ2 attached to a solid support. N-terminal, C-terminal or internal 1× and 2× nNOS PDZ-BH Domain PDZ affinity chromatography tags (Type B) have been shown to function well as affinity tags when fused to easily purified MBP or GST proteins. FIG. 39 relates to how the 2× nNOS PDZ-BH Domain PDZ affinity chromatography tags (Type B) function when fused to more difficult to purify proteins, such as DiHydroFolate Reductase (DHFR), Dasher Green Fluorescent Protein (GFP), Beta Galactosidase (LacZ) and Chloramphenicol Acetyl Transferase (CAT).

As shown in the experimental, when DHFR, CAT, LacZ and Dasher GFP proteins were tagged with Type B PDZ affinity tags (2× nNOS PDZ-BH) and incubated with PSD-95 PDZ2 coupled to a solid support, DHFR and Dasher GFP were purified to more than 95% homogeneity when assessed by SDS-PAGE, whereas LacZ and CAT fused to Type B PDZ affinity tags were unable to be purified. Purified Dasher GFP proteins were then tested for fluorescence to confirm the effects of fusing Type B PDZ affinity tags on their activity. The difficulty in purifying LacZ and CAT when fused to 2× nNOS PDZ-BH (Type B PDZ affinity tags) likely stems from occlusion of the BH domain by the LacZ and CAT proteins.

For the experiment, fusion proteins containing DHFR, Dasher GFP, CAT or LacZ in frame with N-terminal 2× nNOS PDZ-BH Domain PDZ affinity chromatography tags (e.g. Type B PDZ affinity tags) were expressed in BL21(DE3) E. coli cells. Single colonies of BL21(DE3) cells harboring pJExpress414 plasmids containing genes of interest were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing fusions of functional proteins (DasherGFP, LacZ, and CAT) to N-terminal 2× nNOS PDZ-BH Domain PDZ affinity chromatography tags (e.g. Type B PDZ affinity tags) were resuspended in 5 ml/g cell-paste Purification Buffer (50 mM HEPES, pH 7.5, 150 mM NaCl, 5 mM TCEP, 0.5 mM EDTA, 10 mM MgCl2, 2 mM PMSF, Complete Protease Inhibitor) supplemented with 25 U/ml Benzonase and 10 U/ml ReadyLyse. The resuspended cells were sonicated (Branson, Danbury Conn.) 2× for 90 s/pass (15% power, 1.0 s on, 1.5 s off) before clarification by centrifugation at 16,000×g for 30 min at 4° C. Clarified lysate (1.8-14 ml) containing POIs was added to 100-200 ul of PSD-95 PDZ2 PDZ affinity chromatography resin pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. The concentration of the eluted protein could be increased by decreasing the volume of buffer added for elution, increasing the concentration of peptide from 400 to 800 ug/ml, and/or by incubating the column resin with Peptide Elution Buffer without flow for 20 min before collecting the eluate.

In FIG. 39, the expressed proteins are DHFR, Dasher GFP, LacZ and CAT fused to N-terminal 2× nNOS PDZ-BH Domain PDZ affinity chromatography tags (e.g. Type B PDZ affinity tags) (3905) and the purification matrix is PSD-95 PDZ2 domain coupled to a solid support. A GelCode Blue stained 4-12% bis-tris SDS-PAGE gel was used. The columns on the gel in FIG. 39 are: DHFR soluble protein (3910) and peptide eluate (3915); Dasher GFP soluble protein (3920) and peptide eluate (3925); LacZ soluble protein (3930) and peptide eluate (3935); CAT soluble protein (3940) and peptide eluate (3945). The arrows relate to DHFR (3950) and Dasher GFP (3955). As shown, DHFR and Dasher GFP were purified to 95% homogeneity, while LacZ and CAT fused to Type B PDZ affinity tags were unable to be purified.

### Example 16

### PSD-95 PDZ2 Affinity Chromatography Purification of DHFR, Dasher GFP, LacZ and CAT Fusion Proteins Containing N-Terminal 2× nNOS PDZ-BH Domains (Type B PDZ Affinity Tags) Heterologously Expressed in E. coli

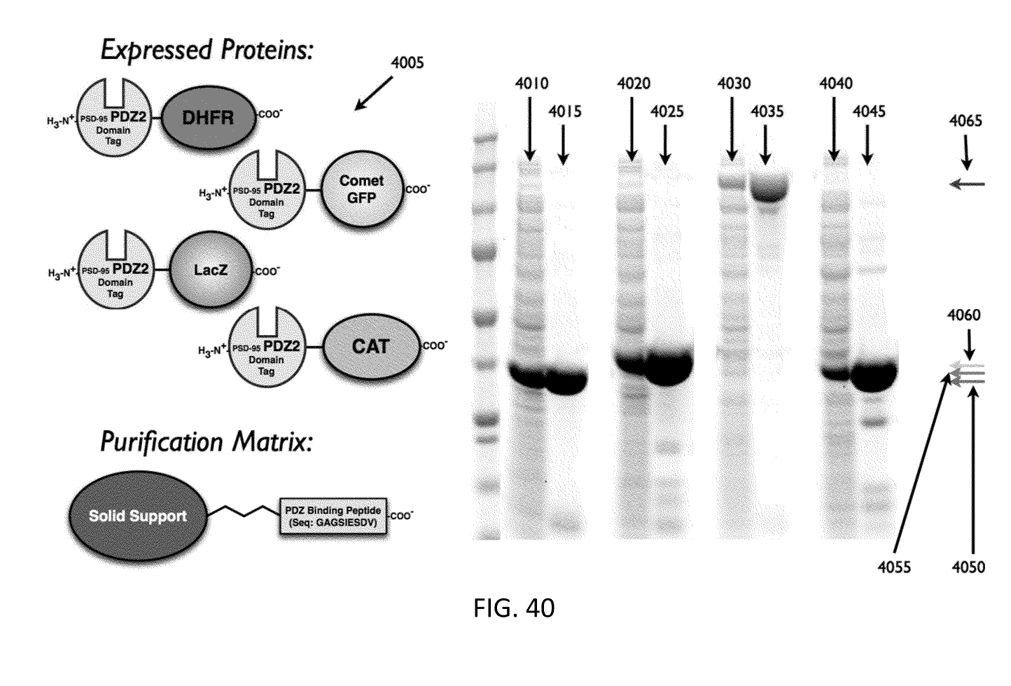

The C-terminal six amino acids (SIESDV) of the N-methyl-D-aspartate (NMDAR) receptor subunit, NR2B, are known in the art to form a high affinity (K_{D} of 9.7 and 1.3 uM, respectively) interaction with the PDZ1 and PDZ2 domains of PSD-95, in vitro and in vivo. Data in FIGS. 30-32 has shown that when the six C-terminal amino acids of NR2B are appended to a Protein of Interest (POI), the POI can be purified using a PDZ affinity resin composed of PSD-95 PDZ2 attached to a solid support. FIG. 36 covers the reverse, in which POI-PSD-95 PDZ2 (Type C Tags) fusion proteins are purified on NR2B PSD-95 PDZ2 domain binding peptide resin. N-terminal, C-terminal or internal PSD-95 PDZ2 domain PDZ affinity chromatography tags (Type C) have been shown to function well as affinity tags when fused to easily purified MBP or GST proteins. FIG. 40 relates to how the PSD-95 PDZ2 Domain PDZ affinity chromatography tags (Type C) function when fused to more difficult to purify proteins, such as DiHydroFolate Reductase (DHFR), Dasher Green Fluorescent Protein (GFP), Beta Galactosidase (LacZ) and Chloramphenicol Acetyl Transferase (CAT).

When DHFR, CAT, LacZ and Dasher GFP proteins were tagged with N-terminal Type C PDZ affinity tags (PSD-95 PDZ2 domains) and incubated with seven C-terminal amino acids of the NR2B Tail coupled to a solid support, they were all purified to more than 95% homogeneity when assessed by SDS-PAGE. Interestingly, yields of purified protein were not directly proportional to expression level, suggesting PDZ affinity resin was saturated under the current conditions. Purified proteins were then tested for enzymatic activity to confirm the affects of fusing PDZ affinity tags to CAT, LacZ and Dasher GFP proteins.

For the experiment, fusion proteins containing DHFR, Dasher GFP, CAT or LacZ in frame with N-terminal PSD-95 PDZ2 PDZ affinity chromatography tags (e.g. Type C PDZ affinity tags) were expressed in BL21(DE3) E. coli cells (3905). Single colonies of BL21(DE3) cells harboring pJExpress414 plasmids containing genes of interest were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing fusions of functional proteins (DasherGFP, LacZ, and CAT) to N-terminal PSD-95 PDZ2 PDZ affinity chromatography tags (e.g. Type C PDZ affinity tags) were resuspended in 5 mug cell-paste Purification Buffer (50 mM HEPES, pH 7.5, 150 mM NaCl, 5 mM TCEP, 0.5 mM EDTA, 10 mM MgCl2, 2 mM PMSF, Complete Protease Inhibitor) supplemented with 25 U/ml Benzonase and 10 U/ml ReadyLyse. The resuspended cells were sonicated (Branson, Danbury Conn.) 2× for 90 s/pass (15% power, 1.0 s on, 1.5 s off) before clarification by centrifugation at 16,000×g for 30 min at 4° C. Clarified lysate (1.8-14 ml) containing POIs was added to 100-200 ul of C-terminal PDZ domain binding peptide (H3N+-GAGSSIESDV-COO—) coupled to a solid support pre-equilibrated with Purification Buffer, and the suspension was agitated at 4° C. for 1 h on an end-over-end mixer to allow the protein to bind to the affinity resin. The mixtures were then subjected to centrifugation at 1000×g for 2 min, and the supernatant was removed by pipetting. The affinity resin with bound protein was then resuspended in 2 column volumes of Purification Buffer, transferred to a BioSpin chromatography column (Cat. No. 732-6008, BioRad, Hercules, Calif.), capped, and allowed to settle for 15 min. The cap was removed and the resin washed with 20 column volumes of Purification Buffer. Bound protein was eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. The concentration of the eluted protein could be increased by decreasing the volume of buffer added for elution, increasing the concentration of peptide from 400 to 800 ug/ml, and/or by incubating the column resin with Peptide Elution Buffer without flow for 20 min before collecting the eluate.

In FIG. 40, the expressed proteins are DHFR, Dasher GFP, LacZ and CAT fused to N-terminal PSD-95 PDZ2 domains (e.g. Type C PDZ affinity tags) (4005) and the purification matrix is PDZ binding peptide (GAGSSIESDV) coupled to a solid support. A GelCode Blue stained 4-12% bis-tris SDS-PAGE gel was used. The columns on the gel in FIG. 40 are: DHFR soluble protein (4010) and peptide eluate (4015); Dasher GFP soluble protein (4020) and peptide eluate (4025); LacZ soluble protein (4030) and peptide eluate (4035); CAT soluble protein (4040) and peptide eluate (4045). The arrows relate to DHFR (4050), Dasher GFP (4060), LacZ (4065), and CAT (4055). As shown, all proteins were purified to 95% homogeneity.

### Example 17

### Function of Dasher GFP

Green fluorescent protein (GFP) is a 238 residue protein isolated from the Pacific Northwest jellyfish, Aequeorea victoria (59). GFP transmutes blue chemiluminescence from a primary photoprotein (aequorin) into green fluorescence (60), utilizing a p-hydroxybenzylidene-imidazolidone chromophore derived from its S65, Y66 and G67 residues (61). It has been used in the production of biosensors for monitoring intracellular pH (62-64), calcium concentration (65), redox potential (66,67), membrane potential (68) and temperature (69). Proper folding of GFP around the chromophore is necessary for fluorescence, as evidenced by the fact that synthetic p-hydroxybenzylidene-imidazolidone chromophores are devoid of fluorescence (70). When fused to the C-terminus of a POI, productive folding of the downstream GFP and formation of the fluorescent chromophore have been shown to depend on the robustness of folding of the upstream protein (71). DasherGFP is a 26.6 kDa, synthetic, non-aequorea fluorescent protein, developed by DNA2.0, with excitation and emission wavelengths of 505 and 525 nm, respectively, and was used to verify the affect of PDZ Affinity Tags on protein folding.

The integrity of the purified DasherGFP fusion protein's fluorophore was assayed on a Hitachi F-4500 FL Fluorescence Spectrometer (Tokyo, Japan) at 22° C. Excitation (300-600 nm) and emission (350-700 nm) spectra were collected to verify fluorophore activity. The fraction of properly folded DasherGFP fusion proteins was measured by comparing the emitted fluorescence intensity at 520 nm of tagged, purified DasherGFP fusion proteins excited at 505 nm to that of an equimolar amount of untagged, purified DasherGFP.

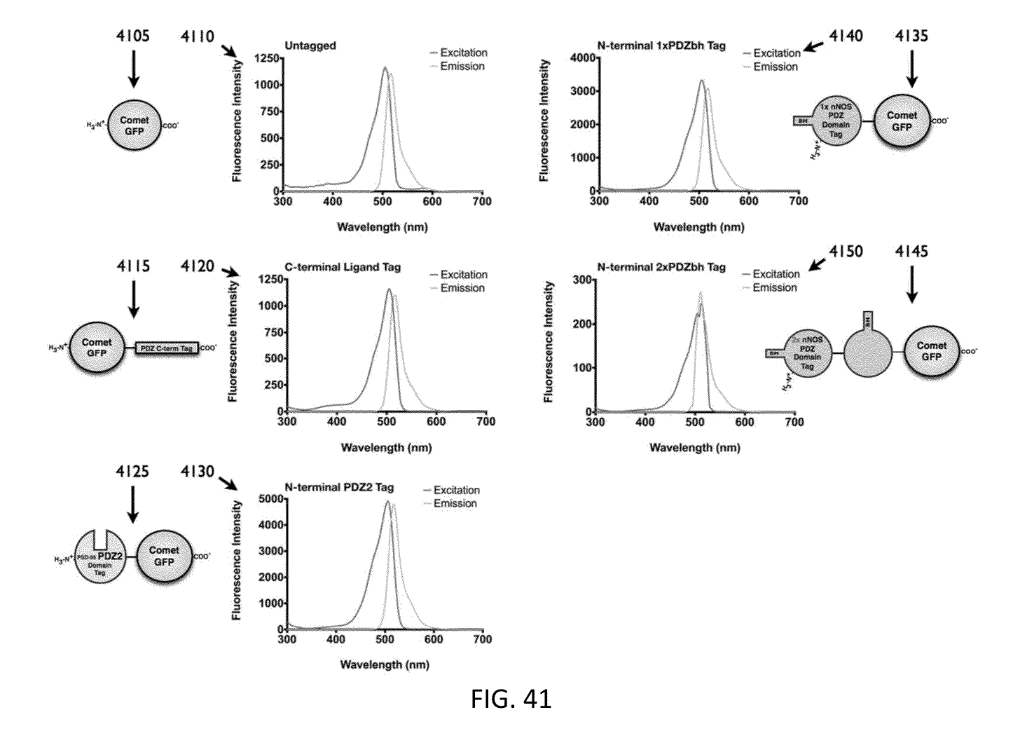

To verify that PDZ Affinity Tags do not inhibit the fluorescence emission of DasherGFP, excitation and emission wavelength scans were performed on each purified, PDZ Affinity Tagged DasherGFP construct (FIG. 41). Purified DasherGFP fused to a PDZ binding C-terminal peptide (Type A PDZ Affinity Tag; FIG. 41 (4115, 4120)), an N-terminal PDZ domain (Type C PDZ Affinity Tag; FIG. 41 (4125, 4130)), or a single N-terminal PDZ domain with a β-hairpin (Type B PDZ Affinity Tag; FIG. 41 (4135, 4140)) exhibited fluorescence excitation and emission spectra that are virtually identical to the unlabeled protein (FIG. 41 (4105, 4110)). In contrast, DasherGFP fused to an N-terminal tandem PDZ domain with a β-hairpin (Type B PDZ Affinity Tag; FIG. 41 (4145, 4150)) had greater overlap between the two spectra than the unlabeled protein with excitation and emission maxima both occurring at 510 nm. To estimate the percentage of folded and functional tagged DasherGFP in each purified sample, the fluorescence intensity at 520 nm of each tagged DasherGFP was compared to that of an equimolar amount of untagged DasherGFP. The results are summarized in Table 5 in which a comparison of the fluorescence emission at 520 nm, relative to untagged DasherGFP, of equimolar amounts of purified DasherGFP fused to PDZ Affinity Tags (Type A, B and C PDZ Affinity Tags) is illustrated.

TABLE 5
Functional analysis of DasherGFP and
purified DasherGFP fusion proteins

| | Relative Fluorescence |
|---|---|
| Affinity Tag | at 520 nm (%) |
| C-terminal PDZ Binding Peptide Tag | 99.2 |
| N-terminal PSD-95 PDZ2 Domain Tag | 97.5 |
| N-terminal 1x nNOS PDZ Domain + Beta | 90.1 |
| Hairpin Tag | |
| N-terminal 2x nNOS PDZ Domain + Beta | 0.3 |
| Hairpin Tag | |

Fluorescence emission at 520 nm, relative to untagged DasherGFP, of equimolar amounts of purified DasherGFP fused to a C-terminal PDZ2 domain peptide ligand, an N-terminal PDZ2 domain, and single or tandem PDZbh domains.

The fusions with a C-terminal PDZ domain protein Ligand, an N-terminal PDZ Domain, or a single N-terminal PDZbh Domain all exhibited relative fluorescence intensities of greater than 90%, indicating that they are almost entirely folded and functional. However, the fusion with tandem PDZbh Domain Tags was almost entirely misfolded (Table 5), again suggesting that the tandem PDZbh Domain Affinity Tag destabilized proper folding.

### Example 18

### Function of Beta Galactosidase (LacZ)

β-Galactosidase (LacZ) is a 1024 residue protein isolated from E. coli (72,73). It catalyzes the cleavage of the bond between the anomeric carbon and glycosyl oxygen of a β-D-galactopyranoside (74). In vivo LacZ catalyzes the cleavage of the disaccharide lactose to form glucose and galactose (75). It is often used experimentally as a reporter of gene expression, of spontaneous or directed genetic changes in coding sequences, and of protein-protein interactions because of its activity in myriad cell lines (76-79), the availability of an array of substrates, inducers and inhibitors (80-82), its structural malleability (83-85) and the large dynamic range of its gene expression (86).

The activity of purified LacZ fusion proteins was assayed by measuring the rate of hydrolysis of ONPG on a VERSAmax tunable microplate reader at 20° C. as described in (135). Briefly, the rate of ON release after addition of LacZ, produced by hydrolysis of ONPG, was measured by continuous monitoring of absorbance at 420 nm. Initial rates of ONPG hydrolysis were entered into Prism (v6.0d, GraphPad Software, La Jolla Calif.), plotted against ONPG substrate concentrations and analyzed by nonlinear regression using the Michaelis-Menten equation to calculate k_{cat} and K_{M} values. These values, calculated for purified LacZ fusion proteins, were compared to values from the literature (136) by ordinary one way ANOVA (Uncorrected Fisher's LSD) in Prism. The purified LacZ fusion proteins were prepared for assay by buffer exchange in an Amicon Ultra-0.5 ml concentrator into 50 mM Tris, pH 7.5, 100 mM NaCl, 10 mM MgCl₂, 0.01 mM MnCl₂ and 100 mM β-mercaptoethanol (137). Aliquots were flash frozen in liquid nitrogen and stored at −80° C.

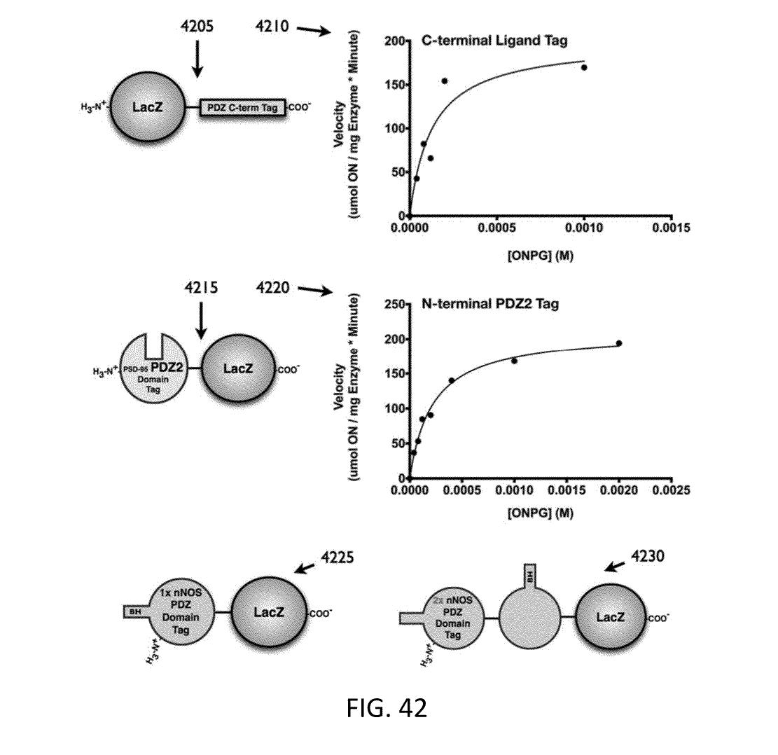

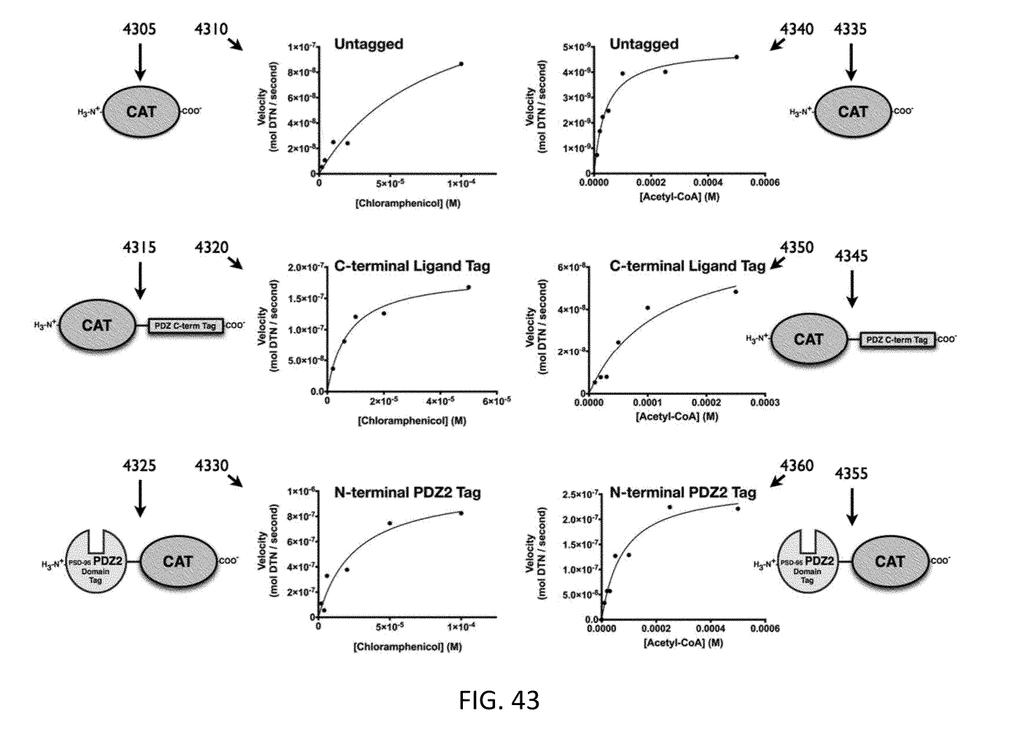

To verify that PDZ Affinity Tags do not inhibit the enzyme activity of LacZ, each tagged LacZ was assayed using ONPG as a substrate, as described in Methods (FIG. 42). The catalytic constants (k_{cat}=Vₘₐₓ/[E]) of LacZ fused to a PDZ binding C-terminal peptide (Type A PDZ Affinity Tag; FIG. 42 (4205, 4210)), or an N-terminal PDZ Domain (Type C PDZ Affinity Tag; FIG. 42 (4215, 4220)), were in good agreement with the published k, for untagged LacZ under identical conditions.

In particular Table 6 reports a comparison of the Michaelis-Menten constants for rates of o-nitrophenol-beta-galactoside (ONPG) hydrolysis by purified LacZ and Type A and C PDZ Affinity Tagged LacZ fusion proteins.

TABLE 6
Michaelis-Menten constants of LacZ
and purified LacZ fusion proteins

| | V_{MAX} (umol | |
|---|---|---|
| | ONPG/mg | K_{M} |
| Affinity Tag | protein*min) | (uM) |
| None | 178* | 161* |
| C-terminal PDZ Binding Peptide Tag | 194 ± 18.3 | 135 ± 44 |
| N-terminal PSD-95 PDZ2 Domain Tag | 198.4 ± 4.73 | 227 ± 21 |

| | |
|---|---|
| N-terminal 1x nNOS PDZ Domain + | Unable to be Purified |
| Beta Hairpin Tag | |
| N-terminal 2x nNOS PDZ Domain + | |
| Beta Hairpin Tag | |

Comparison of k_{cat} and K_{M} values for LacZ and purified LacZ fused to a PDZ binding C-terminal peptide or an N-terminal PDZ domain.
*Wallenfels K. & Malhotra P. (1962) Advanced Carbohydrate Chemistry: 239-298 (138)

The K_{M}'s for ONPG of these two tagged versions of LacZ were also in the same range as those published for untagged LacZ (Table 6). When compared by ordinary one way ANOVA, the k_{cal} (p≧0.16) and K_{M} (p≧0.21) values of tagged LacZ were not significantly different from those of untagged LacZ. Thus, LacZ engineered to contain either of these affinity tags and purified by PDZ Affinity Chromatography is folded and functional. LacZ fused N-terminally to single or tandem nNOS PDZ domain tags with 13-Hairpins (Type B PDZ Affinity Tags) could not be purified by Affinity Chromatography and so were not assayed.

### Example 19

### Function of Chloramphenicol Acetyl Transferase (CAT)

Chloramphenicol acetyltransferase (CAT) is a ˜219 residue protein isolated from E. coli and S. aureus. It catalyzes the inactivation of the antibiotic chloramphenicol, by acylating chloramphenicol in the presence of acetyl-CoA to produce chloramphenicol-3-acetate and reduced CoA (87,88). It has been used extensively as an in vitro reporter of gene expression levels in eukaryotic cell lines because of its stability, absence of competing activities in eukaryotic cells, ease of use and sensitivity (89-91).

The activity of purified CAT fusion proteins was determined by measuring the rate of production of 5-thio-2-nitrobenzoic acid (DTN) resulting from the reduction of 5,5′-dithiobis-2-nitrobenzoic acid (DTNB) by free CoA generated during acylation of chloramphenicol. The rate of reduction was measured by continuously monitoring absorbance at 412 nm after addition of CAT on a VERSAmax tunable microplate reader at 37° C. using a DTNB reduction assay described in (139). Initial rates of chloramphenicol acylation were entered into Prism (v6.0d; GraphPad Software, La Jolla Calif.), plotted against acetyl-CoA or chloramphenicol substrate concentrations and analyzed by nonlinear regression using the Michaelis-Menten equation to calculate k_{cal} and K_{M} values. These values, calculated for purified CAT fusion proteins and for equimolar amounts of untagged CAT (Sigma), were compared by ordinary one way ANOVA (Uncorrected Fisher's LSD) in Prism. The activity of purified CAT fusion proteins and equimolar amounts of untagged CAT (Sigma) were compared. CAT fusion proteins were prepared for assay by buffer exchange in an Amicon Ultra-0.5 ml concentrator into 10 mM Tris, pH 7.8, 200 mM NaCl, 0.2 mM chloramphenicol, 0.1 mM β-mercaptoethanol (140). Aliquots were flash frozen with liquid nitrogen and stored at −80° C.

To verify that PDZ Affinity Tags do not inhibit the enzymatic activity of CAT, measured activity of untagged CAT was measured and each tagged construct using chloramphenicol and acetyl-CoA as substrates, as described in Methods (FIG. 42). CAT fused to a PDZ binding C-terminal peptide (Type A PDZ Affinity Tag; FIG. 42 (4315, 4320)), or an N-terminal PDZ domain (Type C PDZ Affinity Tag; FIG. 42 (4325, 4330)) were assayed with a fixed concentration of acetyl-CoA (500 μM) and 0-100 μM chloramphenicol. Their measured catalytic constants were in good agreement with the k_{cat}'s of untagged CAT purchased from Sigma Aldrich (FIG. 42 (4305, 4310)).

In particular, Table 7 compares the Michaelis-Menten constants for rates of 5,5′-dithiobis-2-nitrobenzoic acid (DTNB) reduction by purified CAT and Type A and C PDZ Affinity Tagged CAT fusion proteins.

TABLE 7
Michaelis-Menten constants of CAT
and purified CAT fusion proteins

| | Chloramphenicol** | | Acetyl-CoA*** | |
|---|---|---|---|---|
| | k_{cat} | K_{M} | k_{cat} | K_{M} |
| | (sec⁻¹) | (uM) | (sec⁻¹) | (uM) |
| None | 0.43 ± 0.11 | 80 ± 40 | 0.085 ± 0.004 | 41 ± 7 |
| C-terminal PDZ | 0.37 ± 0.03 | 8 ± 2 | 0.074 ± 0.02 | 120 ± 50 |
| Binding Peptide Tag | | | | |
| N-terminal PSD-95 | 0.37 ± 0.06 | 26 ± 10 | 0.093 ± 0.008 | 78 ± 20 |
| PDZ2 Domain Tag | | | | |

| | |
|---|---|
| N-terminal 1x | Unable to be Purified |
| nNOS PDZ | |
| Domain + | |
| Beta Hairpin Tag | |
| N-terminal 2x | |
| nNOS PDZ | |
| Domain + | |
| Beta Hairpin Tag | |

Comparison of k_{cat} and K_{M} values for CAT and purified CAT fused to a PDZ binding C-terminal peptide or an N-terminal PDZ domain.
* CAT was purchased from Sigma Aldrich (Cat. No. C8413, St. Louis, MO).
**Chloramphenicol concentration ranged from 0 to 100 uM. Acetyl-CoA concentration was fixed at 500 uM.
***Acetyl-CoA concentration ranged from 0 to 500 uM. Chloramphenicol concentration was fixed at 100 uM.

The K_{M}'s for chloramphenicol of these two tagged versions of CAT are similar to those measured for untagged CAT (Table 7) and published values [10 μM; (139)]. When compared by ordinary one way ANOVA, the k_{cal} (p≧0.61) and K_{M} (p≧0.12) values of tagged CAT for chloramphenicol were not significantly different from those of untagged CAT.

When CAT fused to a PDZ binding C-terminal peptide (Type A PDZ Affinity Tag; FIG. 42 (4345, 4350)) or an N-terminal PDZ Domain (Type C PDZ Affinity Tag; FIG. 42 (4355, 4360)) were assayed with a fixed concentration of chloramphenciol (100 μM) and 0-500 μM acetyl-CoA, their catalytic constants were also in good agreement with the k_{cat}'s of untagged CAT purchased from Sigma (FIG. 42 (4335, 4340); Table 7) and published values [50 μM; (139)]. The K_{M}'s for acetyl-CoA of these two tagged versions of CAT were also similar to published values for untagged CAT (Table 7). When compared by ordinary one way ANOVA, the k_{cal} (p≧0.37) and K_{M} (p≧0.17) values of tagged CAT for acetyl-CoA were not significantly different from those of untagged CAT. Thus, CAT engineered to contain either of these affinity tags and purified by PDZ Domain Affinity Chromatography is folded and functional. As was the case for LacZ, CAT fused N-terminally to single or tandem PDZ domain with β-hairpin tags (Type B Tags) could not be purified by Affinity Chromatography and so were not assayed.

### Example 20

### Purification of PDZ2 Domain of PSD-95 and Synthesis of PDZ Domain-NHS-Agarose Affinity Resin

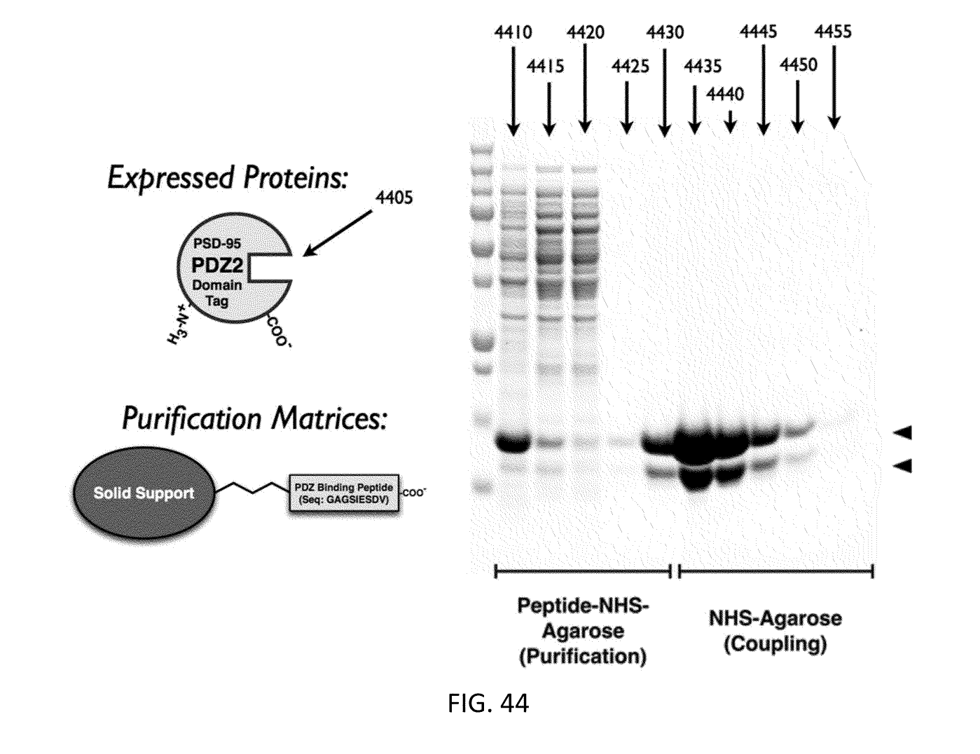

PDZ Affinity resin (PDZ domain coupled to a solid support) can be prepared using many different coupling chemistries and solid supports. Here, the purification of the PDZ2 domain of PSD-95 is described and the synthesis of 5 ml of PSD-95 PDZ2 domain-NHS-Agarose by purifying PSD-95 PDZ2 and subsequently coupling purified PSD-95 PDZ2 to Agarose activated by NHS (FIG. 44).

The PDZ2 domain of PSD-95 was expressed in E. coli and purified by chromatography on GluN2B ligand-NHS-Agarose Affinity Resin (synthesis described below in Example 21). To express PDZ2 of PSD-95, single colonies of BL21(DE3) cells harboring pJExpress414 plasmids containing a gene coding for the PDZ2 domain of PSD-95 were grown overnight at 37° C. in 5 ml of lysogeny broth (LB) (Cat. No. L9110, Teknova, Hollister, Calif.) supplemented with 100 ug/ml carbenicillin. Overnight cultures were diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (˜20 grams) containing recombinant PDZ2 domains were resuspended in 10 ml of Purification Buffer, supplemented with 25 U/ml Benzonase and 10 U/ml ReadyLyse, per gram of cells. Cells were evenly suspended in a Teflon-glass homogenizer and then lysed by three passes through a microfluidizer. The cell lysate was clarified by centrifugation at 30,000×g for 60 minutes at 4° C. The clarified lysate was added to 15 ml of settled GluN2B ligand-NHS-Agarose that had been pre-equilibrated in Purification Buffer, and the suspension was incubated for 1 hour at 4° C. with continuous agitation on an end-over-end mixer. Unbound protein was separated from the affinity resin by centrifugation at 2,000×g for 2 minutes at 4° C. The resin was resuspended in 1 column volume of Purification Buffer and transferred to a Glass Econo-Column Chromatography Column, capped, and allowed to settle for 60 minutes before the cap was removed. The resin was washed with 20 column volumes of Purification Buffer. The PDZ2 domain protein was eluted by application of 4 column volumes of Peptide Elution Buffer (50 mM HEPES, pH 7.5, 150 mM NaCl, 5 mM TCEP, Complete Protease Inhibitor, and 400 μg/ml SIESDV peptide). Aliquots (17.5 ml) of eluted PDZ2 domain protein were pipetted into 20 ml Pierce 9K Protein Concentrators (Cat. No. 89885A, Thermo Scientific, Rockford Ill.) which were then subjected to centrifugation at 3,000×g for 25 minutes at 4° C. in a swinging bucket rotor. The filtrate was discarded, and another 17.5 ml aliquot of eluted PDZ2 domain protein was added to the concentrators and subjected to centrifugation. These steps were repeated until the final total volume of PDZ2 domain protein was reduced to 10 ml. The concentrated PDZ2 domain pool (11.3 mg/ml, 723 μM) was subjected to 5 cycles of buffer exchange into Coupling/Wash Buffer lacking free peptide in preparation for coupling to NHS-Activated Agarose. The five cycles of buffer exchange also served to remove bound GluN2B peptide from the purified PDZ2 domains. Purified PDZ2 domains in Coupling/Wash Buffer (11.3 mg/ml, 723 μM) were coupled to NHS-Activated Agarose resin by the same procedure described for coupling of the GluN2B ligand to NHS-Activated Agarose resin. The density of PDZ2 domains on the resin was approximately 0.724 nmol protein/μl resin (724 μM; 9.4 mg/ml). No loss of binding capacity was observed after 5 cycles of elution, denaturation and renaturation or after storage for 6 months at 4° C.

As shown, FIG. 44 relates to the above experiment of the PDZ domain peptide ligand affinity chromatography purification of the PDZ2 domain of PSD-95 and the synthesis of 5 ml of PSD-95 PDZ2 domain-NHS-Agarose. In FIG. 44, the expressed protein is PSD-95 PDZ2 (4405) and the purification matrix is PDZ domain peptide ligand (GAGSSIESDV) coupled to a solid support. A GelCode Blue stained 4-12% bis-tris SDS-PAGE gel was used. The columns on the gel in FIG. 44 are: unclarified cell lysate (4410); soluble or clarified cell lysate (4415); unbound protein (4420); wash (4425); peptide eluate (4430), concentrated peptide eluate or NHS-Agarose load (4435), NHS-Agarose unbound (4440), NHS-Agarose wash 1 (4445, NHS-Agarose wash 2 (4450) and NHS-Agarose quenched (4455).

### Example 21

### Synthesis of NR2B (GluN2B) Ligand-NHS-Agarose Affinity Resin

PDZ Affinity resin (PDZ domain binding peptide coupled to a solid support) can be prepared by many means. Here, the synthesis of 10 ml of GluN2B ligand-NHS-Agarose is described by amine coupling NR2B (GluN2B) peptide ligand (GAGS SIESDV) to Agarose activated by NHS. Twenty ml of a 50% slurry of N-hydroxysuccinimide (NHS)-Activated Agarose Resin in acetone storage solution (Cat. No. 26200, Pierce, Rockford Ill.) was added to a 50 ml Falcon Tube. The storage solution was removed by centrifugation at 1,000×g for 1 minute. The 10 ml of settled NHS-Activated Agarose resin was washed twice with 3 column volumes of ultrapure water followed by centrifugation at 1,000×g for 1 minute. The NHS-Activated Agarose was then washed twice with 3 column volumes of Coupling/Wash Buffer (100 mM NaHPO₄, pH 7.2, 150 mM NaCl) followed by centrifugation at 1,000×g for 1 minute. Peptide Buffer (2.5 column volumes of 100 mM NaHPO₄, pH 7.2, 150 mM NaCl, 10 mg/ml GAGSSIESDV peptide) was added to the resin, and the mixture was incubated for 2 hours at room temperature with continuous agitation on an end-over-end mixer. The GluN2B ligand-NHS-Activated Agarose was pelleted by centrifugation at 1,000×g for 1 minute, and washed twice with 3 column volumes of Coupling/Wash Buffer followed by centrifugation at 1,000×g for 1 minute. The remaining active sites on the GluN2B ligand-NHS-Agarose were blocked by incubation with 3 column volumes of Quenching Buffer (1 M ethanolamine, pH 7.4) for 20 minutes at room temperature with end-over-end mixing. Quenching Buffer was removed by centrifugation at 1,000×g for 1 minute. GluN2B ligand-NHS-Agarose resin was then washed twice with 4 column volumes of Coupling/Wash Buffer separated by centrifugation at 1,000×g for 1 minute. For storage, the GluN2B ligand-NHS-Agarose was washed twice with 4 column volumes of Storage Buffer (100 mM NaHPO₄, pH 7.2, 150 mM NaCl, 0.05% NαN₃), pelleted by centrifugation at 1,000×g for 1 minute, resuspended in 1 column volume of Storage Buffer, and stored at 4° C. Coupling of peptide to NHS-Agarose and the ligand density of immobilized peptide were monitored by the 660 nm Protein Assay (Cat. No. 22660, Pierce, Rockford Ill.) (The NHS leaving group interferes with BCA assays and absorbance assays at 280 nm). Ligand densities varied from 20 to 26 nmol peptide/μl resin (20-26 mM), which represents coupling efficiencies of 74 to 96%, respectively. No loss of binding capacity was observed after 5 cycles of elution, denaturation and renaturation or after storage for 6 months at 4° C.

### Example 22 (Prophetic)

### C-Terminal PDZ Domain Binding Peptide Pull Down of a POI Fusion Protein Containing a C-Terminal PSD-95 PDZ2 Domain (e.g. Type C PDZ Affinity Tags) Heterologously Expressed in E. coli

The C-terminal six amino acids (SIESDV) of the N-methyl-D-aspartate (NMDAR) receptor subunit, NR2B, are known in the art to form a high affinity (K_{D} of 9.7 and 1.3 uM, respectively) interaction with the PDZ1 and PDZ2 domains of PSD-95, in vitro and in vivo. Data in FIGS. 30-32 has shown that when the six C-terminal amino acids of NR2B are appended to a Protein of Interest (POI), the POI can be purified using a PDZ affinity resin composed of PSD-95 PDZ2 attached to a solid support. FIG. 36 covers the reverse, in which POI-PSD-95 PDZ2 (Type C Tags) fusion proteins are purified on NR2B PSD-95 PDZ2 domain binding peptide resin. N-terminal, C-terminal or internal PSD-95 PDZ2 domain PDZ affinity chromatography tags (Type C) have been shown to function well as affinity tags when fused to easily purified MBP or GST proteins. FIG. 40 covered how the PSD-95 PDZ2 Domain PDZ affinity chromatography tags (Type C) functioned when fused to more difficult to purify proteins, such as DiHydroFolate Reductase (DHFR), Dasher Green Fluorescent Protein (GFP), Beta Galactosidase (LacZ) and Chloramphenicol Acetyl Transferase (CAT).

As shown in FIG. 17, a POI-PSD-95 PDZ2 (Type C Tags) fusion proteins can be expressed in a cell line and purified on NR2B PSD-95 PDZ2 domain binding peptide resin. As an alternative to purification, if a POI was tagged with C-terminal Type C PDZ affinity tags (e.g. PSD-95 PDZ2 domains) and expressed and incubated with seven C-terminal amino acids of the NR2B Tail coupled to a solid support, they were all purified to more than 95% homogeneity when assessed by SDS-PAGE. Interestingly, yields of purified protein were not directly proportional to expression level, suggesting PDZ affinity resin was saturated under the current conditions. Purified proteins were then tested for enzymatic activity to confirm the effects of fusing PDZ affinity tags to CAT, LacZ and Dasher GFP proteins.

For the experiment, fusion proteins containing a POI in frame with C-terminal PSD-95 PDZ2 PDZ affinity chromatography tags (e.g. Type C PDZ affinity tags) could be expressed in BL21(DE3) E. coli cells. Single colonies of BL21(DE3) cells harboring pJExpress414 plasmids containing genes of interest could be grown overnight at 37° C. in 5 ml of lysogeny broth (LB) (Cat. No. L9110, Teknova, Hollister, Calif.) supplemented with 100 ug/ml carbenicillin. Overnight cultures could be diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing fusions of the POI to a C-terminal PSD-95 PDZ2 PDZ affinity chromatography tags (e.g. Type C PDZ affinity tags) could be resuspended in 5 ml/g cell-paste Purification Buffer (50 mM HEPES, pH 7.5, 150 mM NaCl, 5 mM TCEP, 0.5 mM EDTA, 10 mM MgCl2, 2 mM PMSF, Complete Protease Inhibitor) supplemented with 25 U/ml Benzonase and 10 U/ml ReadyLyse. The resuspended cells could be sonicated (Branson, Danbury Conn.) 2× for 90 s/pass (15% power, 1.0 s on, 1.5 s off) before clarification by centrifugation at 16,000×g for 30 min at 4° C. Clarified lysate (1.8-14 ml) containing the POI fusion could be added to 100-200 ul of C-terminal PDZ domain binding peptide (H3N+-GAGSSIESDV-COO—) coupled to a solid support pre-equilibrated with Purification Buffer, and the suspension could be agitated at 4° C. for 1 h on an end-over-end mixer to allow the protein to bind to the affinity resin. The mixtures could then subjected to centrifugation at 1000×g for 2 min, and the supernatant removed by pipetting. The affinity resin with bound protein could then be resuspended in 2 column volumes of Purification Buffer, transferred to a BioSpin chromatography column (Cat. No. 732-6008, BioRad, Hercules, Calif.), capped, and allowed to settle for 15 min. The cap could be removed and the resin washed with 20 column volumes of Purification Buffer. Instead of eluting the bound POI-PSD-95 PDZ2 domain (e.g. Type C PDZ affinity tags) fusion protein from the resin as described for affinity purification, a mixture comprised of human embryonic kidney cell lysate could be added to 100-200 ul of the complex comprised of a POI-C-terminal PSD-95 PDZ2 domain (e.g. Type C PDZ affinity tags)-PDZ domain binding peptide (H3N+-GAGSSIESDV-COO—) coupled to a solid support, and the suspension could be agitated at 4° C. for 1 h on an end-over-end mixer to allow POI binding partners present in the cell lysate to bind to the POI-C-terminal PSD-95 PDZ2 domain (e.g. Type C PDZ affinity tags) fusion protein non-covalently affixed to the affinity resin. The mixtures could then subjected to centrifugation at 1000×g for 2 min, and the supernatant removed by pipetting. The affinity resin with POI-C-terminal PSD-95 PDZ2 domain (e.g. Type C PDZ affinity tags) fusion bound to POI binding partners could then be resuspended in 2 column volumes of Purification Buffer, transferred to a BioSpin chromatography column (Cat. No. 732-6008, BioRad, Hercules, Calif.), capped, and allowed to settle for 15 min. The cap could be removed and the resin washed with 20 column volumes of Purification Buffer. The POI and its binding partner(s) could then be eluted by application of 4 column volumes of Peptide Elution Buffer [50 mM HEPES, pH 7.5, 150 mM NaCl, 5 mM TCEP, Roche Complete Protease Inhibitor, and 400 ug/ml SIESDV peptide. The concentration of the eluted protein could be increased by decreasing the volume of buffer added for elution, increasing the concentration of peptide from 400 to 800 ug/ml, and/or by incubating the column resin with Peptide Elution Buffer without flow for 20 min before collecting the eluate. Samples could then be run on an SDS-PAGE gel and stained with Coomassie Blue to detect POI binding partners.

### Example 23 (Prophetic)

### C-Terminal PDZ Domain Binding Peptide Depletion of Contaminate PDZ Domains Present in a Mixture with a POI

The C-terminal six amino acids (SIESDV) of the N-methyl-D-aspartate (NMDAR) receptor subunit, NR2B, are known in the art to form a high affinity (K_{D} of 9.7 and 1.3 uM, respectively) interaction with the PDZ1 and PDZ2 domains of PSD-95, in vitro and in vivo. Data in FIGS. 30-32 has shown that when the six C-terminal amino acids of NR2B are appended to a Protein of Interest (POI), the POI can be purified using a PDZ affinity resin composed of PSD-95 PDZ2 attached to a solid support. FIG. 36 covers the reverse, in which POI-PSD-95 PDZ2 (Type C Tags) fusion proteins are purified on NR2B PSD-95 PDZ2 domain binding peptide resin. N-terminal, C-terminal or internal PSD-95 PDZ2 domain PDZ affinity chromatography tags (Type C) have been shown to function well as affinity tags when fused to easily purified MBP or GST proteins. FIG. 40 covered how the PSD-95 PDZ2 Domain PDZ affinity chromatography tags (Type C) functioned when fused to more difficult to purify proteins, such as DiHydroFolate Reductase (DHFR), Dasher Green Fluorescent Protein (GFP), Beta Galactosidase (LacZ) and Chloramphenicol Acetyl Transferase (CAT).

As shown in FIG. 36, a PSD-95 PDZ2 (Type C Tags) can be expressed in a cell line and purified on NR2B PSD-95 PDZ2 domain binding peptide resin. In addition to being used to purify POIs fused to PSD-95 PDZ2 (Type C Tags), PDZ domain binding peptide resin (e.g. NR2B PSD-95 PDZ2 domain binding peptide resin) could be used to bind contaminate PDZ domains present in a mixture with a POI. As a method to deplete substances in a mixture and purify a POI, free PDZ domains (Type C PDZ affinity tags; PSD-95 PDZ2 domains) and a POI present in a mixture can be incubated with PDZ domain binding peptide resin (e.g. NR2B PSD-95 PDZ2 domain binding peptide resin). Binding to the PDZ domain binding peptide resin could remove all of the contaminate PDZ domains in the mixture, leaving the POI that lacks affinity for PDZ binding peptides in solution, effectively purifying the POI by depleting free contaminate PDZ domains.

For the experiment, a resin comprised of multiple different PDZ domain binding peptides must first be prepared. PDZ Affinity resin (PDZ domain binding peptides coupled to a solid support) can be prepared by many means. Here, the synthesis of 10 ml of multi ligand-NHS-Agarose is described by amine coupling multiple PDZ domain binding peptide ligands (e.g. GAGSSIESDV from the NR2B (GluN2B) peptide ligand, GAGQEELII from cadherin-5, etc.) to Agarose activated by NHS. Twenty ml of a 50% slurry of N-hydroxysuccinimide (NHS)-Activated Agarose Resin in acetone storage solution (Cat. No. 26200, Pierce, Rockford Ill.) is added to a 50 ml Falcon Tube. The storage solution is removed by centrifugation at 1,000×g for 1 minute. The 10 ml of settled NHS-Activated Agarose resin is washed twice with 3 column volumes of ultrapure water followed by centrifugation at 1,000×g for 1 minute. The NHS-Activated Agarose is then washed twice with 3 column volumes of Coupling/Wash Buffer (100 mM NaHPO₄, pH 7.2, 150 mM NaCl) followed by centrifugation at 1,000×g for 1 minute. Peptide Buffer (2.5 column volumes of 100 mM NaHPO₄, pH 7.2, 150 mM NaCl, 10 mg/ml of each peptide (e.g. GAGSSIESDV peptide, GAGQEELII peptide, etc.) is added to the resin, and the mixture is incubated for 2 hours at room temperature with continuous agitation on an end-over-end mixer. The PDZ domain binding peptide ligand-NHS-Activated Agarose is pelleted by centrifugation at 1,000×g for 1 minute, and washed twice with 3 column volumes of Coupling/Wash Buffer followed by centrifugation at 1,000×g for 1 minute. The remaining active sites on the PDZ domain binding peptide ligand-NHS-Activated Agarose are blocked by incubation with 3 column volumes of Quenching Buffer (1 M ethanolamine, pH 7.4) for 20 minutes at room temperature with end-over-end mixing. Quenching Buffer is removed by centrifugation at 1,000×g for 1 minute. PDZ domain binding peptide ligand-NHS-Activated Agarose resin is then washed twice with 4 column volumes of Coupling/Wash Buffer separated by centrifugation at 1,000×g for 1 minute. For storage, the PDZ domain binding peptide ligand-NHS-Activated Agarose is washed twice with 4 column volumes of Storage Buffer (100 mM NaHPO₄, pH 7.2, 150 mM NaCl, 0.05% NαN₃), pelleted by centrifugation at 1,000×g for 1 minute, resuspended in 1 column volume of Storage Buffer, and stored at 4° C. Coupling of peptide to NHS-Agarose and the ligand density of immobilized peptide were monitored by the 660 nm Protein Assay (Cat. No. 22660, Pierce, Rockford Ill.) (The NHS leaving group interferes with BCA assays and absorbance assays at 280 nm). Ligand densities are expected to be on the order of 20 to 26 nmol peptide/μl resin (20-26 mM), which represents coupling efficiencies of 74 to 96%, respectively. No loss of binding capacity is expected after 5 cycles of elution, denaturation and renaturation or after storage for 6 months at 4° C.

After preparing the PDZ domain binding peptide ligand-NHS-Activated Agarose matrix, a mixture of proteins containing the POI is added to 100-200 ul of PDZ domain binding peptide ligand-NHS-Activated Agarose pre-equilibrated with Purification Buffer, and the suspension is be agitated at 4° C. for 1 h on an end-over-end mixer to allow the protein to bind to the affinity resin. The mixtures are then subjected to centrifugation at 1000×g for 2 min, and the supernatant removed by pipetting. The supernatant will contain the POI since it lacks PDZ domain binding peptide affinity, while the free PDZ domains in the mixture are now bound to the matrix. The affinity resin with bound protein could then be mixed with an additional 2 column volumes of Purification Buffer, transferred to a BioSpin chromatography column (Cat. No. 732-6008, BioRad, Hercules, Calif.), capped, and allowed to settle for 15 min and drained to collect any additional POI that was loosely associated with the column. The enriched POI can then be used as desired.

### Example 24 (Prophetic)

### Detection of a POI Fusion Protein Containing a C-Terminal PSD-95 PDZ2 Domain (e.g. Type C PDZ Affinity Tags) Present in a Mixture Using a C-Terminal PDZ Domain Binding Peptide Coupled to a Detection Agent

For the experiment, fusion proteins containing a POI in frame with C-terminal PSD-95 PDZ2 PDZ affinity chromatography tags (e.g. Type C PDZ affinity tags) could be expressed in BL21(DE3) E. coli cells. Single colonies of BL21(DE3) cells harboring pJExpress414 plasmids containing genes of interest could be grown overnight at 37° C. in 5 ml of lysogeny broth (LB) (Cat. No. L9110, Teknova, Hollister, Calif.) supplemented with 100 ug/ml carbenicillin. Overnight cultures could be diluted 1:500 into Overnight Express Instant Terrific Broth (TB) Media (Cat. No. 71491, EMD Millipore, Billerica, Mass.), grown for 16 h at 37° C. Cells are then pelleted by centrifugation at 10,000×g, and resuspended in PBS and incubated with 400 ug/ml GAGSIESDV PDZ domain binding C-terminal peptide amine (N-terminally) coupled to an Alexa Fluor 680 fluorophore (as known in the art), for 60 minutes at 4° C. to allow the Alexa Fluor 680 fluorophore tagged C-terminal peptide to permeate the cell and bind the Type C PDZ affinity Tag attached to the POI. Cells are then pelleted by centrifugation at 10,000×g, rinsed with PBS buffer and centrifuged again at 10,000× g (Repeated 2×). Cells are then resuspended in PBS and imaged on a fluorescence microscope (684 nm Excitation, 707 nm Emission, Extinction coefficient 183,000 cm⁻¹ M⁻¹) to determine protein expression levels and cell localization.

### Example 25 (Prophetic)

### Detection of a POI Fusion Protein Containing a C-Terminal PDZ Domain Binding Peptide Tag (e.g. Type a PDZ Affinity Tags) Present in a Mixture Using a Protein Array Consisting of a PDZ Domain Coupled to an Array Surface and a Monoclonal Antibody Coupled to a Fluorophore

For the experiment, the array surface is activated with tosylchloride as described in (141). Following activation by tosylchloride, purified PDZ domain (e.g. PSD-95 PDZ2), purified as described in Example 20 is incubated with the activated array surface in order to form a covalent amide bond between the PDZ domain and Array surface.

Fusion proteins containing a POI in frame with C-terminal NR2B Tail PDZ binding peptide affinity chromatography tags (e.g. Type A PDZ affinity tags) are then expressed in BL21(DE3) E. coli cells. Single colonies of BL21(DE3) cells harboring pJExpress414 plasmids containing genes of interest are be grown overnight at 37° C. in 5 ml of lysogeny broth (LB) (Cat. No. L9110, Teknova, Hollister, Calif.) supplemented with 100 ug/ml carbenicillin. Overnight cultures are be diluted 1:500 into Overnight Express Instant Terrific Broth (TB) Media (Cat. No. 71491, EMD Millipore, Billerica, Mass.), grown for 16 h at 37° C., pelleted by centrifugation, and flash frozen in liquid nitrogen. Bacterial cell pellets (0.4-2.5 grams) containing fusions of the POI to a C-terminal PSD-95 PDZ2 PDZ affinity chromatography tags (e.g. Type A PDZ affinity tags) could be resuspended in 5 ml/g cell-paste Purification Buffer (50 mM HEPES, pH 7.5, 150 mM NaCl, 5 mM TCEP, 0.5 mM EDTA, 10 mM MgCl2, 2 mM PMSF, Complete Protease Inhibitor) supplemented with 25 U/ml Benzonase and 10 U/ml ReadyLyse. The resuspended cells could be sonicated (Branson, Danbury Conn.) 2× for 90 s/pass (15% power, 1.0 s on, 1.5 s off) before clarification by centrifugation at 16,000×g for 30 min at 4° C. Clarified lysate (1.8-14 ml) containing the POI fusion could be spotted onto the array surface containing covalently bound PDZ domains (e.g. PSD-95 PDZ2) and left to dry for 1 hr at 4° C. for to allow the protein to bind to the affinity resin. The array would then be subject to washing to remove weakly bound contaminants. Following washing, monoclonal antibodies (coupled to Alexa Fluor 680 fluorophores) specific to the POIs are then incubated with the array before washing and imaging on a fluorescence microscope (684 nm Excitation, 707 nm Emission, Extinction coefficient 183,000 cm⁻¹ M⁻¹) to determine protein expression levels.

The examples set forth above are provided to give those of ordinary skill in the art a complete disclosure and description of how to make and use the embodiments of the materials, compositions, systems and methods of the disclosure, and are not intended to limit the scope of what the inventors regard as their disclosure.

All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

In particular, it will be understood that various modifications may be made without departing from the spirit and scope of the present disclosure. Accordingly, other embodiments are within the scope of the following claims

### REFERENCES

1. Doyle, D. A., Lee, A., Lewis, J., Kim, E., Sheng, M., and MacKinnon, R. (1996) Crystal structures of a complexed and peptide-free membrane protein-binding domain: molecular basis of peptide recognition by PDZ. Cell 85, 1067-1076
2. Hillier, B. J., Christopherson, K. S., Prehoda, K. E., Bredt, D. S., and Lim, W. A. (1999) Unexpected modes of PDZ domain scaffolding revealed by structure of nNOS-syntrophin complex. Science (New York, N.Y.) 284, 812-815
3. Walkup, W. G. t., and Kennedy, M. B. (2014) PDZ affinity chromatography: A general method for affinity purification of proteins based on PDZ domains and their ligands. Protein expression and purification 98, 46-62
4. Klock, H. E., Koesema, E. J., Knuth, M. W., and Lesley, S. A. (2008) Combining the polymerase incomplete primer extension method for cloning and mutagenesis with microscreening to accelerate structural genomics efforts. Proteins 71, 982-994
5. Klock, H. E., and Lesley, S. A. (2009) The Polymerase Incomplete Primer Extension (PIPE) method applied to high-throughput cloning and site-directed mutagenesis. Methods in molecular biology (Clifton, N.J.) 498, 91-103
6. Gibson, D. G. (2011) Enzymatic assembly of overlapping DNA fragments. Methods in enzymology 498, 349-361
7. Gibson, D. G., Young, L., Chuang, R. Y., Venter, J. C., Hutchison, C. A., 3rd, and Smith, H. O. (2009) Enzymatic assembly of DNA molecules up to several hundred kilobases. Nature methods 6, 343-345
8. Kodumal, S. J., Patel, K. G., Reid, R., Menzella, H. G., Welch, M., and Santi, D. V. (2004) Total synthesis of long DNA sequences: synthesis of a contiguous 32-kb polyketide synthase gene cluster. Proceedings of the National Academy of Sciences of the United States of America 101, 15573-15578
9. Kodumal, S. J., and Santi, D. V. (2004) DNA ligation by selection. BioTechniques 37, 34, 36, 38 passim
10. Kim, C. H., Axup, J. Y., and Schultz, P. G. (2013) Protein conjugation with genetically encoded unnatural amino acids. Current opinion in chemical biology 17, 412-419
11. Liu, C. C., and Schultz, P. G. (2010) Adding new chemistries to the genetic code. Annual review of biochemistry 79, 413-444
12. Young, T. S., and Schultz, P. G. (2010) Beyond the canonical 20 amino acids: expanding the genetic lexicon. The Journal of biological chemistry 285, 11039-11044
13. Kolb, H. C., Finn, M. G., and Sharpless, K. B. (2001) Click Chemistry: Diverse Chemical Function from a Few Good Reactions. Angewandte Chemie 40, 2004-2021
14. Blackman, M. L., Royzen, M., and Fox, J. M. (2008) Tetrazine ligation: fast bioconjugation based on inverse-electron-demand Diels-Alder reactivity. Journal of the American Chemical Society 130, 13518-13519
15. Devaraj, N. K., Weissleder, R., and Hilderbrand, S. A. (2008) Tetrazine-based cycloadditions: application to pretargeted live cell imaging. Bioconjugate chemistry 19, 2297-2299
16. Decreau, R. A., Collman, J. P., Yang, Y., Yan, Y., and Devaraj, N. K. (2007) Syntheses of hemoprotein models that can be covalently attached onto electrode surfaces by click chemistry. The Journal of organic chemistry 72, 2794-2802
17. Devaraj, N. K., Thurber, G. M., Keliher, E. J., Marinelli, B., and Weissleder, R. (2012) Reactive polymer enables efficient in vivo bioorthogonal chemistry. Proceedings of the National Academy of Sciences of the United States of America 109, 4762-4767
18. Han, H. S., Devaraj, N. K., Lee, J., Hilderbrand, S. A., Weissleder, R., and Bawendi, M. G. (2010) Development of a bioorthogonal and highly efficient conjugation method for quantum dots using tetrazine-norbornene cycloaddition. Journal of the American Chemical Society 132, 7838-7839
19. Stockmann, H., Neves, A. A., Stairs, S., Brindle, K. M., and Leeper, F. J. (2011) Exploring isonitrile-based click chemistry for ligation with biomolecules. Organic & biomolecular chemistry 9, 7303-7305
20. Warden-Rothman, R., Caturegli, I., Popik, V., and Tsourkas, A. (2013) Sortase-tag expressed protein ligation: combining protein purification and site-specific bioconjugation into a single step. Analytical chemistry 85, 11090-11097
21. Guimaraes, C. P., Witte, M. D., Theile, C. S., Bozkurt, G., Kundrat, L., Blom, A. E., and Ploegh, H. L. (2013) Site-specific C-terminal and internal loop labeling of proteins using sortase-mediated reactions. Nature protocols 8, 1787-1799
22. Theile, C. S., Witte, M. D., Blom, A. E., Kundrat, L., Ploegh, H. L., and Guimaraes, C. P. (2013) Site-specific N-terminal labeling of proteins using sortase-mediated reactions. Nature protocols 8, 1800-1807
23. Witte, M. D., Theile, C. S., Wu, T., Guimaraes, C. P., Blom, A. E., and Ploegh, H. L. (2013) Production of unnaturally linked chimeric proteins using a combination of sortase-catalyzed transpeptidation and click chemistry. Nature protocols 8, 1808-1819
24. Seedorff, S., Appelt, C., Beyermann, M., and Schmieder, P. (2010) Design, synthesis, structure and binding properties of PDZ binding, cyclic beta-finger peptides. Biochemical and biophysical research communications 395, 535-539
25. Gee, S. H., Sekely, S. A., Lombardo, C., Kurakin, A., Froehner, S. C., and Kay, B. K. (1998) Cyclic peptides as non-carboxyl-terminal ligands of syntrophin PDZ domains. The Journal of biological chemistry 273, 21980-21987
26. Grandy, D., Shan, J., Zhang, X., Rao, S., Akunuru, S., Li, H., Zhang, Y., Alpatov, I., Zhang, X. A., Lang, R. A., Shi, D. L., and Zheng, J. J. (2009) Discovery and characterization of a small molecule inhibitor of the PDZ domain of dishevelled. The Journal of biological chemistry 284, 16256-16263
27. Ivarsson, Y., Wawrzyniak, A. M., Wuytens, G., Kosloff, M., Vermeiren, E., Raport, M., and Zimmermann, P. (2011) Cooperative phosphoinositide and peptide binding by PSD-95/discs large/ZO-1 (PDZ) domain of polychaetoid, Drosophila zonulin. The Journal of biological chemistry 286, 44669-44678
28. Morais Cabral, J. H., Petosa, C., Sutcliffe, M. J., Raza, S., Byron, 0., Poy, F., Marfatia, S. M., Chishti, A. H., and Liddington, R. C. (1996) Crystal structure of a PDZ domain. Nature 382, 649-652
29. Kennedy, M. B. (1995) Origin of PDZ (DHR, GLGF) domains. Trends in biochemical sciences 20, 350
30. Cho, K. O., Hunt, C. A., and Kennedy, M. B. (1992) The rat brain postsynaptic density fraction contains a homolog of the Drosophila discs-large tumor suppressor protein. Neuron 9, 929-942
31. Kornau, H. C., Schenker, L. T., Kennedy, M. B., and Seeburg, P. H. (1995) Domain interaction between NMDA receptor subunits and the postsynaptic density protein PSD-95. Science (New York, N.Y.) 269, 1737-1740
32. Kornau, H. C., Seeburg, P. H., and Kennedy, M. B. (1997) Interaction of ion channels and receptors with PDZ domain proteins. Current opinion in neurobiology 7, 368-373
33. Hung, A. Y., and Sheng, M. (2002) PDZ domains: structural modules for protein complex assembly. The Journal of biological chemistry 277, 5699-5702
34. Brenman, J. E., Chao, D. S., Gee, S. H., McGee, A. W., Craven, S. E., Santillano, D. R., Wu, Z., Huang, F., Xia, H., Peters, M. F., Froehner, S. C., and Bredt, D. S. (1996) Interaction of nitric oxide synthase with the postsynaptic density protein PSD-95 and alpha1-syntrophin mediated by PDZ domains. Cell 84, 757-767
35. Songyang, Z., Fanning, A. S., Fu, C., Xu, J., Marfatia, S. M., Chishti, A. H., Crompton, A., Chan, A. C., Anderson, J. M., and Cantley, L. C. (1997) Recognition of unique carboxyl-terminal motifs by distinct PDZ domains. Science (New York, N.Y) 275, 73-77
36. Webb, B., and Sali, A. (2014) Protein structure modeling with MODELLER. Methods in molecular biology (Clifton, N.J.) 1137, 1-15
37. Baker, D. (2014) Protein folding, structure prediction and design. Biochemical Society transactions 42, 225-229
38. Kim, D. E., Chivian, D., and Baker, D. (2004) Protein structure prediction and analysis using the Robetta server. Nucleic acids research 32, W526-531
39. Roy, A., Kucukural, A., and Zhang, Y. (2010) I-TASSER: a unified platform for automated protein structure and function prediction. Nature protocols 5, 725-738
40. Zhang, Y. (2008) I-TASSER server for protein 3D structure prediction. BMC bioinformatics 9, 40
41. Bolon, D. N., and Mayo, S. L. (2001) Enzyme-like proteins by computational design. Proceedings of the National Academy of Sciences of the United States of America 98, 14274-14279
42. Dahiyat, B. I., and Mayo, S. L. (1997) De novo protein design: fully automated sequence selection. Science (New York, N.Y.) 278, 82-87
43. Higgins, D. G., and Sharp, P. M. (1988) CLUSTAL: a package for performing multiple sequence alignment on a microcomputer. Gene 73, 237-244
44. Edgar, R. C. (2004) MUSCLE: multiple sequence alignment with high accuracy and high throughput. Nucleic acids research 32, 1792-1797
45. Edgar, R. C. (2004) MUSCLE: a multiple sequence alignment method with reduced time and space complexity. BMC bioinformatics 5, 113
46. Passafaro, M., Sala, C., Niethammer, M., and Sheng, M. (1999) Microtubule binding by CRIPT and its potential role in the synaptic clustering of PSD-95. Nature neuroscience 2, 1063-1069
47. Niethammer, M., Valtschanoff, J. G., Kapoor, T. M., Allison, D. W., Weinberg, R. J., Craig, A. M., and Sheng, M. (1998) CRIPT, a novel postsynaptic protein that binds to the third PDZ domain of PSD-95/SAP90. Neuron 20, 693-707
48. Harris, B. Z., Hillier, B. J., and Lim, W. A. (2001) Energetic determinants of internal motif recognition by PDZ domains. Biochemistry 40, 5921-5930
49. Lim, I. A., Hall, D. D., and Hell, J. W. (2002) Selectivity and promiscuity of the first and second PDZ domains of PSD-95 and synapse-associated protein 102. The Journal of biological chemistry 277, 21697-21711
50. Marfatia, S. M., Morais Cabral, J. H., Lin, L., Hough, C., Bryant, P. J., Stolz, L., and Chishti, A. H. (1996) Modular organization of the PDZ domains in the human discs-large protein suggests a mechanism for coupling PDZ domain-binding proteins to ATP and the membrane cytoskeleton. The Journal of cell biology 135, 753-766
51. Muller, B. M., Kistner, U., Kindler, S., Chung, W. J., Kuhlendahl, S., Fenster, S. D., Lau, L. F., Veh, R. W., Huganir, R. L., Gundelfinger, E. D., and Garner, C. C. (1996) SAP102, a novel postsynaptic protein that interacts with NMDA receptor complexes in vivo. Neuron 17, 255-265
52. Lim, I. A., Merrill, M. A., Chen, Y., and Hell, J. W. (2003) Disruption of the NMDA receptor-PSD-95 interaction in hippocampal neurons with no obvious physiological short-term effect. Neuropharmacology 45, 738-754
53. Adams, M. E., Butler, M. H., Dwyer, T. M., Peters, M. F., Murnane, A. A., and Froehner, S. C. (1993) Two forms of mouse syntrophin, a 58 kd dystrophin-associated protein, differ in primary structure and tissue distribution. Neuron 11, 531-540
54. Fabrini, R., De Luca, A., Stella, L., Mei, G., Orioni, B., Ciccone, S., Federici, G., Lo Bello, M., and Ricci, G. (2009) Monomer-dimer equilibrium in glutathione transferases: a critical re-examination. Biochemistry 48, 10473-10482
55. Ivanetich, K. M., and Goold, R. D. (1989) A rapid equilibrium random sequential bi-bi mechanism for human placental glutathione S-transferase. Biochimica et biophysica acta 998, 7-13
56. Ivanetich, K. M., Goold, R. D., and Sikakana, C. N. (1990) Explanation of the non-hyperbolic kinetics of the glutathione S-transferases by the simplest steady-state random sequential Bi Bi mechanism. Biochemical pharmacology 39, 1999-2004
57. Ivanetich, K. M., Thumser, A. E., and Harrison, G. G. (1988) Halothane: inhibition and activation of rat hepatic glutathione S-transferases. Biochemical pharmacology 37, 1903-1908
58. Schramm, V. L., McCluskey, R., Emig, F. A., and Litwack, G. (1984) Kinetic studies and active site-binding properties of glutathione S-transferase using spin-labeled glutathione, a product analogue. The Journal of biological chemistry 259, 714-722
59. Morise, H., Shimomura, 0., Johnson, F. H., and Winant, J. (1974) Intermolecular energy transfer in the bioluminescent system of Aequorea. Biochemistry 13, 2656-2662
60. Prendergast, F. G., and Mann, K. G. (1978) Chemical and physical properties of aequorin and the green fluorescent protein isolated from Aequorea forskalea. Biochemistry 17, 3448-3453
61. Tsien, R. Y. (1998) The green fluorescent protein. Annual review of biochemistry 67, 509-544
62. Hanson, G. T., McAnaney, T. B., Park, E. S., Rendell, M. E., Yarbrough, D. K., Chu, S., Xi, L., Boxer, S. G., Montrose, M. H., and Remington, S. J. (2002) Green fluorescent protein variants as ratiometric dual emission pH sensors. 1. Structural characterization and preliminary application. Biochemistry 41, 15477-15488
63. McAnaney, T. B., Park, E. S., Hanson, G. T., Remington, S. J., and Boxer, S. G. (2002) Green fluorescent protein variants as ratiometric dual emission pH sensors. 2. Excited-state dynamics. Biochemistry 41, 15489-15494
64. Miesenbock, G., De Angelis, D. A., and Rothman, J. E. (1998) Visualizing secretion and synaptic transmission with pH-sensitive green fluorescent proteins. Nature 394, 192-195
65. Mank, M., Santos, A. F., Direnberger, S., Mrsic-Flogel, T. D., Hofer, S. B., Stein, V., Hendel, T., Reiff, D. F., Levelt, C., Borst, A., Bonhoeffer, T., Hubener, M., and Griesbeck, O. (2008) A genetically encoded calcium indicator for chronic in vivo two-photon imaging. Nature methods 5, 805-811
66. Ostergaard, H., Henriksen, A., Hansen, F. G., and Winther, J. R. (2001) Shedding light on disulfide bond formation: engineering a redox switch in green fluorescent protein. The EMBO journal 20, 5853-5862
67. Hanson, G. T., Aggeler, R., Oglesbee, D., Cannon, M., Capaldi, R. A., Tsien, R. Y., and Remington, S. J. (2004) Investigating mitochondrial redox potential with redox-sensitive green fluorescent protein indicators. The Journal of biological chemistry 279, 13044-13053
68. Baker, B. J., Mutoh, H., Dimitrov, D., Akemann, W., Perron, A., Iwamoto, Y., Jin, L., Cohen, L. B., Isacoff, E. Y., Pieribone, V. A., Hughes, T., and Knopfel, T. (2008) Genetically encoded fluorescent sensors of membrane potential. Brain cell biology 36, 53-67
69. Wong, F. H., Banks, D. S., Abu-Arish, A., and Fradin, C. (2007) A molecular thermometer based on fluorescent protein blinking. Journal of the American Chemical Society 129, 10302-10303
70. Follenius-Wund, A., Bourotte, M., Schmitt, M., Iyice, F., Lami, H., Bourguignon, J. J., Haiech, J., and Pigault, C. (2003) Fluorescent derivatives of the GFP chromophore give a new insight into the GFP fluorescence process. Biophysical journal 85, 1839-1850
71. Waldo, G. S., Standish, B. M., Berendzen, J., and Terwilliger, T. C. (1999) Rapid protein-folding assay using green fluorescent protein. Nature biotechnology 17, 691-695
72. Kalnins, A., Otto, K., Ruther, U., and Muller-Hill, B. (1983) Sequence of the lacZ gene of Escherichia coli. The EMBO journal 2, 593-597
73. Fowler, A. V., and Zabin, I. (1978) Amino acid sequence of beta-galactosidase. XI. Peptide ordering procedures and the complete sequence. The Journal of biological chemistry 253, 5521-5525
74. Huber, R. E., Gupta, M. N., and Khare, S. K. (1994) The active site and mechanism of the beta-galactosidase from Escherichia coli. The International journal of biochemistry 26, 309-318
75. Huber, R. E., Kurz, G., and Wallenfels, K. (1976) A quantitation of the factors which affect the hydrolase and transgalactosylase activities of beta-galactosidase (E. coli) on lactose. Biochemistry 15, 1994-2001
76. Guarente, L. (1983) Yeast promoters and lacZ fusions designed to study expression of cloned genes in yeast. Methods in enzymology 101, 181-191
77. Guarente, L., and Ptashne, M. (1981) Fusion of Escherichia coli lacZ to the cytochrome c gene of Saccharomyces cerevisiae. Proceedings of the National Academy of Sciences of the United States of America 78, 2199-2203
78. Fire, A., Harrison, S. W., and Dixon, D. (1990) A modular set of lacZ fusion vectors for studying gene expression in Caenorhabditis elegans. Gene 93, 189-198
79. Lis, J. T., Simon, J. A., and Sutton, C. A. (1983) New heat shock puffs and beta-galactosidase activity resulting from transformation of Drosophila with an hsp70-lacZ hybrid gene. Cell 35, 403-410
80. Hall, C. V., Jacob, P. E., Ringold, G. M., and Lee, F. (1983) Expression and regulation of Escherichia coli lacZ gene fusions in mammalian cells. Journal of molecular and applied genetics 2, 101-109
81. Miller, J. H. (1992) A Short Course in Bacterial Genetics, Cold Spring Harbor Laboratory Press, Cold Spring Harbor, N.Y.
82. Davies, J., and Jacob, F. (1968) Genetic mapping of the regulator and operator genes of the lac operon. Journal of molecular biology 36, 413-417
83. Ullmann, A., Jacob, F., and Monod, J. (1967) Characterization by in vitro complementation of a peptide corresponding to an operator-proximal segment of the beta-galactosidase structural gene of Escherichia coli. Journal of molecular biology 24, 339-343
84. Goldberg, M. E., and Edelstein, S. J. (1969) Sedimentation equilibrium of paucidisperse systems. Subunit structure of complemented beta-galactosidase. Journal of molecular biology 46, 431-440
85. Muller-Hill, B., and Kania, J. (1974) Lac repressor can be fused to beta-galactosidase. Nature 249, 561-563
86. Fowler, A. V. (1972) High-level production of-galactosidase by Escherichia coli merodiploids. Journal of bacteriology 112, 856-860
87. Shaw, W. V. (1967) The enzymatic acetylation of chloramphenicol by extracts of R factor-resistant Escherichia coli. The Journal of biological chemistry 242, 687-693
88. Suzuki, Y., and Okamoto, S. (1967) The enzymatic acetylation of chloramphenicol by the multiple drug-resistant Escherichia coli carrying R factor. The Journal of biological chemistry 242, 4722-4730
89. Gorman, C. M., Moffat, L. F., and Howard, B. H. (1982) Recombinant genomes which express chloramphenicol acetyltransferase in mammalian cells. Molecular and cellular biology 2, 1044-1051
90. Kain, S. R., and Ganguly, S. (2001) Overview of genetic reporter systems. Current protocols in molecular biology/edited by Frederick M. Ausubel . . . [et al.] Chapter 9, Unit9 6
91. Angehagen, M., Ronnback, L., Hansson, E., and Ben-Menachem, E. (2005) Topiramate reduces AMPA-induced Ca(2+) transients and inhibits GluR1 subunit phosphorylation in astrocytes from primary cultures. Journal of neurochemistry 94, 1124-1130
92. Orchard, S., Ammari, M., Aranda, B., Breuza, L., Briganti, L., Broackes-Carter, F., Campbell, N. H., Chavali, G., Chen, C., del-Toro, N., Duesbury, M., Dumousseau, M., Galeota, E., Hinz, U., Iannuccelli, M., Jagannathan, S., Jimenez, R., Khadake, J., Lagreid, A., Licata, L., Lovering, R. C., Meldal, B., Melidoni, A. N., Milagros, M., Peluso, D., Perfetto, L., Porras, P., Raghunath, A., Ricard-Blum, S., Roechert, B., Stutz, A., Tognolli, M., van Roey, K., Cesareni, G., and Hermjakob, H. (2014) The MlntAct project—IntAct as a common curation platform for 11 molecular interaction databases. Nucleic acids research 42, D358-363
93. Dinkel, H., Van Roey, K., Michael, S., Davey, N. E., Weatheritt, R. J., Born, D., Speck, T., Kruger, D., Grebnev, G., Kuban, M., Strumillo, M., Uyar, B., Budd, A., Altenberg, B., Seiler, M., Chemes, L. B., Glavina, J., Sanchez, I. E., Diella, F., and Gibson, T. J. (2014) The eukaryotic linear motif resource ELM: 10 years and counting. Nucleic acids research 42, D259-266
94. UniProt, C. (2014) Activities at the Universal Protein Resource (UniProt). Nucleic acids research 42, D191-198
95. Rose, P. W., Bi, C., Bluhm, W. F., Christie, C. H., Dimitropoulos, D., Dutta, S., Green, R. K., Goodsell, D. S., Prlic, A., Quesada, M., Quinn, G. B., Ramos, A. G., Westbrook, J. D., Young, J., Zardecki, C., Berman, H. M., and Bourne, P. E. (2013) The RCSB Protein Data Bank: new resources for research and education. Nucleic acids research 41, D475-482
96. Berman, H. M., Westbrook, J., Feng, Z., Gilliland, G., Bhat, T. N., Weissig, H., Shindyalov, I. N., and Bourne, P. E. (2000) The Protein Data Bank. Nucleic acids research 28, 235-242
97. Axen, R., and Ernback, S. (1971) Chemical fixation of enzymes to cyanogen halide activated polysaccharide carriers. European journal of biochemistry/FEBS 18, 351-360
98. Wilchek, M., Miron, T., and Kohn, J. (1984) Affinity chromatography. Methods in enzymology 104, 3-55
99. Miron, T., and Wilchek, M. (1985) Activation of trisacryl gels with chloroformates and their use for affinity chromatography and protein immobilization. Applied Biochemistry and Biotechnology 11, 445-456
100. Hearn, M. T. (1987) 1,1′-Carbonyldiimidazole-mediated immobilization of enzymes and affinity ligands. Methods in enzymology 135, 102-117
101. Nilsson, K., and Mosbach, K. (1987) Tresyl chloride-activated supports for enzyme immobilization. Methods in enzymology 135, 65-78
102. Sundberg, L., and Porath, J. (1974) Preparation of adsorbents for biospecific affinity chromatography. Attachment of group-containing ligands to insoluble polymers by means of bifunctional oxiranes. Journal of chromatography 90, 87-98
103. Porath, J., and Fornstedt, N. (1970) Group fractionation of plasma proteins on dipolar ion exchangers. Journal of chromatography 51, 479-489
104. Chi, C. N., Engstrom, A., Gianni, S., Larsson, M., and Jemth, P. (2006) Two conserved residues govern the salt and pH dependencies of the binding reaction of a PDZ domain. The Journal of biological chemistry 281, 36811-36818
105. Thompson, N. E., Foley, K. M., Stalder, E. S., and Burgess, R. R. (2009) Identification, production, and use of polyol-responsive monoclonal antibodies for immunoaffinity chromatography. Methods in enzymology 463, 475-494
106. Kennedy, M. B. (2000) Signal-processing machines at the postsynaptic density. Science (New York, N.Y.) 290, 750-754
107. Kennedy, M. B., Beale, H. C., Carlisle, H. J., and Washburn, L. R. (2005) Integration of biochemical signalling in spines. Nature reviews. Neuroscience 6, 423-434
108. Hunt, C. A., Schenker, L. J., and Kennedy, M. B. (1996) PSD-95 is associated with the postsynaptic density and not with the presynaptic membrane at forebrain synapses. The Journal of neuroscience: the official journal of the Society for Neuroscience 16, 1380-1388
109. Sheng, M., and Kim, E. (2011) The postsynaptic organization of synapses. Cold Spring Harbor perspectives in biology 3
110. Mayer, M. L., and Armstrong, N. (2004) Structure and function of glutamate receptor ion channels. Annual review of physiology 66, 161-181
111. Nakanishi, S. (1992) Molecular diversity of glutamate receptors and implications for brain function. Science (New York, N.Y.) 258, 597-603
112. Seeburg, P. H. (1993) The TINS/TiPS Lecture. The molecular biology of mammalian glutamate receptor channels. Trends in neurosciences 16, 359-365
113. Tsien, J. Z., Huerta, P. T., and Tonegawa, S. (1996) The essential role of hippocampal CA1 NMDA receptor-dependent synaptic plasticity in spatial memory. Cell 87, 1327-1338
114. Mayer, M. L. (2006) Glutamate receptors at atomic resolution. Nature 440, 456-462
115. Kohr, G., Jensen, V., Koester, H. J., Mihaljevic, A. L., Utvik, J. K., Kvello, A., Ottersen, O. P., Seeburg, P. H., Sprengel, R., and Hvalby, O. (2003) Intracellular domains of NMDA receptor subtypes are determinants for long-term potentiation induction. The Journal of neuroscience: the official journal of the Society for Neuroscience 23, 10791-10799
116. Chen, H. J., Rojas-Soto, M., Oguni, A., and Kennedy, M. B. (1998) A synaptic Ras-GTPase activating protein (p135 SynGAP) inhibited by CaM kinase II. Neuron 20, 895-904
117. Kim, J. H., Lee, H. K., Takamiya, K., and Huganir, R. L. (2003) The role of synaptic
GTPase-activating protein in neuronal development and synaptic plasticity. The Journal of neuroscience: the official journal of the Society for Neuroscience 23, 1119-1124
118. Vazquez, L. E., Chen, H. J., Sokolova, I., Knuesel, I., and Kennedy, M. B. (2004) SynGAP regulates spine formation. The Journal of neuroscience: the official journal of the Society for Neuroscience 24, 8862-8872
119. Komiyama, N. H., Watabe, A. M., Carlisle, H. J., Porter, K., Charlesworth, P., Monti, J., Strathdee, D. J., O'Carroll, C. M., Martin, S. J., Morris, R. G., O'Dell, T. J., and Grant, S. G. (2002) SynGAP regulates ERK/MAPK signaling, synaptic plasticity, and learning in the complex with postsynaptic density 95 and NMDA receptor. The Journal of neuroscience: the official journal of the Society for Neuroscience 22, 9721-9732
120. McMahon, A. C., Barnett, M. W., O'Leary, T. S., Stoney, P. N., Collins, M. 0., Papadia, S., Choudhary, J. S., Komiyama, N. H., Grant, S. G., Hardingham, G. E., Wyllie, D. J., and Kind, P. C. (2012) SynGAP isoforms exert opposing effects on synaptic strength. Nature communications 3, 900
121. Hamdan, F. F., Gauthier, J., Spiegelman, D., Noreau, A., Yang, Y., Pellerin, S., Dobrzeniecka, S., Cote, M., Perreau-Linck, E., Carmant, L., D'Anjou, G., Fombonne, E., Addington, A. M., Rapoport, J. L., Delisi, L. E., Krebs, M. O., Mouaffak, F., Joober, R., Mottron, L., Drapeau, P., Marineau, C., Lafreniere, R. G., Lacaille, J. C., Rouleau, G. A., Michaud, J. L., and Synapse to Disease, G. (2009) Mutations in SYNGAP1 in autosomal nonsyndromic mental retardation. The New England journal of medicine 360, 599-605
122. Oh, J. S., Manzerra, P., and Kennedy, M. B. (2004) Regulation of the neuron-specific Ras GTPase-activating protein, synGAP, by Ca2+/calmodulin-dependent protein kinase II. The Journal of biological chemistry 279, 17980-17988
123. Pena, V., Hothorn, M., Eberth, A., Kaschau, N., Parret, A., Gremer, L., Bonneau, F., Ahmadian, M. R., and Scheffzek, K. (2008) The C2 domain of SynGAP is essential for stimulation of the Rap GTPase reaction. EMBO reports 9, 350-355
124. Firestein, B. L., Firestein, B. L., Brenman, J. E., Aoki, C., Sanchez-Perez, A. M., El-Husseini, A. E., and Bredt, D. S. (1999) Cypin: a cytosolic regulator of PSD-95 postsynaptic targeting. Neuron 24, 659-672
125. Charych, E. I., Akum, B. F., Goldberg, J. S., Jornsten, R. J., Rongo, C., Zheng, J. Q., and Firestein, B. L. (2006) Activity-independent regulation of dendrite patterning by postsynaptic density protein PSD-95. The Journal of neuroscience: the official journal of the Society for Neuroscience 26, 10164-10176
126. Akum, B. F., Chen, M., Gunderson, S. I., Riefler, G. M., Scerri-Hansen, M. M., and Firestein, B. L. (2004) Cypin regulates dendrite patterning in hippocampal neurons by promoting microtubule assembly. Nature neuroscience 7, 145-152
127. Brenman, J. E., Topinka, J. R., Cooper, E. C., McGee, A. W., Rosen, J., Milroy, T., Ralston, H. J., and Bredt, D. S. (1998) Localization of postsynaptic density-93 to dendritic microtubules and interaction with microtubule-associated protein 1A. The Journal of neuroscience: the official journal of the Society for Neuroscience 18, 8805-8813
128. Kimple, M. E., and Sondek, J. (2002) Affinity tag for protein purification and detection based on the disulfide-linked complex of InaD and NorpA. BioTechniques 33, 578, 580, 584-578 passim
129. Bredt, D. S., and Snyder, S. H. (1990) Isolation of nitric oxide synthetase, a calmodulin-requiring enzyme. Proceedings of the National Academy of Sciences of the United States of America 87, 682-685
130. Brenman, J. E., and Bredt, D. S. (1997) Synaptic signaling by nitric oxide. Current opinion in neurobiology 7, 374-378
131. Bredt, D. S., and Snyder, S. H. (1992) Nitric oxide, a novel neuronal messenger. Neuron 8, 3-11
132. Tochio, H., Mok, Y. K., Zhang, Q., Kan, H. M., Bredt, D. S., and Zhang, M. (2000)
Formation of nNOS/PSD-95 PDZ dimer requires a preformed beta-finger structure from the nNOS PDZ domain. Journal of molecular biology 303, 359-370
133. Tochio, H., Zhang, Q., Mandal, P., Li, M., and Zhang, M. (1999) Solution structure of the extended neuronal nitric oxide synthase PDZ domain complexed with an associated peptide. Nature structural biology 6, 417-421
134. Wang, L., Piserchio, A., and Mierke, D. F. (2005) Structural characterization of the intermolecular interactions of synapse-associated protein-97 with the NR2B subunit of N-methyl-D-aspartate receptors. The Journal of biological chemistry 280, 26992-26996
135. Wallenfels, K. (1962) Beta-Galactosidase (crystalline). Methods in enzymology 5, 212-219
136. Wallenfels, K., Lehmann, J., and Malhotra, 0. P. (1960) [Research on lactose-splitting enzymes. VII. The specificity of beta-galactosidase from E. coli ML 309]. Biochemische Zeitschrift 333, 209-225
137. Craven, G. R., Steers, E., Jr., and Anfinsen, C. B. (1965) Purification, Composition, and Molecular Weight of the Beta-Galactosidase of Escherichia Coli K12. The Journal of biological chemistry 240, 2468-2477
138. Wallenfels, K., and Malhotra, 0. P. (1961) Galactosidases. Advances in carbohydrate chemistry 16, 239-298
139. Shaw, W. V. (1975) Chloramphenicol acetyltransferase from chloramphenicol-resistant bacteria. Methods in enzymology 43, 737-755
140. Winshell, E., and Shaw, W. V. (1969) Kinetics of induction and purification of chloramphenicol acetyltransferase from chloramphenicol-resistant Staphylococcus aureus. Journal of bacteriology 98, 1248-1257
141. Nilsson, K., and Mosbach, K. (1980) p-Toluenesulfonyl chloride as an activating agent of agarose for the preparation of immobilized affinity ligands and proteins. European journal of biochemistry/FEBS 112, 397-402

1. A PDZ domain peptide of 70 to 130 amino acids, the PDZ domain peptide comprising a βA strand, a βB strand, a βC strand, a βD strand, a βE strand, a βF strand, an αA helix and an αB helix linked one to another by loop regions in a configuration wherein the βB strand, the βC strand and the αB helix form a ligand binding pocket ranging in size from approximately 400 Å³ to approximately 900 Å³and wherein a GLGF loop linking the βA strand and the βB strand provides a steric block at the end of the ligand binding groove, the PDZ domain peptide having sequence SEQ ID NO: 1.

2. A PDZ binding carboxy-terminal peptide of sequence SEQ ID NO: 2.

3. An affinity tag essentially consisting of the PDZ domain peptide of claim 1, or of a PDZ binding carboxy terminal peptide having sequence SEQ ID NO: 2,
wherein the PDZ domain peptide is in a configuration in which the PDZ domain peptide is presented for specific binding to one or more corresponding PDZ domain peptides and/or to one or more corresponding PDZ binding C-terminal peptides presented in one or more affinity ligands, and
wherein the PDZ binding carboxy terminal peptide is in a configuration in which the PDZ binding carboxy terminal peptide is presented for specific binding to a corresponding PDZ domain peptide presented in one or more affinity ligands.

4. An affinity ligand comprising the PDZ domain peptide of claim 1 or a PDZ binding carboxy terminal peptide having sequence SEQ ID NO: 2,
wherein the PDZ domain peptide is in a configuration in which the PDZ domain peptide is presented for specific binding to one or more corresponding PDZ domain peptides and/or to one or more corresponding PDZ binding C-terminal peptide tags presented in one or more affinity tags, and
wherein the PDZ binding carboxy terminal peptide is in a configuration in which the PDZ binding carboxy terminal peptide is presented for specific binding to a corresponding PDZ domain peptide presented in one or more affinity tags.

5. An engineered protein comprising a target protein attaching at least one affinity tag according to claim 3,
wherein when the at least one affinity tag comprises a PDZ domain peptide, the PDZ domain peptide is comprised in the engineered protein in a configuration in which the PDZ domain peptide is presented for specific binding to one or more of corresponding PDZ domain peptides and/or to a corresponding PDZ binding carboxy terminal peptides presented in one or more affinity ligands; and
wherein when the at least one affinity tag comprises a PDZ binding carboxy terminal peptide, the PDZ binding carboxy terminal peptide is comprised in the engineered protein in a configuration in which the PDZ binding carboxy terminal peptide is presented for specific binding to a corresponding PDZ domain peptide comprised in one or more affinity ligands.

6. An engineered label comprising a label attaching at least one affinity ligand according to claim 4,
wherein when the affinity ligand is a PDZ domain peptide ligand, the PDZ domain peptide ligand is presented for specific binding to one or more of corresponding PDZ domain peptide and/or to a corresponding PDZ binding carboxy terminal peptide comprised in one or more affinity tags; and
wherein, when the at least one affinity ligand is comprises a PDZ binding carboxy terminal peptide, the PDZ binding carboxy terminal ligand is presented for specific binding to a corresponding PDZ domain peptide comprised in one or more affinity tags.

7. A chromatography stationary phase comprising a solid support attaching at least one affinity ligand according to claim 4,
wherein when the at least one affinity ligand comprises a PDZ domain peptide, the solid support attaches one or more PDZ domain peptides in a configuration presenting the one or more PDZ domain peptides for specific binding to one or more of corresponding PDZ domain peptide and/or to a corresponding PDZ binding carboxy terminal peptide comprised in one or more affinity tags, and
wherein when the solid support attaches one or more PDZ binding carboxy terminal peptides, the solid support attaches the one or more PDZ binding carboxy terminal peptides in a configuration wherein the one or more PDZ binding carboxy-terminal peptides are presented for specific binding to a corresponding PDZ domain peptide comprised in one or more affinity tags.

8. A reagent for detaching a PDZ tag-PDZ ligand complex, wherein
the PDZ tag is an affinity tag essentially consisting of the PDZ domain peptide of claim 1, or of a PDZ binding carboxy terminal peptide having sequence SEQ ID NO: 2, and
the PDZ ligand is a corresponding affinity ligand comprising the PDZ domain peptide of claim 1 or a PDZ binding carboxy terminal peptide having sequence SEQ ID NO: 2,
the reagent being selected from the group consisting of a PDZ domain peptide and a PDZ binding carboxy terminal peptide,
wherein the PDZ tag specifically binds the PDZ ligand with a first binding affinity K_{D}, and the PDZ ligand or PDZ tag specifically binds the reagent with a second binding affinity K_{Dcomp}, the second binding affinity K_{Dcomp} lower, equal to, or higher than the first binding affinity K_{D} and allowing detachment of the PDZ ligand from the PDZ affinity tag following contacting of the PDZ affinity tag-PDZ ligand complex with the reagent at set elution conditions wherein ratio of eluted target with respect to total bound target is a function of Kd, KDcomp and L0, wherein L0 is the concentration of the PDZ ligand.

9. The reagent of claim 8 wherein the ratio of eluted target with respect to total bound target is a function of (KD comp*L0)/Kd.

10. The reagent of claim 9, wherein the ratio of eluted target with respect to total target is a maximized by minimizing (KD comp*L0)/Kd.

11. The reagent of claim 8, wherein the ratio of eluted target with respect to total target is further a function of the ratio between the volume of reagent added and the pore volume of a porous support.

12. An affinity chromatography system comprising two or more of:
one or more engineered proteins according to claim 5, each presenting an affinity tag
one or more one or more chromatography stationary phases according to claim 7, wherein the at least one affinity ligand is capable of specifically bind the affinity tag presented on each of the one or more engineered proteins; and/or
one or more reagents for detaching a PDZ tag-PDZ ligand complex according to claim 8,
wherein the PDZ tag specifically binds the PDZ ligand with a first binding affinity, and the PDZ ligand or PDZ Tag specifically binds the reagent with a second binding affinity, the second binding affinity lower, equal to, or higher than the first binding affinity and allowing detachment of the PDZ ligand from the PDZ affinity tag following contacting of the PDZ affinity tag-PDZ ligand complex with the reagent at set elution conditions,
for simultaneous, combined or sequential use in a method to separating a target protein from a mixture.

13. A method for separating a target protein from a biochemical mixture, the method comprising
providing the target protein in an engineered protein according to claim 5 by attaching a PDZ affinity tag presented for binding to a corresponding PDZ affinity ligand;
contacting the engineered protein with an affinity chromatography stationary phase comprising a solid support attaching the PDZ affinity ligand to allow specific binding between PDZ affinity tag and the PDZ affinity ligand; and
separating the engineered protein by detaching the PDZ affinity tag from the PDZ affinity ligand.

14. A protein array comprising a solid support surface attaching at least one affinity ligand selected from the group consisting of a PDZ domain peptide, and a PDZ domain binding carboxy terminal peptide,
wherein the solid support surface attaches one or more PDZ domain peptide ligand in a configuration presenting the one or more PDZ domain peptide ligand for specific binding to one or more of corresponding PDZ domain peptide tags and/or corresponding PDZ binding carboxy terminal peptide tags, and
wherein the solid support surface attaches one or more PDZ binding carboxy-terminal ligand in a configuration wherein the PDZ binding carboxy-terminal ligand is presented for specific binding to a corresponding PDZ domain peptide tag.

15. A method for detecting a target in a biochemical mixture, the method comprising
contacting the biochemical mixture with one or more engineered proteins of claim 5 capable of specific binding to the target to form an engineered protein-target complex presenting a PDZ domain protein tag or a PDZ binding carboxy terminal peptide tag for binding to a corresponding PDZ domain protein ligand and/or to a PDZ binding carboxy terminal peptide ligand;
contacting the engineered protein-target complex with an engineered label presenting the corresponding PDZ domain protein ligand and/or the corresponding PDZ binding carboxy terminal peptide ligand; and
detecting a signal from the engineered label following contacting the engineered protein-target complex with the engineered label.

16. A target detection system, the target detection system comprising two or more of;
one or more engineered proteins of claim 5 presenting a PDZ domain protein tag or a PDZ binding carboxy terminal peptide tag for binding to a corresponding PDZ domain protein ligand and/or to a PDZ binding carboxy terminal peptide ligand;
one or more engineered labels of claim presenting the corresponding PDZ domain protein ligand and/or the corresponding PDZ binding carboxy terminal peptide ligand; and
one or more one or more chromatography stationary phases presenting the corresponding PDZ domain protein ligand and/or the corresponding PDZ binding carboxy terminal peptide ligand
for simultaneous combined or sequential use to detect target in a biochemical mixture.
